(19) United States
(12) Patent Application Publication
(10) Pub. No.: US 2023/0057568 A1
(43) Pub. Date: Feb. 23, 2023

(54) COMPOUNDS FOR TREATING FAMILIAL DYSAUTONOMIA

(71) Applicant: PTC THERAPEUTICS, INC., South Plainfield, NJ (US)

(72) Inventors: Nanjing ZHANG, Princeton, NJ (US); Michael A. ARNOLD, Flemington, NJ (US); Amal DAKKA, Whitehouse Station, NJ (US); Gary Mitchell KARP, Princeton Junction, NJ (US); Tom Tuan LUONG, San Carlos, CA (US); Christie MORRILL, Green Brook, NJ (US); Jana NARASIMHAN, Scotch Plains, NJ (US); Nikolai A. NARYSHKIN, East Brunswick, NJ (US); Anthony TURPOFF, Hillsborough, NJ (US); Jiashi WANG, West New York, NJ (US); Xiaoyan ZHANG, Belle Mead, NJ (US)

(73) Assignee: PTC THERAPEUTICS, INC., South Plainfield, NJ (US)

(21) Appl. No.: 17/783,941

(22) PCT Filed: Dec. 7, 2020
(86) PCT No.: PCT/US2020/063612

§ 371 Date: Jun. 9, 2022

Related U.S. Application Data
(60) Provisional application No. 62/947,049, filed on Dec. 12, 2019.

(51) Int. Cl. C07D 495/04

(52) U.S. Cl. CPC C07D 495/04

(57) ABSTRACT

The present description relates to compounds useful for improving pre-mRNA splicing in a cell. In particular, another aspect of the present description relates to substituted thieno [3,2-d]pyrimidine compounds, forms, and pharmaceutical compositions thereof and methods of use for treating or ameliorating familial dysautonomia.

### CROSS-REFERENCE TO RELATED APPLICATIONS

This application claims priority to U.S. Provisional Application No. 62/947,049 filed on Dec. 12, 2019.

### STATEMENT OF JOINT RESEARCH AGREEMENT

The subject matter disclosed was developed and the claimed invention was made by, or on behalf of, one or more parties to a joint research agreement that was in effect on or before the effective filing date of the claimed invention. The claimed invention was made as a result of activities undertaken within the scope of the joint research agreement. The parties of the joint research agreement are PTC Therapeutics, Inc. and The General Hospital Corporation, d/b/a Massachusetts General Hospital.

### TECHNICAL FIELD

An aspect of the present description relates to compounds useful for improving pre-mRNA splicing in a cell. In particular, another aspect of the present description relates to substituted thieno[3,2-d]pyrimidine compounds, forms, and pharmaceutical compositions thereof and methods of use for treating or ameliorating familial dysautonomia.

### BACKGROUND

Familial dysautonomia (FD) is a congenital sensory and autonomic neuropathy (HSAN) of the central and peripheral nervous system characterized by widespread sensory and variable autonomic dysfunction. FD affects neuronal development and is associated with progressive neuronal degeneration. Multiple systems are affected resulting in a markedly reduced quality of life and premature death. FD is caused by mutations in the IKBKAP (also referred to as ELP1) gene and in all cases described to date there is at least one allele carrying a T to C mutation at position 6 in intron 20 that results in a unique pattern of tissue-specific exon skipping.

Kinetin derivatives useful for therapeutically targeting pre-mRNA splicing mechanisms and the treatment of FD have been described in International Patent Application No. WO2016/115434, the disclosure of which is incorporated by reference in its entirety.

All other documents referred to herein are incorporated by reference into the present application as though fully set forth herein.

### SUMMARY

An aspect of the present description includes compounds comprising, a compound of Formula (I):

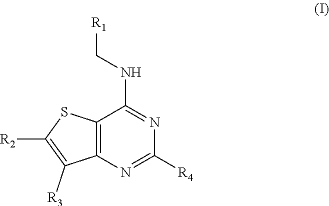

or a form thereof, wherein R₁, R₂, R₃, and R₄ are defined herein.

An aspect of the present description includes a method for use of a compound of Formula (I) or a form or composition thereof for treating or ameliorating FD in a subject in need thereof comprising, administering to the subject an effective amount of the compound of Formula (I) or a form or composition thereof.

An aspect of the present description includes a use for a compound of Formula (I) or a form thereof for treating or ameliorating FD in a subject in need thereof comprising, administering to the subject an effective amount of the compound of Formula (I) or a form thereof.

An aspect of the present description includes a use for a compound of Formula (I) or a form thereof in the manufacture of a medicament for treating or ameliorating FD in a subject in need thereof comprising, administering to the subject an effective amount of the medicament.

### DETAILED DESCRIPTION

An aspect of the present description relates to compounds comprising, a compound of Formula (I):

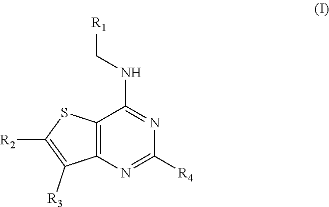

or a form thereof, wherein:

R₁ is aryl or heteroaryl, optionally substituted with one, two, three, or four independently selected R₁ₐ substituents;

R₁ₐ is cyano, halo, hydroxy, C₁₋₆alkyl, halo-C₁₋₆alkyl, deutero-C₁₋₆alkyl, or C₁₋₆alkoxy;

R₂ is hydrogen, C₁₋₆alkyl, C₂₋₆alkenyl, C₂₋₆alkynyl, C₃₋₁₀cycloalkyl, aryl, heterocyclyl, or heteroaryl,

wherein each instance of C₁₋₆alkyl, C₂₋₆alkenyl, C₂₋₆alkynyl, C₃₋₁₀cycloalkyl, aryl, heterocyclyl, and heteroaryl is optionally substituted with one, two, three, or four independently selected R₂ₐ substituents, and

wherein each instance of C₁₋₆alkyl, C₂₋₆alkenyl, C₂₋₆alkynyl and heterocyclyl optionally contains a chiral carbon having an (R) or (S) configuration;

R₂ₐ is cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀cycloalkyl-amino, aryl-amino, heterocyclyl-amino, heteroaryl-amino, C₁₋₆alkyl-thio, C₁₋₆alkyl-sulfonyl, C₃₋₁₀cycloalkyl, aryl, heterocyclyl, or heteroaryl,

wherein each instance of C₃₋₁₀cycloalkyl, aryl, heterocyclyl and heteroaryl is optionally substituted with one, two, three or four independently selected R_{2a′} substituents;

R_{2a′} is cyano, halo, hydroxy, oxo, C₁₋₆alkyl, halo-C₁₋₆alkyl, deutero-C₁₋₆alkyl, or C₁₋₆alkoxy;

R₃ is hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀cycloalkyl, aryl, heterocyclyl, or heteroaryl,

wherein each instance of C₁₋₆alkyl, C₃₋₁₀cycloalkyl, aryl, heterocyclyl, or heteroaryl are optionally substituted with one, two, three, or four independently selected R₃ₐ substituents;

R₃ₐ is cyano, halo, hydroxy, C₁₋₆alkyl, halo-C₁₋₆alkyl, or C₁₋₆alkoxy; and

R₄ is hydrogen, cyano, halo, hydroxy, C₁₋₆alkyl, halo-C₁₋₆alkyl, C₁₋₆alkoxy, carbamoyl, C₃₋₁₀cycloalkyl, aryl, or heterocyclyl,

wherein the form of the compound is selected from the group consisting of a salt, hydrate, solvate, racemate, enantiomer, diastereomer, stereoisomer, and tautomer form thereof.

One aspect includes a compound of Formula (I), wherein R₁ is aryl or heteroaryl, optionally substituted with one, two, three, or four independently selected R₁ₐ substituents.

Another aspect includes a compound of Formula (I), wherein R₁ is aryl or heteroaryl, optionally substituted with one or two, independently selected R₁ₐ substituents.

Another aspect includes a compound of Formula (I), wherein R₁ is aryl, optionally substituted with one, two, three, or four independently selected R₁ₐ substituents.

Another aspect includes a compound of Formula (I), wherein R₁ is aryl, optionally substituted with one R₁ₐ substituent.

Another aspect of includes a compound of Formula (I), wherein R₁ is aryl selected from phenyl and naphthyl, optionally substituted with one, two, three, or four independently selected R₁ₐ substituents.

Another aspect includes a compound of Formula (I), wherein R₁ is phenyl, wherein phenyl is optionally substituted with one, two, three, or four independently selected R₁ₐ substituents.

Another aspect includes a compound of Formula (I), wherein R₁ is phenyl, wherein phenyl is optionally substituted with one R₁ₐ substituent.

Another aspect includes a compound of Formula (I), wherein R₁is heteroaryl, optionally substituted with one, two, three, or four independently selected R₁ₐ substituents.

Another aspect includes a compound of Formula (I), wherein R₁ is heteroaryl, optionally substituted with one or two, independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein each instance of heteroaryl is optionally substituted with one, two, three, or four independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein each instance of heteroaryl is optionally substituted with one or two independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein each instance of heteroaryl is optionally substituted with one R₁ₐ substituent.

Another aspect of includes a compound of Formula (I), wherein R₁ is heteroaryl selected from furanyl, thiophenyl, 1H-pyrrolyl, 1H-pyrazolyl, 1H-imidazolyl, 1H-1,2,3-triazolyl, 2H-1,2,3-triazolyl, 1H-tetrazolyl, 1,2-thiazolyl, 1,3-thiazolyl, 1,2-oxazolyl, 1,3-oxazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein each instance of heteroaryl is optionally substituted with two independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, wherein each instance of heteroaryl is optionally substituted with one, two, three, or four independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, wherein each instance of heteroaryl is optionally substituted with one or two independently selected R₁ₐ substituents.

Another aspect of includes a compound of Formula (I), wherein R₁ is heteroaryl selected from furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, wherein each instance of heteroaryl is optionally substituted with one R₁ₐ substituent.

Another aspect of includes a compound of Formula (I), wherein R₁ is heteroaryl selected from furanyl, thiophenyl, 1H-pyrrolyl, 1H-pyrazolyl, 1H-imidazolyl, 2H-1,2,3-triazolyl, 1H-tetrazolyl, 1,2-thiazolyl, 1,3-thiazolyl, 1,2-oxazolyl, 1,3-oxazolyl, 1,2-thiazolyl, 1,3-thiazolyl, 1,2-oxazolyl, 1,3-oxazolyl, pyridinyl, pyrimidinyl, and pyrazinyl, wherein each instance of heteroaryl is optionally substituted with two independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl wherein, each instance of heteroaryl is optionally substituted with one, two, three, or four independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl wherein, each instance of heteroaryl is optionally substituted with one or two independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl wherein, each instance of heteroaryl is optionally substituted with one R₁ₐ substituent.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-1-yl, 1H-pyrazol-3-yl, 1H-pyrazol-4-yl, 1H-pyrazol-5-yl, 1H-imidazol-1-yl, 1H-imidazol-2-yl, 1H-imidazol-4-yl, 1H-imidazol-5-yl, 1H-1,2,3-triazol-1-yl, 1H-1,2,3-triazol-4-yl, 2H-1,2,3-triazol-2-yl, 2H-1,2,3-triazol-4-yl, 1H-tetrazol-1-yl, 1H-tetrazol-5-yl, 1,2-thiazol-3-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, 1,2,4-oxadiazol-3-yl, 1,3,4-oxadiazol-2-yl, tetrazol-5-yl, 1,2,3-triazol-4-yl, 1,2,3-triazol-5-yl, 1,2,3-thiadiazol-4-yl, 1,2,3-thiadiazol-5-yl, pyridin-2-yl, pyridin-3-yl, pyridin-4-yl, pyrimidin-2-yl, pyrimidin-4-yl, pyrazin-2-yl, pyridazin-3-yl, pyridazin-4-yl, benzofuran-2-yl, benzofuran-5-yl, and quinoline-4-yl wherein, each instance of heteroaryl is optionally substituted with two independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, wherein each instance of heteroaryl is optionally substituted with one, two, three, or four independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, wherein each instance of heteroaryl is optionally substituted with one or two independently selected R₁ₐ substituents.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, wherein each instance of heteroaryl is optionally substituted with one R₁ₐ substituent.

Another aspect includes a compound of Formula (I), wherein R₁ is heteroaryl selected from furan-2-yl, furan-3-yl, thiophen-2-yl, thiophen-3-yl, 1H-pyrrol-2-yl, 1H-pyrrol-3-yl, 1H-pyrazol-3-yl, 1H-pyrazol-4-yl, 1H-pyrazol-5-yl, 1H-imidazol-2-yl, 1H-imidazol-4-yl, 1H-imidazol-5-yl, 2H-1,2,3-triazol-4-yl, 1H-tetrazol-5-yl, 1,2-thiazol-4-yl, 1,2-thiazol-5-yl, 1,3-thiazol-2-yl, 1,3-thiazol-4-yl, 1,3-thiazol-5-yl, 1,2-oxazol-3-yl, 1,2 oxazol-4-yl, 1,2-oxazol-5-yl, 1,3-oxazol-2-yl, 1,3-oxazol-4-yl, 1,3-oxazol-5-yl, pyridin-2-yl, pyridin-3-yl, pyridin-4-yl, pyrimidin-2-yl, pyrimidin-4-yl, and pyrazin-2-yl, wherein each instance of heteroaryl is optionally substituted with two independently selected R₁ₐ substituents.

One aspect includes a compound of Formula (I), wherein R₁ₐ is cyano, halo, hydroxy, C₁₋₆alkyl, halo-C₁₋₆alkyl, deutero-C₁₋₆alkyl, or C₁₋₆alkoxy.

Another aspect includes a compound of Formula (I), wherein R₁ₐ is halo or C₁₋₆alkyl.

Another aspect includes a compound of Formula (I), wherein R₁ₐ is halo selected from fluoro, chloro, bromo, and iodo.

Another aspect includes a compound of Formula (I), wherein R₁ₐ is fluoro.

Another aspect includes a compound of Formula (I), wherein R₁ₐ is C₁₋₆alkyl selected from methyl, ethyl, propyl, butyl, pentyl, and hexyl.

Another aspect includes a compound of Formula (I), wherein R₁ₐ is methyl.

One aspect includes a compound of Formula (I), wherein R₂ is hydrogen, C₁₋₆alkyl, C₁₋₆alkenyl, C₁₋₆alkynyl, C₃₋₁₀cycloalkyl, aryl, heterocyclyl, or heteroaryl, wherein each instance of C₁₋₆alkyl, C₂₋₆alkenyl, C₂₋₆alkynyl, C₃₋₁₀cycloalkyl, aryl, heterocyclyl, and heteroaryl is optionally substituted with one, two, three, or four independently selected R₂ₐ substituents, and wherein, each instance of C₁₋₆alkyl, C₂₋₆alkenyl, C₂₋₆alkynyl and heterocyclyl may optionally contain a chiral carbon having an (R) or (S) configuration wherein each instance of C₁₋₆alkyl, C₂₋₆alkenyl, C₂₋₆alkynyl and heterocyclyl may optionally contain a chiral carbon having an (R) or (S) configuration.

Another aspect includes a compound of Formula (I), wherein R₂ is hydrogen.

Another aspect includes a compound of Formula (I), wherein R₂ is C₁₋₆alkyl, optionally substituted with one, two, three, or four independently selected R₂ₐ substituents, and wherein, C₁₋₆alkyl optionally contains a chiral carbon having an (R) or (S) configuration.

Another aspect includes a compound of Formula (I), wherein R₂ is C₁₋₆alkyl, optionally substituted with one, two, three, or four independently selected R₂ₐ substituents, and wherein, C₁₋₆alkyl contains a chiral carbon having an (R) configuration.

Another aspect includes a compound of Formula (I), wherein R₂ is C₁₋₆alkyl, optionally substituted with one, two, three, or four independently selected R₂ₐ substituents, and wherein, C₁₋₆alkyl contains a chiral carbon having an (S) configuration.

Another aspect includes a compound of Formula (I), wherein R₂ is C₁₋₆alkyl selected from methyl, ethyl, propyl, butyl, pentyl, and hexyl optionally substituted with one, two, three, or four independently selected R₂ₐ substituents, and wherein, C₁₋₆alkyl optionally contains a chiral carbon having an (R) or (S) configuration.

Another aspect includes a compound of Formula (I), wherein R₂ is C₁₋₆alkyl selected from methyl, ethyl, propyl, butyl, pentyl, and hexyl optionally substituted with one, two, three, or four independently selected R₂ₐ substituents, and wherein, C₁₋₆alkyl contains a chiral carbon having an (R) configuration.

Another aspect includes a compound of Formula (I), wherein R₂ is C₁₋₆alkyl selected from methyl, ethyl, propyl, butyl, pentyl, and hexyl optionally substituted with one, two, three, or four independently selected R₂ₐ substituents, and wherein, C₁₋₆alkyl contains a chiral carbon having an or (S) configuration.

Another aspect includes a compound of Formula (I), wherein R₂ is C₁₋₆alkyl selected from methyl, ethyl, propyl, butyl, and pentyl, optionally substituted with one, two, three, or four independently selected R₂ₐ substituents, and wherein, C₁₋₆alkyl optionally contains a chiral carbon having an (R) or (S) configuration.

Another aspect includes a compound of Formula (I), wherein R₂ is C₁₋₆alkyl selected from methyl, ethyl, propyl, butyl, and pentyl, optionally substituted with one, two, three, or four independently selected R₂ₐ substituents, and wherein, C₁₋₆alkyl contains a chiral carbon having an (R) configuration.

Another aspect includes a compound of Formula (I), wherein R₂ is C₁₋₆alkyl selected from methyl, ethyl, propyl, butyl, and pentyl, optionally substituted with one, two, three, or four independently selected R₂ₐ substituents, and wherein, C₁₋₆alkyl contains a chiral carbon having an (S) configuration.

Another aspect includes a compound of Formula (I), wherein R₂ is heterocyclyl, optionally substituted with one, two, three, or four independently selected R₂ₐ substituents, and wherein heterocyclyl optionally contains a chiral carbon having an (R) or (S) configuration.

Another aspect includes a compound of Formula (I), wherein R₂ is heterocyclyl, optionally substituted with one, two, three, or four independently selected R₂ₐ substituents, and wherein heterocyclyl contains a chiral carbon having an (R) configuration.

Another aspect includes a compound of Formula (I), wherein R₂ is heterocyclyl, optionally substituted with one, two, three, or four independently selected R₂ₐ substituents, and wherein heterocyclyl contains a chiral carbon having an (S) configuration.

Another aspect includes a compound of Formula (I), wherein R₂ is heterocyclyl selected from azetidinyl, oxetanyl, pyrazolidinyl, tetrahydrofuranyl, oxazolidinyl, thiazolidinyl, isothiazolidinyl, pyrrolidinyl, piperidinyl, piperazinyl, 2H-pyranyl, tetrahydropyranyl, morpholinyl, 1,3-oxazinanyl, 1,3-oxazinan-2-on-yl, and azepanyl, optionally substituted with one, two, three, or four independently selected R₂ₐ substituents.

Another aspect includes a compound of Formula (I), wherein R₂ is heterocyclyl selected from azetidinyl and pyrrolidinyl, optionally substituted with one, two, three, or four independently selected R₂ₐ substituents.

Another aspect includes a compound of Formula (I), wherein R₂ is heterocyclyl selected from azetidin-2-yl, azetidin-3-yl, oxetan-2-yl, oxetan-3-yl, pyrazolidin-1-yl, pyrazolidin-2-yl, pyrazolidin-3-yl, pyrazolidin-4-yl, pyrazolidin-5-yl, tetrahydrofuran-1-yl, tetrahydrofuran-2-yl, oxazolidin-2-yl, oxazolidin-4-yl, oxazolidin-5-yl, thiazolidin-2-yl, thiazolidin-4-yl, thiazolidin-5-yl, isothiazolidin-3-yl, isothiazolidin-4-yl, isothiazolidin-5-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, piperazin-3-yl, 2H-pyran-2-yl, 2H-pyran-3-yl, 2H-pyran-4-yl, 2H-pyran-5-yl, 2H-pyran-6-yl, tetrahydropyran-2-yl, tetrahydropyran-3-yl, tetrahydropyran-4-yl, morpholin-2-yl, morpholin-3-yl, morpholin-4-yl, 1,3-oxazinan-2-yl, 1,3-oxazinan-3-yl, 1,3-oxazinan-4-yl, 1,3-oxazinan-2-on-6-yl, azepan-1-yl, azepan-2-yl, azepan-3-yl, and azepan-4-yl, optionally substituted with one, two, three, or four independently selected R₂ₐ substituents.

Another aspect includes a compound of Formula (I), wherein R₂ is heterocyclyl selected from azetidin-3-yl and pyrrolidin-3-yl, optionally substituted with one, two, three, or four independently selected R₂ₐ substituents.

One aspect includes a compound of Formula (I), wherein R₂ₐ is cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀ocycloalkyl-amino, aryl-amino, heterocyclyl-amino, heteroaryl-amino, C₁₋₆alkyl-thio, C₁₋₆alkyl-sulfonyl, C₃₋₁₀cycloalkyl, aryl, heterocyclyl, or heteroaryl, wherein, each instance of C₃₋₁₀cycloalkyl, aryl, heterocyclyl and heteroaryl is optionally substituted with one, two, three or four independently selected R_{2a′} substituents.

Another aspect includes a compound of Formula (I), wherein R₂ₐ is halo, hydroxy, C₁₋₆alkyl, C₁₋₆alkoxy, amino, C₁₋₆alkyl-amino, C₃₋₁₀cycloalkyl-amino, C₃₋₁₀cycloalkyl, or heterocyclyl, wherein each instance of C₃₋₁₀cycloalkyl or heterocyclyl is optionally substituted with one, two, three or four independently selected R_{2a′} substituents.

Another aspect includes a compound of Formula (I), wherein R₂ₐ is halo selected from fluoro, chloro, bromo, and iodo.

Another aspect includes a compound of Formula (I), wherein R₂ₐ is fluoro.

Another aspect includes a compound of Formula (I), wherein R₂ₐ is hydroxy.

Another aspect includes a compound of Formula (I), wherein R₂ₐ is C₁₋₆alkyl selected from methyl, ethyl, propyl, butyl, pentyl, and hexyl.

Another aspect includes a compound of Formula (I), wherein R₂ₐ is methyl.

Another aspect includes a compound of Formula (I), wherein R₂ₐis C₁₋₆alkoxy selected from methoxy, ethoxy, propoxy, isopropoxy, butoxy, and tert-butoxy.

Another aspect includes a compound of Formula (I), wherein R₂ₐ is methoxy.

Another aspect includes a compound of Formula (I), wherein R₂ₐ is amino.

Another aspect includes a compound of Formula (I), wherein R₂ₐ is C₁₋₆alkyl-amino, wherein C₁₋₆alkyl is selected from methyl, ethyl, propyl, isopropyl, butyl, sec-butyl, isobutyl, and tert-butyl.

Another aspect includes a compound of Formula (I), wherein R₂ₐ is methyl-amino.

Another aspect includes a compound of Formula (I), wherein R₂ₐ is C₃₋₁₀cycloalkyl-amino, wherein C₃₋₁₀cycloalkyl is selected from cyclopropyl, cyclobutyl, cyclopentyl, cyclohexyl, cycloheptyl, and cyclooctyl, wherein each instance of C₃₋₁₀cycloalkyl is optionally substituted with one, two, three or four independently selected R_{2a′} substituents.

Another aspect includes a compound of Formula (I), wherein R₂ₐ is cyclobutyl-amino.

Another aspect includes a compound of Formula (I), wherein R₂ₐ is C₃₋₁₀cycloalkyl, wherein C₃₋₁₀cycloalkyl is selected from cyclopropyl, cyclobutyl, cyclopentyl, cyclohexyl, cycloheptyl, and cyclooctyl, wherein each instance of C₃₋₁₀cycloalkyl is optionally substituted with one, two, three or four independently selected R_{2a′} substituents.

Another aspect includes a compound of Formula (I), wherein R₂ₐ is cyclopropyl, wherein each instance of C₃₋₁₀cycloalkyl is optionally substituted with one, two, three or four independently selected R_{2a′} substituents.

Another aspect includes a compound of Formula (I), wherein R₂ₐ is heterocyclyl selected from heterocyclyl selected from azetidinyl, oxetanyl, pyrazolidinyl, tetrahydrofuranyl, oxazolidinyl, thiazolidinyl, isothiazolidinyl, pyrrolidinyl, piperidinyl, piperazinyl, 2H-pyranyl, tetrahydropyranyl, morpholinyl, 1,3-oxazinanyl, 1,3-oxazinan-2-on-yl, and azepanyl, wherein each instance of heterocyclyl is optionally substituted with one, two, three or four independently selected R_{2a′} substituents.

Another aspect includes a compound of Formula (I), wherein R₂ₐ is 1,3-oxazinan-2-on-yl.

Another aspect includes a compound of Formula (I), wherein R₂ₐ is heterocyclyl selected from heterocyclyl selected from azetidin-2-yl, azetidin-3-yl, oxetan-2-yl, oxetan-3-yl, pyrazolidin-1-yl, pyrazolidin-2-yl, pyrazolidin-3-yl, pyrazolidin-4-yl, pyrazolidin-5-yl, tetrahydrofuran-1-yl, tetrahydrofuran-2-yl, oxazolidin-2-yl, oxazolidin-4-yl, oxazolidin-5-yl, thiazolidin-2-yl, thiazolidin-4-yl, thiazolidin-5-yl, isothiazolidin-3-yl, isothiazolidin-4-yl, isothiazolidin-5-yl, pyrrolidin-2-yl, pyrrolidin-3-yl, piperidin-1-yl, piperidin-2-yl, piperidin-3-yl, piperidin-4-yl, piperazin-1-yl, piperazin-2-yl, piperazin-3-yl, 2H-pyran-2-yl, 2H-pyran-3-yl, 2H-pyran-4-yl, 2H-pyran-5-yl, 2H-pyran-6-yl, tetrahydropyran-2-yl, tetrahydropyran-3-yl, tetrahydropyran-4-yl, morpholin-2-yl, morpholin-3-yl, morpholin-4-yl, 1,3-oxazinan-2-yl, 1,3-oxazinan-3-yl, 1,3-oxazinan-4-yl, 1,3-oxazinan-2-on-6-yl, azepan-1-yl, azepan-2-yl, azepan-3-yl, and azepan-4-yl, wherein each instance of heterocyclyl is optionally substituted with one, two, three or four independently selected R_{2a′} substituents.

Another aspect includes a compound of Formula (I), wherein R₂ₐ is 1,3-oxazinan-2-on-6-yl.

One aspect includes a compound of Formula (I), wherein R₃ is hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀cycloalkyl, aryl, heterocyclyl, or heteroaryl, wherein each instance of C₁₋₆alkyl, C₃₋₁₀cycloalkyl, aryl, heterocyclyl, or heteroaryl are optionally substituted with one, two, three, or four independently selected R₃ₐ substituents.

Another aspect includes a compound of Formula (I), wherein R₃ is hydrogen, cyano, halo, C₁₋₆alkyl, C₁₋₆alkoxy, C₃₋₁₀cycloalkyl, or aryl, wherein each instance of C₁₋₆alkyl, C₃₋₁₀cycloalkyl, or aryl, are optionally substituted with one, two, three, or four independently selected R₃ₐ substituents.

Another aspect includes a compound of Formula (I), wherein R₃ is hydrogen.

Another aspect includes a compound of Formula (I), wherein R₃ is cyano.

Another aspect includes a compound of Formula (I), wherein R₃ is halo selected from fluoro, chloro, bromo, and iodo.

Another aspect includes a compound of Formula (I), wherein R₃ is bromo.

Another aspect includes a compound of Formula (I), wherein R₃ is hydroxy.

Another aspect includes a compound of Formula (I), wherein R₃ is C₁₋₆alkyl selected from methyl, ethyl, propyl, butyl, pentyl, and hexyl, optionally substituted with one, two, three, or four independently selected R₃ₐ substituents.

Another aspect includes a compound of Formula (I), wherein R₃ is C₁₋₆alkyl selected from methyl and ethyl, optionally substituted with one, two, three, or four independently selected R₃ₐ substituents.

Another aspect includes a compound of Formula (I), wherein R₃ is C₁₋₆alkoxy selected from methoxy, ethoxy, propoxy, isopropoxy, butoxy, and tert-butoxy.

Another aspect includes a compound of Formula (I), wherein R₃ is methoxy.

Another aspect includes a compound of Formula (I), wherein R₃ is C₃₋₁₀cycloalkyl selected from cyclopropyl, cyclobutyl, cyclopentyl, cyclohexyl, cycloheptyl, and cyclooctyl, optionally substituted with one, two, three, or four independently selected R₃ₐ substituents.

Another aspect includes a compound of Formula (I), wherein R₃ is cyclopropyl, optionally substituted with one, two, three, or four independently selected R₃ₐ substituents.

Another aspect includes a compound of Formula (I), wherein R₃ is aryl selected from phenyl and naphthyl, optionally substituted with one, two, three, or four independently selected R₃ₐ substituents.

Another aspect includes a compound of Formula (I), wherein R₃ is phenyl, optionally substituted with one, two, three, or four independently selected R₃ₐ substituents.

One aspect includes a compound of Formula (I), wherein R₃ₐ is cyano, halo, hydroxy, C₁₋₆alkyl, halo-C₁₋₆alkyl, or C₁₋₆alkoxy.

Another aspect includes a compound of Formula (I), wherein R₃ₐ is halo or C₁₋₆alkoxy.

Another aspect includes a compound of Formula (I), wherein R₃ₐ is halo selected from fluoro, chloro, bromo, and iodo.

Another aspect includes a compound of Formula (I), wherein R₃ₐ is chloro.

Another aspect includes a compound of Formula (I), wherein R₃ₐ is C₁₋₆alkoxy selected from methoxy, ethoxy, propoxy, isopropoxy, butoxy, and tert-butoxy.

Another aspect includes a compound of Formula (I), wherein R₃ₐ is methoxy.

One aspect includes a compound of Formula (I), wherein R₄ is hydrogen, cyano, halo, hydroxy, C₁₋₆alkyl, halo-C₁₋₆alkyl, C₁₋₆alkoxy, carbamoyl, C₃₋₁₀cycloalkyl, aryl, or heterocyclyl.

Another aspect includes a compound of Formula (I), wherein R₄ is hydrogen, cyano, halo, C₁₋₆alkyl, halo-C₁₋₆alkyl, carbamoyl, C₃₋₁₀cycloalkyl, or aryl.

Another aspect includes a compound of Formula (I), wherein R₄ is hydrogen.

Another aspect includes a compound of Formula (I), wherein R₄ is cyano.

Another aspect includes a compound of Formula (I), wherein R₄ is halo selected from fluoro, chloro, bromo, and iodo.

Another aspect includes a compound of Formula (I), wherein R₄ is halo selected from chloro and bromo.

Another aspect includes a compound of Formula (I), wherein R₄ is C₁₋₆alkyl selected from methyl, ethyl, propyl, butyl, pentyl, and hexyl.

Another aspect includes a compound of Formula (I), wherein R₄ is C₁₋₆alkyl selected from methyl and ethyl.

Another aspect includes a compound of Formula (I), wherein R₄ is halo-C₁₋₆alkyl wherein C₁₋₆alkyl is selected from methyl, ethyl, propyl, butyl, pentyl, and hexyl partially or completely substituted with one or more halogen atoms where allowed by available valences.

Another aspect includes a compound of Formula (I), wherein R₄ is halo-C₁₋₆alkyl, wherein C₁₋₆alkyl is methyl substituted with three fluorine atoms.

Another aspect includes a compound of Formula (I), wherein R₄ is carbamoyl.

Another aspect includes a compound of Formula (I), wherein R₄ is C₃₋₁₀cycloalkyl selected from cyclopropyl, cyclobutyl, cyclopentyl, cyclohexyl, cycloheptyl, and cyclooctyl.

Another aspect includes a compound of Formula (I), wherein R₄ is cyclopropyl.

Another aspect includes a compound of Formula (I), wherein R₄ is aryl selected from phenyl and naphthyl.

Another aspect includes a compound of Formula (I), wherein R₄ is phenyl.

One aspect of the compound of Formula (I) or a form thereof includes a compound selected from the group consisting of:

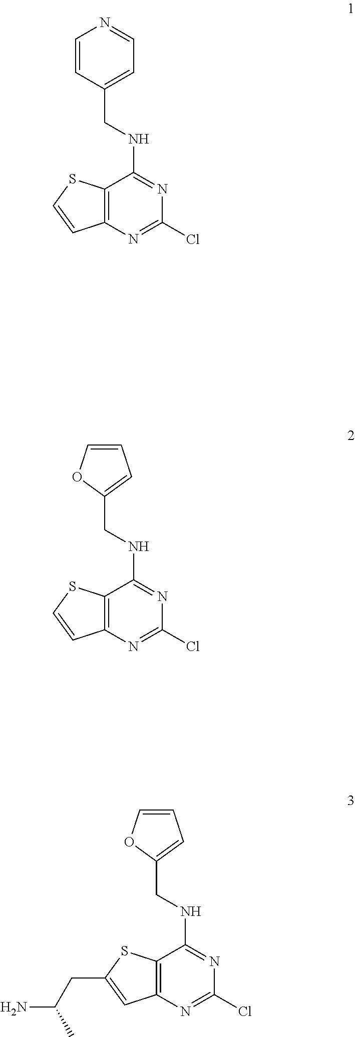 [SMILES: Clc1nc(NCc2ccncc2)c2sccc2n1 ; Clc1nc(NCc2ccco2)c2sccc2n1 ; C[C@H](N)Cc1cc2nc(Cl)nc(NCc3ccco3)c2s1]

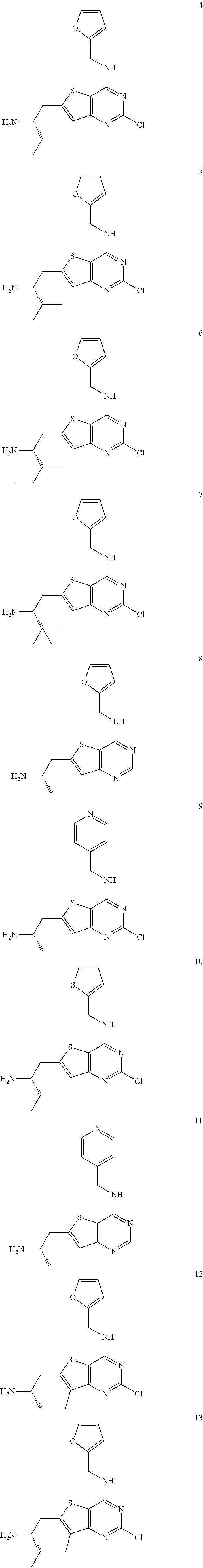

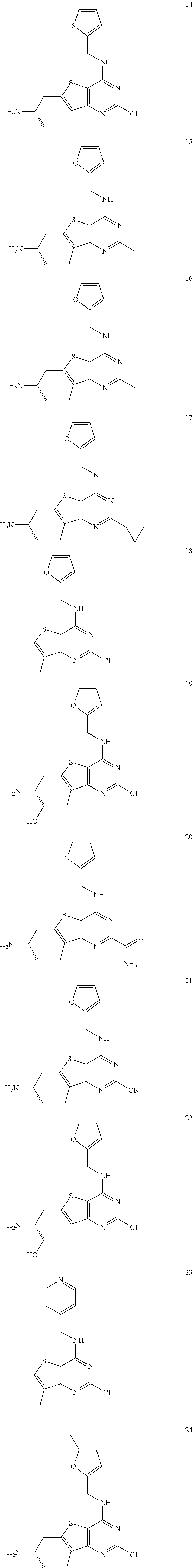

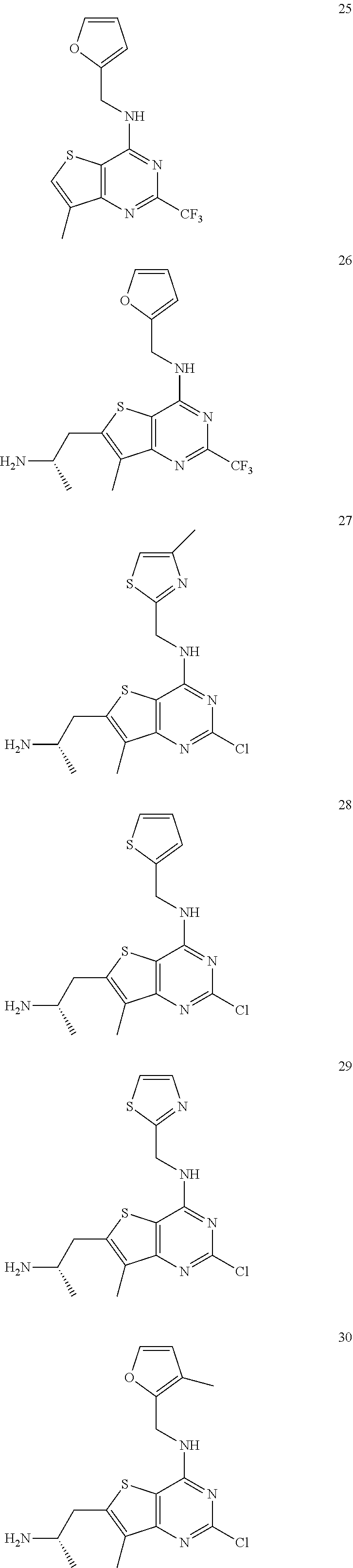 [SMILES: Cc1csc2c(NCc3ccco3)nc(C(F)(F)F)nc12 ; Cc1c(C[C@H](C)N)sc2c(NCc3ccco3)nc(C(F)(F)F)nc12 ; Cc1csc(CNc2nc(Cl)nc3c(C)c(C[C@H](C)N)sc23)n1 ; Cc1c(C[C@H](C)N)sc2c(NCc3cccs3)nc(Cl)nc12 ; Cc1c(C[C@H](C)N)sc2c(NCc3nccs3)nc(Cl)nc12 ; Cc1ccoc1CNc1nc(Cl)nc2c(C)c(C[C@H](C)N)sc12]

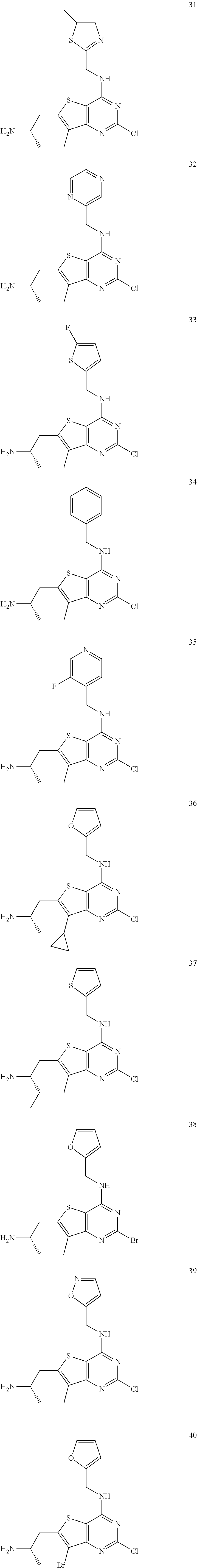

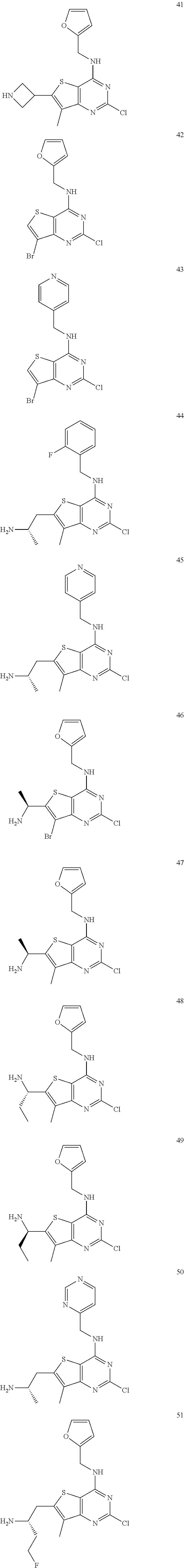

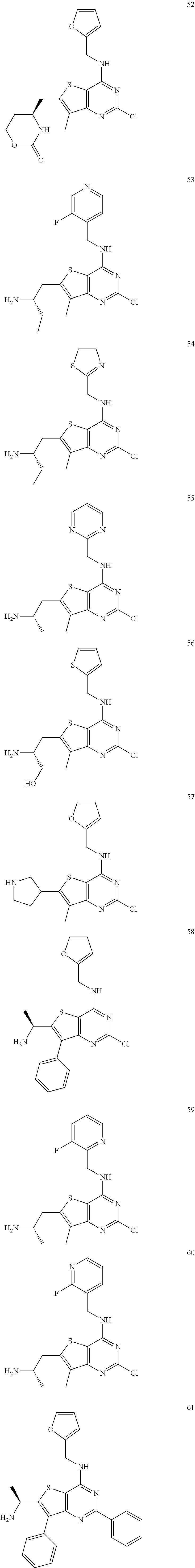

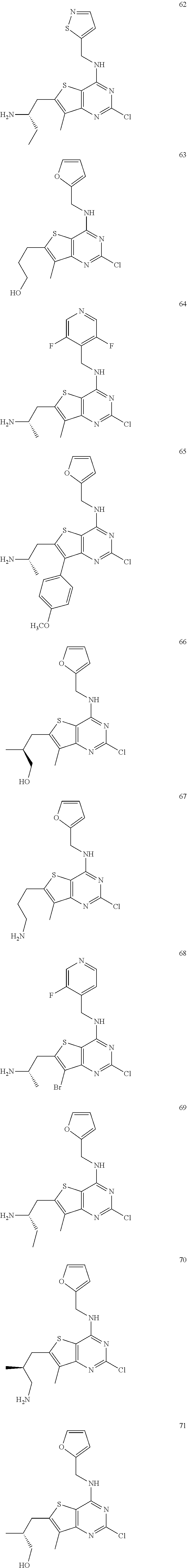

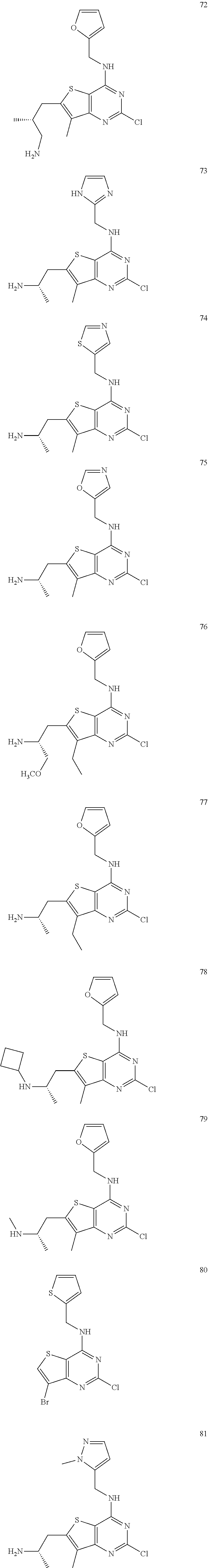

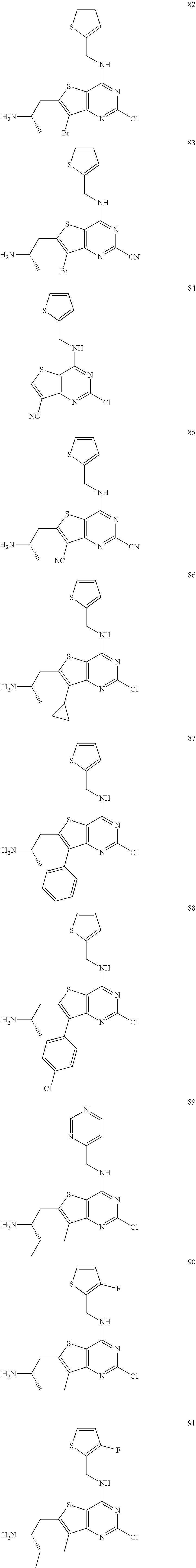

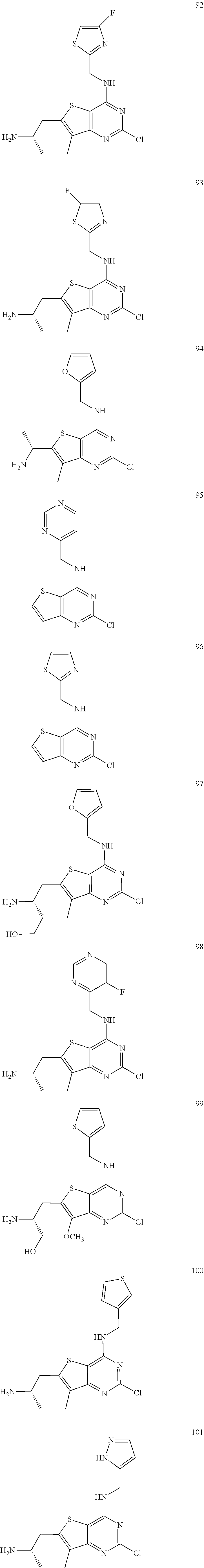

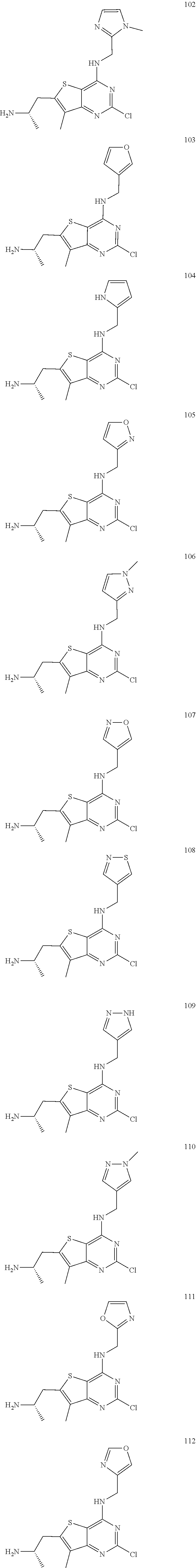

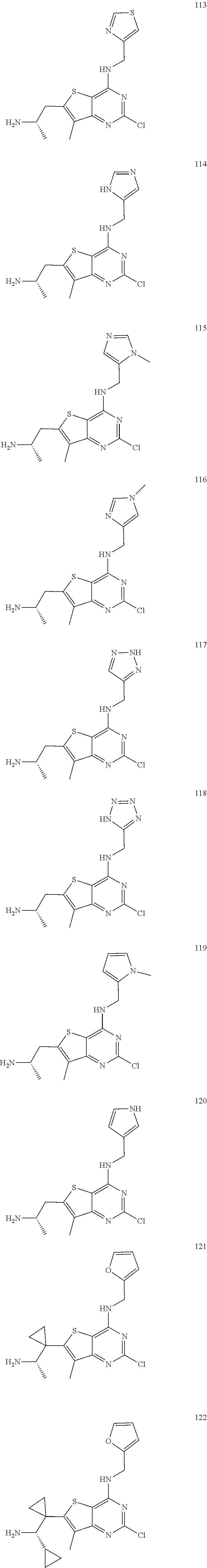

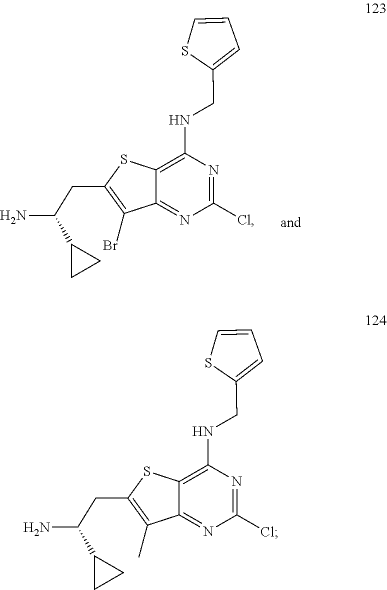 [SMILES: N[C@H](Cc1sc2c(NCc3cccs3)nc(Cl)nc2c1Br)C1CC1 ; Cc1c(C[C@@H](N)C2CC2)sc2c(NCc3cccs3)nc(Cl)nc12]

wherein the form of the compound is selected from the group consisting of a salt, hydrate, solvate, racemate, enantiomer, diastereomer, stereoisomer, and tautomer form thereof.

An aspect the compound of Formula (I) or a form thereof (wherein compound number (#¹) indicates that the salt form was isolated) includes a compound selected from the group consisting of:

| Cpd | Name |
|---|---|
| 1 | 2-chloro-N-[(pyridin-4-yl)methyl]thieno[3,2-d]pyrimidin-4-amine |
| 2 | 2-chloro-N-[(furan-2-yl)methyl]thieno[3,2-d]pyrimidin-4-amine |
| 3¹ | 6-[(2S)-2-aminopropyl]-2-chloro-N-[(furan-2-yl)methyl]thieno[3,2-d]pyrimidin-4-amine |
| 4¹ | 6-[(2S)-2-aminobutyl]-2-chloro-N-[(furan-2-yl)methyl]thieno[3,2-d]pyrimidin-4-amine |
| 5¹ | 6-[(2R)-2-amino-3-methylbutyl]-2-chloro-N-[(furan-2-yl)methyl]thieno[3,2-d]pyrimidin- |
| | 4-amine |
| 6¹ | 6-[(2R,3S)-2-amino-3-methylpentyl]-2-chloro-N-[(furan-2-yl)methyl]thieno[3,2- |
| | d]pyrimidin-4-amine |
| 7¹ | 6-[(2R)-2-amino-3,3-dimethylbutyl]-2-chloro-N-[(furan-2-yl)methyl]thieno[3,2- |
| | d]pyrimidin-4-amine |
| 8¹ | 6-[(2S)-2-aminopropyl]-N-[(furan-2-yl)methyl]thieno[3,2-d]pyrimidin-4-amine |
| 9¹ | 6-[(2S)-2-aminopropyl]-2-chloro-N-[(pyridin-4-yl)methyl]thieno[3,2-d]pyrimidin-4- |
| | amine |
| 10¹ | 6-[(2S)-2-aminobutyl]-2-chloro-N-[(thiophen-2-yl)methyl]thieno[3,2-d]pyrimidin-4- |
| | amine |
| 11¹ | 6-[(2S)-2-aminopropyl]-N-[(pyridin-4-yl)methyl]thieno[3,2-d]pyrimidin-4-amine |
| 12¹ | 6-[(2S)-2-aminopropyl]-2-chloro-N-[(furan-2-yl)methyl]-7-methylthieno[3,2- |
| | d]pyrimidin-4-amine |
| 13¹ | 6-[(2S)-2-aminobutyl]-2-chloro-N-[(furan-2-yl)methyl]-7-methylthieno[3,2-d]pyrimidin- |
| | 4-amine |
| 14¹ | 6-[(2S)-2-aminopropyl]-2-chloro-N-[(thiophen-2-yl)methyl]thieno[3,2-d]pyrimidin-4- |
| | amine |
| 15¹ | 6-[(2S)-2-aminopropyl]-N-[(furan-2-yl)methyl]-2,7-dimethylthieno[3,2-d]pyrimidin-4- |
| | amine |
| 16¹ | 6-[(2S)-2-aminopropyl]-2-ethyl-N-[(furan-2-yl)methyl]-7-methylthieno[3,2-d]pyrimidin- |
| | 4-amine |
| 17¹ | 6-[(2S)-2-aminopropyl]-2-cyclopropyl-N-[(furan-2-yl)methyl]-7-methylthieno[3,2- |
| | d]pyrimidin-4-amine |
| 18¹ | 2-chloro-N-[(furan-2-yl)methyl]-7-methylthieno[3,2-d]pyrimidin-4-amine |
| 19¹ | (2R)-2-amino-3-(2-chloro-4-{[(furan-2-yl)methyl]amino}-7-methylthieno[3,2- |
| | d]pyrimidin-6-yl)propan-l-ol |
| 20¹ | 6-[(2S)-2-aminopropyl]-4-{[(furan-2-yl)methyl]amino}-7-methylthieno[3,2- |
| | d]pyrimidine-2-carboxamide |
| 21¹ | 6-[(2S)-2-aminopropyl]-4-{[(furan-2-yl)methyl]amino}-7-methylthieno[3,2- |
| | d]pyrimidine-2-carbonitrile |
| 22 | (2R)-2-amino-3-(2-chloro-4-{[(furan-2-yl)methyl]amino}thieno[3,2-d]pyrimidin-6- |
| | yl)propan-l-ol |
| 23 | 2-chloro-7-methyl-N-[(pyridin-4-yl)methyl]thieno[3,2-d]pyrimidin-4-amine |
| 24¹ | 6-[(2S)-2-aminopropyl]-2-chloro-7-methyl-N-[(5-methylfuran-2-yl)methyl]thieno[3,2- |
| | d]pyrimidin-4-amine |
| 25 | N-[(furan-2-yl)methyl]-7-methyl-2-(trifluoromethyl)thieno[3,2-d]pyrimidin-4-amine |
| 26¹ | 6-[(2S)-2-aminopropyl]-N-[(furan-2-yl)methyl]-7-methyl-2-(trifluoromethyl)thieno[3,2- |
| | d]pyrimidin-4-amine |
| 27¹ | 6-[(2S)-2-aminopropyl]-2-chloro-7-methyl-N-[(4-methyl-1,3-thiazol-2- |
| | yl)methyl]thieno[3,2-d]pyrimidin-4-amine |
| 28¹ | 6-[(2S)-2-aminopropyl]-2-chloro-7-methyl-N-[(thiophen-2-yl)methyl]thieno[3,2- |
| | d]pyrimidin-4-amine |
| 29¹ | 6-[(2S)-2-aminopropyl]-2-chloro-7-methyl-N-[(1,3-thiazol-2-yl)methyl]thieno[3,2- |
| | d]pyrimidin-4-amine |
| 30¹ | 6-[(2S)-2-aminopropyl]-2-chloro-7-methyl-N-[(3-methylfuran-2-yl)methyl]thieno[3,2- |
| | d]pyrimidin-4-amine |
| 31¹ | 6-[(2S)-2-aminopropyl]-2-chloro-7-methyl-N-[(5-methyl-1,3-thiazol-2- |
| | yl)methyl]thieno[3,2-d]pyrimidin-4-amine |
| 32¹ | 6-[(2S)-2-aminopropyl]-2-chloro-7-methyl-N-[(pyrazin-2-yl)methyl]thieno[3,2- |
| | d]pyrimidin-4-amine |
| 33¹ | 6-[(2S)-2-aminopropyl]-2-chloro-N-[(5-fluorothiophen-2-yl)methyl]-7-methylthieno[3,2- |
| | d]pyrimidin-4-amine |
| 34¹ | 6-[(2S)-2-aminopropyl]-N-benzyl-2-chloro-7-methylthieno[3,2-d]pyrimidin-4-amine |
| 35¹ | 6-[(2S)-2-aminopropyl]-2-chloro-N-[(3-fluoropyridin-4-yl)methyl]-7-methylthieno[3,2- |
| | d]pyrimidin-4-amine |
| 36¹ | 6-[(2S)-2-aminopropyl]-2-chloro-7-cyclopropyl-N-[(furan-2-yl)methyl]thieno[3,2- |
| | d]pyrimidin-4-amine |
| 37¹ | 6-[(2S)-2-aminobutyl]-2-chloro-7-methyl-N-[(thiophen-2-yl)methyl]thieno[3,2- |
| | d]pyrimidin-4-amine |
| 38¹ | 6-[(2S)-2-aminopropyl]-2-bromo-N-[(furan-2-yl)methyl]-7-methylthieno[3,2- |
| | d]pyrimidin-4-amine |
| 39¹ | 6-[(2S)-2-aminopropyl]-2-chloro-7-methyl-N-[(1,2-oxazol-5-yl)methyl]thieno[3,2- |
| | d]pyrimidin-4-amine |
| 40¹ | 6-[(2S)-2-aminopropyl]-7-bromo-2-chloro-N-[(furan-2-yl)methyl]thieno[3,2-d]pyrimidin- |
| | 4-amine |
| 41¹ | 6-(azetidin-3-yl)-2-chloro-N-[(furan-2-yl)methyl]-7-methylthieno[3,2-d]pyrimidin-4- |
| | amine |
| 42 | 7-bromo-2-chloro-N-[(furan-2-yl)methyl]thieno[3,2-d]pyrimidin-4-amine |
| 43 | 7-bromo-2-chloro-N-[(pyridin-4-yl)methyl]thieno[3,2-d]pyrimidin-4-amine |
| 44¹ | 6-[(2S)-2-aminopropyl]-2-chloro-N-[(2-fluorophenyl)methyl]-7-methylthieno[3,2- |
| | d]pyrimidin-4-amine |
| 45¹ | 6-[(2S)-2-aminopropyl]-2-chloro-7-methyl-N-[(pyridin-4-yl)methyl]thieno[3,2- |
| | d]pyrimidin-4-amine |
| 46¹ | 6-[(1S)-1-aminoethyl]-7-bromo-2-chloro-N-[(furan-2-yl)methyl]thieno[3,2-d]pyrimidin- |
| | 4-amine |
| 47¹ | 6-[(1S)-1-aminoethyl]-2-chloro-N-[(furan-2-yl)methyl]-7-methylthieno[3,2-d]pyrimidin- |
| | 4-amine |
| 48¹ | 6-[(1S)-1-aminopropyl]-2-chloro-N-[(furan-2-yl)methyl]-7-methylthieno[3,2- |
| | d]pyrimidin-4-amine |
| 49¹ | 6-[(1S)-1-aminopropyl]-2-chloro-N-[(furan-2-yl)methyl]-7-methylthieno[3,2- |
| | d]pyrimidin-4-amine |
| 50¹ | 6-[(2S)-2-aminopropyl]-2-chloro-7-methyl-N-[(pyrimidin-4-yl)methyl]thieno[3,2- |
| | d]pyrimidin-4-amine |
| 51¹ | 6-[(2S)-2-amino-4-fluorobutyl]-2-chloro-N-[(furan-2-yl)methyl]-7-methylthieno[3,2- |
| | d]pyrimidin-4-amine |
| 52 | (4S)-4-[(2-chloro-4-{[(furan-2-yl)methyl]amino}-7-methylthieno[3,2-d]pyrimidin-6- |
| | yl)methyl]-1,3-oxazinan-2-one |
| 53¹ | 6-[(2S)-2-aminobutyl]-2-chloro-N-[(3-fluoropyridin-4-yl)methyl]-7-methylthieno[3,2- |
| | d]pyrimidin-4-amine |
| 54¹ | 6-[(2S)-2-aminobutyl]-2-chloro-7-methyl-N-[(1,3-thiazol-2-yl)methyl]thieno[3,2- |
| | d]pyrimidin-4-amine |
| 55¹ | 6-[(2S)-2-aminopropyl]-2-chloro-7-methyl-N-[(pyrimidin-2-yl)methyl]thieno[3,2- |
| | d]pyrimidin-4-amine |
| 56¹ | (2R)-2-amino-3-(2-chloro-7-methyl-4-{[(thiophen-2-yl)methyl]amino}thieno[3,2- |
| | d]pyrimidin-6-yl)propan-l-ol |
| 57¹ | 2-chloro-N-[(furan-2-yl)methyl]-7-methyl-6-(pyrrolidin-3-yl)thieno[3,2-d]pyrimidin-4- |
| | amine |
| 58¹ | 6-[(1S)-1-aminoethyl]-2-chloro-N-[(furan-2-yl)methyl]-7-phenylthieno[3,2-d]pyrimidin- |
| | 4-amine |
| 59¹ | 6-[(2S)-2-aminopropyl]-2-chloro-N-[(3-fluoropyridin-2-yl)methyl]-7-methylthieno[3,2- |
| | d]pyrimidin-4-amine |
| 60¹ | 6-[(2S)-2-aminopropyl]-2-chloro-N-[(2-fluoropyridin-3-yl)methyl]-7-methylthieno[3,2- |
| | d]pyrimidin-4-amine |
| 61¹ | 6-[(1S)-1-aminoethyl]-N-[(furan-2-yl)methyl]-2,7-diphenylthieno[3,2-d]pyrimidin-4- |
| | amine |
| 62¹ | 6-[(2S)-2-aminobutyl]-2-chloro-7-methyl-N-[(1,2-thiazol-5-yl)methyl]thieno[3,2- |
| | d]pyrimidin-4-amine |
| 63 | 3-(2-chloro-4-{[(furan-2-yl)methyl]amino}-7-methylthieno[3,2-d]pyrimidin-6-yl)propan- |
| | l-ol |
| 64¹ | 6-[(2S)-2-aminopropyl]-2-chloro-N-[(3,5-difluoropyridin-4-yl)methyl]-7- |
| | methylthieno[3,2-d]pyrimidin-4-amine |
| 65¹ | 6-[(2S)-2-aminopropyl]-2-chloro-N-[(furan-2-yl)methyl]-7-(4-methoxyphenyl)thieno[3,2- |
| | d]pyrimidin-4-amine |
| 66 | (2S)-3-(2-chloro-4-{[(furan-2-yl)methyl]amino}-7-methylthieno[3,2-d]pyrimidin-6-yl)-2- |
| | methylpropan-1-ol |
| 67¹ | 6-(3-aminopropyl)-2-chloro-N-[(furan-2-yl)methyl]-7-methylthieno[3,2-d]pyrimidin-4- |
| | amine |
| 68¹ | 6-[(2S)-2-aminopropyl]-7-bromo-2-chloro-N-[(3-fluoropyridin-4-yl)methyl]thieno[3,2- |
| | d]pyrimidin-4-amine |
| 69¹ | 6-[(2S)-2-aminobutyl]-2-chloro-7-methyl-N-[(1,3-oxazol-2-yl)methyl]thieno[3,2- |
| | d]pyrimidin-4-amine |
| 70¹ | 6-[(2S)-3-amino-2-methylpropyl]-2-chloro-N-[(furan-2-yl)methyl]-7-methylthieno[3,2- |
| | d]pyrimidin-4-amine |
| 71 | (2R)-3-(2-chloro-4-{[(furan-2-yl)methyl]amino}-7-methylthieno[3,2-d]pyrimidin-6-yl)- |
| | 2-methylpropan-1-ol |
| 72¹ | 6-[(2R)-3-amino-2-methylpropyl]-2-chloro-N-[(furan-2-yl)methyl]-7-methylthieno[3,2- |
| | d]pyrimidin-4-amine |
| 73¹ | 6-[(2S)-2-aminopropyl]-2-chloro-N-[(1H-imidazol-2-yl)methyl]-7-methylthieno[3,2- |
| | d]pyrimidin-4-amine |
| 74¹ | 6-[(2S)-2-aminopropyl]-2-chloro-7-methyl-N-[(1,3-thiazol-5-yl)methyl]thieno[3,2- |
| | d]pyrimidin-4-amine |
| 75¹ | 6-[(2S)-2-aminopropyl]-2-chloro-7-methyl-N-[(1,3-oxazol-5-yl)methyl]thieno[3,2- |
| | d]pyrimidin-4-amine |
| 76¹ | 6-[(2R)-2-amino-3-methoxypropyl]-2-chloro-N-[(furan-2-yl)methyl]-7-methylthieno[3,2- |
| | d]pyrimidin-4-amine |
| 77¹ | 6-[(2S)-2-aminopropyl]-2-chloro-7-ethyl-N-[(furan-2-yl)methyl]thieno[3,2-d]pyrimidin- |
| | 4-amine |
| 78¹ | 2-chloro-6-[(2S)-2-(cyclobutylamino)propyl]-N-[(furan-2-yl)methyl]-7-methylthieno[3,2- |
| | d]pyrimidin-4-amine |
| 79¹ | 2-chloro-N-[(furan-2-yl)methyl]-7-methyl-6-[(2S)-2-(mcthylamino)propylJthieno[3,2- |
| | d]pyrimidin-4-amine |
| 80 | 7-bromo-2-chloro-N-[(thiophen-2-yl)methyl]thieno[3,2-d]pyrimidin-4-amine |
| 81¹ | 6-[(2S)-2-aminopropyl]-2-chloro-7-methyl-N-[(1-methyl-1H-pyrazol-5- |
| | yl)methyl]thieno[3,2-d]pyrimidin-4-amine |
| 82¹ | 6-[(2S)-2-aminopropyl]-7-bromo-2-chloro-N-[(thiophen-2-yl)methyl]thieno[3,2- |
| | d]pyrimidin-4-amine |
| 83¹ | 6-[(2S)-2-aminopropyl]-7-bromo-4-{[(thiophen-2-yl)methyl]amino]thieno[3,2- |
| | d]pyrimidine-2-carbonitrile |
| 84 | 2-chloro-4-{[(thiophen-2-yl)methyl]amino}thieno[3,2-d]pyrimidine-7-carbonitrile |
| 85¹ | 6-[(2S)-2-aminopropyl]-4-{[(thiophen-2-yl)methyl]amino}thieno[3,2-d]pyrimidine-2,7- |
| | dicarbonitrile |
| 86¹ | 6-[(2S)-2-aminopropyl]-2-chloro-7-cyclopropyl-N-[(thiophen-2-yl)methyl]thieno[3,2- |
| | d]pyrimidin-4-amine |
| 87¹ | 6-[(2S)-2-aminopropyl]-2-chloro-7-phenyl-N-[(thiophen-2-yl)methyl]thieno[3,2- |
| | d]pyrimidin-4-amine |
| 88¹ | 6-[(2S)-2-aminopropyl]-2-chloro-7-(4-chlorophenyl)-N-[(thiophen-2- |
| | yl)methyl]thieno[3,2-d]pyrimidin-4-amine |
| 89¹ | 6-[(2S)-2-aminobutyl]-2-chloro-7-methyl-N-[(pyrimidin-4-yl)methyl]thieno[3,2- |
| | d]pyrimidin-4-amine |
| 90¹ | 6-[(2S)-2-aminopropyl]-2-chloro-N-[(3-fluorothiophen-2-yl)methyl]-7-methylthieno[3,2- |
| | d]pyrimidin-4-amine |
| 91¹ | 6-[(2S)-2-aminobutyl]-2-chloro-N-[(3-fluorothiophen-2-yl)methyl]-7-methylthieno[3,2- |
| | d]pyrimidin-4-amine |
| 92¹ | 6-[(2S)-2-aminopropyl]-2-chloro-N-[(4-fluoro-1,3-thiazol-2-yl)methyl]-7- |
| | methylthieno[3,2-d]pyrimidin-4-amine |
| 93¹ | 6-[(2S)-2-aminopropyl]-2-chloro-N-[(5-fluoro-1,3-thiazol-2-yl)methyl]-7- |
| | methylthieno[3,2-d]pyrimidin-4-amine |
| 94¹ | 6-[(1R)-1-aminoethyl]-2-chloro-N-[(furan-2-yl)methyl]-7-methylthieno[3,2-d]pyrimidin- |
| | 4-amine |
| 95 | 2-chloro-N-[(pyrimidin-4-yl)methyl]thieno[3,2-d]pyrimidin-4-amine |
| 96 | 2-chloro-N-[(1,3-thiazol-2-yl)methyl]thieno[3,2-d]pyrimidin-4-amine |
| 97¹ | (3S)-3-amino-4-(2-chloro-4-{[(furan-2-yl)methyl]amino}-7-methylthieno[3,2- |
| | d]pyrimidin-6-yl)butan-l-ol |
| 98¹ | 6-[(2S)-2-aminopropyl]-2-chloro-N-[(5-fluoropyrimidin-4-yl)methyl]-7- |
| | methylthieno[3,2-d]pyrimidin-4-amine |
| 99¹ | (2R)-2-amino-3-(2-chloro-7-methoxy-4-{[(thiophen-2-yl)methyl]amino}thieno[3,2- |
| | d]pyrimidin-6-yl)propan-l-ol |
| 100 | 6-[(2S)-2-aminopropyl]-2-chloro-7-methyl-N-[(thiophen-3-yl)methyl]thieno[3,2- |
| | d]pyrimidin-4-amine |
| 101 | 6-[(2S)-2-aminopropyl]-2-chloro-7-methyl-N-[(IH-pyrazol-5-yl)methyl]thieno[3,2- |
| | d]pyrimidin-4-amine |
| 102 | 6-[(2S)-2-aminopropyl]-2-chloro-7-methyl-N-[(1-methyl-lH-imidazol-2- |
| | yl)methyl]thieno[3,2-d]pyrimidin-4-amine |
| 103 | 6-[(2S)-2-aminopropyl]-2-chloro-N-[(furan-3-yl)methyl]-7-methylthieno[3,2- |
| | d]pyrimidin-4-amine |
| 104 | 6-[(2S)-2-aminopropyl]-2-chloro-7-methyl-N-[(IH-pyrrol-2-yl)methyl]thieno[3,2- |
| | d]pyrimidin-4-amine |
| 105 | 6-[(2S)-2-aminopropyl]-2-chloro-7-methyl-N-[(1,2-oxazol-3-yl)methyl]thieno[3,2- |
| | d]pyrimidin-4-amine |
| 106 | 6-[(2S)-2-aminopropyl]-2-chloro-7-methyl-N-[(1-methyl-lH-pyrazol-3- |
| | yl)methyl]thieno[3,2-d]pyrimidin-4-amine |
| 107 | 6-[(2S)-2-aminopropyl]-2-chloro-7-methyl-N-[(1,2-oxazol-4-yl)methyl]thieno[3,2- |
| | d]pyrimidin-4-amine |
| 108 | 6-[(2S)-2-aminopropyl]-2-chloro-7-methyl-N-[(1,2-thiazol-4-yl)methyl]thieno[3,2- |
| | d]pyrimidin-4-amine |
| 109 | 6-[(2S)-2-aminopropyl]-2-chloro-7-methyl-N-[(IH-pyrazol-4-yl)methyl]thieno[3,2- |
| | d]pyrimidin-4-amine |
| 110 | 6-[(2S)-2-aminopropyl]-2-chloro-7-methyl-N-[(1-methyl-lH-pyrazol-4- |
| | yl)methyl]thieno[3,2-d]pyrimidin-4-amine |
| 111 | 6-[(2S)-2-aminopropyl]-2-chloro-7-methyl-N-[(1,3-oxazol-2-yl)methyl]thieno[3,2- |
| | d]pyrimidin-4-amine |
| 112 | 6-[(2S)-2-aminopropyl]-2-chloro-7-methyl-N-[(1,3-oxazol-4-yl)methyl]thieno[3,2- |
| | d]pyrimidin-4-amine |
| 113 | 6-[(2S)-2-aminopropyl]-2-chloro-7-methyl-N-[(1,3-thiazol-4-yl)methyl]thieno[3,2- |
| | d]pyrimidin-4-amine |
| 114 | 6-[(2S)-2-aminopropyl]-2-chloro-N-[(IH-imidazol-5-yl)methyl]-7-methylthieno[3,2- |
| | d]pyrimidin-4-amine |
| 115 | 6-[(2S)-2-aminopropyl]-2-chloro-7-methyl-N-[(1-methyl-1H-imidazol-5- |
| | yl)methyl]thieno[3,2-d]pyrimidin-4-amine |
| 116 | 6-[(2S)-2-aminopropyl]-2-chloro-7-methyl-N-[(1-methyl-lH-imidazol-4- |
| | yl)methyl]thieno[3,2-d]pyrimidin-4-amine |
| 117 | 6-[(2S)-2-aminopropyl]-2-chloro-7-methyl-N-[(2H-1,2,3-triazol-4-yl)methyl]thieno[3,2- |
| | d]pyrimidin-4-amine |
| 118 | 6-[(2S)-2-aminopropyl]-2-chloro-7-methyl-N-[(IH-tetrazol-5-yl)methyl]thieno[3,2- |
| | d]pyrimidin-4-amine |
| 119 | 6-[(2S)-2-aminopropyl]-2-chloro-7-methyl-N-[(l-methyl-1H-pyrrol-2- |
| | yl)methyl]thieno[3,2-d]pyrimidin-4-amine |
| 120¹ | 6-[(2S)-2-aminopropyl]-2-chloro-7-methyl-N-[(IH-pyrrol-3-yl)methyl]thieno[3,2- |
| | d]pyrimidin-4-amine |
| 121¹ | 6-[(S)-(1-(1-aminoethyl)cyclopropyl)]-2-chloro-N-[(furan-2-yl)methyl]-7- |
| | methylthieno[3,2-d]pyrimidin-4-amine |
| 122¹ | 6-[(S)-(1-(amino(cyclopropyl)methyl)cyclopropyl)]-2-chloro-N-[(furan-2-yl)methyl]-7- |
| | methylthieno[3,2-d]pyrimidin-4-amine |
| 123¹ | 6-[(2R)-2-amino-2-cyclopropylethyl]-7-bromo-2-chloro-N-[(thiophen-2- |
| | yl)methyl]thieno[3,2-d]pyrimidin-4-amine,and |
| 124¹ | 6-[(2R)-2-amino-2-cyclopropylethyl]-2-chloro-7-methyl-N-[(thiophen-2- |
| | yl)methyl]thieno[3,2-d]pyrimidin-4-amine; |

wherein the form of the compound is selected from the group consisting of a salt, hydrate, solvate, racemate, enantiomer, diastereomer, stereoisomer, and tautomer form thereof.

Another aspect of the compound of Formula (I) or a form thereof is a compound salt selected from the group consisting of:

| Cpd | Name |
|---|---|
| 3 | 6-[(2S)-2-aminopropyl]-2-chloro-N-[(furan-2-yl)methyl]thieno[3,2-d]pyrimidin-4-amine |
| | dihydrochloride |
| 4 | 6-[(2S)-2-aminobutyl]-2-chloro-N-[(furan-2-yl)methyl]thieno[3,2-d]pyrimidin-4-amine |
| | dihydrochloride |
| 5 | 6-[(2R)-2-amino-3-methylbutyl]-2-chloro-N-[(furan-2-yl)methyl]thieno[3,2- |
| | d]pyrimidin-4-aminedihydrochloride |
| 6 | 6-[(2R,3S)-2-amino-3-methylpentyl]-2-chloro-N-[(furan-2-yl)methyl]thieno[3,2- |
| | d]pyrimidin-4-aminedihydrochloride |
| 7 | 6-[(2R)-2-amino-3,3-dimethylbutyl]-2-chloro-N-[(furan-2-yl)methyl]thieno[3,2- |
| | d]pyrimidin-4-aminedihydrochloride |
| 8 | 6-[(2S)-2-aminopropyl]-N-[(furan-2-yl)methyl]thieno[3,2-d]pyrimidin-4-amine |
| | dihydrochloride |
| 9 | 6-[(2S)-2-aminopropyl]-2-chloro-N-[(pyridin-4-yl)methyl]thieno[3,2-d]pyrimidin-4- |
| | aminedihydrochloride |
| 10 | 6-[(2S)-2-aminobutyl]-2-chloro-N-[(thiophen-2-yl)methyl]thieno[3,2-d]pyrimidin-4- |
| | aminedihydrochloride |
| 11 | 6-[(2S)-2-aminopropyl]-N-[(pyridin-4-yl)methyl]thieno[3,2-d]pyrimidin-4-amine |
| | dihydrochloride |
| 12 | 6-[(2S)-2-aminopropyl]-2-chloro-N-[(furan-2-yl)methyl]-7-methylthieno[3,2- |
| | d]pyrimidin-4-aminedihydrochloride |
| 13 | 6-[(2S)-2-aminobutyl]-2-chloro-N-[(furan-2-yl)methyl]-7-methylthieno[3,2- |
| | d]pyrimidin-4-aminedihydrochloride |
| 14 | 6-[(2S)-2-aminopropyl]-2-chloro-N-[(thiophen-2-yl)methyl]thieno[3,2-d]pyrimidin-4- |
| | aminedihydrochloride |
| 15 | 6-[(2S)-2-aminopropyl]-N-[(furan-2-yl)methyl]-2,7-dimethylthieno[3,2-d]pyrimidin-4- |
| | aminedihydrochloride |
| 16 | 6-[(2S)-2-aminopropyl]-2-ethyl-N-[(furan-2-yl)methyl]-7-methylthieno[3,2- |
| | d]pyrimidin-4-aminedihydrochloride |
| 17 | 6-[(2S)-2-aminopropyl]-2-cyclopropyl-N-[(furan-2-yl)methyl]-7-methylthieno[3,2- |
| | d]pyrimidin-4-aminedihydrochloride |
| 18 | 2-chloro-N-[(furan-2-yl)methyl]-7-methylthieno[3,2-d]pyrimidin-4-amine |
| | hydrochloride |
| 19 | (2R)-2-amino-3-(2-chloro-4-{[(furan-2-yl)methyl]amino}-7-methylthieno[3,2- |
| | d]pyrimidin-6-yl)propan-l-oldihydrochloride |
| 20 | 6-[(2S)-2-aminopropyl]-4-{[(furan-2-yl)methyl]amino}-7-methylthieno[3,2- |
| | d]pyrimidine-2-carboxamidetrifluoroacetate |
| 21 | 6-[(2S)-2-aminopropyl]-4-{[(furan-2-yl)methyl]amino}-7-methylthieno[3,2- |
| | d]pyrimidine-2-carbonitriletrifluoroacetate |
| 24 | 6-[(2S)-2-aminopropyl]-2-chloro-7-methyl-N-[(5-methylfuran-2-yl)methyl]thieno[3,2- |
| | d]pyrimidin-4-aminedihydrochloride |
| 26 | 6-[(2S)-2-aminopropyl]-N-[(furan-2-yl)methyl]-7-methyl-2-(trifluoromethyl)thieno[3,2- |
| | d]pyrimidin-4-aminedihydrochloride |
| 27 | 6-[(2S)-2-aminopropyl]-2-chloro-7-methyl-N-[(4-methyl-1,3-thiazol-2- |
| | yl)methyl]thieno[3,2-d]pyrimidin-4-aminedihydrochloride |
| 28 | 6-[(2S)-2-aminopropyl]-2-chloro-7-methyl-N-[(thiophen-2-yl)methyl]thieno[3,2- |
| | d]pyrimidin-4-aminedihydrochloride |
| 29 | 6-[(2S)-2-aminopropyl]-2-chloro-7-methyl-N-[(1,3-thiazol-2-yl)methyl]thieno[3,2- |
| | d]pyrimidin-4-aminetrifluoroacetate |
| 30 | 6-[(2S)-2-aminopropyl]-2-chloro-7-methyl-N-[(3-methylfuran-2-yl)methyl]thieno[3,2- |
| | d]pyrimidin-4-aminedihydrochloride |
| 31 | 6-[(2S)-2-aminopropyl]-2-chloro-7-methyl-N-[(5-methyl-1,3-thiazol-2- |
| | yl)methyl]thieno[3,2-d]pyrimidin-4-aminedihydrochloride |
| 32 | 6-[(2S)-2-aminopropyl]-2-chloro-7-methyl-N-[(pyrazin-2-yl)methyl]thieno[3,2- |
| | d]pyrimidin-4-aminedihydrochloride |
| 33 | 6-[(2S)-2-aminopropyl]-2-chloro-N-[(5-fluorothiophen-2-yl)methyl]-7- |
| | methylthieno[3,2-d]pyrimidin-4-aminedihydrochloride |
| 34 | 6-[(2S)-2-aminopropyl]-N-benzyl-2-chloro-7-methylthieno[3,2-d]pyrimidin-4-amine |
| | dihydrochloride |
| 35 | 6-[(2S)-2-aminopropyl]-2-chloro-N-[(3-fluoropyridin-4-yl)methyl]-7-methylthieno[3,2- |
| | d]pyrimidin-4-aminedihydrochloride |
| 36 | 6-[(2S)-2-aminopropyl]-2-chloro-7-cyclopropyl-N-[(furan-2-yl)methyl]thieno[3,2- |
| | d]pyrimidin-4-aminetrifluoroacetate |
| 37 | 6-[(2S)-2-aminobutyl]-2-chloro-7-methyl-N-[(thiophen-2-yl)methyl]thieno[3,2- |
| | d]pyrimidin-4-aminedihydrochloride |
| 38 | 6-[(2S)-2-aminopropyl]-2-bromo-N-[(furan-2-yl)methyl]-7-methylthieno[3,2- |
| | d]pyrimidin-4-aminetrifluoroacetate |
| 39 | 6-[(2S)-2-aminopropyl]-2-chloro-7-methyl-N-[(1,2-oxazol-5-yl)methyl]thieno[3,2- |
| | d]pyrimidin-4-aminehydrochloride |
| 40 | 6-[(2S)-2-aminopropyl]-7-bromo-2-chloro-N-[(furan-2-yl)methyl]thieno[3,2- |
| | d]pyrimidin-4-aminedihydrochloride |
| 41 | 6-(azetidin-3-yl)-2-chloro-N-[(furan-2-yl)methyl]-7-methylthieno[3,2-d]pyrimidin-4- |
| | aminetrifluoroacetate |
| 44 | 6-[(2S)-2-aminopropyl]-2-chloro-N-[(2-fluorophenyl)methyl]-7-methylthieno[3,2- |
| | d]pyrimidin-4-aminedihydrochloride |
| 45 | 6-[(2S)-2-aminopropyl]-2-chloro-7-methyl-N-[(pyridin-4-yl)methyl]thieno[3,2- |
| | d]pyrimidin-4-aminedihydrochloride |
| 46 | 6-[(1S)-1-aminoethyl]-7-bromo-2-chloro-N-[(furan-2-yl)methyl]thieno[3,2-d]pyrimidin- |
| | 4-aminehydrochloride |
| 47 | 6-[(1S)-1-aminoethyl]-2-chloro-N-[(furan-2-yl)methyl]-7-methylthieno[3,2- |
| | d]pyrimidin-4-aminehydrochloride |
| 48 | 6-[(1S)-1-aminopropyl]-2-chloro-N-[(furan-2-yl)methyl]-7-methylthieno[3,2- |
| | d]pyrimidin-4-aminehydrochloride |
| 49 | 6-[(1R)-1-aminopropyl]-2-chloro-N-[(furan-2-yl)methyl]-7-methylthieno[3,2- |
| | d]pyrimidin-4-aminehydrochloride |
| 50 | 6-[(2S)-2-aminopropyl]-2-chloro-7-methyl-N-[(pyrimidin-4-yl)methyl]thieno[3,2- |
| | d]pyrimidin-4-aminedihydrochloride |
| 51 | 6-[(2S)-2-amino-4-fluorobutyl]-2-chloro-N-[(furan-2-yl)methyl]-7-methylthieno[3,2- |
| | d]pyrimidin-4-aminedihydrochloride |
| 53 | 6-[(2S)-2-aminobutyl]-2-chloro-N-[(3-fluoropyridin-4-yl)methyl]-7-methylthieno[3,2- |
| | d]pyrimidin-4-aminedihydrochloride |
| 54 | 6-[(2S)-2-aminobutyl]-2-chloro-7-methyl-N-[(1,3-thiazol-2-yl)methyl]thieno[3,2- |
| | d]pyrimidin-4-aminedihydrochloride |
| 55 | 6-[(2S)-2-aminopropyl]-2-chloro-7-methyl-N-[(pyrimidin-2-yl)methyl]thieno[3,2- |
| | d]pyrimidin-4-aminedihydrochloride |
| 56 | (2R)-2-amino-3-(2-chloro-7-methyl-4-{[(thiophen-2-yl)methyl]amino}thieno[3,2- |
| | d]pyrimidin-6-yl)propan-l-oldihydrochloride |
| 57 | 2-chloro-N-[(furan-2-yl)methyl]-7-methyl-6-(pyrrolidin-3-yl)thieno[3,2-d]pyrimidin-4- |
| | amineformate |
| 58 | 6-[(1S)-1-aminoethyl]-2-chloro-N-[(furan-2-yl)methyl]-7-phenylthieno[3,2-d]pyrimidin- |
| | 4-amineformate |
| 59 | 6-[(2S)-2-aminopropyl]-2-chloro-N-[(3-fluoropyridin-2-yl)methyl]-7-methylthieno[3,2- |
| | d]pyrimidin-4-aminedihydrochloride |
| 60 | 6-[(2S)-2-aminopropyl]-2-chloro-N-[(2-fluoropyridin-3-yl)methyl]-7-methylthieno[3,2- |
| | d]pyrimidin-4-aminedihydrochloride |
| 61 | 6-[(15)-1-aminoethyl]-N-[(furan-2-yl)methy1]-2,7-diphenylthieno[3,2-d]pyrimidin-4- |
| | amineformate |
| 62 | 6-[(2S)-2-aminobutyl]-2-chloro-7-methyl-N-[(1,2-thiazol-5-yl)methyl]thieno[3,2- |
| | d]pyrimidin-4-amineformate |
| 64 | 6-[(2S)-2-aminopropyl]-2-chloro-N-[(3,5-difluoropyridin-4-yl)methyl]-7- |
| | methylthieno[3,2-d]pyrimidin-4-aminedihydrochloride |
| 65 | 6-[(2S)-2-aminopropyl]-2-chloro-N-[(furan-2-yl)methyl]-7-(4- |
| | methoxyphenyl)thieno[3,2-d]pyrimidin-4-amineformate |
| 67 | 6-(3-aminopropyl)-2-chloro-N-[(furan-2-yl)methyl]-7-methylthieno[3,2-d]pyrimidin-4- |
| | aminehydrochloride |
| 68 | 6-[(2S)-2-aminopropyl]-7-bromo-2-chloro-N-[(3-fluoropyridin-4-yl)methyl]thieno[3,2- |
| | d]pyrimidin-4-aminedihydrochloride |
| 69 | 6-[(2S)-2-aminobutyl]-2-chloro-7-methyl-N-[(1,3-oxazol-2-yl)methyl]thieno[3,2- |
| | d]pyrimidin-4-aminedihydrochloride |
| 70 | 6-[(2S)-3-amino-2-methylpropyl]-2-chloro-N-[(furan-2-yl)methyl]-7-methylthieno[3,2- |
| | d]pyrimidin-4-aminehydrochloride |
| 72 | 6-[(2R)-3-amino-2-methylpropyl]-2-chloro-N-[(furan-2-yl)methyl]-7-methylthieno[3,2- |
| | d]pyrimidin-4-aminehydrochloride |
| 73 | 6-[(2S)-2-aminopropyl]-2-chloro-N-[(1H-imidazol-2-yl)methyl]-7-methylthieno[3,2- |
| | d]pyrimidin-4-aminedihydrochloride |
| 74 | 6-[(2S)-2-aminopropyl]-2-chloro-7-methyl-N-[(1,3-thiazol-5-yl)methyl]thieno[3,2- |
| | d]pyrimidin-4-aminedihydrochloride |
| 75 | 6-[(2S)-2-aminopropyl]-2-chloro-7-methyl-N-[(1,3-oxazol-5-yl)methyl]thieno[3,2- |
| | d]pyrimidin-4-aminedihydrochloride |
| 76 | 6-[(2R)-2-amino-3-methoxypropyl]-2-chloro-N-[(furan-2-yl)methyl]-7- |
| | methylthieno[3,2-d]pyrimidin-4-aminedihydrochloride |
| 77 | 6-[(2S)-2-aminopropyl]-2-chloro-7-ethyl-N-[(furan-2-yl)methyl]thieno[3,2-d]pyrimidin- |
| | 4-aminehydrochloride |
| 78 | 2-chloro-6-[(2S)-2-(cyclobutylamino)propyl]-N-[(furan-2-yl)methyl]-7- |
| | methylthieno[3,2-d]pyrimidin-4-amineformate |
| 79 | 2-chloro-N-[(furan-2-yl)methyl]-7-methyl-6-[(2S)-2-(methylamino)propyl]thieno[3,2- |
| | d]pyrimidin-4-aminehydrochloride |
| 81 | 6-[(2S)-2-aminopropyl]-2-chloro-7-methyl-N-[(1-methyl-1H-pyrazol-5- |
| | yl)methyl]thieno[3,2-d]pyrimidin-4-aminehydrochloride |
| 82 | 6-[(2S)-2-aminopropyl]-7-bromo-2-chloro-N-[(thiophen-2-yl)methyl]thieno[3,2- |
| | d]pyrimidin-4-aminehydrochloride |
| 83 | 6-[(2S)-2-aminopropyl]-7-bromo-4-{[(thiophen-2-yl)methyl]amino}thieno[3,2- |
| | d]pyrimidine-2-carbonitrileformate |
| 85 | 6-[(2S)-2-aminopropyl]-4-{[(thiophen-2-yl)methyl]amino}thieno[3,2-d]pyrimidine-2,7- |
| | dicarbonitrilehydrochloride |
| 86 | 6-[(2S)-2-aminopropyl]-2-chloro-7-cyclopropyl-N-[(thiophen-2-yl)methyl]thieno[3,2- |
| | d]pyrimidin-4-amineformate |
| 87 | 6-[(2S)-2-aminopropyl]-2-chloro-7-phenyl-N-[(thiophen-2-yl)methyl]thieno[3,2- |
| | d]pyrimidin-4-aminehydrochloride |
| 88 | 6-[(2S)-2-aminopropyl]-2-chloro-7-(4-chlorophenyl)-N-[(thiophen-2- |
| | yl)methyl]thieno[3,2-d]pyrimidin-4-aminehydrochloride |
| 89 | 6-[(2S)-2-aminobutyl]-2-chloro-7-methyl-N-[(pyrimidin-4-yl)methyl]thieno[3,2- |
| | d]pyrimidin-4-aminedihydrochloride |
| 90 | 6-[(2S)-2-aminopropyl]-2-chloro-N-[(3-fluorothiophen-2-yl)methyl]-7- |
| | methylthieno[3,2-d]pyrimidin-4-aminedihydrochloride |
| 91 | 6-[(2S)-2-aminobutyl]-2-chloro-N-[(3-fluorothiophen-2-yl)methyl]-7-methylthieno[3,2- |
| | d]pyrimidin-4-aminedihydrochloride |
| 92 | 6-[(2S)-2-aminopropyl]-2-chloro-N-[(4-fluoro-1,3-thiazol-2-yl)methyl]-7- |
| | methylthieno[3,2-d]pyrimidin-4-aminedihydrochloride |
| 93 | 6-[(2S)-2-aminopropyl]-2-chloro-N-[(5-fluoro-1,3-thiazol-2-yl)methyl]-7- |
| | methylthieno[3,2-d]pyrimidin-4-aminedihydrochloride |
| 94 | 6-[(1R)-1-aminoethyl]-2-chloro-N-[(furan-2-yl)methyl]-7-methylthieno[3,2- |
| | d]pyrimidin-4-aminehydrochloride |
| 97 | (3S)-3-amino-4-(2-chloro-4-{[(furan-2-yl)methyl]amino}-7-methylthieno[3,2- |
| | d]pyrimidin-6-yl)butan-1-oldihydrochloride |
| 98 | 6-[(2S)-2-aminopropyl]-2-chloro-N-[(5-fluoropyrimidin-4-yl)methyl]-7- |
| | methylthieno[3,2-d]pyrimidin-4-amineformate |
| 99 | (27?)-2-amino-3-(2-chloro-7-methoxy-4-{[(thiophen-2-yl)methyl]amino}thieno[3,2- |
| | d]pyrimidin-6-yl)propan-l-oldihydrochloride |
| 121 | 6-[(S)-(1-(1-aminoethyl)cyclopropyl)]-2-chloro-N-[(furan-2-yl)methyl]-7- |
| | methylthieno[3,2-d]pyrimidin-4-amineformate |
| 122 | 6-[(S)-(l-(amino(cyclopropyl)methyl)cyclopropyl)]-2-chloro-N-[(furan-2-yl)methyl]-7- |
| | methylthieno[3,2-d]pyrimidin-4-amineformate |
| 123 | 6-[(2R)-2-amino-2-cyclopropylethyl]-7-bromo-2-chloro-N-[(thiophen-2- |
| | yl)methyl]thieno[3,2-d]pyrimidin-4-aminedihydrochloride,and |
| 124 | 6-[(27?)-2-amino-2-cyclopropylethyl]-2-chloro-7-methyl-N-[(thiophen-2- |
| | yl)methyl]thieno[3,2-d]pyrimidin-4-aminedihydrochloride; |

wherein the form of the compound is selected from the group consisting of hydrate, solvate, racemate, enantiomer, diastereomer, stereoisomer, and tautomer form thereof.

The present application further provides a pharmaceutical composition comprising a compound provided herein, or a pharmaceutically acceptable salt thereof, and at least one pharmaceutically acceptable carrier.

The present application further provides a method of treating familial dysautonomia, a disease of the central and peripheral nervous system associated with one or more pre-mRNA splicing defects in a subject in need thereof, the method comprising administering to the subject a therapeutically effective amount of a compound provided herein, or a pharmaceutically acceptable salt thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used.

The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### CHEMICAL DEFINITIONS

The chemical terms used above and throughout the description herein, unless specifically defined otherwise, shall be understood by one of ordinary skill in the art to have the following indicated meanings.

As used herein, the term “C₁₋₆alkyl” generally refers to saturated hydrocarbon radicals having from one to eight carbon atoms in a straight or branched chain configuration, including, but not limited to, methyl, ethyl, n-propyl (also referred to as propyl or propanyl), isopropyl, n-butyl (also referred to as butyl or butanyl), isobutyl, sec-butyl, tert-butyl, n-pentyl (also referred to as pentyl or pentanyl), n-hexyl (also referred to as hexyl or hexanyl), and the like. In certain aspects, C₁₋₆alkyl includes, but is not limited to, C₁₋₆alkyl, C₁₋₄alkyl and the like. A C₁₋₆alkyl radical is optionally substituted with substituent species as described herein where allowed by available valences.

As used herein, the terms “deutero” or “deutero-C₁₋₆alkyl” generally refer to saturated hydrocarbon radicals having from one to six carbon atoms in a straight or branched chain configuration, in which one or more carbon atom members have been substituted, where allowed by structural stability, with one or more deuterium atoms, including, but not limited to, but not limited to, deutero-methyl, deutero-ethyl, deutero-propyl, deutero-butyl, deutero-pentyl, deutero-hexyl and the like. In certain aspects, deutero-C₁₋₆alkyl includes, but is not limited to, deutero-C₁₋₄alkyl and the like. A deutero-C₁₋₆alkyl radical is optionally substituted with substituent species as described herein where allowed by available valences.

As used herein, the term “hetero-C₁₋₆alkyl” generally refers to saturated hydrocarbon radicals having from one to six carbon atoms in a straight or branched chain configuration, in which one or more heteroatoms, such as an O, S or N atom, are members in the chain, including, but not limited to, but not limited to, hetero-methyl, hetero-ethyl, hetero-propyl, hetero-butyl, hetero-pentyl, hetero-hexyl and the like. In certain aspects, hetero-C₁₋₆alkyl includes, but is not limited to, hetero-C₂₋₆alkyl, hetero-C₁₋₄alkyl, hetero-C₂₋₄alkyl and the like. A hetero-C₁₋₆alkyl radical is optionally substituted with substituent species as described herein where allowed by available valences.

As used herein, the term “C₂₋₆alkenyl” generally refers to partially unsaturated hydrocarbon radicals having from two to eight carbon atoms in a straight or branched chain configuration and one or more carbon-carbon double bonds therein, including, but not limited to, ethenyl (also referred to as vinyl), allyl, propenyl and the like. In certain aspects, C₂₋₆alkenyl includes, but is not limited to, C₂₋₆alkenyl, C₂₋₄alkenyl and the like. A C₂₋₆alkenyl radical is optionally substituted with substituent species as described herein where allowed by available valences.

As used herein, the term “C₂₋₆alkynyl” generally refers to partially unsaturated hydrocarbon radicals having from two to eight carbon atoms in a straight or branched chain configuration and one or more carbon-carbon triple bonds therein, including, but not limited to, ethynyl (also referred to as acetylenyl), propynyl, butynyl and the like. In certain aspects, C₂₋₆alkynyl includes, but is not limited to, C₂₋₆alkynyl, C₂₋₄alkynyl and the like. A C₂₋₆alkynyl radical is optionally substituted with substituent species as described herein where allowed by available valences.

As used herein, the term “C₁₋₆alkoxy” generally refers to saturated hydrocarbon radicals having from one to eight carbon atoms in a straight or branched chain configuration of the formula: —O—C₁₋₆alkyl, including, but not limited to, methoxy, ethoxy, n-propoxy, isopropoxy, n-butoxy, isobutoxy, sec-butoxy, tert-butoxy, n-pentoxy, n-hexoxy and the like. In certain aspects, C₁₋₆alkoxy includes, but is not limited to, C₁₋₆alkoxy, C₁₋₄alkoxy and the like. A C₁₋₆alkoxy radical is optionally substituted with substituent species as described herein where allowed by available valences.

As used herein, the term “oxo” refers to a radical of the formula: ═O.

As used herein, the term “carboxyl” refers to a radical of the formula: —COOH, —C(O)OH or —CO₂H.

As used herein, the term “carbamoyl” refers to a radical of the formula: —C(O)NH₂.

As used herein, the term “C₃₋₁₀cycloalkyl” generally refers to a saturated or partially unsaturated monocyclic, bicyclic or polycyclic hydrocarbon radical, including, but not limited to, cyclopropyl, cyclobutyl, cyclopentyl, cyclohexyl, cyclohexenyl, cycloheptyl, cyclooctyl, 1H-indanyl, indenyl, tetrahydro-naphthalenyl and the like. In certain aspects, C₃₋₁₀cycloalkyl includes, but is not limited to, C₃₋₈cycloalkyl, C₅₋₈cycloalkyl, C₃₋₁₀cycloalkyl and the like. A C₃₋₁₀cycloalkyl radical is optionally substituted with substituent species as described herein where allowed by available valences.

As used herein, the term “aryl” generally refers to a monocyclic, bicyclic or polycyclic aromatic carbon atom ring structure radical, including, but not limited to, phenyl, naphthyl, anthracenyl, fluorenyl, azulenyl, phenanthrenyl and the like. An aryl radical is optionally substituted with substituent species as described herein where allowed by available valences.

As used herein, the term “heteroaryl” generally refers to a monocyclic, bicyclic or polycyclic aromatic carbon atom ring structure radical in which one or more carbon atom ring members have been replaced, where allowed by structural stability, with one or more heteroatoms, such as an O, S or N atom, including, but not limited to, furanyl, thiophenyl, pyrrolyl, pyrazolyl, imidazolyl, isoxazolyl, isothiazolyl, oxazolyl, 1,3-thiazolyl, triazolyl, oxadiazolyl, thiadiazolyl, tetrazolyl, pyridinyl, pyrimidinyl, pyrazinyl, pyridazinyl, triazinyl, indolyl, indazolyl, indolizinyl, isoindolyl, benzofuranyl, benzothiophenyl, benzoimidazolyl, 1,3-benzothiazolyl, 1,3-benzoxazolyl, purinyl, quinolinyl, isoquinolinyl, quinazolinyl, quinoxalinyl and the like. A heteroaryl radical is optionally substituted on a carbon or nitrogen atom ring member with substituent species as described herein where allowed by available valences.

In certain aspects, the nomenclature for a heteroaryl radical may differ, such as in non-limiting examples where furanyl may also be referred to as furyl, thiophenyl may also be referred to as thienyl, pyridinyl may also be referred to as pyridyl, benzothiphenyl may also be referred to as benzothienyl and 1,3-benzoxazolyl may also be referred to as 1,3-benzooxazolyl.

In certain other aspects, the term for a heteroaryl radical may also include other regioisomers, such as in non-limiting examples where the term pyrrolyl may also include 2H-pyrrolyl, 3H-pyrrolyl and the like, the term pyrazolyl may also include 1H-pyrazolyl and the like, the term imidazolyl may also include 1H-imidazolyl and the like, the term triazolyl may also include 1H-1,2,3-triazolyl and the like, the term oxadiazolyl may also include 1,2,4-oxadiazolyl, 1,3,4-oxadiazolyl and the like, the term tetrazolyl may also include 1H-tetrazolyl, 2H-tetrazolyl and the like, the term indolyl may also include 1H-indolyl and the like, the term indazolyl may also include 1H-indazolyl, 2H-indazolyl and the like, the term benzoimidazolyl may also include 1H-benzoimidazolyl and the term purinyl may also include 9H-purinyl and the like.

As used herein, the term “heterocyclyl” generally refers to a saturated or partially unsaturated monocyclic, bicyclic or polycyclic carbon atom ring structure radical in which one or more carbon atom ring members have been replaced, where allowed by structural stability, with a heteroatom, such as an O, S or N atom, including, but not limited to, oxiranyl, oxetanyl, azetidinyl, tetrahydrofuranyl, pyrrolinyl, pyrrolidinyl, pyrazolinyl, pyrazolidinyl, imidazolinyl, imidazolidinyl, isoxazolinyl, isoxazolidinyl, isothiazolinyl, isothiazolidinyl, oxazolinyl, oxazolidinyl, thiazolinyl, thiazolidinyl, triazolinyl, triazolidinyl, oxadiazolinyl, oxadiazolidinyl, thiadiazolinyl, thiadiazolidinyl, tetrazolinyl, tetrazolidinyl, pyranyl, dihydro-2H-pyranyl, tetrahydropyranyl, thiopyranyl, 1,3-dioxanyl, 1,3-oxazinanyl, 1,2,5,6-tetrahydropyridinyl, 1,2,3,6-tetrahydropyridinyl, piperidinyl, piperazinyl, morpholinyl, thiomorpholinyl, 1,4-diazepanyl, 1,3-benzodioxolyl, 1,4-benzodioxanyl and the like. A heterocyclyl radical is optionally substituted on a carbon or nitrogen atom ring member with substituent species as described herein where allowed by available valences.

As used herein, the term “C₁₋₆alkyl-amino” refers to a radical of the formula: —NH—C₁₋₆alkyl.

As used herein, the term “halo-C₁₋₆alkyl-amino” refers to a radical of the formula: —NH—C₁₋₆alkyl, wherein C₁₋₆alkyl is partially or completely substituted with one or more halogen atoms where allowed by available valences.

As used herein, the term “deutero-C₁₋₆alkyl-amino” refers to a radical of the formula: —NH—C₁₋₆alkyl, wherein C₁₋₆alkyl is partially or completely substituted with one or more deuterium atoms where allowed by available valences.

As used herein, the term “(C₁₋₆alkyl)₂-amino” refers to a radical of the formula: —N(C₁₋₆alkyl)_{2.}

As used herein, the term “C₁₋₆alkyl-carboxyl-amino” refers to a radical of the formula: —NH—C(O)—.

As used herein, the term “aryl-amino” refers to a radical of the formula: —NH-aryl.

As used herein, the term “heterocyclyl-amino” refers to a radical of the formula: —NH-heterocyclyl.

As used herein, the term “heteroaryl-amino” refers to a radical of the formula: —NH-heteroaryl.

As used herein, the term “C₁₋₆alkyl-thio” refers to a radical of the formula: —S—C₁₋₆alkyl.

As used herein, the term “C₁₋₆alkyl-sulfonyl” refers to a radical of the formula: —SO₂—C₁₋₆alkyl.

As used herein, the term “halo” or “halogen” generally refers to a halogen atom radical, including fluoro, chloro, bromo and iodo.

As used herein, the term “halo-C₁₋₆alkoxy” refers to a radical of the formula: —O—C₁₋₆alkyl-halo, wherein C₁₋₆alkyl is partially or completely substituted with one or more halogen atoms where allowed by available valences.

As used herein, the term “halo-C₁₋₆alkyl” refers to a radical of the formula: —C₁₋₆alkyl-halo, wherein C₁₋₆alkyl is partially or completely substituted with one or more halogen atoms where allowed by available valences.

As used herein, the term “deutero-C₁₋₆alkyl” refers to a radical of the formula: —C₁₋₆alkyl-deutero, wherein C₁₋₆alkyl is partially or completely substituted with one or more deuterium atoms where allowed by available valences.

As used herein, the term “hydroxy” refers to a radical of the formula: —OH.

As used herein, the term “hydroxy-C₁₋₆alkyl” refers to a radical of the formula: —C₁₋₆alkyl-OH, wherein C₁₋₆alkyl is partially or completely substituted with one or more hydroxy radicals where allowed by available valences.

As used herein, the term “substituent” means positional variables on the atoms of a core molecule that are substituted at a designated atom position, replacing one or more hydrogens on the designated atom, provided that the designated atom's normal valency is not exceeded, and that the substitution results in a stable compound. Combinations of substituents and/or variables are permissible only if such combinations result in stable compounds. A person of ordinary skill in the art should note that any carbon as well as heteroatom with valences that appear to be unsatisfied as described or shown herein is assumed to have a sufficient number of hydrogen atom(s) to satisfy the valences described or shown. In certain instances, one or more substituents having a double bond (e.g., “oxo” or “═O”) as the point of attachment may be described, shown or listed herein within a substituent group, wherein the structure may only show a single bond as the point of attachment to the core structure of Formula (I). A person of ordinary skill in the art would understand that, while only a single bond is shown, a double bond is intended for those substituents.

As used herein, the term “and the like,” with reference to the definitions of chemical terms provided herein, means that variations in chemical structures that could be expected by one skilled in the art include, without limitation, isomers (including chain, branching or positional structural isomers), hydration of ring systems (including saturation or partial unsaturation of monocyclic, bicyclic or polycyclic ring structures) and all other variations where allowed by available valences which result in a stable compound.

For the purposes of this description, where one or more substituent variables for a compound of Formula (I) or a form thereof encompass functionalities incorporated into a compound of Formula (I), each functionality appearing at any location within the disclosed compound may be independently selected, and as appropriate, independently and/or optionally substituted.

As used herein, the terms “independently selected,” or “each selected” refer to functional variables in a substituent list that may occur more than once on the structure of Formula (I), the pattern of substitution at each occurrence is independent of the pattern at any other occurrence. Further, the use of a generic substituent variable on any formula or structure for a compound described herein is understood to include the replacement of the generic substituent with species substituents that are included within the particular genus, e.g., aryl may be replaced with phenyl or naphthalenyl and the like, and that the resulting compound is to be included within the scope of the compounds described herein.

As used herein, the terms “each instance of” or “in each instance, when present,” when used preceding a phrase such as “. . . C₃₋₁₀cycloalkyl, C₃₋₁₀cycloalkyl-C₁₋₄alkyl, aryl, aryl-C₁₋₄alkyl, heteroaryl, heteroaryl-C₁₋₄alkyl, heterocyclyl and heterocyclyl-C₁₋₄alkyl,” are intended to refer to the C₃₋₁₀cycloalkyl, aryl, heteroaryl and heterocyclyl ring systems when each are present either alone or as a substituent.

As used herein, the term “optionally substituted” means optional substitution with the specified substituent variables, groups, radicals or moieties.

### COMPOUND FORMS

As used herein, the term “form” means a compound of Formula (I) having a form selected from the group consisting of a free acid, free base, prodrug, salt, hydrate, solvate, clathrate, isotopologue, racemate, enantiomer, diastereomer, stereoisomer, polymorph and tautomer form thereof.

In certain aspects described herein, the form of the compound of Formula (I) is a free acid, free base or salt thereof.

In certain aspects described herein, the form of the compound of Formula (I) is a salt thereof.

In certain aspects described herein, the form of the compound of Formula (I) is an isotopologue thereof.

In certain aspects described herein, the form of the compound of Formula (I) is a stereoisomer, racemate, enantiomer or diastereomer thereof.

In certain aspects described herein, the form of the compound of Formula (I) is a tautomer thereof.

In certain aspects described herein, the form of the compound of Formula (I) is a pharmaceutically acceptable form.

In certain aspects described herein, the compound of Formula (I) or a form thereof is isolated for use.

As used herein, the term “isolated” means the physical state of a compound of Formula (I) or a form thereof after being isolated and/or purified from a synthetic process (e.g., from a reaction mixture) or natural source or combination thereof according to an isolation or purification process or processes described herein or which are well known to the skilled artisan (e.g., chromatography, recrystallization and the like) in sufficient purity to be characterized by standard analytical techniques described herein or well known to the skilled artisan.

As used herein, the term “protected” means that a functional group in a compound of Formula (I) or a form thereof is in a form modified to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, N.Y. Such functional groups include hydroxy, phenol, amino and carboxylic acid. Suitable protecting groups for hydroxy or phenol include trialkylsilyl or diarylalkylsilyl (e.g., t-butyldimethylsilyl, t-butyldiphenylsilyl or trimethylsilyl), tetrahydropyranyl, benzyl, substituted benzyl, methyl, methoxymethanol, and the like. Suitable protecting groups for amino, amidino and guanidino include t-butoxycarbonyl, benzyloxycarbonyl, and the like. Suitable protecting groups for carboxylic acid include alkyl, aryl or arylalkyl esters. In certain instances, the protecting group may also be a polymer resin, such as a Wang resin or a 2-chlorotrityl-chloride resin. Protecting groups may be added or removed in accordance with standard techniques, which are well-known to those skilled in the art and as described herein. It will also be appreciated by those skilled in the art, although such protected derivatives of compounds described herein may not possess pharmacological activity as such, they may be administered to a subject and thereafter metabolized in the body to form compounds described herein which are pharmacologically active. Such derivatives may therefore be described as “prodrugs”. All prodrugs of compounds described herein are included within the scope of the use described herein.

As used herein, the term “prodrug” means a form of an instant compound (e.g., a drug precursor) that is transformed in vivo to yield an active compound of Formula (I) or a form thereof. The transformation may occur by various mechanisms (e.g., by metabolic and/or non-metabolic chemical processes), such as, for example, by hydrolysis and/or metabolism in blood, liver and/or other organs and tissues. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

In one example, when a compound of Formula (I) or a form thereof contains a carboxylic acid functional group, a prodrug can comprise an ester formed by the replacement of the hydrogen atom of the acid group with a functional group such as alkyl and the like. In another example, when a compound of Formula (I) or a form thereof contains a hydroxyl functional group, a prodrug form can be prepared by replacing the hydrogen atom of the hydroxyl with another functional group such as alkyl, alkylcarbonyl or a phosphonate ester and the like. In another example, when a compound of Formula (I) or a form thereof contains an amine functional group, a prodrug form can be prepared by replacing one or more amine hydrogen atoms with a functional group such as alkyl or substituted carbonyl. Pharmaceutically acceptable prodrugs of compounds of Formula (I) or a form thereof include those compounds substituted with one or more of the following groups: carboxylic acid esters, sulfonate esters, amino acid esters, phosphonate esters and mono-, di- or triphosphate esters or alkyl substituents, where appropriate. As described herein, it is understood by a person of ordinary skill in the art that one or more of such substituents may be used to provide a compound of Formula (I) or a form thereof as a prodrug.

One or more compounds described herein may exist in unsolvated as well as solvated forms with pharmaceutically acceptable solvents such as water, ethanol, and the like, and the description herein is intended to embrace both solvated and unsolvated forms.

As used herein, the term “solvate” means a physical association of a compound described herein with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. As used herein, “solvate” encompasses both solution-phase and isolatable solvates. Non-limiting examples of suitable solvates include ethanolates, methanolates, and the like.

As used herein, the term “hydrate” means a solvate wherein the solvent molecule is water.

The compounds of Formula (I) can form salts, which are intended to be included within the scope of this description. Reference to a compound of Formula (I) or a form thereof herein is understood to include reference to salt forms thereof, unless otherwise indicated. The term “salt(s)”, as employed herein, denotes acidic salts formed with inorganic and/or organic acids, as well as basic salts formed with inorganic and/or organic bases. In addition, when a compound of Formula (I) or a form thereof contains both a basic moiety, such as, without limitation an amine moiety, and an acidic moiety, such as, but not limited to a carboxylic acid, zwitterions (“inner salts”) may be formed and are included within the term “salt(s)” as used herein.

The term “pharmaceutically acceptable salt(s)”, as used herein, means those salts of compounds described herein that are safe and effective (i.e., non-toxic, physiologically acceptable) for use in mammals and that possess biological activity, although other salts are also useful. Salts of the compounds of the Formula (I) may be formed, for example, by reacting a compound of Formula (I) or a form thereof with an amount of acid or base, such as an equivalent amount, in a medium such as one in which the salt precipitates or in an aqueous medium followed by lyophilization.

Pharmaceutically acceptable salts include one or more salts of acidic or basic groups present in compounds described herein. Particular aspects of acid addition salts include, and are not limited to, acetate, ascorbate, benzoate, benzenesulfonate, bisulfate, bitartrate, borate, bromide, butyrate, chloride, citrate, camphorate, camphorsulfonate, ethanesulfonate, formate, fumarate, gentisinate, gluconate, glucaronate, glutamate, iodide, isonicotinate, lactate, maleate, methanesulfonate, naphthalenesulfonate, nitrate, oxalate, pamoate, pantothenate, phosphate, propionate, saccharate, salicylate, succinate, sulfate, tartrate, thiocyanate, toluenesulfonate (also known as tosylate), trifluoroacetate salts and the like. Certain particular aspects of acid addition salts include chloride or dichloride.

Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33, 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Suitable basic salts include, but are not limited to, aluminum, ammonium, calcium, lithium, magnesium, potassium, sodium and zinc salts.

All such acid salts and base salts are intended to be included within the scope of pharmaceutically acceptable salts as described herein. In addition, all such acid and base salts are considered equivalent to the free forms of the corresponding compounds for purposes of this description.

Compounds of Formula (I) and forms thereof, may further exist in a tautomeric form. All such tautomeric forms are contemplated and intended to be included within the scope of the compounds of Formula (I) or a form thereof as described herein.

The compounds of Formula (I) or a form thereof may contain asymmetric or chiral centers, and, therefore, exist in different stereoisomeric forms. The present description is intended to include all stereoisomeric forms of the compounds of Formula (I) as well as mixtures thereof, including racemic mixtures.

The compounds described herein may include one or more chiral centers, and as such may exist as racemic mixtures (R/S) or as substantially pure enantiomers and diastereomers. The compounds may also exist as substantially pure (R) or (S) enantiomers (when one chiral center is present). In one particular aspect, the compounds described herein are (S) isomers and may exist as enantiomerically pure compositions substantially comprising only the (S) isomer. In another particular aspect, the compounds described herein are (R) isomers and may exist as enantiomerically pure compositions substantially comprising only the (R) isomer. As one of skill in the art will recognize, when more than one chiral center is present, the compounds described herein may also exist as a (R,R), (R,S), (S,R) or (S,S) isomer, as defined by IUPAC Nomenclature Recommendations.

As used herein, the term “chiral” refers to a carbon atom bonded to four nonidentical substituents. Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. In describing an optically active compound, the prefixes D and L, or R and S, are used to denote the absolute configuration of the molecule about its chiral center(s). The substituents attached to the chiral center under consideration are ranked in accordance with the Sequence Rule of Cahn, Ingold and Prelog. (Cahn et al. Angew. Chem. Inter. Edit. 1966, 5, 385; errata 511).

As used herein, the term “substantially pure” refers to compounds consisting substantially of a single isomer in an amount greater than or equal to 90%, in an amount greater than or equal to 92%, in an amount greater than or equal to 95%, in an amount greater than or equal to 98%, in an amount greater than or equal to 99%, or in an amount equal to 100% of the single isomer.

In one aspect of the description, a compound of Formula (I) or a form thereof is a substantially pure (S) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

In one aspect of the description, a compound of Formula (I) or a form thereof is a substantially pure (R) enantiomer form present in an amount greater than or equal to 90%, in an amount greater than or equal to 92%, in an amount greater than or equal to 95%, in an amount greater than or equal to 98%, in an amount greater than or equal to 99%, or in an amount equal to 100%.

As used herein, a “racemate” is any mixture of isometric forms that are not “enantiomerically pure”, including mixtures such as, without limitation, in a ratio of about 50/50, about 60/40, about 70/30, or about 80/20.

In addition, the present description embraces all geometric and positional isomers. For example, if a compound of Formula (I) or a form thereof incorporates a double bond or a fused ring, both the cis- and trans-forms, as well as mixtures, are embraced within the scope of the description. Diastereomeric mixtures can be separated into their individual diastereomers on the basis of their physical chemical differences by methods well known to those skilled in the art, such as, for example, by chromatography and/or fractional crystallization. Enantiomers can be separated by use of chiral HPLC column or other chromatographic methods known to those skilled in the art. Enantiomers can also be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this description.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this description, as are positional isomers (such as, for example, 4-pyridyl and 3-pyridyl). Individual stereoisomers of the compounds described herein may, for example, be substantially free of other isomers, or may be present in a racemic mixture, as described supra.

The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or isotopologues of the instant compounds.

The term “isotopologue” refers to isotopically-enriched compounds described herein which are identical to those recited herein, but for the fact that one or more atoms are replaced by an atom having an atomic mass or mass number different from the atomic mass or mass number usually found in nature. Examples of isotopes that can be incorporated into compounds described herein include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ²⁵S, ¹⁸F, ³⁵Cl and ³⁶Cl, respectively, each of which are also within the scope of this description.

Certain isotopically-enriched compounds described herein (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances.

Polymorphic crystalline and amorphous forms of the compounds of Formula (I) and of the salts, solvates, hydrates, esters and prodrugs of the compounds of Formula (I) are further intended to be included in the present description.

### COMPOUND USES

Provided herein are methods of treating a disease in a subject in need thereof. As used herein, the term “subject,” refers to any animal, including mammals. For example, mice, rats, other rodents, rabbits, dogs, cats, swine, cattle, sheep, horses, primates, and humans. In some aspects, the subject is a human. In some aspects, the method comprises administering to the subject a therapeutically effective amount of a compound provided herein (e.g., a compound of Formula (I)), or a pharmaceutically acceptable salt thereof. In a particular aspect, the disease is familial dysautonomia, a disease of the central and peripheral nervous system associated with one or more pre-mRNA splicing defects.

The present application further provides a method of treating familial dysautonomia in a subject in need thereof, comprising administering to the subject a therapeutically effective amount of a compound provided herein (i.e., a compound of Formula (I)).

In some aspects of the methods provided herein, the compound is selected from the group of compounds of Formula (I), or a pharmaceutically acceptable salt thereof.

In some aspects, the method of improving pre-mRNA splicing of the IKBKAP gene comprises contacting the gene (e.g., in a cell or subject expressing the gene) with a compound provided herein (e.g., a compound of Formula (I)).

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician. In some aspects, the dosage of the compound, or a pharmaceutically acceptable salt thereof, administered to a subject or individual is about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

Also provided herein are methods for increasing IKBKAP (also referred to as ELP1) protein expression in a patient in need thereof, the method comprising administering an effective amount of a compound provide herein, (i.e., a compound of Formula (I), or a pharmaceutically acceptable salt thereof), to the patient. For example, such methods include increasing IKBKAP protein expression in serum samples from the patient. Further provided herein are methods for increasing the mean percentage of IKBKAP protein expression in a patient in need thereof, the method comprising administering an effective amount of a compound provided herein (i.e., a compound of Formula (I), or a pharmaceutically acceptable salt thereof, to the patient.

Also provided herein are methods for increasing IKBKAP protein expression in a cell (e.g., ex vivo or in vivo), the method comprising contacting the cell with a therapeutically effective amount of a compound provided herein, (i.e., a compound of Formula (I), or a pharmaceutically acceptable salt thereof). In some aspects the method is an in vitro method. In some aspects, the method is an in vivo method. In some aspects, the amount IKBKAP protein expression is increased in a cell selected from the group consisting of a lung cell, a muscle cell, a liver cell, a heart cell, a brain cell, a kidney cell, and a nerve cell (e.g., a sciatic nerve cell or a trigeminal nerve cell), or any combination thereof. In some aspects thereof, the amount of IKBKAP protein expression is increased in plasma.

Also provided herein are methods for increasing IKBKAP protein level in a patient in need thereof, the method comprising administering an effective amount of a compound provide herein, (i.e., a compound of Formula (I), or a pharmaceutically acceptable salt thereof), to the patient. For example, such methods include increasing IKBKAP protein level in serum samples from the patient. Further provided herein are methods for increasing the mean percentage of IKBKAP protein level in a patient in need thereof, the method comprising administering an effective amount of a compound provided herein (i.e., a compound of Formula (I), or a pharmaceutically acceptable salt thereof, to the patient.

Also provided herein are methods for increasing IKBKAP protein level in a cell (e.g., ex vivo or in vivo), the method comprising contacting the cell with a therapeutically effective amount of a compound provided herein, (i.e., a compound of Formula (I), or a pharmaceutically acceptable salt thereof).

In some aspects, the method is an in vitro method. In some aspects, the method is an in vivo method. In some aspects, the amount IKBKAP protein level is increased in a cell selected from the group consisting of a lung cell, a muscle cell, a liver cell, a heart cell, a brain cell, a kidney cell, and a nerve cell (e.g., a sciatic nerve cell or a trigeminal nerve cell), or any combination thereof. In some aspects thereof, the amount of IKBKAP protein level is increased in plasma.

Also provided herein are methods for increasing full-length IKBKAP mRNA in a patient in need thereof, the method comprising administering an effective amount of a compound provided herein, (i.e., a compound of Formula (I), or a pharmaceutically acceptable salt thereof), to the patient. For example, such methods include increasing full-length IKBKAP mRNA concentration in serum samples from the patient. Further provided herein are methods for increasing the mean percentage exon inclusion (i.e. the percentage of correctly spliced or full-length IKBKAP mRNA) in a patient in need thereof, the method comprising administering an effective amount of a compound provided herein (i.e., a compound of Formula (I), or a pharmaceutically acceptable salt thereof, to the patient.

In some aspects, full-length IKBKAP mRNA can be measured in the serum, for example, in blood samples obtained from the patient prior to administration of a compound as provided herein and in blood samples obtained from the patient following administration of a compound as provided herein. In some aspects, the blood samples obtained from the patient following administration are obtained after one day, two days, three days, four days, five days, six days, seven days, eight days, nine days, ten days, fourteen days, twenty-one days, twenty-eight days, and/or thirty days of administration of the compound as provided herein. See, for example, F. B. Axelrod et al., Pediatr Res (2011) 70(5): 480-483; and R. S. Shetty et al., Human Molecular Genetics (2011) 20(21): 4093-4101, both of which are incorporated by reference in their entirety.

Further provided herein is a method of increasing full-length IKBKAP mRNA in a cell, the method comprising contacting the cell with a therapeutically effective amount of a compound provided herein (i.e., a compound of Formula (I)). The amount of full-length IKBKAP mRNA in the treated cell is increased relative to a cell in a subject in the absence of a compound provided herein. The method of increasing the amount of full-length IKBKAP mRNA in a cell may be performed by contacting the cell with a compound provided herein (i.e., a compound of Formula (I), or a pharmaceutically acceptable salt form thereof), in vitro, thereby increasing the amount full-length IKBKAP mRNA of a cell in vitro. Uses of such an in vitro method of increasing the amount of full-length IKBKAP mRNA include, but are not limited to, use in a screening assay (for example, wherein a compound provided herein is used as a positive control or standard compared to a compound or compounds of unknown activity or potency in increasing the amount full-length IKBKAP mRNA).

In some aspects, the amount of full-length IKBKAP mRNA is increased in a cell selected from the group consisting of a lung cell, a muscle cell, a liver cell, a heart cell, a brain cell, a kidney cell, and a nerve cell (e.g., a sciatic nerve cell or a trigeminal nerve cell), or any combination thereof. In some aspects thereof, the amount of full-length IKBKAP mRNA is increased in plasma.

The method of increasing full-length IKBKAP mRNA in a cell may be performed, for example, by contacting a cell, (e.g., a lung cell, a muscle cell, a liver cell, a heart cell, a brain cell, a kidney cell, or a nerve cell), with a compound provided herein (i.e. a compound of Formula (I), or a pharmaceutically acceptable salt thereof), in vivo, thereby increasing the amount of full-length IKBKAP mRNA in a subject in vivo. The contacting is achieved by causing a compound provided herein, or a pharmaceutically acceptable salt form thereof, to be present in a subject in an amount effective to achieve an increase in the amount of full-length IKBKAP mRNA. This may be achieved, for example, by administering an effective amount of a compound provided herein, or a pharmaceutically acceptable salt form thereof, to a subject. Uses of such an in vivo method of increasing the amount of full-length IKBKAP mRNA include, but are not limited to, use in methods of treating a disease or condition, wherein an increase in the amount of full-length IKBKAP mRNA is beneficial.

In some aspects thereof, the amount of full-length IKBKAP mRNA is increased in a cell selected from the group consisting of a lung cell, a muscle cell, a liver cell, a heart cell, a brain cell, a kidney cell, and a nerve cell (e.g., a sciatic nerve cell or a trigeminal nerve cell), or any combination thereof, for example in a patient suffering from a disease or disorder provided herein (e.g., familial dysautonomia). The method is preferably performed by administering an effective amount of a compound provided herein, or a pharmaceutically acceptable salt form thereof, to a subject who is suffering from familial dysautonomia.

In some aspects, one or more of the compounds provided herein may be administered to a subject in need thereof in combination with at least one additional pharmaceutical agent. In some embodiments, the additional pharmaceutical agent is a compound provided herein (e.g., a compound of Formula (I)).

Additional examples of suitable additional pharmaceutical agents for use in combination with the compounds of the present application for treatment of the diseases provided herein include, but are not limited to, antioxidants, anti-inflammatory agents, steroids, immunosuppressants, or other agents such as therapeutic antibodies. In some aspects, the compounds provided herein may be administered to a subject in need thereof in combination with at least one additional pharmaceutical agent for the treatment of familial dysautonomia. In some embodiments, the additional pharmaceutical agent is phosphatidylserine.

When employed as a therapeutic agent, the compounds provided herein can be administered in the form of a pharmaceutical composition; thus, the methods described herein can include administering a pharmaceutical composition. These compositions can be prepared as described herein or elsewhere, and can be administered by a variety of routes, depending upon whether local or systemic treatment is desired and upon the area to be treated. Administration may be pulmonary (e.g., by inhalation or insufflation of powders or aerosols, including by nebulizer; intratracheal or intranasal), oral, or parenteral. Parenteral administration may include, but is not limited to intravenous, intraarterial, subcutaneous, intraperitoneal, intramuscular injection or infusion; or intracranial, (e.g., intrathecal, intraocular, or intraventricular) administration. Parenteral administration can be in the form of a single bolus dose, or may be, for example, by a continuous perfusion pump. Conventional pharmaceutical carriers, aqueous, powder or oily bases, thickeners and the like may be necessary or desirable. In some aspects, the compounds provided herein are suitable for oral and parenteral administration. In some aspects, the compounds provided herein are suitable for oral administration. In some aspects, the compounds provided herein are suitable for parenteral administration. In some aspects, the compounds provided herein are suitable for intravenous administration. In some aspects, the compounds provided herein are suitable for transdermal administration (e.g., administration using a patch or microneedle). Pharmaceutical compositions for topical administration may include transdermal patches (e.g., normal or electrostimulated), ointments, lotions, creams, gels, drops, suppositories, sprays, liquids and powders. Conventional pharmaceutical carriers, aqueous, powder or oily bases, thickeners and the like may be necessary or desirable.

Also provided are pharmaceutical compositions which contain, as the active ingredient, a compound provided herein (e.g., a compound of Formula (I)), or a pharmaceutically acceptable salt thereof, in combination with one or more pharmaceutically acceptable carriers (excipients). In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include, without limitation, lubricating agents such as talc, magnesium stearate, and mineral oil; wetting agents; emulsifying and suspending agents; preserving agents such as methyl-and propylhydroxy-benzoates; sweetening agents; flavoring agents, or combinations thereof.

The active compound can be effective over a wide dosage range and is generally administered in a pharmaceutically effective amount. It will be understood, that the amount of compound to be administered and the schedule of administration will usually be determined by a physician, according to the relevant circumstances, including the condition to be treated, the chosen route of administration, the actual compound administered, the age, weight, and response of the individual subject, the severity of the subject's symptoms, and the like.

Also provided herein are kits including a compound provided herein, more particularly to a compound of Formula (I), or a pharmaceutically acceptable salt thereof. In some embodiments, a kit can include one or more delivery systems, e.g., for a compound provided herein, or a pharmaceutically acceptable salt thereof, and directions for use of the kit (e.g., instructions for treating a subject). In some embodiments, a kit can include a compound provided herein, or a pharmaceutically acceptable salt thereof, and one or more additional agents as provided herein.

In some aspects, the kit can include one or more compounds or additional pharmaceutical agents as provided herein, or a pharmaceutically acceptable salt thereof, and a label that indicates that the contents are to be administered to a subject resistant to a standard of care agent or adjuvant used for the treatment of familial dysautonomia. In some aspects, the additional pharmaceutical agent is phosphatidylserine. In another aspect, the kit can include a compound provided herein, or a pharmaceutically acceptable salt thereof, and a label that indicates that the contents are to be administered to a subject with cells expressing abnormal IKBKAP pre-mRNA splicing. In another aspect, the kit can include one or more compounds or additional pharmaceutical agents as provided herein, or a pharmaceutically acceptable salt thereof, and a label that indicates that the contents are to be administered to a subject having a disease of the central nervous system or peripheral nervous system resulting from abnormal pre-mRNA splicing.

In another aspect, the kit can include one or more compounds or additional pharmaceutical agents as provided herein, or a pharmaceutically acceptable salt thereof, and a label that indicates that the contents are to be administered to a subject having familial dysautonomia. In some aspects, a kit can include one or more compounds as provided herein, or a pharmaceutically acceptable salt thereof and a label that indicates that the contents are to be administered with one or more additional pharmaceutical agents as provided herein.

In another aspect, the concentration-biological effect relationship observed with regard to a compound of Formula (I) or a form thereof indicate a target plasma concentration ranging from approximately 0.001 μg·hr/mL to approximately 50 μg·hr/mL, from approximately 0.01 μg·hr/mL to approximately 20 μg·hr/mL, from approximately 0.05 μg·hr/mL to approximately 10 μg·hr/mL, or from approximately 0.1 μg·hr/mL to approximately 5 μg·hr/mL. To achieve such plasma concentrations, the compounds described herein may be administered at doses that vary, such as, for example, without limitation, from 1.0 ng to 10,000 mg.

In one aspect, the dose administered to achieve an effective target plasma concentration may be administered based upon subject or patient specific factors, wherein the doses administered on a weight basis may be in the range of from about 0.001 mg/kg/day to about 3500 mg/kg/day, or about 0.001 mg/kg/day to about 3000 mg/kg/day, or about 0.001 mg/kg/day to about 2500 mg/kg/day, or about 0.001 mg/kg/day to about 2000 mg/kg/day, or about 0.001 mg/kg/day to about 1500 mg/kg/day, or about 0.001 mg/kg/day to about 1000 mg/kg/day, or about 0.001 mg/kg/day to about 500 mg/kg/day, or about 0.001 mg/kg/day to about 250 mg/kg/day, or about 0.001 mg/kg/day to about 200 mg/kg/day, or about 0.001 mg/kg/day to about 150 mg/kg/day, or about 0.001 mg/kg/day to about 100 mg/kg/day, or about 0.001 mg/kg/day to about 75 mg/kg/day, or about 0.001 mg/kg/day to about 50 mg/kg/day, or about 0.001 mg/kg/day to about 25 mg/kg/day, or about 0.001 mg/kg/day to about 10 mg/kg/day, or about 0.001 mg/kg/day to about 5 mg/kg/day, or about 0.001 mg/kg/day to about 1 mg/kg/day, or about 0.001 mg/kg/day to about 0.5 mg/kg/day, or about 0.001 mg/kg/day to about 0.1 mg/kg/day, or from about 0.01 mg/kg/day to about 3500 mg/kg/day, or about 0.01 mg/kg/day to about 3000 mg/kg/day, or about 0.01 mg/kg/day to about 2500 mg/kg/day, or about 0.01 mg/kg/day to about 2000 mg/kg/day, or about 0.01 mg/kg/day to about 1500 mg/kg/day, or about 0.01 mg/kg/day to about 1000 mg/kg/day, or about 0.01 mg/kg/day to about 500 mg/kg/day, or about 0.01 mg/kg/day to about 250 mg/kg/day, or about 0.01 mg/kg/day to about 200 mg/kg/day, or about 0.01 mg/kg/day to about 150 mg/kg/day, or about 0.01 mg/kg/day to about 100 mg/kg/day, or about 0.01 mg/kg/day to about 75 mg/kg/day, or about 0.01 mg/kg/day to about 50 mg/kg/day, or about 0.01 mg/kg/day to about 25 mg/kg/day, or about 0.01 mg/kg/day to about 10 mg/kg/day, or about 0.01 mg/kg/day to about 5 mg/kg/day, or about 0.01 mg/kg/day to about 1 mg/kg/day, or about 0.01 mg/kg/day to about 0.5 mg/kg/day, or about 0.01 mg/kg/day to about 0.1 mg/kg/day, or from about 0.1 mg/kg/day to about 3500 mg/kg/day, or about 0.1 mg/kg/day to about 3000 mg/kg/day, or about 0.1 mg/kg/day to about 2500 mg/kg/day, or about 0.1 mg/kg/day to about 2000 mg/kg/day, or about 0.1 mg/kg/day to about 1500 mg/kg/day, or about 0.1 mg/kg/day to about 1000 mg/kg/day, or about 0.1 mg/kg/day to about 500 mg/kg/day, or about 0.1 mg/kg/day to about 250 mg/kg/day, or about 0.1 mg/kg/day to about 200 mg/kg/day, or about 0.1 mg/kg/day to about 150 mg/kg/day, or about 0.1 mg/kg/day to about 100 mg/kg/day, or about 0.1 mg/kg/day to about 75 mg/kg/day, or about 0.1 mg/kg/day to about 50 mg/kg/day, or about 0.1 mg/kg/day to about 25 mg/kg/day, or about 0.1 mg/kg/day to about 10 mg/kg/day, or about 0.1 mg/kg/day to about 5 mg/kg/day, or about 0.1 mg/kg/day to about 1 mg/kg/day, or about 0.1 mg/kg/day to about 0.5 mg/kg/day.

Effective amounts for a given subject may be determined by routine experimentation that is within the skill and judgment of a clinician or a practitioner skilled in the art in light of factors related to the subject. Dosage and administration may be adjusted to provide sufficient levels of the active agent(s) or to maintain the desired effect. Factors which may be taken into account include genetic screening, severity of the disease state, status of disease progression, general health of the subject, ethnicity, age, weight, gender, diet, time of day and frequency of administration, drug combination(s), reaction sensitivities, experience with other therapies, and tolerance/response to therapy.

The dose administered to achieve an effective target plasma concentration may be orally administered once (once in approximately a 24 hour period; i.e., “q.d.”), twice (once in approximately a 12 hour period; i.e., “b.i.d.” or “q.12h”), thrice (once in approximately an 8 hour period; i.e., “t.i.d.” or “q.8h”), or four times (once in approximately a 6 hour period; i.e., “q.d.s.”, “q.i.d.” or “q.6h”) daily.

In certain aspects, the dose administered to achieve an effective target plasma concentration may also be administered in a single, divided, or continuous dose for a patient or subject having a weight in a range of between about 40 to about 200 kg (which dose may be adjusted for patients or subjects above or below this range, particularly children under 40 kg). The typical adult subject is expected to have a median weight in a range of about 70 kg. Long-acting pharmaceutical compositions may be administered every 2, 3 or 4 days, once every week, or once every two weeks depending on half-life and clearance rate of the particular formulation.

The compounds and compositions described herein may be administered to the subject via any drug delivery route known in the art. Nonlimiting examples include oral, ocular, rectal, buccal, topical, nasal, sublingual, transdermal, subcutaneous, intramuscular, intraveneous (bolus and infusion), intracerebral, and pulmonary routes of administration.

In another aspect, the dose administered may be adjusted based upon a dosage form described herein formulated for delivery at about 0.02, 0.025, 0.03, 0.05, 0.06, 0.075, 0.08, 0.09, 0.10, 0.20, 0.25, 0.30, 0.50, 0.60, 0.75, 0.80, 0.90, 1.0, 1.10, 1.20, 1.25, 1.50, 1.75, 2.0, 3.0, 5.0, 10, 20, 30, 40, 50, 100, 150, 200, 250, 300, 400, 500, 1000, 1500, 2000, 2500, 3000 or 4000 mg/day.

For any compound, the effective amount can be estimated initially either in cell culture assays or in relevant animal models, such as a mouse, guinea pig, chimpanzee, marmoset or tamarin animal model. Relevant animal models may also be used to determine the appropriate concentration range and route of administration. Such information can then be used to determine useful doses and routes for administration in humans. Therapeutic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is therapeutic index, and can be expressed as the ratio, LD₅₀/ED₅₀. In certain aspects, the effective amount is such that a large therapeutic index is achieved. In further particular aspects, the dosage is within a range of circulating concentrations that include an ED₅₀ with little or no toxicity. The dosage may vary within this range depending upon the dosage form employed, sensitivity of the patient, and the route of administration.

Another aspect included within the scope of the present description are the use of in vivo metabolic products of the compounds described herein. Such products may result, for example, from the oxidation, reduction, hydrolysis, amidation, esterification and the like of the administered compound, primarily due to enzymatic processes. Accordingly, the description includes the use of compounds produced by a process comprising contacting a compound described herein with a mammalian tissue or a mammal for a period of time sufficient to yield a metabolic product thereof.

Such products typically are identified by preparing a radio-labeled isotopologue (e.g., ¹⁴C or ³H) of a compound described herein, administering the radio-labeled compound in a detectable dose (e.g., greater than about 0.5 mg/kg) to a mammal such as a rat, mouse, guinea pig, dog, monkey or human, allowing sufficient time for metabolism to occur (typically about 30 seconds to about 30 hours), and identifying the metabolic conversion products from urine, bile, blood or other biological samples. The conversion products are easily isolated since they are “radiolabeled” by virtue of being isotopically-enriched (others are isolated by the use of antibodies capable of binding epitopes surviving in the metabolite). The metabolite structures are determined in conventional fashion, e.g., by MS or NMR analysis. In general, analysis of metabolites may be done in the same way as conventional drug metabolism studies well-known to those skilled in the art. The conversion products, so long as they are not otherwise found in vivo, are useful in diagnostic assays for therapeutic dosing of the compounds described herein even if they possess no biological activity of their own.

### PREPARATION OF COMPOUNDS

### GENERAL SYNTHETIC EXAMPLES

As disclosed herein, the methods for preparing the compounds of Formula (I) or a form thereof described herein commonly use standard, well-known synthetic methodology. Many of the starting materials are commercially available or can be prepared in the Specific Synthetic Examples that follow using techniques known to those skilled in the art. Functional transformations to modify substituents may also be undertaken where chemically feasible and are considered to be included within the scope of the General Schemes and the knowledge of a person of ordinary skill in the art. Compounds of Formula (I) or a form thereof can be prepared as described in the Schemes below.

Compounds of Formula (I) may be prepared as described in Scheme A below.

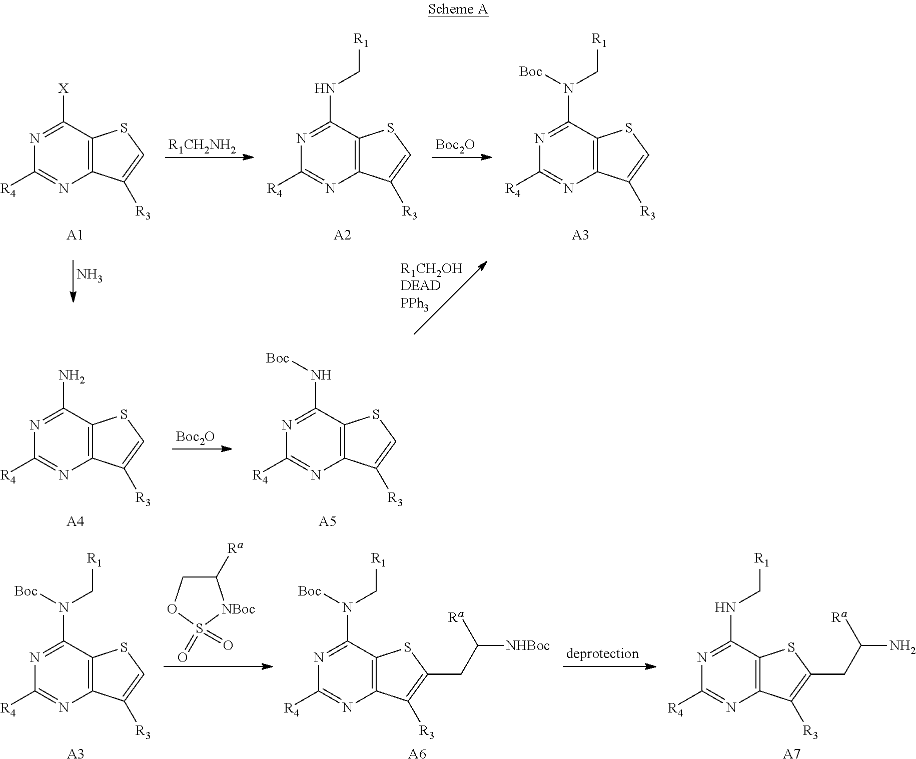

Compound A1 (X=halogen) is treated with an optionally substituted aryl/heteroarylmethylamine in the presence of a base (such as TEA and the like) using a suitable solvent (such as DMSO and the like) at an appropriate temperature to afford compound A2.

Protection of A2 with Boc₂O in the presence of DMAP as a catalyst afforded A3. Alternatively, compound Al may be treated with ammonia to give compound A4, which is followed by subsequent protection with Boc₂O in the presence of DMAP as a catalyst to give A5. Reaction of A5 with an optionally substituted aryl/heteroarylmethyl alcohol under typical Mitsunobu reaction conditions (such as DEAD/PPh₃ and the like) in a suitable solvent (such as THF and the like) affords A3.

Compound A3 can be reacted with an optionally substituted cyclic sulfamidate, prepared from the corresponding amino alcohol, in the presence of a strong base (such as LDA and the like) in a suitable solvent (such as THF and the like) at an appropriate temperature such as −78° C. to give A6. Deprotection may be accomplished by treatment with an acid (such as HCl in dioxane or TFA and the like) to afford compound A7.

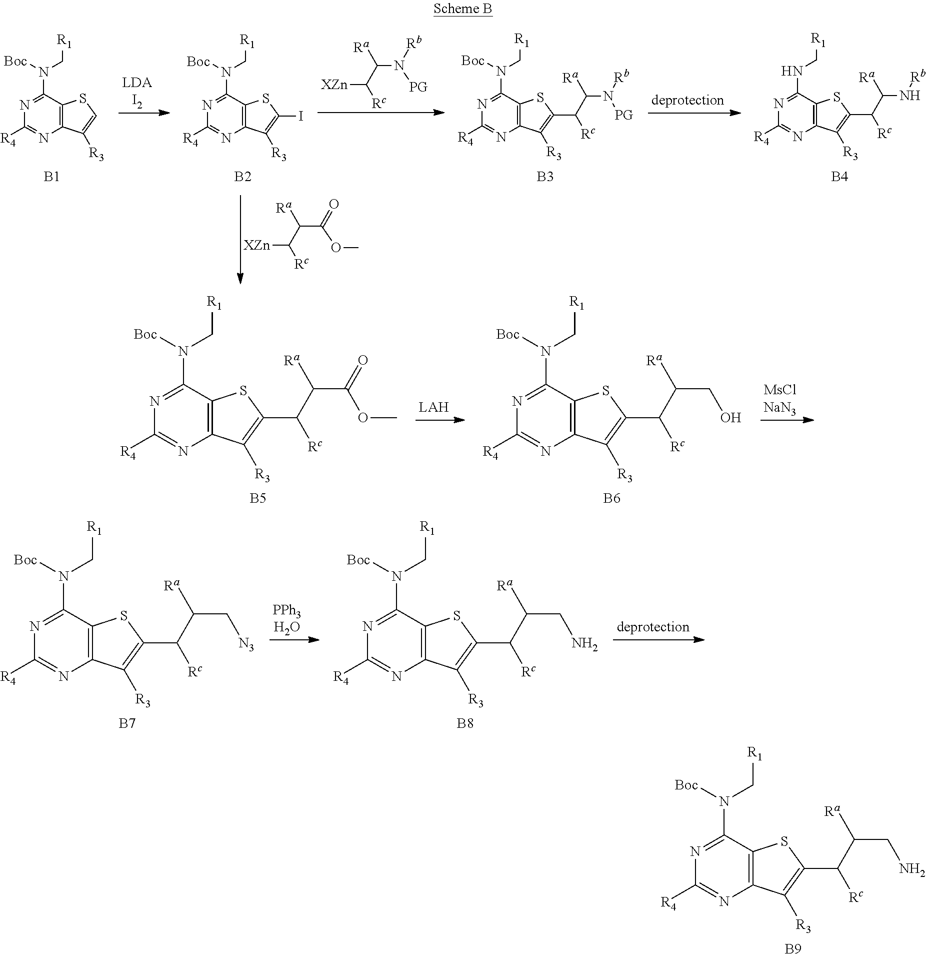

Compounds of Formula (I) may be prepared as described in Scheme B below.

Compound B1 is reacted with iodine in the presence of a strong base (such as LDA and the like) in a suitable solvent (such as THF and the like) at an appropriate temperature such as −78° C. to give B2. Compound B2 may be converted to compound B3 by a Negeshi reaction with an optionally substituted and appropriately protected amino-containing alkyl/cycloalkyl zinc reagent in the presence of a catalyst (such as Pd(dppf)Cl₂ and the like) in a suitable solvent (such as THF and the like) at an appropriate temperature. Treatment of B3 with with an acid (such as HCl in dioxane or TFA and the like) to afford the compound B4.

Alternatively, compound B2 may be converted to compound B5 by a Negeshi reaction with an optionally substituted and appropriately protected ester-containing alkyl/cycloalkyl zinc reagent in the presence of a catalyst (such as Pd(dppf)Cl₂ and the like) in a suitable solvent (such as THF and the like) at an appropriate temperature. Compound B5 may be further converted to the corresponding alcohol B6 by a reducing reagent (such as LAH and the like) in a suitable solvent (such as THF and the like). Further transformation of the alcohol B6 to the azide B7 can be achieved by reaction with mesyl chloride in the presence of a base (such as TEA and the like) in a suitable solvent (such as DCM and the like) followed by reaction with sodium azide in a suitable solvent (such as DMF and the like). Subjection of the azide B7 to the typical Staudinger reaction condition (PPh₃ in water and THF) afforded the corresponding amine B8 which may be deprotected with an acid (such as HCl in dioxane or TFA and the like) to give the compound B9.

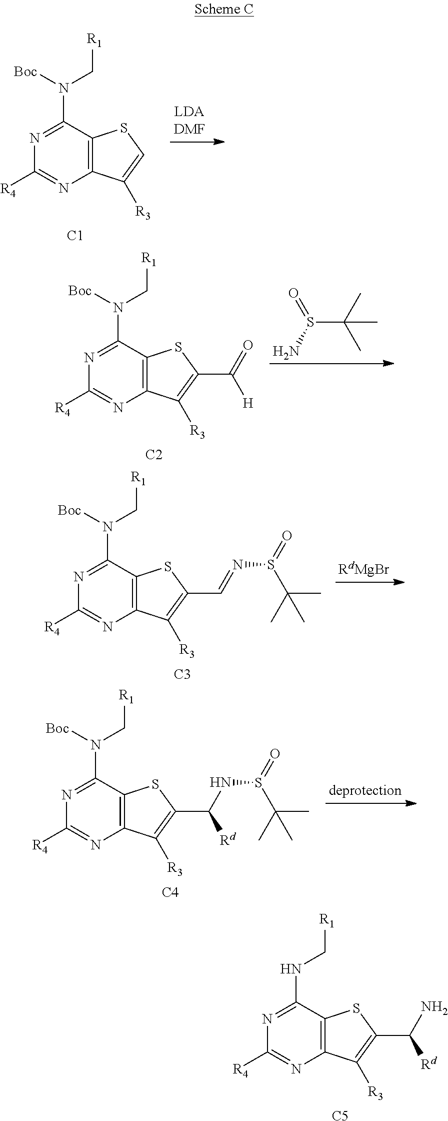

Compounds of Formula (I) may be prepared as described in Scheme C below.

Compound C1 can be converted to the corresponding aldehyde C2 by treatment with a strong base (such as LDA and the like) at an appropriate temperature such as −78° C. followed by DMF in a suitable solvent (such as THF and the like). Compound C2 may be condensed with Ellman's sulfinamide in the presence of a Lewis acid (such as CuSO₄ and the like) in a suitable solvent (such as DCE and the like) at an appropriate temperature to give compound C3. Reaction of C3 with a Grignard reagent in a suitable solvent (such as THF and the like) afforded compound C4, which may be further deprotected with an acid (such as HCl in dioxane or TFA and the like) to give the compound C5.

Compounds of Formula (I) may be prepared as described in Scheme D below.

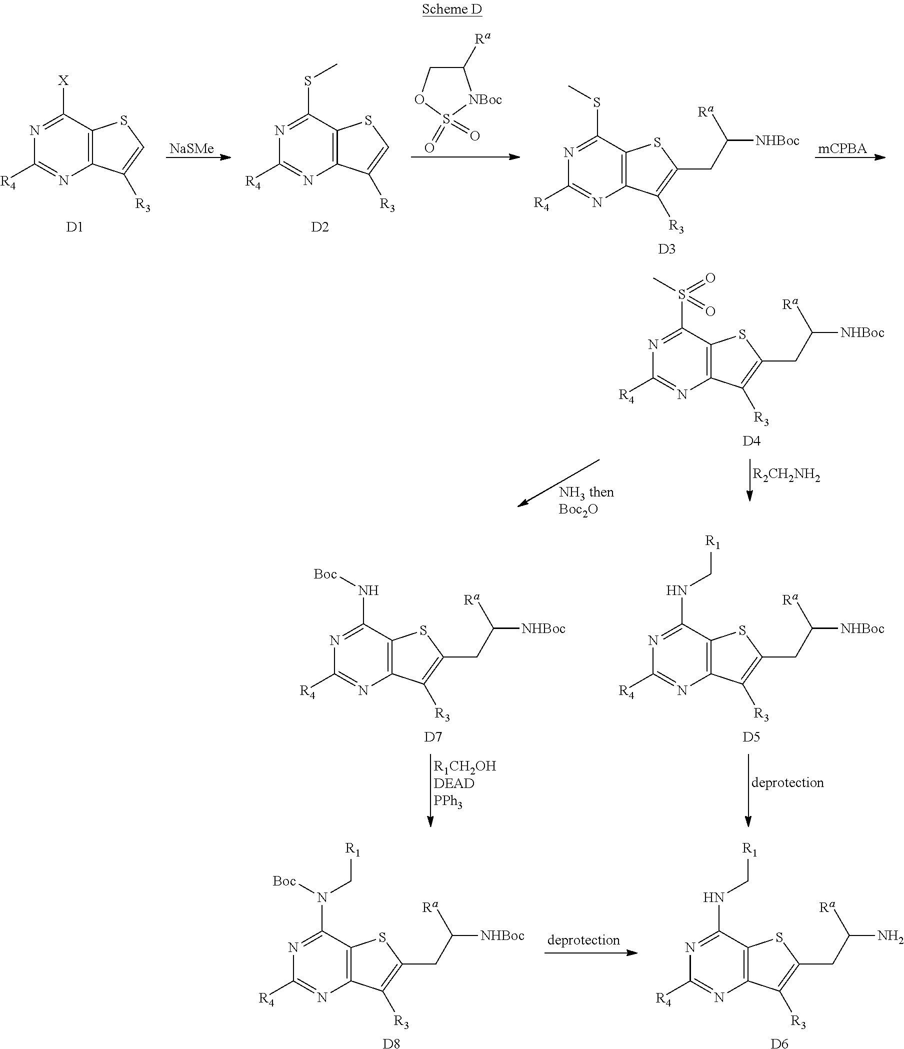

Compound D1 (X=halogen) is converted to D2 by reaction with sodium thiomethoxide in a suitable solvent (such as THF and the like) at an appropriate temperature. Compound D2 is reacted with an optionally substituted cyclic sulfamidate, prepared from the corresponding amino alcohol, in the presence of a strong base (such as LDA and the like) in a suitable solvent such as THF at an appropriate temperature such as −78° C. to give compound D3. Compound D3 is then oxidized to D4 by an oxidant (such as mCPBA and the like) in a suitable solvent (such as DCM and the like). Reaction of D4 with an optionally substituted aryl/heteroarylmethylamine in the presence of a base (such as TEA and the like) using a suitable solvent (such as DMSO and the like) at an appropriate temperature afforded compound D5. Deprotection of D5 may be effected by treatment with an acid (such as HCl in dioxane or TFA and the like) to afford compound D6.

Alternatively, compound D4 may be treated with ammonia in a solvent such as dioxane followed by subsequent protection with Boc₂O in the presence of DMAP as a catalyst to give D7. Reaction of D7 with an optionally substituted aryl/heteroarylmethyl alcohol under typical Mitsunobu reaction conditions (such as DEAD/PPh₃ and the like) in a suitable solvent (such as THF and the like) affords D8, which may be deprotected by using an acid (such as HCl in dioxane or TFA and the like) to afford compound D6.

### PREPARATION OF COMPOUNDS

### SPECIFIC SYNTHETIC EXAMPLES

To describe in more detail and assist in understanding, the following non-limiting examples are offered to more fully illustrate the scope of compounds described herein and are not to be construed as specifically limiting the scope thereof. Such variations of the compounds described herein that may be now known or later developed, which would be within the purview of one skilled in the art to ascertain, are considered to fall within the scope of the compounds as described herein and hereinafter claimed. These examples illustrate the preparation of certain compounds. Those of skill in the art will understand that the techniques described in these examples represent techniques, as described by those of ordinary skill in the art, that function well in synthetic practice, and as such constitute preferred modes for the practice thereof. However, it should be appreciated that those of skill in the art should, in light of the present disclosure, appreciate that many changes can be made in the specific methods that are disclosed and still obtain a like or similar result without departing from the spirit and scope of the present description.

Other than in the following examples of the embodied compounds, unless indicated to the contrary, all numbers expressing quantities of ingredients, reaction conditions, experimental data, and so forth used in the specification and claims are to be understood as being modified by the term “about”. Accordingly, all such numbers represent approximations that may vary depending upon the desired properties sought to be obtained by a reaction or as a result of variable experimental conditions. Therefore, within an expected range of experimental reproducibility, the term “about” in the context of the resulting data, refers to a range for data provided that may vary according to a standard deviation from the mean. As well, for experimental results provided, the resulting data may be rounded up or down to present data consistently, without loss of significant figures. At the very least, and not as an attempt to limit the application of the doctrine of equivalents to the scope of the claims, each numerical parameter should be construed in light of the number of significant digits and rounding techniques used by those of skill in the art.

While the numerical ranges and parameters setting forth the broad scope of the present description are approximations, the numerical values set forth in the examples set forth below are reported as precisely as possible. Any numerical value, however, inherently contains certain errors necessarily resulting from the standard deviation found in their respective testing measurements.

### COMPOUND EXAMPLES

As used above, and throughout the present description, the following abbreviations, unless otherwise indicated, shall be understood to have the following meanings:

| Abbreviation | Meaning |
|---|---|
| Δ | heating (chemistry), deletion (biology), |
| | lack of (biology) |
| AcOH or HOAc | acetic acid |
| Ac₂O | acetic anhydride |
| Ar | argon |
| ACN or CH₃CN | acetonitrile |
| atm | atmosphere(s) |
| Boc | tert-butoxy-carbonyl |
| Boc₂O | di-tert-butyldicarbonate |
| n-BuLi | n-butyl lithium |
| I-Bu₃PBF₄ | tri-t-butylphosphonium tetrafluoroborate |
| ° C. | degrees Celsius |
| CaCl₂ | calcium chloride |
| Celite ® or Celite | diatomaceous earth |
| CS₂CO₃ | cesium carbonate |
| CuI | copper (I) iodide |
| CuSO₄ | copper (II) sulfate |
| d/h/hr/hrs/min/s | day(d)/hour(h, hr or hrs)/minute(min)/second(s) |
| DABCO | 1,4-diazabicyclo[2.2.2]octane |
| DCE | 1,2-dichlorethane |
| DCM or CH₂Cl₂ | dichloromethane |
| DEAD | diethylazodicarboxylate |
| DMA | N,N-dimethylaniline |
| DMAP | 4-(dimethylamino)pyridineorN,N- |
| | dimethylpyridin-4-amine |
| DMF | dimethylformamide |
| DMSO | dimethylsulfoxide |
| EtOAc | ethylacetate |
| EtOH | ethanol |
| EtO | diethyl ether |
| EtZn | diethylzinc |
| eq. | equivalent(s) |
| HCl | hydrochloric acid |
| H₂SO₄ | sulfuricacid |
| K₂CO₃ | potassiumcarbonate |
| LC/MS, LCMS or | liquid chromatographic mass spectroscopy |
| LC-MS | |
| LDA | lithium diisopropylamide |
| LHMDS | lithium bis(trimethylsilyl)amide |
| mCPBA | meta-chloroperoxybenzoic acid |
| MeOH | methanol |
| MgSO₄ | magnesium sulfate |
| MS | mass spectroscopy |
| MSH | methanesulfonic acid |
| MTBE | methyl t-butyl ether |
| NBS | N-bromosuccinimide |
| NEt₃ | triethylamine |
| NH₄Cl | ammonium chloride |
| NaBH₄ | sodium borohydride |
| NaH | sodium hydride |
| NaHCO₃ | sodium bicarbonate |
| NaIO₄ | sodium periodate |
| NaOEt | sodium ethoxide |
| NaOH | sodium hydroxide |
| Na₂SO₄ | sodium sulfate |
| N₂ | nitrogen |
| NH₄C1 | ammonium chloride |
| NMM | N-methyl morpholine |
| NMR | nuclear magnetic resonance |
| Pd₂(dba)₃ | fris(dibenzylideneacetone)dipalladium(0) |
| Pd(dppf)Cl₂ or | [1,1′- |
| Pd(dppf)Cl₂—CH₂Cl₂ | bis(diphenylphosphino) |
| | ferrocene]dichloropalladium(II), |
| | complex with dichloromethane |
| PhMe or PhCH₃ | toluene |
| POCl₃ | phosphoryl chloride or |
| | phosphorous(V) oxychloride |
| psi | pounds per square inch pressure |
| PTFE | polytetrafluoroethylene |
| PPh₃ | triphenylphosphine |
| Rtorrt | room temperature |
| RT | retention time |
| RuCl₃ | ruthenium (III) chloride |
| SO₂C1₂ | sulfuryl chloride |
| TBAF | tetrabutylammonium fluoride |
| TBSCl | tert-butyldimethylsilyl chloride |
| TEA,Et₃N or NEt₃ | triethylamine |
| TFA | trifluoroacetic acid |
| THF | tetrahydrofuran |
| TLC | thin layer chromatography |
| TMS | trimethylsilyl |
| TMSCl | trimethylsilyl chloride |
| t-Bu | tert-butyl |
| XantPhos | 4,5-bis(diphenylphosphino)-9,9-dimethylxanthene |

### Intermediate 1

### tert-Butyl (S)-4-methyl-1,2,3-oxathiazolidine-3-carboxylate 2,2-dioxide

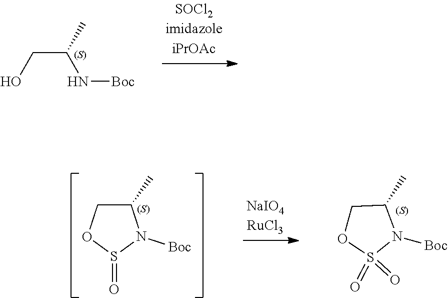

A solution of imidazole (3108 g, 45.66 mol, 8.0 eq.) in i-PrOAc (10 L) in a 30 L round bottom flask was cooled to −20-−25° C. with a dry ice/acetone bath, to which was added SOCl₂ (2040 g, 17.13 mol, 3.0 eq,) dropwise over 15 min, followed by the dropwise addition of tert-butyl (S)-(1-hydroxypropan-2-yl)carbamate (1000 g, 5.71 mol, 1.0 eq,) in i-PrOAc (10 L) over 20 min. The temperature was allowed to rise to 15-25° C., and the mixture was then stirred at that temperature for 15 h. The mixture was then poured into 7.5 kg of ice and 1.5 kg of water. The organic phase was separated and washed with brine (5 L×2). The resulting organic phase was transferred to a 50 L jacket reactor, to which MeCN (17 L) and H₂O (3 L) was added. The mixture was cooled to 4° C., to which was added RuCl₃.3H₂O (29.8 g, 0.11 mol, 0.02 eq), followed by NaIO₄ (1342 g, 6.2 mol, 1.1 eq,) in portions over 1 h while maintaining the temperature at 8-10° C. The mixture was poured into water (10 L). The organic phase was separated, washed with aq. 20% Na₂SO₃ (5 L×2) and brine (5 L×2), dried over Na₂SO₄ and then filtered through a silica gel pad. The filtrate was evaporated to give a residual solid, which was triturated with MTBE/petroleum ether (v/v=1:1, 1.2 L) for 30 min and filtered through a Buchner funnel. The solid cake was washed with petroleum ether (1 L), re-dissolved in CH₂Cl₂ (5.4 L), and the solution was filtered through a silica gel pad. The filtrate was evaporated followed by azeotropic evaporation with MTBE (1000 mL×2) at 43° C. to give tert-butyl (S)-4-methyl-1,2,3-oxathiazolidine-3-carboxylate 2,2-dioxide (1040 g, 76.8% yield) as a white solid. ¹H NMR (400 MHz, CDCl₃) δ ppm 4.69 (dd, J=9.2, 6.0 Hz, 1H), 4.47-4.40 (m, 1H), 4.22 (dd, J=8.8, 2.8 Hz, 1H), 1.57 (s, 9H), 1.53 (s, J=6.4 Hz, 3H).

### Intermediate 2

### tert-Butyl (S)-4-(((tert-butyldimethylsilyl)oxy)methyl)-1,2,3-oxathiazolidine-3-carboxylate 2,2-dioxide

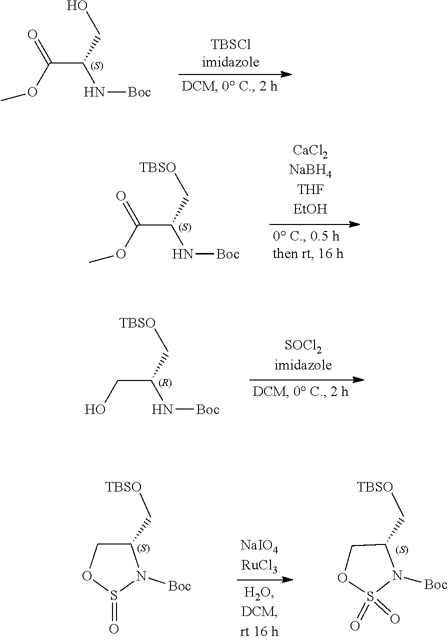

### Step 1: N-(tert-Butoxycarbonyl)-O-(tert-butyldimethylsilyl)-L-serinate

To a solution of methyl (tert-butoxycarbonyl)-L-serinate (25 g, 114.0 mmol) in CH₂Cl₂ (250 mL) was added imidazole (62.1 g, 912 mmol) and TBSCl (32 g, 205.9 mmol) at 0° C. The mixture was stirred for 2 h and then poured into a mixture of CH₂C₂ (300 mL) and water (200 mL). The organic phase was separated, washed with water (2×100 mL) and brine (1×100 mL), dried over Na₂SO₄ and filtered. The filtrate was concentrated in vacuo to give methyl N-(tert-butoxycarbonyl)-O-(tert-butyldimethylsilyl)-L-serinate (35.5 g, 93.3% yield) as an oil. LC-MS: m/z: 356.2 [M+Na]⁺.

### Step 2: tert-Butyl (R)-(1-((tert-butyldimethylsilyl)oxy)-3-hydroxypropan-2-yl)carbamate

To a solution of methyl N-(tert-butoxycarbonyl)-O-(tert-butyldimethylsilyl)-L-serinate (35.5 g, 106 mmol) in THF (200 mL) and EtOH (100 mL) was added CaCl₂ (23.6 g, 213 mmol) followed by NaBH₄ (16.1 g, 426 mmol) at 0° C. The mixture was stirred for 0.5 h at 0° C. to room temperature for 16 h and then poured into a mixture of EtOAc (200 mL) and water (150 mL). The organic phase was separated and washed with water (2×200 mL) and brine (1×150 mL), dried over anhydrous Na₂SO₄ and filtered. The filtrate was concentrated in vacuo to give tert-butyl (R)-(1-((tert-butyldimethylsilyl)oxy)-3-hydroxypropan-2-yl)carbamate (30 g, 92.3% yield) as a white solid. LC-MS: m/z: 328.2 [M+Na]⁺; RT=1.93 min.

### Step 3: tert-Butyl (4S)-4-(((tert-butyldimethylsilyl)oxy)methyl)-1,2,3-oxathiazolidine-3-carboxylate 2-oxide

To a solution of imidazole (54 g, 785.3 mmol) in CH₂Cl₂ (300 mL) at 0° C. was added SOCl₂ (12.9 mL, 176.0 mmol). The mixture was stirred at 0° C. for 1 h and tert-butyl (R)-(1-((tert-butyldimethylsilyl)oxy)-3-hydroxypropan-2-yl)carbamate (30 g, 98.2 mmol) was added. The mixture was stirred for another 1 h at 0° C., then poured into a mixture of EtOAc (500 mL) and water (400 mL). The organic layer was separated and washed with water (2×800 mL) and brine (800 mL), then dried over Na₂SO₄ and filtered. The filtrate was concentrated in vacuo to give tert-butyl (4S)-4-(((tert-butyldimethylsilyl)oxy)methyl)-1,2,3-oxathiazolidine-3-carboxylate 2-oxide (32.6 g, 94.4% yield) as a white solid. LC-MS: m/z: 374.1 [M+Na]⁺; RT=2.08 min.

### Step 4: tert-Butyl (4S)-4-[[tert-butyl(dimethyl)silyl]oxymethyl]-2,2-dioxo-oxathiazolidine-3-carboxylate

To a solution of tert-butyl (4S)-4-(((tert-butyldimethylsilyl)oxy)methyl)-1,2,3-oxathiazolidine-3-carboxylate 2-oxide (32.6 g, 92.7 mmol) in water (300 mL) and CH₂Cl₂ (300 mL) was added NaIO₄ (31.8 g, 148.0 mmol) and RuCl₃ (1.94 g, 9.3 mmol) at room temperature. The mixture was stirred overnight and then extracted with CH₂Cl₂ (3×500 mL). The combined organic phases were washed with saturated NaHSO₃ (aq. 500 mL), dried over anhydrous Na₂SO₄ and filtered. The filtrate was concentrated in vacuo. The residue was purified by flash chromatography (silica), eluting with CH₂Cl₂ hexanes (50-100%) to give tert-butyl (4S)-4-[[tert-butyl(dimethyl)silyl]oxymethyl]-2,2-dioxo-oxathiazolidine-3-carboxylate (18 g, 52.8% yield) as a white solid. LC-MS: m/z: 390.2 [M+Na]⁺; RT=2.05 min; ¹H NMR (400 MHz, CDCl₃) δ ppm 4.53-4.51 (m, 2H), 4.2 (s, 1H), 3.76-3.69 (m, 2H), 1.46 (s, 9H), 0.81 (s, 9H), 0.00 (s, 6H).

### Intermediate 3

### tert-Butyl (S)-4-(2-((tert-butyldimethylsilyl)oxy)ethyl)-1,2,3-oxathiazolidine-3-carboxylate 2,2-dioxide

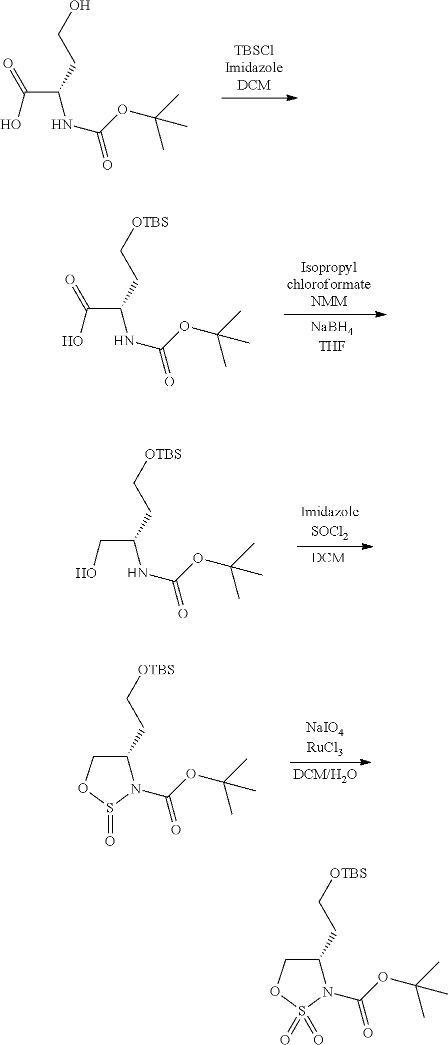 [SMILES: CC(C)(C)OC(=O)N[C@@H](CCO)C(=O)O ; CC(C)(C)OC(=O)N[C@@H](CCO[Si](C)(C)C(C)(C)C)C(=O)O ; CC(C)(C)OC(=O)N[C@H](CO)CCO[Si](C)(C)C(C)(C)C ; CC(C)(C)OC(=O)N1[C@@H](CCO[Si](C)(C)C(C)(C)C)COS1=O ; CC(C)(C)OC(=O)N1[C@@H](CCO[Si](C)(C)C(C)(C)C)COS1(=O)=O]

### Step 1: N-(tert-Butoxycarbonyl)-O-(tert-butyldimethylsilyl)-L-homoserine

To a solution of (tert-butoxycarbonyl)-L-homoserine (21 g, 96.0 mmol) and imidazole (52 g, 770 mmol) in CH₂Cl₂ (210 mL) was added TBSCl (23 g, 153 mmol). The mixture was stirred at room temperature for 5 h. Water (100 mL) was then added, and the organic phase was separated and washed with brine (80 mL), dried over anhydrous Na₂SO₄ and concentrated in vacuo to give N-(tert-butoxycarbonyl)-O-(tert-butyldimethylsilyl)-L-homoserine (31.9 g, 99% yield) as a colorless oil. ¹H NMR (400 MHz, CDCl₃) δ ppm 5.85 (d, 1H), 4.22 (m, 1H), 3.69-3.75 (m, 2H), 1.93-2.01 (m, 2H), 1.36 (s, 9H), 0.83 (s, 9H), 0.00 (s, 6H); COOH not observed.

### Step 2: tert-Butyl (S)-(4-((tert-butyldimethylsilyl)oxy)-1-hydroxybutan-2-yl)carbamate

To a solution of N-(tert-butoxycarbonyl)-O-(tert-butyldimethylsilyl)-L-homoserine (31.9 g, 96 mmol) and N-methyl morpholine (10.7 g, 105 mmol) in THF (300 mL) at 0° C. was added isopropyl chloroformate (12.8 g, 105 mmol). The mixture was stirred at 0° C. for 1 h and then filtered. The filtrate was cooled to 0° C., into which was slowly added a solution of NaBH₄ (4 g, 105.0 mmol) in water. The mixture was stirred for 2 h at 0° C., then diluted with water (100 mL). The organic phase was separated and washed with brine (2×100 mL), dried over Na₂SO₄ and filtered. The filtrate was concentrated in vacuo to give tert-butyl (S)-(4-((tert-butyldimethylsilyl)oxy)-1-hydroxybutan-2-yl)carbamate (20 g, 57% yield) as a colorless oil. ¹H NMR (400 MHz, CDCl₃) δ ppm 5.41 (s, 1H), 3.75-3.79 (m, 1H), 3.66 (t, 1H), 3.55-3.58 (m, 2H), 1.69-1.99 (m, 2H), 1.85-1.66 (m, 2H), 1.36 (s, 9H), 0.83 (s, 9H), 0.00 (s, 6H).

### Step 3: tert-Butyl (4S)-4-(2-((tert-butyldimethylsilyl)oxy)ethyl)-1,2,3-oxathiazolidine-3-carboxylate 2-oxide

To a solution of imidazole (22 g, 313 mmol) in CH₂C₁₂ (200 mL) at 0° C. was added SOCl₂ (13.5 g, 113 mmol). The mixture was stirred at room temperature for 1 h, cooled to 0° C., and a solution of tert-butyl (S)-(4-((tert-butyldimethylsilyl)oxy)-1-hydroxybutan-2-yl)carbamate (20 g, 62.7 mmol) in CH₂Cl₂ (100 mL) was added. The mixture was stirred at room temperature for 2 h and diluted with water (100 mL). The organic phase was separated and washed with brine (100 mL), dried over Na₂SO₄ and filtered. The filtrate was concentrated in vacuo to give tert-butyl (4S)-4-(2-((tert-butyldimethylsilyl)oxy)ethyl)-1,2,3-oxathiazolidine-3-carboxylate 2-oxide as a colorless oil (23 g, 99% yield). ¹H NMR (400 MHz, CDCl₃) δ ppm 3.83-4.05 (m, 1H), 3.62-3.69 (m, 2H), 3.53-3.59 (m, 2H), 1.60-1.78 (m, 2H), 1.36 (d, 9H), 0.81 (d, 9H), 0.02 (d, 6H).

### Step 4: tert-Butyl (S)-4-(2-((tert-butyldimethylsilyl)oxy)ethyl)-1,2,3-oxathiazolidine-3-carboxylate 2,2-dioxide

To a mixture of tert-butyl (4S)-4-(2-((tert-butyldimethylsilyl)oxy)ethyl)-1,2,3-oxathiazolidine-3-carboxylate 2-oxide (23 g, 62.7 mmol) and NaIO₄ (31 g, 144 mmol) in CH₂Cl₂ (300 mL) and water (310 mL) was added RuCl₃ (0.83 g, 4 mmol). The reaction was stirred at room temperature for 5 h. The organic phase was separated and washed with 10% NaHSO₃ (4×150 mL) and brine (150 mL), dried over Na₂SO₄ and filtered. The filtrate was concentrated in vacuo. The residue was purified by silica gel column chromatography, eluting with petroleum ether and ethyl acetate (20:1) to afford tert-butyl (S)-4-(2-((tert-butyldimethylsilyl)oxy)ethyl)-1,2,3-oxathiazolidine-3-carboxylate 2,2-dioxide as a white solid (5 g, 21% yield). ¹H NMR (400 MHz, CDCl₃) δ ppm 4.95 (q, 2H), 4.30-3.35 (m, 1H), 3.64-3.77 (m, 2H), 1.96-2.11 (m, 2H), 1.52 (s, 9H), 0.83 (s, 9H), 0.00 (s, 6H).

### Intermediate 4

### 7-Bromo-2,4-dichlorothieno[3,2-d]pyrimidine

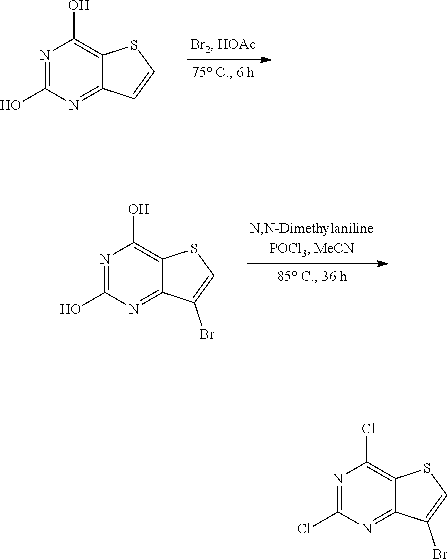 [SMILES: Oc1nc(O)c2sccc2n1 ; Oc1nc(O)c2scc(Br)c2n1 ; Clc1nc(Cl)c2scc(Br)c2n1]

### Step 1: 7-Bromothieno[3,2-d]pyrimidine-2,4-diol

To a 5 L round bottom flask at 16-20° C. under N₂ gas was added thieno[3,2-d]pyrimidine-2,4-diol (250.0 g; 1.48 mol, 1.00 eq.) and AcOH (3.0 L). The mixture was heated to 75° C., to which was added Br₂ (594.0 g, 3.71 mol, 2.5 eq.) dropwise over 60 min. The reaction continued at this temperature for 6 h before it was cooled to 16° C. The mixture was poured into a stirring solution of Na₂SO₃ (856.8 g, 6.8 mol, 5.00 eq.) in water (8 L). The mixture was filtered, and the filter cake was washed with water, then dried in the air blasting oven at 90° C. for 12 h to afford 7-bromothieno[3,2-d]pyrimidine-2,4-diol (328 g, 89% yield) as an off-white solid. LC-MS: m/z=248.9 [M+H]⁺ (83.6% purity (UV 214 nm, 1.64 min); ¹H NMR (DMSO-d₆) δ: 11.54 (s, 1H), 11.44 (s, 1H), 8.37 (s, 1H).

### Step 2: 7-Bromo-2,4-dichlorothieno[3,2-d]pyrimidine

To a mixture of 7-bromothieno[3,2-d]pyrimidine-2,4-diol (297.0 g, 1.20 mol, 1.00 eq.) in MeCN (2.7 L) at 20° C. was added N,N-Dimethylaniline (98.00 g, 0.82 mol, 0.68 eq.) and POCl₃ (1836.6 g, 5.95 mol, 5.5 eq.) dropwise over 30 min. The mixture was then heated to 85° C., stirred at that temperature for 36 h, and then cooled and poured into stirring ice (4 Kg) over 20 min. After stirring at 10° C. for an additional 30 min, the mixture was filtered, and the filter cake was washed with water and dried in the air-blasting oven at 60° C. for 12 h to afford 7-bromo-2,4-dichlorothieno[3,2-d]pyrimidine (272 g, 80% yield) as off-white solid. LC-MS: m/z 284.9 [M+H]⁺ (88.0% purity, RT=3.66 min); ¹H NMR (DMSO-d₆) δ: 8.85 (s, 1H).

### Example 1 (Compound 2)

### 2-Chloro-N-[(furan-2-yl)methyl]thieno[3,2-d]pyrimidin-4-amine

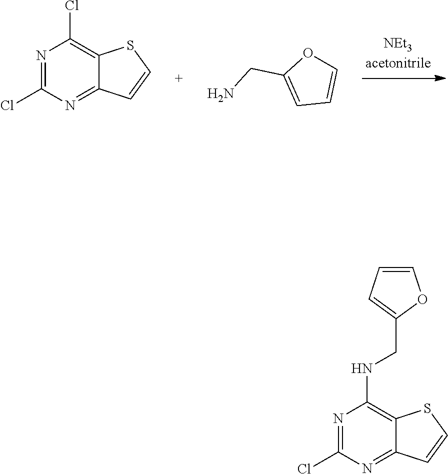 [SMILES: Clc1nc(Cl)c2sccc2n1 ; NCc1ccco1 ; Clc1nc(NCc2ccco2)c2sccc2n1]

### Step 1: 2-Chloro-N-(furan-2-ylmethyl)thieno[3,2-d]pyrimidin-4-amine

A mixture of 2,4-dichlorothieno[3,2-d]pyrimidine (59 mg, 0.28 mmol, 1.0 eq.), 2-furylmethanamine (33 mg, 0.030 ml, 0.33 mmol, 1.2 eq.) and NEt₃ (85 mg, 0.12 ml, 0.84 mmol, 3.0 eq.) in acetonitrile (0.5 ml) was stirred at room temperature for 1 h, then diluted with ethyl acetate and washed with water and brine, and dried and evaporated. The residue was purified over silica gel with ethyl acetate in dichloromethane (0 to 10% gradient) to give 2-chloro-N-(2-furylmethyl)thieno[3,2-d]pyrimidin-4-amine (71 mg, 96% yield). MS m/z 266.0, 268.0 [M+H]⁺; ¹H NMR (CDCl₃) δ: 7.77 (d, J=5.4 Hz, 1H), 7.43 (dd, J=1.6, 0.9 Hz, 1H), 7.39 (d, J=5.4 Hz, 1H), 6.40 (qd, J=3.4, 1.3 Hz, 2H), 5.43 (br s, 1H), 4.88 (d, J=5.4 Hz, 2H).

The compounds below were prepared according to the procedure of Example 1 by substituting the appropriate starting materials, reagents and reaction conditions.

| Com- | |
|---|---|
| pound | Spectral Data |
| 1 | MS m/z 275.1, 277.1 [M − H]⁻, ¹H NMR (DMSO-d₆) δ: 9.00 |
| | (br t, 6.0 Hz, 1H), 8.52 (d, J = 5.3 Hz, 2H), 8.23 (d, J = 5.4 Hz, |
| | 2H), 7.38 (d, J = 5.5 Hz, 1H), 7.33-7.34 (m, 1H), 4.72 (d, J = 6.0 |
| | Hz, 2H). |
| 18 | MS m/z 280.2, 282.2 [M + H]⁺; ¹H NMR (methanol-d4) δ: 7.90 |
| | (s, 1H), 7.48 (s, 1H), 6.37-6.46 (m, 2H), 3.68 (s, 2H), 2.44 (s, |
| | 3H); 1 NH not observed. |
| 23 | MS m/z 291.1, 293.1 [M + H]⁺; ¹H NMR (CDCl₃) δ: 8.52-8.58 |
| | (m, 2H), 7.40 (d, J = 0.9 Hz, 1H), 7.31 (d, J = 6.0 Hz, 2H), 4.80- |
| | 4.97 (m, 2H), 2.44 (d, J = 0.9 Hz, 3H); 1 NH not observed. |
| 42 | MS m/z 344.1, 346.1, 348.1 [M + H]⁺; ¹H NMR (CDCl₃) δ: |
| | 7.39-7.44 (m, 1H), 7.22 (d, J = 0.6 Hz, 1H), 6.38 (s, 2H), 5.38- |
| | 5.51 (m, 1H), 4.83 (d, J = 5.2 Hz, 2H). |
| 43 | MS m/z 355.1, 357.1, 359.1 [M + H]⁺; ¹H NMR δ: (DMSO-d6) |
| | δ: 9.22 (t, J = 5.8 Hz, 1H), 8.52 (d, J = 5.6 Hz, 2H), 8.42 (s, 1H), |
| | 7.35 (d, J = 5.8 Hz, 2H), 4.74 (d, J = 6.0 Hz, 2H).. |
| 80 | MS m/z 359.9, 361.9, 363.9 [M + H]⁺; ¹H NMR (methanol-d₄) |
| | δ: 8.10 (s, 1H), 7.30 (dd, J = 5.0, 1.1 Hz, 1H), 7.12 (d, J = 3.4 |
| | Hz, 1H), 6.97 (dd, J = 5.0, 3.5 Hz, 1H), 4.96 (s, 2H); 1 NH not |
| | observed. |
| 95 | MS m/z 278.2, 280.2 [M + H]⁺; ¹H NMR (DMSO-d₆) δ: 9.13 (d, |
| | J = 1.6 Hz, 1H), 9.05 (br s, 1H), 8.74 (d, J = 5.0 Hz, 1H), 8.25 |
| | (d, J = 5.4 Hz, 1H), 7.47 (dd, J = 5.2, 1.4 Hz, 1H), 7.39, (d, J = |
| | 5.4 Hz, 1H), 4.69-4.83 (m, 2H). |
| 96 | MS m/z 283.1, 285.1 [M + H]⁺; ¹H NMR (DMSO-d₆) δ: 9.27 |
| | (br t, J = 6.0 Hz, 1H), 8.25 (d, J = 5.4 Hz, 1H), 7.76 (d, J = 3.5 |
| | Hz, 1H), 7.64 (d, J = 3.2 Hz, 1H), 7.40 (d, J = 5.4 Hz, 1H), 4.97 |
| | (d, J = 6.0 Hz, 2H). |

### Example 2 (Compound 25)

### N-[(Furan-2-yl)methyl]-7-methyl-2-(trifluoromethyl)thieno[3,2-d]pyrimidin-4-amine

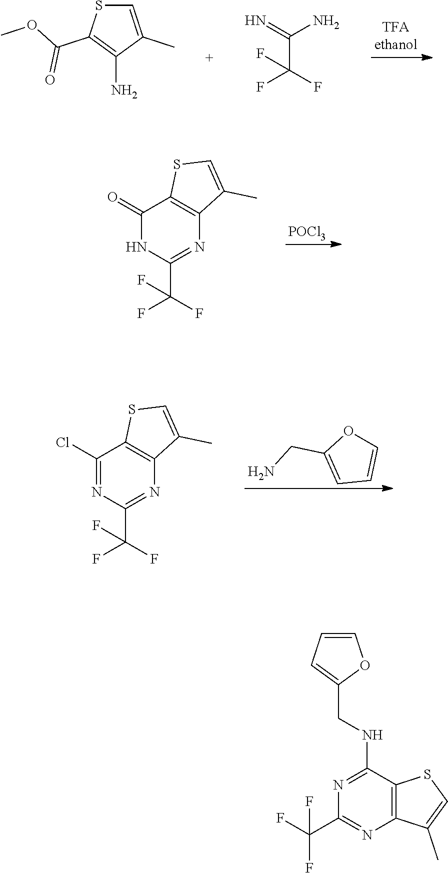

### Step 1: 7-Methyl-2-(trifluoromethyl)-3H-thieno[3,2-d]pyrimidin-4-one

A mixture of methyl 3-amino-4-methylthiophene-2-carboxylate (1770 mg, 10.0 mmol, 1.0 eq.), 2,2,2-trifluoroacetamidine (2640 mg, 20.0 mmol, 2.0 eq.) and TFA (2280 mg, 1.53 mL, 20.0 mmol, 2.0 eq.) in ethanol (12 mL) was stirred at 150° C. for 4 h and then cooled. The mixture was evaporated and the residue was treated with ethyl acetate and water. The organic layer was separated, washed with water and brine and then evaporated. The residue was purified over silica gel with methanol in dichloromethane (0 to 10% gradient) to give 7-methyl-2-(trifluoromethyl)-3H-thieno[3,2-d]pyrimidin-4-one (830 mg, 35% yield). MS m/z: 235.1 [M+H]⁺.

### Step 2: 4-Chloro-7-methyl-2-(trifluoromethyl)thieno[3,2-d]pyrimidine

A mixture of 7-methyl-2-(trifluoromethyl)-3H-thieno[3,2-d]pyrimidin-4-one (560 mg, 2.4 mmol, 1.0 eq.) and POCl₃ (4900 mg, 3.0 mL, 32 mmol, 13 eq.) was stirred at 105° C. for 8 h and then evaporated. The residue was partitioned between ethyl acetate and aq. sodium bicarbonate. The organic layer was separated, washed with brine, dried over sodium sulfate and evaporated. The residue was purified over silica gel with ethyl acetate in hexanes (3 to 15% gradient) to give 4-chloro-7-methyl-2-(trifluoromethyl)thieno[3,2-d]pyrimidine (170 mg, 28% yield). MS: m/z: 253.1, 255.1 [M+H]⁺; ¹H NMR (CDl₃) δ: 7.85 (d, J=1.1 Hz, 1H), 2.59 (d, J=0.9 Hz, 3H).

### Step 3: N-(2-Furylmethyl)-7-methyl-2-(trifluoromethyl)thieno[3,2-d]pyrimidin-4-amine

To a solution of 4-chloro-7-methyl-2-(trifluoromethyl)thieno[3,2-d]pyrimidine (170 mg, 0.67 mmol, 1.0 eq.) in acetonitrile (2.0 mL) was added furfurylamine (330 mg, 0.30 mL, 3.4 mmol, 5.0 eq.). The mixture was then stirred at 60° C. for 1 h, then cooled, diluted with ethyl acetate and washed with water and brine, and then dried and evaporated. The residue was purified over silica gel with ethyl acetate in hexanes (5 to 30% gradient) to give N-(2-furylmethyl)-7-methyl-2-(trifluoromethyl)thieno[3,2-d]pyrimidin-4-amine. MS: m/z: 314.1 [M+H]⁺. ¹H NMR (CDCl₃) δ: 7.46 (d, J=1.1 Hz, 1H), 7.42 (d, J=1.1 Hz, 1H), 6.41 (d, J=3.2 Hz, 1H), 6.38 (dd, J=3.2, 1.8 Hz, 1H), 5.41 (br s, 1H), 4.91 (d, J=5.5 Hz, 2H), 2.51 (d, J=1.1 Hz, 3H).

### Example 3 (Compound 3)

### 6-[(2S)-2-Aminopropyl]-2-chloro-N-[(furan-2-yl)methyl]thieno[3,2-d]pyrimidin-4-amine

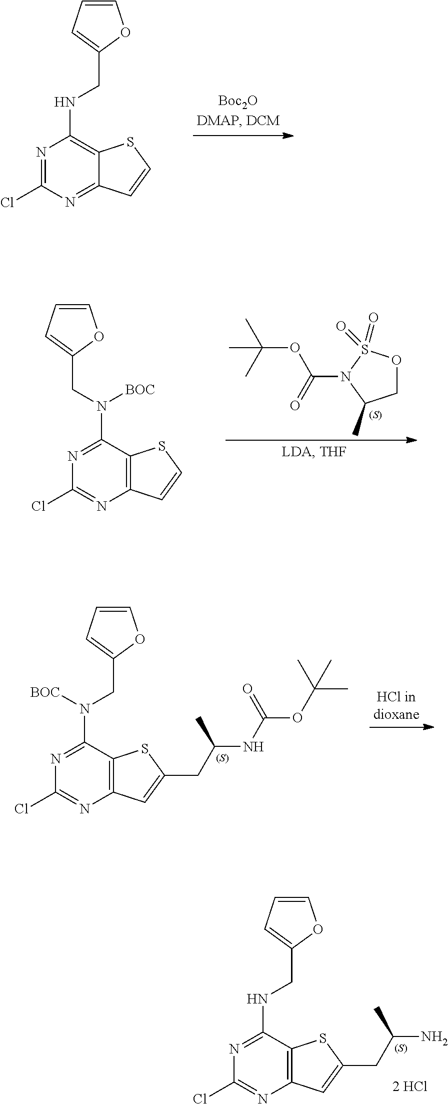

### Step 1: tert-Butyl (2-chlorothieno[3,2-d]pyrimidin-4-yl)(furan-2-ylmethyl)carbamate

A mixture of 2-chloro-N-(2-furylmethyl)thieno[3,2-d]pyrimidin-4-amine (0.265 g, 1.0 mmol, 1.0 eq.), prepared according to the procedure in example 1, was dissolved in dichloromethane (3.0 mL) followed by the addition of di-tert-butyl dicarbonate (0.467 g, 2.12 mmol, 2.0 eq.) and 4-dimethylaminopyridine (13 mg, 0.11 mmol, 0.1 eq.) and was allowed to stir for 1.5 h until completion. The reaction was then diluted with ethyl acetate and washed with water and brine, and dried and evaporated. The material was purified by silica gel column chromatography with ethyl acetate in hexanes (5 to 25% gradient) to give tert-butyl (2-chlorothieno[3,2-d]pyrimidin-4-yl)(furan-2-ylmethyl)carbamate (0.374 g, 96.6% yield). MS m/z 366.2, 368.6 [M+H]⁺; ¹H NMR (CDCl₃) δ: 7.98 (d, J=5.5 Hz, 1H), 7.43 (d, J=5.6 Hz, 1H), 6.30 (ddd, J=14.0, 3.2, 1.2 Hz, 2H), 5.24 (s, 2H), 1.54 (s, 9H).

### Step 2: tert-Butyl N-[6-[(2S)-2-(tert-butoxycarbonylamino)propyl]-2-chloro-thieno[3,2-d]pyrimidin-4-yl]-N-(2-furylmethyl)carbamate

To a solution of tert-butyl (2-chlorothieno[3,2-d]pyrimidin-4-yl)(furan-2-ylmethyl)carbamate (128 mg, 0.35 mmol, 1.0 eq.) in THF (1.0 mL) at −78° C. was added LDA (2.0 M, 0.24 mL, 0.49 mmol, 1.4 eq.) dropwise. After 15 min, a solution of tert-butyl (4S)-4-methyl-2,2-dioxo-oxathiazolidine-3-carboxylate (103 mg, 0.42 mmol, 1.2 eq.) in THF (0.5 mL) was added and the temperature was allowed to rise to 0° C. over 1 h. The reaction was quenched with 1.0 M citric acid. The mixture was stirred at room temperature for 30 min, then extracted with ethyl acetate. The organic layer was washed with water, saturated NaHCO₃ solution and brine, then dried and evaporated. The residue was purified over silica gel with ethyl acetate in dichloromethane (0 to 10% gradient) to provide tert-butyl N-[6-[(2S)-2-(tert-butoxycarbonylamino)propyl]-2-chloro-thieno[3,2-d]pyrimidin-4-yl]-N-(2-furylmethyl)carbamate (51 mg, 28% yield). MS m/z 545.2, 547.1 [M+Na]⁺; ¹H NMR (CDCl₃) δ: 7.30 (dd, J=1.8, 0.8 Hz, 1H), 7.15 (s, 1H), 6.28-6.33 (m, 2H), 5.21 (s, 2H), 4.32 -4.56 (m, 1H), 3.95-4.10 (m, 1H), 3.14 (d, J=5.0 Hz, 2H), 1.54 (s, 9H), 1.43-1.47 (m, 9H), 1.19 (d, J=6.7 Hz, 3H).

### Step 3: tert-Butyl N-[(1S)-2-(2,4-dichlorothieno[3,2-d]pyrimidin-6-yl)-1-methyl-ethyl]carbamate

General Boc removal procedure using HCl in dioxane. A mixture of tert-butyl N-[6-[(2S)-2-(tert-butoxycarbonylamino)propyl]-2-chloro-thieno[3,2-d]pyrimidin-4-yl]-N-(2-furylmethyl)carbamate (21 mg, 0.040 mmol), anisole (0.25 mL) and HCl in dioxane (4.0 N, 1.0 mL) was stirred at room temperature for 1 h, then diluted with ether and filtered. The solid was collected and dried to give tert-butyl N-[(1S)-2-(2,4-dichlorothieno[3,2-d]pyrimidin-6-yl)-1-methyl-ethyl]carbamate (15 mg, 94% yield). MS m/z 323.2, 325.2 [M+H]⁺; ¹H NMR (methanol-d₄) δ: 7.46-7.50 (m, 1H), 7.29-7.31 (m, 1H), 6.38-6.43 (m, 2H), 4.85 (s, 2H), 3.69-3.75 (m, 1H), 3.35-3.41 (m, 1H), 3.27-3.31 (m, 1H), 1.40 (d, J=6.6 Hz, 3H); 3 NHs not observed.

The compounds below were prepared according to the procedure of Example 3 by substituting the appropriate starting materials, reagents and reaction conditions.

| Com- | |
|---|---|
| pound | Spectral Data |
| 4 | MS m/z 337.3, 339.3 [M + H]⁺; ¹H NMR (methanol-d₄) δ: 7.47 |
| | (d, J = 1.07 Hz, 1H), 7.32 (s, 1H), 6.41 (d, J = 1.00 Hz, 1H), |
| | 6.39 (d, J = 1.83 Hz, 1H), 3.57 (quin, J = 6.56 Hz, 1H), 3.39- |
| | 3.44 (m, 1H), 3.34 (d, J = 7.32 Hz, 1H), 1.69-1.85 (m, 2H), 1.09 |
| | (t, J = 7.55 Hz, 3H); 2 Hs obscured by water peak, 3 NHs not |
| | observed. |
| 5 | MS m/z 351.3, 353.3 [M + H]⁺; ¹H NMR (methanol-d₄) δ: 7.47 |
| | (d, J = 1.22 Hz, 1H), 7.39-7.41 (m, 1H), 6.43 (d, J = 3.20 Hz, |
| | 1H), 6.40 (d, J = 1.83 Hz, 1H), 4.88 (s, 2H), 3.51-3.56 (m, 1H), |
| | 3.43-3.49 (m, 1H), 3.32-3.36 (m, 1H), 2.04-2.13 (m, 1H), 1.12 |
| | (t, J = 7.48 Hz, 6H); 3 NHs not observed. |
| 6 | MS m/z 365.4, 367.4 [M + H]⁺; ¹H NMR (methanol-d₄) δ: 7.47 |
| | (d, J = 1.07 Hz, 1H), 7.35 (s, 1H), 6.41-6.43 (m, 1H), 6.39 (d, |
| | J = 1.83 Hz, 1H), 3.60 (dt, J = 9.12, 4.52 Hz, 1H), 3.42 (dd, |
| | J = 15.72, 4.58 Hz, 1H), 3.27 (d, J = 6.26 Hz, 1H), 1.76-1.91 |
| | (m, 1H), 1.58-1.70 (m, 1H), 1.24-1.36 (m, 1H), 1.10 (d, J = 7.02 |
| | Hz, 3H), 1.02 (t, J = 7.32 Hz, 3H); 2 Hs obscured by water |
| | peak, 3 NHs not observed. |
| 7 | MS m/z 365.4, 367.4 [M + H]⁺; ¹H NMR (methanol-d₄) δ: 7.47 |
| | (d, J = 1.22 Hz, 1H), 7.40 (s, 1H), 6.42 (d, J = 3.05 Hz, 1H), |
| | 6.39 (d, J = 1.83 Hz, 1H), 3.57 (dd, J = 15.79, 2.37 Hz, 1H), |
| | 3.46 (br dd, J = 10.76, 2.52 Hz, 1H), 3.22 (dd, J = 15.79, 10.60 |
| | Hz, 1H), 1.15 (s, 9H); 2 Hs obscured by water peak, 3 NHs not |
| | observed. |
| 9 | MS m/z 334.3, 336.3 [M + H]⁺; ¹H NMR (methanol-d₄) δ: 8.82 |
| | (d, J = 6.56 Hz, 2H), 8.11 (d, J = 6.26 Hz, 2H), 7.29 (s, 1H), |
| | 5.11 (s, 2H), 3.69-3.75 (m, 1H), 3.36-3.42 (m, 1H), 3.27-3.29 |
| | (m, 1H), 1.40 (d, J = 6.56 Hz, 3H); 3 NHs not observed. |
| 10 | MS m/z 353.3, 355.3 [M + H]⁺; ¹H NMR (methanol-d₄) δ: 7.32- |
| | 7.36 (m, 1H), 7.30-7.32 (m, 1H), 7.14 (br d, J = 2.75 Hz, 1H), |
| | 6.98 (dd, J = 4.96, 3.59 Hz, 1H), 5.01 (s, 2H), 3.52-3.61 (m, |
| | 1H), 3.37-3.45 (m, 1H), 1.69-1.85 (m, 2H), 1.09 (t, J = 7.50 Hz, |
| | 3H); 1 H obscured by MeOD peak, 3 NHs not observed. |
| 12 | MS m/z 337.3, 339.3 [M + H]⁺; ¹H NMR (methanol-d4) δ: 7.44- |
| | 7.51 (m, 1H), 6.35-6.46 (m, 2H), 4.86 (s, 2H), 3.62-3.71 (m, |
| | 1H), 3.36-3.43 (m, 1H), 3.22-3.30 (m, 1H), 2.41 (s, 3H), 1.38 |
| | (d, J = 6.6 Hz, 3H); 3 NHs not observed. |
| 13 | MS m/z 351.1, 353.4 [M + H]⁺; ¹H NMR (methanol-d4) δ: 7.36- |
| | 7.44 (m, 1H), 6.31 (s, 2H), 4.71-4.75 (m, 2H), 3.40-3.49 (m, |
| | 1H), 3.17-3.25 (m, 2H), 2.31 (s, 3H), 1.63-1.79 (m, 2H), 1.01- |
| | 1.07 (m, 3H); 3 NHs not observed. |
| 14 | MS m/z 339.2, 341.2 [M + H]⁺; ¹H NMR (methanol-d₄) δ: 8.20 |
| | (dd, J = 5.11, 0.99 Hz, 1H), 8.08 (s, 1H), 7.88 (d, J = 3.20 Hz, |
| | 1H), 7.78 (dd, J = 5.04, 3.51 Hz, 1H), 5.62 (d, J = 5.80 Hz, 2H), |
| | 4.28-4.35 (m, 1H), 4.09 (dd, J = 14.65, 5.95 Hz, 1H), 3.96 (dd, |
| | J = 14.65, 7.78 Hz, 1H), 2.04 (d, J = 6.41 Hz, 3H); 3 NHs not |
| | observed. |
| 26 | MS m/z 371.2 [M + H]⁺; ¹H NMR (methanol-d4) δ: 7.43 (t, J = |
| | 1.3 Hz, 1H), 6.33-6.38 (m, 2H), 4.81 (s, 2H), 3.62-3.72 (m, 1H), |
| | 3.34-3.40 (m, 1H), 3.21-3.28 (m, 1H), 2.43 (s, 3H), 1.38 (d, |
| | J = 6.6 Hz, 3H); 3 NHs not observed. |
| 36 | MS m/z 363.2, 365.2 [M + H]⁺; ¹H NMR (methanol-d₄) δ: 7.42 |
| | (dd, J = 1.83, 0.76 Hz, 1H), 6.34 (dd, J = 3.20, 1.83 Hz, 1H), |
| | 6.31 (dd, J = 3.20, 0.76 Hz, 1H), 4.72 (s, 2H), 3.68-3.71 (m, |
| | 1H), 3.33-3.37 (m, 1H), 3.25-3.28 (m, 1H), 1.82-1.86 (m, 1H), |
| | 1.36 (d, J = 6.56 Hz, 3H), 1.17-1.20 (m, 2H), 0.99-1.01 |
| | (m, 2H); 3 NHs not observed. |
| 38 | MS m/z 381.0, 383.0 [M + H]⁺; ¹H NMR (methanol-d4) δ: 7.44 |
| | (dd, J = 1.7, 0.8 Hz, 1H), 6.32-6.40 (m, 2H), 4.75 (s, 2H), 3.58- |
| | 3.67 (m, 1H), 3.27-3.31 (m, 1H), 3.14-3.22 (m, 1H), 2.33 (s, |
| | 3H), 1.36 (d, J = 6.6 Hz, 3H); 3 NHs not observed. |
| 40 | MS m/z 401.0, 403.0, 405.1 [M + H]⁺; ¹H NMR (methanol-d4) |
| | δ: 7.46 (s, 1H), 6.38 (s, 2H), 4.79 (s, 2H), 3.73-3.79 (m, 1H), |
| | 3.37-3.44 (m, 1H), 3.34-3.35 (m, 1H), 1.40 (d, J = 6.6 Hz, 3H); |
| | 3 NHs not observed. |
| 82 | MS m/z 417.0, 419.0, 421.0 [M + H]⁺; ¹H NMR (methanol-d₄) |
| | δ: 7.31 (dd, J = 5.0, 0.8 Hz, 1H), 7.12 (br d, J = 3.1 Hz, 1H), |
| | 6.97 (dd, J = 5.0, 3.5 Hz, 1H), 4.95 (s, 2H), 3.71-3.80 (m, 1H), |
| | 3.44-3.57 (m, 1H), 3.36-3.42 (m, 1H), 1.39 (d, J = 6.7 Hz, 3H); |
| | 3 NHs not observed. |
| 123 | MS m/z 443.0, 445.0, 447.0 [M + H]⁺; ¹H NMR (methanol-d₄) |
| | δ: 7.30 (d, 7 = 5 Hz, 1H), 7.11 (s, 1H), 6.97 (t, 7 = 5 Hz, 1H), |
| | 4.95 (s, 2H), 3.50-3.58 (m, 2H), 2.85-2.91 (m, 1H), 1.05-1.10 |
| | (m, 1H), 0.60-0.75 (m, 2H), 0.45-0.52 (m, 1H), 0.16-0.22 (m, |
| | 1H). 3 NHs not observed. |
| 124 | MS m/z 379.1, 381.1 [M + H]⁺; ¹H NMR (methanol-d₄) δ: 7.33 |
| | (d, J = 5 Hz, 1H), 7.15 (s, 1H), 6.99 (t, 7 = 5 Hz, 1H), 5.01 (s, |
| | 2H), 3.45-3.51 (m, 2H), 2.75-2.85 (m, 1H), 2.41 (s, 3H), 1.00- |
| | 1.15 (m, 1H), 0.80-0.75 (m, 2H), 0.49-0.52 (m, 1H), 0.10-0.20 |
| | (m, 1H). 3 NHs not observed |

### Example 4 (Compound 19)

### (2R)-2-Amino-3-(2-chloro-4-{[(furan-2-yl)methyl]amino}-7-methylthieno[3,2-d]pyrimidin-6-yl)propan-1-ol dihydrochloride

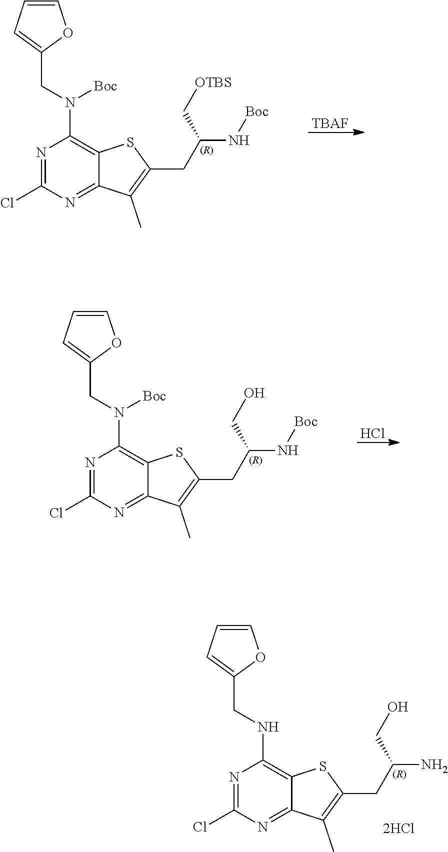

### Step 1: tert-Butyl N-[(1R)-1-[[2-chloro-4-(2-furylmethylamino)-7-methyl-thieno[3,2-d]pyrimidin-6-yl]methyl]-2-hydroxy-ethyl]carbamate

To a solution of tert-butyl N-[(1R)-1-[[tert-butyl(dimethyl)silyl]oxymethyl]-2-[2-chloro-4-(2-furylmethylamino)-7-methyl-thieno[3,2-d]pyrimidin-6-yl]ethyl]carbamate (50 mg, 0.088 mmol, 1.0 eq., prepared according to the procedure in example 3, step 2) in THF (0.5 mL) at 0° C. was added TBAF (1.0 M in THF) (0.18 mL, 0.18 mmol, 2.0 eq.). After 2 h at room temperature, the mixture was diluted with ether, washed with water and brine, dried over sodium sulfate and evaporated. The residue was purified over silica gel with ethyl acetate in hexanes (5 to 50% gradient) to give tert-butyl N-[(1R)-1-[[2-chloro-4-(2-furylmethylamino)-7-methyl-thieno[3,2-d]pyrimidin-6-yl]methyl]-2-hydroxy-ethyl]carbamate (31 mg, 78% yield). MS m/z 575.5, 577.5 [M+Na]⁺; ¹H NMR (CDCl₃) δ: 7.29 (s, 1H), 6.26-6.31 (m, 2H), 5.20 (s, 2H), 4.86-5.00 (m, 1H), 3.88-3.99 (m, 1H), 3.70 -3.77 (m, 1H), 3.60-3.69 (m, 1H), 3.22 (d, J=7.3 Hz, 2H), 2.42 (s, 3H), 1.53 (s, 9H), 1.44 (s, 9H), 1 OH not observed.

### Step 2: (2R)-2-Amino-3-[2-chloro-4-(2-furylmethylamino)-7-methyl-thieno[3,2-d]pyrimidin-6-yl]propan-1-ol dihydrochloride

The general de-Boc procedure using HCl in dioxane was followed to give (2R)-2-amino-3-[2-chloro-4-(2-furylmethylamino)-7-methyl-thieno[3,2-d]pyrimidin-6-yl]propan-1-ol dihydrochloride (10 mg, 34% yield). MS m/z 353.3, 355.3 [M+H]⁺; ¹H NMR (Methanol-d₄) δ: 7.23-7.37 (m, 1H), 6.17-6.27 (m, 2H), 4.63 (s, 2H), 3.60-3.69 (m, 1H), 3.49 (s, 1H), 3.40-3.45 (m, 1H), 3.21-3.26 (m, 1H), 3.09-3.15 (m, 1H), 2.21 (s, 3H); 3 NHs and 1 OH not observed.

The compounds below were prepared according to the procedure of Example 4 by substituting the appropriate starting materials, reagents and reaction conditions.

| Com- | |
|---|---|
| pound | Spectral Data |
| 22 | MS m/z 339.2, 341.2 [M + H]⁺; ¹H NMR (methanol-d₄) δ: 7.42 |
| | (d, J = 0.9 Hz, 1H), 7.07 (s, 1H), 6.31-6.37 (m, 2H), 4.74 (s, 2H), |
| | 3.56 (dd, J = 10.9, 4.5 Hz, 1H), 3.45-3.49 (m, 1H), 3.17 (s, 2H), |
| | 2.89-2.99 (m, 1H); 3 NHs and 1 OH not observed. |
| 56 | MS m/z 369.1, 371.1 [M + H]⁺; ¹H NMR (methanol-d4) δ: 7.28- |
| | 7.36 (m, 1H), 7.10-7.18 (m, 1H), 6.95-7.02 (m, 1H), 4.97-5.03 |
| | (m, 2H), 3.74-3.82 (m, 1H), 3.55-3.64 (m, 2H), 3.37-3.44 (m, |
| | 1H), 3.26-3.32 (m, 1H), 2.40 (s, 3H); 3 NHs and 1 OH not |
| | observed. |
| 97 | MS m/z 367.2, 369.2 [M + H]⁺; ¹H NMR (methanol-d4) δ: 7.43- |
| | 7.52 (m, 1H), 6.35-6.46 (m, 2H), 4.87 (s, 2H), 3.80-3.87 (m, |
| | 1H), 3.72-3.79 (m, 2H), 3.36-3.41 (m, 2H), |
| | 2.42 (s, 3H), 1.88-1.95 (m, 2H); 3 NHs and 1 OH not observed. |

### Example 5 (Compound 79)

### 6-[(2S)-2-Aminopropyl]-2-chloro-N-[(3-fluoropyridin-2-yl)methyl]-7-methylthieno[3,2-d]pyrimidin-4-amine dihydrochloride

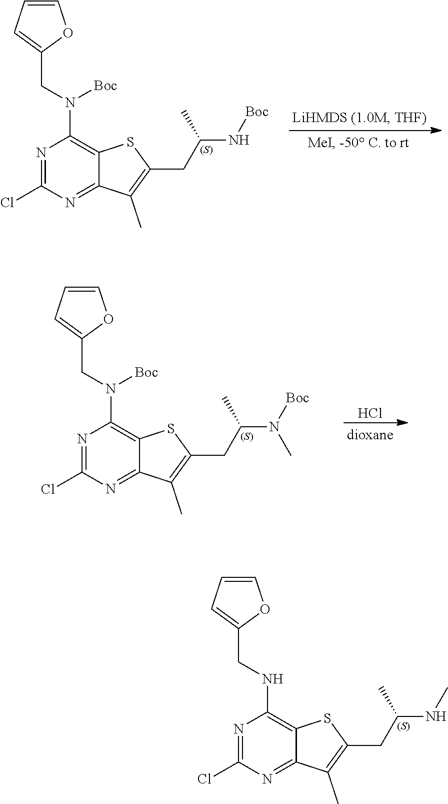

### Step 1: tert-Butyl (S)-(6-(2-((tert-butoxycarbonyl)(methyl)amino)propyl)-2-chloro-7-methylthieno[3,2-d]pyrimidin-4-yl)(furan-2-ylmethyl)carbamate

To a solution of tert-butyl (S)-(6-(2-((tert-butoxycarbonyl)amino)propyl)-2-chloro-7-methylthieno[3,2-d]pyrimidin-4-yl)(furan-2-ylmethyl)carbamate (111 mg, 0.21 mmol, 1.0 eq., prepared according to the procedure in example 3, step 2), in THF (1 mL) was added LiHMDS (1.0 M in THF, 0.23 mL, 1.1 eq.) at −50° C. dropwise. After 30 min, Mel (35 mg, 0.25 mmol, 1.2 eq.) in THF (1 mL) was added, and the mixture was gradually warmed to room temperature over a period of 1 h. The mixture was stirred at room temperature for an additional 1 h, then cooled to −50° C. and quenched with a few drops of citric acid (1.0 M, aq.). After warming to room temperature, the reaction was diluted with water and EtOAc. The organic layer was washed with water, brine and dried over sodium sulfate and concentrated. The crude material was purified by flash column chromatography on silica gel eluting with 0-10% EtOAc in CH₂Cl₂ to provide a mixture of unreacted starting material and desired product, which was further purified on prep-HPLC, eluting with 20-100% CH₃CN in water containing 0.1% of formic acid to provide tert-butyl (S)-(6-(2-((tert-butoxycarbonyl)(methyl)amino)propyl)-2-chloro-7-methylthieno[3,2-d]pyrimidin-4-yl)(furan-2-ylmethyl)carbamate (67 mg, 59% yield) as a white solid. MS m/z 573.3, 575.3 [M+Na]⁺.

### Step 2: (S)-2-Chloro-N-(furan-2-ylmethyl)-7-methyl-6-(2-(methylamino)propyl)thieno[3,2-d]pyrimidin-4-amine

(S)-(6-(2-((tert-butoxycarbonyl)(methyl)amino)propyl)-2-chloro-7-methylthieno[3,2-d]pyrimidin-4-yl)(furan-2-ylmethyl)carbamate (67 mg, 0.12 mmol) was stirred in a solution of HCl (4 M in dioxane, 1 mL) at room temperature for 1 h and then the organic volatiles were removed. The crude solid was triturated with diethyl ether and filtered to afford (S)-2-chloro-N-(furan-2-ylmethyl)-7-methyl-6-(2-(methylamino)propyl)thieno[3,2-d]pyrimidin-4-amine (33 mg, 50% yield) as the hydrochloride salt. MS m/z 351.1, 353.1 [M+H]⁺;¹H NMR (methanol-d₄) δ: 7.46 (dd, J=1.8, 0.9 Hz, 1H), 6.32-6.45 (m, 2H), 4.80 (s, 2H), 3.55-3.63 (m, 1H), 3.42-3.48 (m, 1H), 3.17-3.23 (m, 1H), 2.80 (s, 3H), 2.38 (s, 3H), 1.27-1.38 (d, J=6.6 Hz, 3H);, 2 NHs not observed.

### Example 6 (Compound 28)

### 6-[(2S)-2-Aminopropyl]-2-chloro-7-methyl-N-[(thiophen-2-yl)methyl]thieno[3,2-d]pyrimidin-4-amine

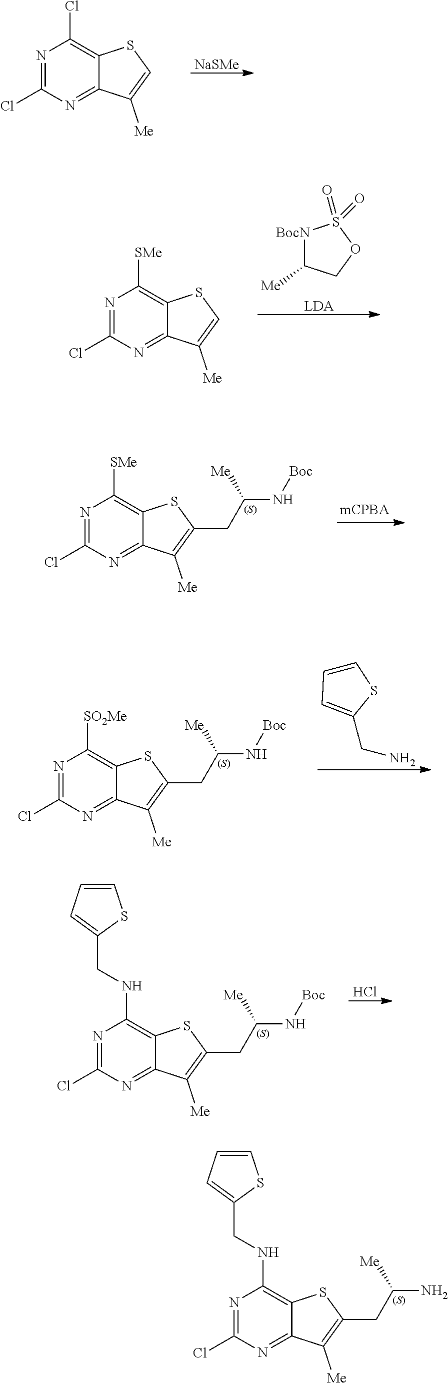

### Step 1: 2-Chloro-7-methyl-4-methylsulfanyl-thieno[3,2-d]pyrimidine

To a round bottom flask with 2,4-dichloro-7-methyl-thieno[3,2-d]pyrimidine (3.0 g, 14 mmol, 1.0 eq.) and sodium methanethiolate (1.1 g, 14 mmol, 1.05 eq.) was placed under vacuum and then charged with nitrogen. THF (55 mL) was then added to the round bottom flask and was stirred at 35° C. for 5.5 h. The mixture was then filtered. The filtrate was evaporated and dried under vacuum to afford 2-chloro-7-methyl-4-methylsulfanyl-thieno[3,2-d]pyrimidine (2.8 g, 84% yield), which was used in the next step without further purification. MS m/z 231.0, 233.0 [M+H]⁺; ¹H NMR (CDCl₃) δ: 7.52 (d, J=0.92 Hz, 1H) 2.77 (s, 3H) 2.48 (d, J=1.07 Hz, 3H).

### Step 2: tert-Butyl N-[(1S)-2-(2-chloro-7-methyl-4-methylsulfanyl-thieno[3,2-d]pyrimidin-6-yl)-1-methyl-ethyl]carbamate

To a solution of 2-chloro-7-methyl-4-methylsulfanyl-thieno[3,2-d]pyrimidine (500 mg, 2.16 mmol, 1.0 eq.) in THF (9.0 mL) at −78° C. was added LDA (2.0 M in THF/heptane/ethylbenzene) (1.3 mL, 2.60 mmol, 1.2 eq.) dropwise. After 15 min, a solution of tert-butyl (4S)-4-methyl-2,2-dioxo-oxathiazolidine-3-carboxylate (617.1 mg, 2.60 mmol, 1.2 eq.) in THF (9.0 mL) was added dropwise. The mixture was stirred at −78° C. for 10 min and then quenched with 1.0 M citric acid, followed by stirring at room temperature for 15 min. The mixture was diluted with ethyl acetate, washed with water, sodium bicarbonate and brine. The organic layer was dried over sodium sulfate, filtered and then concentrated under reduced pressure. The crude residue was purified over silica gel with ethyl acetate in hexanes (2-25%) to afford tert-butyl N-[(1S)-2-(2-chloro-7-methyl-4-methylsulfanyl-thieno[3,2-d]pyrimidin-6-yl)-1-methyl-ethyl]carbamate (605 mg, 72% yield) as a white solid. MS m/z 388.4, 390.4 [M+H]⁺; ¹H NMR (CDCl₃) δ: 4.42-4.50 (m, 1H), 3.98-4.08 (m, 1H), 3.05-3.21 (m, 2H), 2.75 (s, 3H), 2.40 (s, 3H), 1.44 (s, 9H), 1.19 (d, J=6.87 Hz, 3H).

### Step 3: tert-Butyl N-[(1S)-2-(2-chloro-7-methyl-4-methylsulfonyl-thieno[3,2-d]pyrimidin-6-yl)-1-methyl-ethyl]carbamate

A solution of tert-butyl N-[(1S)-2-(2-chloro-7-methyl-4-methylsulfanyl-thieno[3,2-d]pyrimidin-6-yl)-1-methyl-ethyl]carbamate (605 mg, 1.6 mmol, 1.0 eq.) and mCPBA (1435 mg, 6.2 mmol, 4.0 eq.) in CH₂Cl₂ (25 mL) was stirred at room temperature for 2 h. The mixture was diluted with ethyl acetate and washed with aq. sodium thiosulfate, sodium bicarbonate and brine. The organic layer was dried over sodium sulfate and filtered and concentrated under reduced pressure. The crude residue was purified over silica gel with ethyl acetate in dichloromethane (0-10%) to afford tert-butyl N-[(1S)-2-(2-chloro-7-methyl-4-methylsulfonyl-thieno[3,2-d]pyrimidin-6-yl)-1-methyl-ethyl]carbamate (286 mg, 44% yield) as an off-white solid. MS m/z 442.1, 444.1 [M+Na]⁺; ¹H NMR (CDCl₃) δ: 4.44-4.61 (m, 1H), 4.05 (br d, J=5.65 Hz, 1H), 3.38 (s, 3H), 3.22-3.29 (m, 1H), 3.15-3.22 (m, 1H), 2.48 (s, 3H), 1.42-1.45 (m, 9H), 1.23 (d, J=6.87 Hz, 3H).

### Step 4: tert-Butyl N-[(1S)-2-[2-chloro-7-methyl-4-(2-thienylmethylamino)thieno[3,2-d]pyrimidin-6-yl]-1-methyl-ethyl]carbamate

To a solution of tert-butyl N-[(1S)-2-(2-chloro-7-methyl-4-methylsulfonyl-thieno[3,2-d]pyrimidin-6-yl)-1-methyl-ethyl]carbamate (147 mg, 0.35 mmol, 1.0 eq.) in DMF (1.5 mL) was added 2-thienylmethanamine (79 mg, 0.70 mmol, 2.0 eq.) and was stirred at room temperature for 1 h. The mixture was diluted with ethyl acetate, washed with saturated NH₄Cl, water and brine. The organic layer was dried over sodium sulfate, filtered and then concentrated under reduced pressure. The crude residue was purified over silica gel with ethyl acetate in hexanes (10-50%) to afford tert-butyl N-[(1S)-2-[2-chloro-7-methyl-4-(2-thienylmethylamino)thieno [3,2-d]pyrimidin-6-yl]-1-methyl-ethyl]carbamate (120 mg, 76% yield) as a yellow solid. MS m/z 453.2, 455.2 [M+H]⁺; ¹H NMR (methanol-d₄) δ: 7.26 (dd, J=5.11, 1.14 Hz, 1H), 7.07 (d, J=2.90 Hz, 1H), 6.94 (dd, J=5.11, 3.43 Hz, 1H), 4.90 (d, J=5.49 Hz, 2H), 3.85-3.94 (m, 1H), 2.99-3.07 (m, 2H), 2.30 (s, 3H), 1.34 (s, 9H), 1.17 (d, J=7.20 Hz, 3H); 2 NHs not observed.

### Step 5: 6-[(2S)-2-Aminopropyl]-2-chloro-7-methyl-N-(2-thienylmethyl)thieno[3,2-d]pyrimidin-4-amine

To a reaction tube with tert-butyl N-[(1S)-2-[2-chloro-7-methyl-4-(2-thienylmethylamino)thieno[3,2-d]pyrimidin-6-yl]-1-methyl-ethyl]carbamate (120 mg, 0.26 mmol, 1.0 eq.) was added HCl in dioxane (4.0 M, 2.5 mL). The reaction mixture was stirred for 30 min, then diluted with diethyl ether and was filtered and rinsed with additional diethyl ether. The solid was placed under vacuum for 24 h to afford 6-[(2S)-2-aminopropyl]-2-chloro-7-methyl-N-(2-thienylmethyl)thieno[3,2-d]pyrimidin-4-amine (90 mg, 96% yield) as a white solid. MS m/z 353.1, 355.1 [M+H]⁺; ¹H NMR (methanol-d₄) δ: 7.32 (dd, J=5.11, 1.14 Hz, 1H), 7.14 (d, J=2.59 Hz, 1H), 6.97 (dd, J=5.04, 3.51 Hz, 1H), 5.01 (s, 2H), 3.61-3.65 (m, 1H), 3.33-3.39 (m, 1H), 3.20-3.27 (m, 1H) 2.40 (s, 3H) 1.36 (d, J=6.56 Hz, 3H); 3 NHs not observed.

The compounds below were prepared according to the procedure of Example 6 by substituting the appropriate starting materials, reagents and reaction conditions.

| Com- | |
|---|---|
| pound | Spectral Data |
| 24 | MS m/z 351.2, 353.2 [M + H]⁺; ¹H NMR (methanol-d₄) δ: 6.24 |
| | (d, J = 2.9 Hz, 1H), 5.95 (d, J = 2.4 Hz, 1H), 4.75 (s, 2H), 3.60- |
| | 3.66 (m, 1H), 3.32-3.36 (m, 1H), 3.19-3.25 (m, 1H), 2.37 (s, |
| | 3H), 2.24 (s, 3H), 1.36 (d, J = 6.4 Hz, 3H); 3 NHs not observed. |
| 27 | MS m/z 368.2, 370.2 [M + H]⁺; ¹H NMR (methanol-d₄) δ: 7.52 |
| | (s, 1H), 5.18 (s, 2H), 3.62-3.65 (m, 1H), 3.33-3.37 (m, J = 6.1 |
| | Hz, 1H), 3.19-3.25 (m, 1H), 2.53 (s, 3H), 2.36 (s, 3H), 1.36 (d, |
| | J = 6.6 Hz, 3H); 3 NHs not observed. |
| 29 | MS m/z 354.2, 356.2 [M + H]⁺; ¹H NMR (methanol-d₄) δ: 7.74 |
| | (d, J = 3.4 Hz, 1H), 7.52 (d, J = 3.4 Hz, 1H), 5.07 (s, 2H), 3.59- |
| | 3.66 (m, 1H), 3.27-3.29 (m, J = 6.6 Hz, 1H), 3.17-3.23 (m, 1H), |
| | 2.35 (s, 3H), 1.36 (d, J = 6.6 Hz, 3H); 3 NHs not observed. |
| 30 | MS m/z 351.2, 353.2 [M + H]⁺; ¹H NMR (methanoL-d₄) δ: 7.31 |
| | (d, J = 1.8 Hz, 1H), 6.22 (d, J = 1.7 Hz, 1H), 4.68 (s, 2H), 3.57- |
| | 3.64 (m, 1H), 3.24-3.26 (m, J = 6.4 Hz, 1H), 3.17-3.20 (m, J = |
| | 8.2 Hz, 1H), 2.32 (s, 3H), 2.16 (s, 3H), 1.34 (d, J = 6.6 Hz, 3H); |
| | 3 NHs not observed. |
| 31 | MS m/z 368.2, 370.2 [M + H]⁺; ¹H NMR (methanol-d₄) δ: 7.76 |
| | (d, J = 1.2 Hz, 1H), 5.13 (s, 2H), 3.62-3.66 (m, 1H), 3.34-3.36 |
| | (m, J = 6.4 Hz, 1H), 3.18-3.22 (m, 1H), 2.53 (s, 3H), 2.36 (s, |
| | 3H), 1.36 (d, J = 6.6 Hz, 3H); 3 NHs not observed. |
| 32 | MS m/z 349.2, 351.2 [M + H]⁺; ¹H NMR (methanol-d₄) δ: 8.70 |
| | (d, J = 1.1 Hz, 1H), 8.59 (t, J = 1.0 Hz, 1H), 8.52 (d, J = 2.6 Hz, |
| | 1H), 4.93 (s, 2H), 3.60-3.65 (m, 1H), 3.21-3.24 (m, J = 8.4 Hz, |
| | 1H), 2.35 (s, 3H), 1.36 (d, J = 6.6 Hz, 3H), 1 H obscured by |
| | MeOD peak; 3 NHs not observed. |
| 33 | MS m/z 371.1, 373.1 [M + H]⁺; ¹H NMR (methanol-d₄) δ: 6.73 |
| | (t, J = 3.7 Hz, 1H), 6.37 (dd, J = 4.0, 2.0 Hz, 1H), 4.78 (d, J = |
| | 2.3 Hz, 2H), 3.58-3.64 (m, 1H), 3.27-3.29 (m, J = 6.6 Hz, 1H), |
| | 3.19-3.22 (m, J = 8.2 Hz, 1H), 2.35 (s, 3H), 1.35 (d, J = 6.6 Hz, |
| | 3H); 3 NHs not observed. |
| 34 | MS m/z 347.2, 349.2 [M + H]⁺; ¹H NMR (methanol-d₄) δ: 7.37- |
| | 7.40 (m, 2H), 7.32 (t, J = 7.6 Hz, 2H), 7.24-7.28 (m, 1H), 4.79 |
| | (s, 2H), 3.59-3.64 (m, 1H), 3.27-3.29 (m, J = 6.4 Hz, 1H), 3.19- |
| | 3.22 (m, J = 8.1 Hz, 1H), 2.35 (s, 3H), 1.35 (d, J = 6.6 Hz, 3H); |
| | 3 NHs not observed. |
| 35 | MS m/z 366.1, 368.1 [M + H]⁺; ¹H NMR (methanol-d₄) δ: 8.70 |
| | (br s, 1H), 8.45-8.50 (m, J = 3.8 Hz, 1H), 7.70-7.76 (m, 1H), |
| | 4.96 (s, 2H), 3.60-3.67 (m, 1H), 3.34-3.36 (m, 1H), 3.20-3.23 |
| | (m, J = 8.4 Hz, 1H), 2.35 (s, 3H), 1.36 (d, J = 6.6 Hz, 3H); |
| | 3 NHs not observed. |
| 37 | MS m/z 367.1, 369.1 [M + H]⁺; ¹H NMR (methanol-d₄) δ: 7.29 |
| | (dd, J = 5.0, 1.2 Hz, 1H), 7.09-7.12 (m, 1H), 6.96 (dd, J = 5.2, |
| | 3.5 Hz, 1H), 4.96 (s, 2H), 3.46-3.51 (m, 1H), 3.28-3.30 (m, 1H), |
| | 3.24-3.26 (m, J = 7.3 Hz, 1H), 2.36 (s, 3H), 1.66-1.83 (m, 2H), |
| | 1.08 (t, J = 7.5 Hz, 3H); 3 NHs not observed. |
| 39 | MS m/z 338.3, 340.3 [M + H]⁺; ¹H NMR (methanol-d₄) δ: 8.33 |
| | (s, 1H), 6.38 (s, 1H), 4.96 (s, 2H), 3.61-3.65 (m, 1H), 3.34-3.36 |
| | (m, J = 6.3 Hz, 1H), 3.19-3.25 (m, 1H), 2.37 (s, 3H), 1.36 (d, J = |
| | 6.3 Hz, 3H); 3 NHs not observed. |
| 44 | MS m/z 365.4, 367.4 [M + H]⁺; ¹H NMR (methanol-d₄) δ: 7.45 |
| | (td, J = 7.6, 1.4 Hz, 1H), 7.31 (tdd, J = 7.7, 5.5, 1.8 Hz, 1H), |
| | 7.09-7.16 (m, 2H), 4.85 (s, 2H), 3.59-3.66 (m, 1H), 3.28-3.30 |
| | (m, 1H), 3.18-3.23 (m, 1H), 2.36 (s, 3H), 1.36 (d, J = 6.6 Hz, |
| | 3H); 3 NHs not observed. |
| 45 | MS m/z 348.3, 350.3 [M + H]⁺; ¹H NMR (methanol-d₄) δ: 8.79 |
| | (d, J = 6.7 Hz, 2H), 8.08 (d, J = 6.6 Hz, 2H), 5.06 (s, 2H), 3.61- |
| | 3.65 (m, 1H), 3.33-3.36 (m, J = 6.1 Hz, 1H), 3.22-3.25 (m, J = |
| | 8.5 Hz, 1H), 2.35 (s, 3H), 1.37 (d, J = 6.6 Hz, 3H); 3 NHs not |
| | observed. |
| 50 | MS m/z 349.3, 351.3 [M + H]⁺; ¹H NMR (methanol-d₄) δ: 9.26 |
| | (s, 1H), 8.86 (d, J = 5.5 Hz, 1H), 7.74 (d, J = 5.6 Hz, 1H), 4.98 |
| | (s, 2H), 3.61-3.64 (m, 1H), 3.32-3.35 (m, 1H), 3.22-3.26 (m, J = |
| | 8.5 Hz, 1H), 2.36 (s, 3H), 1.37 (d, J = 6.4 Hz, 3H); 3 NHs not |
| | observed. |
| 53 | MS m/z 380.4, 382.4 [M + H]⁺; ¹H NMR (methanol-d₄) δ: 8.98 |
| | (d, J = 3.1 Hz, 1H), 8.66 (d, J = 5.8 Hz, 1H), 8.04 (t, J = 6.0 Hz, |
| | 1H), 5.06 (s, 2H), 3.51 (quin, J = 6.5 Hz, 1H), 3.27-3.29 (m, |
| | 1H), 2.35 (s, 3H), 1.66-1.86 (m, 2H), 1.09 (t, J = 7.5 Hz, 3H), |
| | 1 H obscured by MeOD peak; 3 NHs not observed. |
| 54 | MS m/z 368.3, 370.3 [M + H]⁺; ¹H NMR (methanol-d₄) δ: 8.06 |
| | (d, J = 3.4 Hz, 1H), 7.89 (d, J = 3.5 Hz, 1H), 5.21 (s, 2H), 3.51 |
| | (quin, J = 6.7 Hz, 1H), 3.27-3.29 (m, 1H), 2.37 (s, 3H), 1.67- |
| | 1.85 (m, 2H), 1.09 (t, J = 7.5 Hz, 3H), 1 H obscured by MeOD |
| | peak; 3 NHs not observed. |
| 55 | MS m/z 349.3, 351.3 [M + H]⁺; ¹H NMR (methanol-d₄) δ: 8.81 |
| | (d, J = 4.9 Hz, 2H), 7.47 (t, J = 5.0 Hz, 1H), 5.02 (s, 2H), 3.63- |
| | 3.67 (m, 1H), 3.24-3.27 (m, J = 7.9 Hz, 1H), 2.37 (s, 3H), 1.40 |
| | (d, J = 6.4 Hz, 3H), 1 H obscured by MeOD peak, 3 NHs not |
| | observed. |
| 59 | MS m/z 366.3, 368.3 [M + H]⁺; ¹H NMR (methanol-d₄) δ: 8.44 |
| | (d, J = 5.2 Hz, 1H), 7.91 (t, J = 8.7 Hz, 1H), 7.59-7.64 (m, 1H), |
| | 5.01 (s, 2H), 3.61-3.66 (m, 1H), 3.21-3.23 (m, J = 8.2 Hz, 1H), |
| | 2.34 (s, 3H), 1.36 (d, J = 6.6 Hz, 3H), 1 H obscured by MeOD |
| | peak; 3 NHs not observed. |
| 60 | MS m/z 366.4, 368.4 [M + H]⁺; ¹H NMR (methanol-d₄) δ: 8.12 |
| | (d, J = 4.6 Hz, 1H), 7.97-8.02 (m, 1H), 7.29 (ddd, J = 7.1, 5.1, |
| | 1.4 Hz, 1H), 4.80 (s, 2H), 3.60-3.64 (m, 1H), 3.20-3.22 (m, J = |
| | 8.1 Hz, 1H), 2.35 (s, 3H), 1.35 (d, J = 6.4 Hz, 3H), 1 H obscured |
| | by MeOD peak; 3 NHs not observed. |
| 69 | MS m/z 352.2, 354.2 [M + H]⁺; ¹H NMR (methanol-d₄) δ: 8.08 |
| | (s, 1H), 7.38 (s, 1H), 5.06 (s, 2H), 3.50-3.57 (m, 1H), 3.36-3.41 |
| | (m, 1H), 2.42 (s, 3H), 1.72-1.87 (m, 2H), 1.09 (t, J = 7.4 Hz, |
| | 3H), 1 H obscured by MeOD peak; 3 NHs not observed. |
| 73 | MS m/z 337.1, 339.1 [M + H]⁺; ¹H NMR (DMSO-d₆) δ: 9.08- |
| | 9.15 (m, 1H), 8.23-8.31 (m, 3H), 7.63 (s, 2H), 4.92-4.98 (m, |
| | 2H), 3.42-3.48 (m, 1H), 3.34-3.39 (m, 1H), 3.09-3.17 (m, 1H), |
| | 2.28 (s, 3H), 1.22 (d, J = 6.4 Hz, 3H). |
| 74 | MS m/z 354.1, 356.1 [M + H]⁺; ¹H NMR (methanol-d₄) δ: 9.81 |
| | (s, 1H), 8.34 (s, 1H), 5.03 (s, 2H), 3.60-3.65 (m, 1H), 3.33-3.36 |
| | (m, 1H), 3.21-3.23 (m, J = 8.4 Hz, 1H), 2.36 (s, 3H), 1.35 (d, J = |
| | 6.6 Hz, 3H); 3 NHs not observed. |
| 75 | MS m/z 338.1, 340.1 [M + H]⁺; ¹H NMR (methanol-d₄) δ: 8.19 |
| | (s, 1H), 7.13 (s, 1H), 4.86 (s, 2H), 3.59-3.64 (m, 1H), 3.28-3.29 |
| | (m, J = 6.6 Hz, 1H), 3.20-3.22 (m, J = 8.2 Hz, 1H), 2.35 (s, 3H), |
| | 1.35 (d, J = 6.6 Hz, 3H); 3 NHs not observed. |
| 81 | MS m/z 351.3, 353.3 [M + H]⁺; ¹H NMR (DMSO-d₆) δ: 8.81- |
| | 8.93 (m, 1H), 8.22-8.34 (m, 3H), 7.27-7.39 (m, 1H), 6.12-6.31 |
| | (m, 1H), 4.64-4.75 (m, 2H), 3.88 (s, 3H), 3.39-3.44 (m, 1H), |
| | 3.31-3.37 (m, 1H), 3.06-3.16 (m, 1H), 2.24 (s, 3H), 1.22 (d, J = |
| | 6.4 Hz, 3H). |
| 89 | MS m/z 363.3, 365.3 [M + H]⁺; ¹H NMR (methanol-d4) δ: 9.47 |
| | (s, 1H), 9.07 (d, J = 5.8 Hz, 1H), 8.06 (d, J = 5.8 Hz, 1H), 5.16 |
| | (s, 2H), 3.52-3.61 (m, 1H), 3.35-3.45 (m, 2H), 2.42 (s, 3H), |
| | 1.72-1.88 (m, 2H), 1.12 (t, J = 7.3 Hz, 3H); 3 NHs not observed. |
| 90 | MS m/z 371.1, 373.1 [M + H]⁺; ¹H NMR (methanol-d₄) δ: 7.25 |
| | (t, J = 4.6 Hz, 1H), 6.80 (d, J = 5.6 Hz, 1H), 4.87 (s, 2H), 3.58- |
| | 3.64 (m, 1H), 3.27-3.29 (m, 1H), 3.19-3.22 (m, J = 8.5 Hz, 1H), |
| | 2.35 (s, 3H), 1.35 (d, J = 6.4 Hz, 3H); 3 NHs not observed. |
| 91 | MS m/z 385.2, 387.2 [M + H]⁺; ¹H NMR (methanol-d₄) δ: 7.28 |
| | (t, J = 4.7 Hz, 1H), 6.82 (d, J = 5.6 Hz, 1H), 4.88 (s, 2H), 3.48 |
| | (quin, J = 6.7 Hz, 1H), 3.26-3.29 (m, J = 7.3 Hz, 1H), 2.37 (s, |
| | 3H), 1.76-1.83 (m, 1H), 1.67-1.74 (m, 1H), 1.08 (t, J = 7.4 Hz, |
| | 3H), 1 H obscured by MeOD peak; 3 NHs not observed. |
| 100 | MS m/z 352.8 [M + H]⁺; ¹H NMR (DMSO-d₆) δ: 8.62 (t, J = |
| | 5.87 Hz, 1H), 7.48 (dd, J = 4.95, 3.00 Hz, 1H), 7.33-7.36 (m, |
| | 1H), 7.10 (dd, J = 4.89, 1.22 Hz, 1H), 4.64 (d, J = 5.87 Hz, 2H), |
| | 3.09 (sxt, J = 6.31 Hz, 1H), 2.85 (d, J = 6.48 Hz, 2H), 2.20 (s, |
| | 3H), 1.97 (br s, 2H), 1.02 (d, J = 6.24 Hz, 3H). |
| 101 | MS m/z 336.9 [M + H]⁺; ¹H NMR (DMSO-d₆) δ: 12.61 (br s, |
| | 1H), 8.57 (br s, 1H), 7.63 (br s, 1H), 6.18 (br s, 1H), 4.63 (br s, |
| | 2H), 3.04-3.27 (m, 1H), 2.87 (br d, J = 6.38 Hz, 2H), 2.20 |
| | (s, 3H), 1.03 (d, J = 6.25 Hz, 3H); 2 NHs not observed. |
| 102 | MS m/z 350.9 [M + H]⁺; ¹H NMR (methanol-d₄) δ: 7.03 (s, |
| | 1H), 6.88 (s, 1H), 4.74-4.82 (m, 2H), 3.83 (s, 3H), 3.20-3.28 |
| | (m, 1H), 2.90-3.08 (m, 2H), 2.30 (s, 3H), 1.16 (d, J = 6.38 Hz, |
| | 3H); 3 NHs not observed. |
| 103 | MS m/z 337.2 [M + H]⁺; ¹H NMR (DMSO-d₆) δ: 8.50 (br s, |
| | 1H), 7.60 (br d, J = 5.13 Hz, 2H), 6.49 (s, 1H), 4.48 (br d, J = |
| | 5.38 Hz, 2H), 3.02-3.26 (m, 1H), 2.85 (br d, J = 6.25 Hz, 2H), |
| | 2.20 (s, 3H), 1.99 (br s, 2H), 1.02 (br d, J = 6.13 Hz, 3H). |
| 104 | MS m/z 335.9 [M + H]⁺; ¹H NMR (methanol-d₄) δ: 6.68 |
| | (dd, J = 2.63, 1.63 Hz, 1H), 6.03-6.09 (m, |
| | 1H), 6.00 (t, J = 3.00 Hz, 1H), 4.62-4.80 (m, 2H), 3.08-3.29 |
| | (m, 1H), 2.84-3.03 (m, 2H), 2.28 (s, 3H), 1.13 (d, J = 6.38 Hz, |
| | 3H); 4 NHs not observed. |
| 105 | MS m/z 337.9 [M + H]⁺; ¹H NMR (chloroform-d) δ: 8.40 |
| | (d, J = 1.59 Hz, 1H), 6.46 (d, J = 1.59 Hz, 1H), 5.52 (br s, |
| | 1H), 4.96 (d, J = 5.62 Hz, 2H), 3.25-3.35 (m, 1H), 2.84-3.01 |
| | (m, 2H), 2.36 (s, 3H), 1.19 (d, J = 6.24 Hz, 3H); 2 NHs not |
| | observed. |
| 106 | MS m/z 350.9 [M + H]⁺; ¹H NMR (chloroform-d) δ: 7.29-7.35 |
| | (m, 1H), 6.26 (s, 1H), 4.78 (br s, 2H), 3.90 (s, 3H), 3.36 (br s, |
| | 1H), 2.99 (br s, 2H), 2.32 (s, 3H), 1.24 (br d, J = 4.4 Hz, 3H); |
| | 3 NHs not observed. |
| 107 | MS m/z 337.9 [M + H]⁺; ¹H NMR (DMSO-d₆) δ: 8.88 (s, |
| | 1H), 8.58-8.60 (m, 1H), 8.52-8.57 (m, 1H), 4.51 (d, J = 5.38 Hz, |
| | 2H), 3.05-3.16 (m, 1H), 2.87 (d, J = 6.48 Hz, 2H), 2.21 (s, |
| | 3H), 1.02 (d, J = 6.24 Hz, 3H); 2 NHs not observed. |
| 108 | MS m/z 353.9 [M + H]⁺; ¹H NMR (chloroform-d) δ: 8.66 (s, |
| | 1H), 8.57 (s, 1H), 5.22 (br s, 1H), 4.94 (d, J = 5.87 Hz, 2H), |
| | 3.25-3.34 (m, 1H), 2.83-3.00 (m, 2H), 2.36 (s, 3H), 1.18 (d, J = |
| | 6.24 Hz, 3H); 2 NHs not observed. |
| 109 | MS m/z 336.9 [M + H]⁺; ¹H NMR (DMSO-d₆) δ: 12.66 (br s, |
| | 1H), 8.46 (t, J = 5.63 Hz, 1H), 7.66 (br s, 1H), 7.48 (br s, |
| | 1H), 4.49 (d, J = 5.63 Hz, 2H), 3.08 (q, J = 6.34 Hz, 1H), 2.84 |
| | (d, J = 6.38 Hz, 2H), 2.19 (s, 3H), 1.51-2.09 (m, 2H), 1.01 |
| | (d, J = 6.38 Hz, 3H). |
| 110 | MS m/z 350.9 [M + H]⁺; ¹H NMR (DMSO-d₆) δ: 8.46 (t, J = |
| | 5.74 Hz, 1H), 7.60 (s, 1H), 7.38 (s, 1H), 4.45 (d, J = 5.62 Hz, |
| | 2H), 3.77 (s, 3H), 3.01-3.14 (m, 1H), 2.84 (d, J = 6.48 Hz, 2H), |
| | 2.19 (s, 3H), 2.03 (br s, 2H), 1.01 (d, J = 6.24 Hz, 3H). |
| 111 | MS m/z 337.8 [M + H]⁺; ¹H NMR (DMSO-d₆) δ: 8.82 (br s, |
| | 1H), 8.05 (s, 1H), 7.15 (s, 1H), 4.77 (br d, J = 5.38 Hz, 2H), |
| | 2.78-3.11 (m, 3H), 2.22 (s, 3H), 1.40 (s, 1H), 1.17-1.29 (m, 1H), |
| | 1.09 (br s, 3H). |
| 112 | MS m/z 337.9 [M + H]⁺; ¹H NMR (DMSO-d₆) δ: 8.59 (t, J = |
| | 5.69 Hz, 1H), 8.33 (s, 1H), 7.97 (s, 1H), 4.55 (d, J = 5.62 |
| | Hz, 2H), 3.05-3.15 (m, 1H), 2.86 (d, J = 6.36 Hz, 2H), 2.20 (s, |
| | 5H), 1.02 (d, J = 6.24 Hz, 3H). |
| 113 | MS m/z 353.9 [M + H]⁺; ¹H NMR (methanol-d₄) δ: 8.97 |
| | (d, J = 1.96 Hz, 1H), 7.46 (d, J = 1.96 Hz, 1H), 4.58 (br d, J = |
| | 1.83 Hz, 2H), 3.01 (br t, J = 7.40 Hz, 3H), 2.31 (s, 3H), 1.18 |
| | (d, J = 6.36 Hz, 3H); 3 NHs not observed. |
| 114 | MS m/z 336.9 [M + H]⁺; ¹H NMR (DMSO-d₆) δ: 11.77-12.01 |
| | (m, 1H), 8.48 (br s, 1H), 7.54-7.58 (m, 1H), 6.97 (s, 1H), 4.53 |
| | (d, J = 5.50 Hz, 2H), 3.02-3.11 (m, 1H), 2.84 (d, J = 6.36 Hz, |
| | 2H), 2.20 (s, 3H), 1.01 (d, J = 6.36 Hz, 3H); 2 NHs not |
| | observed. |
| 115 | MS m/z 350.9 [M + H]⁺; ¹H NMR (DMSO-d₆) δ: 8.51-8.55 (m, |
| | 1H), 7.54 (s, 1H), 6.86 (s, 1H), 4.61 (d, J = 5.14 Hz, 2H), 3.64 |
| | (s, 3H), 3.05-3.20 (m, 1H), 2.83-2.91 (m, 2H), 2.20 (s, 3H), 1.02 |
| | (d, J = 6.24 Hz, 3H); 2 NHs not observed. |
| 116 | MS m/z 351.1 [M + H]⁺; ¹H NMR (DMSO-d₆) δ: 8.49 (br s, |
| | 1H), 7.48 (s, 1H), 6.94 (s, 1H), 4.49 (br d, J = 5.50 Hz, 2H), |
| | 3.58 (s, 3H), 2.98-3.22 (m, 1H), 2.85 (br d, J = 6.50 Hz, 2H), |
| | 2.20 (s, 3H), 1.02 (br d, J = 5.75 Hz, 3H); 2 NHs not observed. |
| 117 | MS m/z 337.9 [M + H]⁺; ¹H NMR (methanol-d₄) δ: 7.76 (s, 1H), |
| | 4.84 (s, 2H), 3.23-3.29 (m, 1H), 2.9-3.05 (m, 2H), 2.28 (s, 3H), |
| | 1.17 (d, J = 6.38 Hz, 3H); 4 NHs not observed. |
| 118 | MS m/z 338.9 [M + H]⁺; ¹H NMR (methanol-d₄) δ: 4.96-5.06 |
| | (m, 2H), 3.53-3.63 (m, 1H), 3.10-3.27 (m, 2H), 2.35 (br s, 3H), |
| | 1.34 (d, J = 6.63 Hz, 3H); 4 NHs not observed. |
| 119 | MS m/z 350.0 [M + H]⁺; ¹H NMR (methanol-d₄) δ: 6.59-6.63 |
| | (m, 1H), 6.09 (dd, J = 3.55, 1.83 Hz, 1H), 5.94-5.98 (m, 1H), |
| | 4.71 (s, 2H), 3.65 (s, 3H), 3.21-3.29 (m, 1H), 2.91-3.03 (m, 2H), |
| | 2.29 (s, 3H), 1.16 (d, J = 6.36 Hz, 3H); 3 NHs not observed. |
| 120 | MS m/z 336.0 [M + H]⁺; ¹H NMR (methanol-d₄) δ: 6.77 (s, |
| | 1H), 6.66 (t, J = 2.38 Hz, 1H), 6.14 (dd, J = 2.57, 1.59 Hz, |
| | 1H), 4.59 (s, 2H), 3.22-3.29 (m, 1H), 2.89-3.05 (m, 2H), 2.29 |
| | (s, 3H), 1.16 (d, J = 6.36 Hz, 3H); 4 NHs not observed. |

### Example 7 (Compound 92)

### 6-[(2S)-2-Aminopropyl]-2-chloro-N-[(4-fluoro-1,3-thiazol-2-yl)methyl]-7-methylthieno[3,2-d]pyrimidin-4-amine

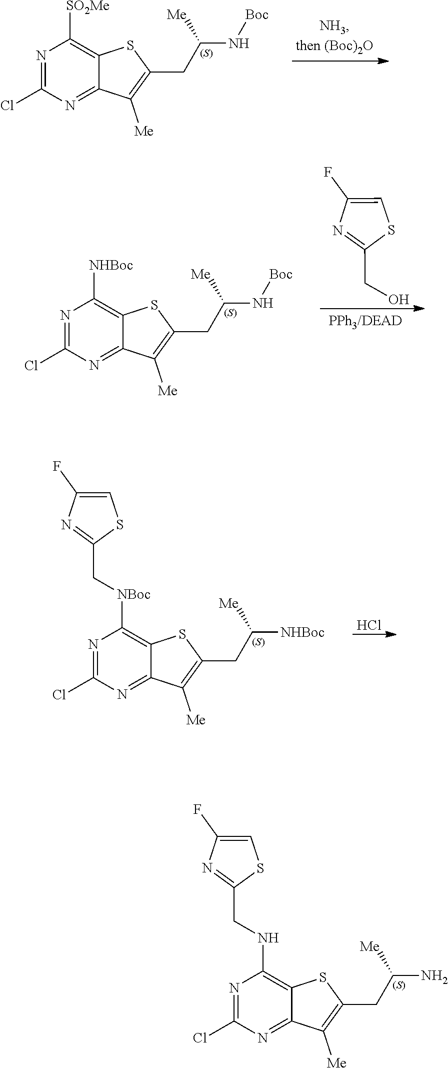

### Step 1: tert-Butyl N-[(1S)-2-[4-(tert-butoxycarbonylamino)-2-chloro-7-methyl-thieno[3,2-d]pyrimidin-6-yl]-1-methyl-ethyl]carbamate

To a round bottom flask tert-butyl N-[(1S)-2-(2-chloro-7-methyl-4-methylsulfonyl-thieno[3,2-d]pyrimidin-6-yl)-1-methyl-ethyl]carbamate (500 mg, 1.2 mmol, 1.0 eq., prepared according to the procedure in example 6), and ammonia (0.5 mol/L) in dioxanes (10 mL, 4.8 mmol, 4.0 eq.) was stirred at room temperature for 2 h. The organic volatiles were removed by a stream of nitrogen. The crude residue was then dissolved in CH₂Cl₂ (10 mL), to which was added di-tert-butyl dicarbonate (660 mg, 3.0 mmol, 2.5 eq.) and 4-(dimethylamino)pyridine (15 mg, 0.12 mmol, 0.10 eq.). The mixture was stirred at room temperature for 2 h and then concentrated under reduced pressure. The residue was dissolved in methanol (10 mL), to which potassium carbonate (1700 mg, 12 mmol, 10 eq.) was added. The mixture was stirred at room temperature for 1 h then concentrated. The residue was partitioned between ethyl acetate and water. The organic layer was separated, dried over sodium sulfate and evaporated. The residue was purified over silica gel with ethyl acetate and dichloromethane (0-10%) to afford tert-butyl N-[(1S)-2-[4-(tert-butoxycarbonylamino)-2-chloro-7-methyl-thieno[3,2-d]pyrimidin-6-yl]-1-methyl-ethyl]carbamate (170 mg, 31% yield) as a white solid. ¹H NMR (CDCl₃) δ: 7.66 (br s, 1H), 4.51 (d, J=7.6 Hz, 1H), 4.03 (br s, 1H), 3.02-3.19 (m, 2H), 2.38 (s, 3H), 1.55 (s, 9H), 1.43 (s, 9H), 1.18 (d, J=6.7 Hz, 3H).

### Step 2: tert-Butyl N-[6-[(2S)-2-(tert-butoxycarbonylamino)propyl]-2-chloro-7-methyl-thieno[3,2-d]pyrimidin-4-yl]-N-[(4-fluorothiazol-2-yl)methyl]carbamate

To a solution of tert-butyl N-[(1S)-2-[4-(tert-butoxycarbonylamino)-2-chloro-7-methyl-thieno[3,2-d]pyrimidin-6-yl]-1-methyl-ethyl]carbamate (170 mg, 0.37 mmol, 1.0 eq.), (4-fluorothiazol-2-yl)methanol (74 mg, 0.56 mmol, 1.5 eq.), and PPh₃ (157 mg, 0.59 mmol, 1.6 eq.) in THF (1 mL) at 0° C. was added DEAD (40% in toluene) (0.25 mL, 0.59 mmol, 1.5 eq.). The mixture was stirred for 2 h at room temperature and then concentrated under reduced pressure. The residue was purified over silica gel with ethyl acetate in dichloromethane (0-20%) to afford tert-butyl N-[6-[(2S)-2-(tert butoxycarbonylamino)propyl]-2-chloro-7-methyl-thieno[3,2-d]pyrimidin-4-yl]-N-[(4-fluorothiazol-2-yl)methyl]carbamate (180 mg, 84% yield). MS m/z 572.2, 574.2 [M+H]⁺; ¹H NMR (CDCl₃) δ: 6.49 (d, J=4.7 Hz, 1H), 5.35 (s, 2H), 4.49 (br d, J=2.3 Hz, 1H), 3.99-4.08 (m, 1H), 3.20 (br dd, J=14.0, 4.6 Hz, 1H), 3.04-3.11 (m, 1H), 2.40 (s, 3H), 1.50 (s, 9H), 1.44 (s, 9H), 1.18 (d, J=6.7 Hz, 3H).

### Step 3: 6-[(2S)-2-Aminopropyl]-2-chloro-N-[(4-fluorothiazol-2-yl)methyl]-7-methyl-thieno[3,2-d]pyrimidin-4-amine

To a reaction tube with tert-butyl N-[6-[(2S)-2-(tert-butoxycarbonylamino)propyl]-2-chloro-7-methyl-thieno[3,2-d]pyrimidin-4-yl]-N-[(4-fluorothiazol-2-yl)methyl]carbamate (180 mg, 0.31 mmol, 1.0 eq.) was added hydrochloric acid (4 mol/L) in dioxane (3 mL). The mixture was stirred at room temperature for 1 h, then diluted with diethyl ether, filtered and then rinsed with diethyl ether. The solid was dried under vacuum to give 6-[(2S)-2-aminopropyl]-2-chloro-N-[(4-fluorothiazol-2-yl)methyl]-7-methyl-thieno[3,2-d]pyrimidin-4-amine (60 mg, 51% yield) as a white solid. MS m/z 372.1, 374.1 [M+H]⁺; ¹H NMR (CDCl₃) δ: 6.82 (d, J=4.7 Hz, 1H), 5.02 (s, 2H), 3.66-3.68 (m, 1H), 3.36-3.39 (m, J=6.7 Hz, 1H), 3.26-3.29 (m, J=8.2 Hz, 1H), 2.41 (s, 3H), 1.41 (d, J=6.6 Hz, 3H); 3 NHs not observed.

The compounds below were prepared according to the procedure of Example 7 by substituting the appropriate starting materials, reagents and reaction conditions.

| Com- | |
|---|---|
| pound | Spectral Data |
| 93 | MS m/z 372.1, 374.1 [M + H]⁺; ¹H NMR (methanol-d₄) δ: 7.41 |
| | (d, J = 2.3 Hz, 1H), 4.99 (s, 2H), 3.62-3.70 (m, 1H), 3.35-3.42 |
| | (m, 1H), 3.23-3.28 (m, 1H), 2.41 (s, 3H), 1.37 (d, J = 6.6 Hz, |
| | 3H); 3 NHs not observed |

### Example 8 (Compound 64)

### 6-[(2S)-2-Aminopropyl]-2-chloro-N-[(3,5-difluoropyridin-4-yl)methyl]-7-methylthieno[3,2-d]pyrimidin-4-amine dihydrochloride

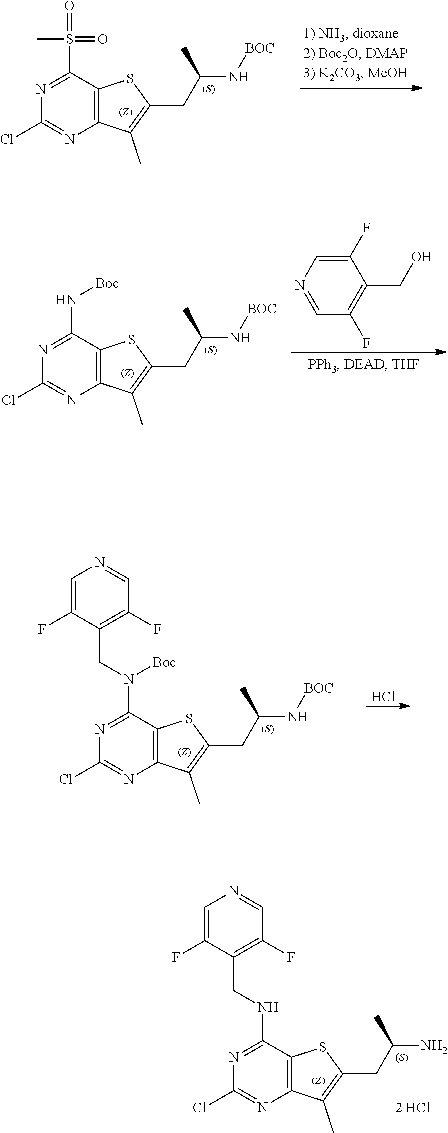

### Step 1: tert-Butyl N-[(1S)-2-[4-(tert-butoxycarbonylamino)-2-chloro-7-methyl-thieno[3,2-d]pyrimidin-6-yl]-1-methyl-ethyl]carbamate

A mixture of tert-butyl N-[(1S)-2-(2-chloro-7-methyl-4-methylsulfonyl-thieno[3,2-d]pyrimidin-6-yl)-1-methyl-ethyl]carbamate (500 mg, 1.2 mmol, 1.0 eq.), prepared according to the procedure in example 6, and ammonia (0.5 mol/L) in dioxane (10 mL, 4.8 mmol, 4.0 eq.) was stirred at room temperature for 2 h. The organic volatiles were removed by a stream of nitrogen. The residue was suspended in CH₂Cl₂ (10 mL), to which was added Boc₂O (660 mg, 0.69 mL, 3.0 mmol, 2.5 eq.) and DMAP (15 mg, 0.12 mmol, 0.10 eq.). After 2 h at room temperature, the mixture was concentrated. The residue was re-dissolved in MeOH (10 mL), to which K₂CO₃ (1.7 g, 12 mmol, 10 eq.) was added. The mixture was stirred at room temperature for 1 h. The organic solvent was then evaporated. The residue was partitioned between ethyl acetate and water. The organic layer was separated, dried over sodium sulfate and evaporated. The residue was purified over silica gel with ethyl acetate and dichloromethane (0 to 10% gradient) to give tert-butyl N-[(1S)-2-[4-(tert-butoxycarbonylamino)-2-chloro-7-methyl-thieno[3,2-d]pyrimidin-6-yl]-1-methyl-ethyl]carbamate (180 mg, 33% yield). MS m/z 457.3, 455.4 [M−H]⁻; ¹H NMR (CDCl₃) δ: 7.56 (br s, 1H), 4.44-4.58 (m, 1H), 3.95-4.11 (m, 1H), 2.99-3.23 (m, 2H), 2.40 (s, 3H), 1.57 (s, 9H), 1.45 (s, 9H), 1.20 (d, J=6.7 Hz, 3H).

### Step 2: tert-Butyl N-[6-[(2S)-2-(tert-butoxycarbonylamino)propyl]-2-chloro-7-methyl-thieno[3,2-d]pyrimidin-4-yl]-N-[(3,5-difluoro-4-pyridyl)methyl]carbamate

To a mixture of tert-butyl N-[(1S)-2-[4-(tert-butoxycarbonylamino)-2-chloro-7-methyl-thieno[3,2-d]pyrimidin-6-yl]-1-methyl-ethyl]carbamate (40 mg, 0.088 mmol, 1.0 eq.), (3,5-difluoro-4-pyridyl)methanol (19 mg, 0.13 mmol, 1.5 eq.), and PPh₃ (37 mg, 0.14 mmol, 1.6 eq.) in THF (1.0 mL) at 0° C. was added DEAD (40% in toluene) (0.060 mL, 0.13 mmol, 1.5 eq.). After 2 h at room temperature, LC/MS showed complete reaction and the mixture was concentrated. The residue was purified over silica gel with ethyl acetate in dichloromethane (0 to 20% gradient) to give tert-butyl N-[6-[(2S)-2-(tert-butoxycarbonylamino)propyl]-2-chloro-7-methyl-thieno[3,2-d]pyrimidin-4-yl]-N-[(3,5-difluoro-4-pyridyl)methyl]carbamate (51 mg, 100% yield). MS m/z 606.2, 608.3 [M+Na]⁺; ¹H NMR (CDCl₃) δ: 8.28 (s, 2H), 5.38 (s, 2H), 4.41-4.57 (m, 1H), 3.93-4.10 (m, 1H), 3.18 (d, J=4.4 Hz, 1H), 2.99-3.12 (m, 1H), 2.41 (s, 3H), 1.51 (s, 9H), 1.45 (s, 9H), 1.18 (d, J=6.7 Hz, 3H).

### Step 3: 6-[(2S)-2-Aminopropyl]-2-chloro-N-[(3,5-difluoro-4-pyridyl)methyl]-7-methyl-thieno[3,2-d]pyrimidin-4-amine dihydrochloride

tert-Butyl N-[6-[(2S)-2-(tert-butoxycarbonylamino)propyl]-2-chloro-7-methyl-thieno[3,2-d]pyrimidin-4-yl]-N-[(3,5-difluoro-4-pyridyl)methyl] carbamate (51 mg, 0.087 mmol) was treated with HCl in dioxane (1.0 mL) at room temperature for 2 h, then diluted with ether and filtered. The solid was collected and dried to give 6-[(2S)-2-aminopropyl]-2-chloro-N-[(3,5-difluoro-4-pyridyl)methyl]-7-methyl-thieno[3,2-d]pyrimidin-4-amine dihydrochloride (40 mg, 100% yield). MS m/z 384.1, 386.1 [M+H]⁺; ¹H NMR (methanol-d₄) δ: 8.57 (br s, 2H), 5.02 (s, 2H), 3.63-3.67 (m, 1H), 3.36-3.45 (m, 1H), 3.30 (s, 1H), 2.41 (s, 3H), 1.38 (d, J=6.3 Hz, 3H); 3 NHs not observed.

The compounds below were prepared according to the procedure of Example 8 by substituting the appropriate starting materials, reagents and reaction conditions.

| Com- | |
|---|---|
| pound | Spectral Data |
| 62 | MS m/z 368.1, 370.1 [M + H]⁺; ¹H NMR (methanol-d4) δ: 8.50 |
| | (br s, 1H), 8.40 (d, J = 1.4 Hz, 1H), 7.34 (s, 1H), 5.04 (s, 2H), |
| | 3.43-3.53 (m, 1H), 3.19-3.31 (m, 2H), 2.36 (s, 3H), 1.64- |
| | 1.87 (m, 2H), 1.09 (t, J = 7.6 Hz, 3H); 3 NHs not observed. |
| 98 | MS m/z 367.2, 369.2 [M + H]⁺; ¹H NMR (methanol-d₄) δ: 8.87 |
| | (d, J = 2.7 Hz, 1H), 8.66 (d, J = 2.0 Hz, 1H), 4.97 (s, 2H), 3.59- |
| | 3.67 (m, 1H), 3.32-3.36 (m, 1H), 3.16-3.23 (m, J = 14.6, 8.4 Hz, |
| | 1H), 2.32 (s, 3H), 1.36 (d, J = 6.6 Hz, 3H); 3 NHs not observed. |

### Example 9 (Compound 8)

### 6-[(2S)-2-Aminopropyl]-N-[(furan-2-yl)methyl]thieno[3,2-d]pyrimidin-4-amine dihydrochloride

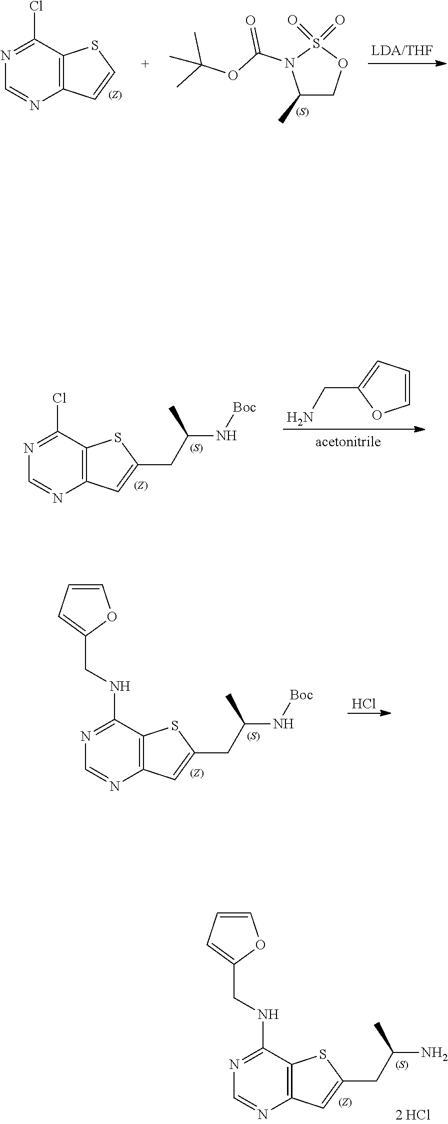

### Step 1: tert-Butyl N-[(1S)-2-(4-chlorothieno[3,2-d]pyrimidin-6-yl)-1-methyl-ethyl]carbamate

To a solution of 4-chlorothieno[3,2-d]pyrimidine (340 mg, 2.0 mmol, 1.0 eq.) in THF (8.0 mL) at −78° C. was added LDA (2.0 M) (970 mg, 1.2 mL, 2.4 mmol, 1.2 eq.). After 30 min, a solution of tert-butyl (4S)-4-methyl-2,2-dioxo-oxathiazolidine-3-carboxylate (620 mg, 2.6 mmol, 1.3 eq.) in THF (8.0 mL) was added dropwise. The mixture was stirred for 1 h while the temperature was warmed slowly to −20° C. The reaction was quenched by addition of 1.0 N citric acid. The mixture was stirred at room temperature for 90 min, then diluted with ethyl acetate, washed with water, saturated sodium bicarbonate, water and brine, and then dried over sodium sulfate and evaporated. The residue was purified over silica gel with ethyl acetate in dichloromethane (0 to 15% gradient) to give tert-butyl N-[(1S)-2-(4-chlorothieno[3,2-d]pyrimidin-6-yl)-1-methyl-ethyl]carbamate (160 mg, 24% yield). MS m/z 328.2, 330.2 [M+H]⁺; ¹H NMR (CDCl₃) δ: 8.86 (s, 1H), 7.26 (s, 1H), 4.36-4.53 (m, 1H), 3.90-4.06 (m, 1H), 3.12 (d, J=6.0 Hz, 2H), 1.37 (s, 9H), 1.15 (d, J=6.7 Hz, 3H).

### Step 2: tert-Butyl N-[(1S)-2-[4-(2-furylmethylamino)thieno[3,2-d]pyrimidin-6-yl]-1-methyl-ethyl]carbamate

A mixture of tert-butyl N-[(1S)-2-(4-chlorothieno[3,2-d]pyrimidin-6-yl)-1-methyl-ethyl]carbamate (50 mg, 0.15 mmol, 1.0 eq.) and 2-furylmethanamine (74 mg, 0.067 mL, 0.76 mmol, 5.0 eq.) in acetonitrile (0.5 mL) was stirred at room temperature overnight. LC/MS showed the reaction proceeding slowly. The mixture was then heated at 70° C. for 4 h, then cooled and evaporated. The mixture was treated with water and ethyl acetate. The organic layer was separated and washed with water and brine, dried over sodium sulfate and evaporated. The residue was purified over silica gel with ethyl acetate in dichloromethane (0 to 100% gradient) to give tert-butyl N-[(1S)-2-[4-(2-furylmethylamino)thieno[3,2-d]pyrimidin-6-yl]-1-methyl-ethyl]carbamate. MS m/z 389.4, 390.4 [M+H]⁺; ¹H NMR (CDCl₃) δ: 8.65 (s, 1H), 7.41 (dd, J=1.7, 0.8 Hz, 1H), 7.25 (s, 1H), 6.33-6.40 (m, 2H), 5.42-5.73 (m, 1H), 4.89 (d, J=5.3 Hz, 2H), 4.43-4.59 (m, 1H), 3.90-4.08 (m, 1H), 3.12 (d, J=5.2 Hz, 2H), 1.43 (s, 9H), 1.19 (d, J=6.7 Hz, 3H).

### Step 3: 6-[(2S)-2-Aminopropyl]-N-(2-furylmethyl)thieno[3,2-d]pyrimidin-4-amine dihydrochloride

tert-Butyl N-[(1S)-2-[4-(2-furylmethylamino)thieno[3,2-d]pyrimidin-6-yl]-1-methyl-ethyl]carbamate obtained above was treated with anisole (0.2 mL) and HCl (4 M in dioxane) (2.0 mL). The mixture was stirred at room temperature for 2 h, then diluted with a large amount of ether and filtered. The solid was collected and dried to give 6-[(2S)-2-aminopropyl]-N-(2-furylmethyl)thieno[3,2-d]pyrimidin-4-amine dihydrochloride (36 mg, 65% yield over 2 steps). MS m/z 289.3, 290.3 [M+H]⁺; ¹H NMR (methanol-d₄) δ: 8.75 (s, 1H), 7.36-7.49 (m, 2H), 6.29-6.45 (m, 2H), 4.92 (s, 2H), 3.65—3.72 (m, 1H), 3.36-3.44 (m, 1H), 3.29-3.35 (m, 1H), 1.35 (d, J=6.6 Hz, 3H); 3 NHs not observed.

The compounds below were prepared according to the procedure of Example 9 by substituting the appropriate starting materials, reagents and reaction conditions.

| Com- | |
|---|---|
| pound | Spectral Data |
| 11 | MS m/z 300.3 [M + H]⁺; ¹H NMR (methanol-d4) δ: 8.79 (d, J = |
| | 6.7 Hz, 2H), 8.72 (s, 1H), 8.11 (d, = 6.4 Hz, 2H), 7.51 (s, 1H), |
| | 5.23 (s, 2H), 3.69-3.79 (m, 1H), 3.44 (d, J = 6.4 Hz, 1H), 3.32- |
| | 3.50 (m, 1H), 1.38 (d, J = 6.6 Hz, 3H), 3 NHs not observed. |

### Example 10 (Compound 15)

### 6-[(2S)-2-Aminopropyl]-N-[(furan-2-yl)methyl]-2,7-dimethylthieno[3,2-d]pyrimidin-4-amine dihydrochloride

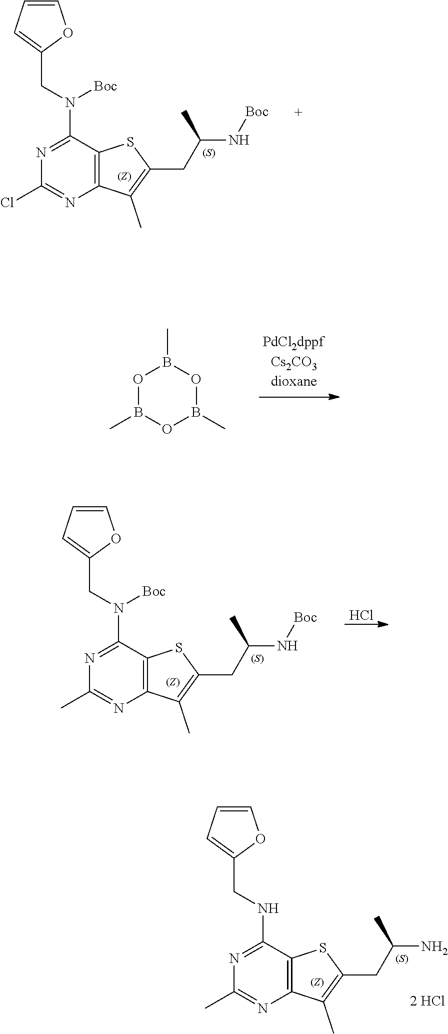

### Step 1: tert-Butyl N-[(1S)-2-[4-(2-furylmethylamino)-2,7-dimethyl-thieno[3,2-d]pyrimidin-6-yl-]1-methyl-ethyl]carbamate

A mixture of tert-butyl N-[(1S)-2-[2-chloro-4-(2-furylmethylamino)-7-methyl-thieno[3,2-d]pyrimidin-6-yl]-1-methyl-ethyl]carbamate (60 mg, 0.14 mmol, 1.0 eq., prepared according to the procedure in example 3), trimethylboroxine (35 mg, 0.039 mL, 0.27 mmol, 2.0 eq.), Cs₂CO₃ (130 mg, 0.41 mmol, 3.0 eq.) and PdCl₂dppf CH₂Cl₂ complex (11 mg, 0.014 mmol, 0.10 eq.) in dioxane (1.0 mL) and water (0.1 mL) was stirred at 120° C. for 2 h under Ar. After cooling, the reaction was diluted with ethyl acetate and washed with brine, and then dried and concentrated. The residue was purified over silica gel with ethyl acetate in hexanes (5 to 50% gradient) to give tert-butyl N-[(1S)-2-[4-(2-furylmethylamino)-2,7-dimethyl-thieno[3,2-d]pyrimidin-6-yl]-1-methyl-ethyl]carbamate (60 mg, 100% yield). MS m/z 417.5 [M+H−Boc]⁺; ¹H NMR (CDCl₃) δ: 7.26 (d, 1H), 6.23-6.27 (m, 1H), 6.20 (d, J=3.1 Hz, 1H), 5.17 (s, 2H), 4.40-4.58 (m, 1H), 3.94-4.11 (m, 1H), 3.17 (br s, 1H), 2.99-3.11 (m, 1H), 2.81 (s, 3H), 2.41 (s, 3H), 1.49 (s, 9H), 1.46 (s, 9H), 1.16 (d, J=6.9 Hz, 3H).

### Step 2: 6-[(2S)-2-Aminopropyl]-N-(2-furylmethyl)-2,7-dimethyl-thieno[3,2-d]pyrimidin-4-amine dihydrochloride

A mixture of tert-butyl N-[6-[(2S)-2-(tert-butoxycarbonylamino)propyl]-2,7-dimethyl-thieno[3,2-d]pyrimidin-4-yl]-N-(2-furylmethyl)carbamate (60 mg, 0.12 mmol, 1.0 eq.), anisole (0.2 mL) and HCl (4 M in dioxane) (1.0 mL) was stirred at room temperature for 2 h. A few drops of methanol was added to make the mixture homogeneous and the mixture was stirred for another hour before it was diluted with a large amount of ether. The mixture was filtered, washed with ether and dried to give 6-[(2S)-2-aminopropyl]-N-(2-furylmethyl)-2,7-dimethyl-thieno[3,2-d]pyrimidin-4-amine dihydrochloride (38 mg, 84% yield). MS m/z 317.3 [M+H]⁺; ¹H NMR (methanol-d₄) δ: 7.36 (s, 1H), 6.26-6.37 (m, 2H), 4.84 (s, 2H), 3.51-3.62 (m, 1H), 3.27-3.34 (m, 1H), 3.14-3.19 (m, 1H), 2.68 (s, 3H), 2.36 (s, 3H), 1.27 (d, J=6.6 Hz, 3H); 3 NHs not observed.

The compounds below were prepared according to the procedure of Example 10 by substituting the appropriate starting materials, reagents and reaction conditions.

| Com- | |
|---|---|
| pound | Spectral Data |
| 16 | MS m/z 331.4 [M + H]⁺; ¹H NMR (methanol-d4) δ: 7.31-7.39 |
| | (m, 1H), 6.25-6.36 (m, 2H), 4.85 (s, 2H), 3.51-3.62 (m, 1H), |
| | 3.26-3.34 (m, 1H), 3.11-3.19 (m, 1H), 2.91-3.00 (m, 2H), |
| | 2.36 (s, 3H), 1.33-1.41 (m, 3H), 1.27 (d, J = 6.6 Hz, 3H); |
| | 3 NHs not observed. |
| 17 | MS m/z 343.3 [M + H]⁺; ¹H NMR (methanol-d4) δ: 7.30-7.39 |
| | (m, 1H), 6.23-6.31 (m, 2H), 4.71 (s, 2H), 3.50-3.60 (m, 1H), |
| | 3.25-3.33 (m, 1H), 3.12-3.18 (m, 1H), 2.37 (s, 3H), 2.27- |
| | 2.33 (m, 1H), 1.20-1.40 (m, 7H); 3 NHs not observed. |

### Example 11 (Compounds 20 and 21)

### 6-[(2S)-2-Aminopropyl]-4-{[(furan-2-yl)methyl]amino}-7-methylthieno[3,2-d]pyrimidine-2-carboxamide trifluoroacetic acid

### and

### 6-[(2S)-2-aminopropyl]-4-{[(furan-2-yl)methyl]amino}-7-methylthieno[3,2-d]pyrimidine-2-carbonitrile trifluoroacetic acid

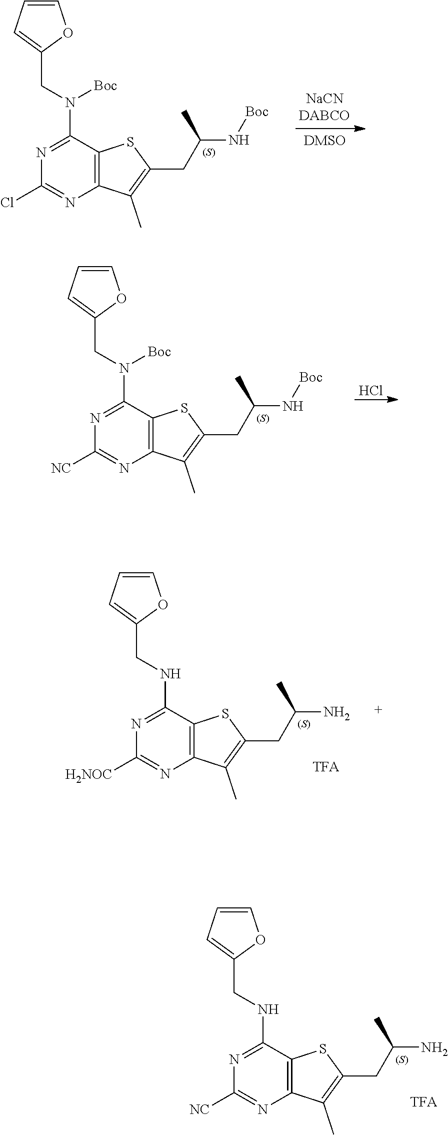

### Step 1: tert-Butyl N-[(1S)-2-[2-cyano-4-(2-furylmethylamino)-7-methyl-thieno[3,2-d]pyrimidin-6-yl]-1-methyl-ethyl]carbamate

A mixture of tert-butyl N-[(1S)-2-[2-chloro-4-(2-furylmethylamino)-7-methyl-thieno[3,2-d]pyrimidin-6-yl]-1-methyl-ethyl]carbamate (60 mg, 0.14 mmol, 1.0 eq., prepared according to the procedure in example 3), 1,4-diazabicyclo[2.2.2]octane (16 mg, 0.14 mmol, 1.0 eq.), and sodium cyanide (10 mg, 0.21 mmol, 1.5 eq.) in DMSO (1.0 mL) and water (0.1 mL) was stirred at 80° C. for 2 h and then at 100° C. for 4 h, and then cooled, diluted with ethyl acetate, washed with brine, dried and evaporated. The residue was purified over silica gel with ethyl acetate in hexanes to give tert-butyl N-[(1S)-2-[2-cyano-4-(2-furylmethylamino)-7-methyl-thieno[3,2-d]pyrimidin-6-yl]-1-methyl-ethyl]carbamate (29 mg, 49% yield). MS m/z 428.5 [M+H−Boc]⁺.

### Step 2: 6-[(2S)-2-Aminopropyl]-4-(2-furylmethylamino)-7-methyl-thieno[3,2-d]pyrimidine-2-carboxamide 2,2,2-trifluoroacetic acid and 6-[(2S)-2-Aminopropyl]-4-(2-furylmethylamino)-7-methyl-thieno[3,2-d]pyrimidine-2-carbonitrile 2,2,2-trifluoroacetic acid

tert-Butyl N-[(1S)-2-[2-cyano-4-(2-furylmethylamino)-7-methyl-thieno[3,2-d]pyrimidin-6-yl]-1-methyl-ethyl]carbamate was stirred with HCl in dioxane (1.0 mL) for 2 h, then diluted with ether and filtered. The solid was collected and purified by prep-HPLC to give 6-[(2S)-2-aminopropyl]-4-(2-furylmethylamino)-7-methyl-thieno[3,2-d]pyrimidine-2-carboxamide 2,2,2-trifluoroacetic acid (4.8 mg, 15% yield) [MS m/z 346.3 [M+H]⁺; ¹H NMR (methanol-d4) δ: 7.38-7.44 (m, 1H), 6.33-6.40 (m, 2H), 4.91 (s, 2H), 3.57-3.67 (m, 1H), 3.16-3.25 (m, 1H), 2.46 (s, 3H), 1.36 (d, J=6.6 Hz, 3H), 1 H obscured by MeOD; 5 NHs not observed] and 6-[(2S)-2-aminopropyl]-4-(2-furylmethylamino)-7-methyl-thieno[3,2-d]pyrimidine-2-carbonitrile 2,2,2-trifluoroacetic acid (10.0 mg, 33% yield). MS m/z 328.3 [M+H]⁺; ¹H NMR (methanol-d₄) δ: 7.38-7.44 (m, 1H), 6.31-6.38 (m, 2H), 4.78 (s, 2H), 3.54-3.64 (m, 1H), 3.28-3.31 (m, 1H), 3.14-3.22 (m, 1H), 2.39 (s, 3H), 1.35 (d, J=6.6 Hz, 3H); 3 NHs not observed.

### Example 12 (Compound 68)

### 6-[(2S)-2-Aminopropyl]-7-bromo-2-chloro-N-[(3-fluoropyridin-4-yl)methyl]thieno[3,2-d]pyrimidin-4-amine dihydrochloride

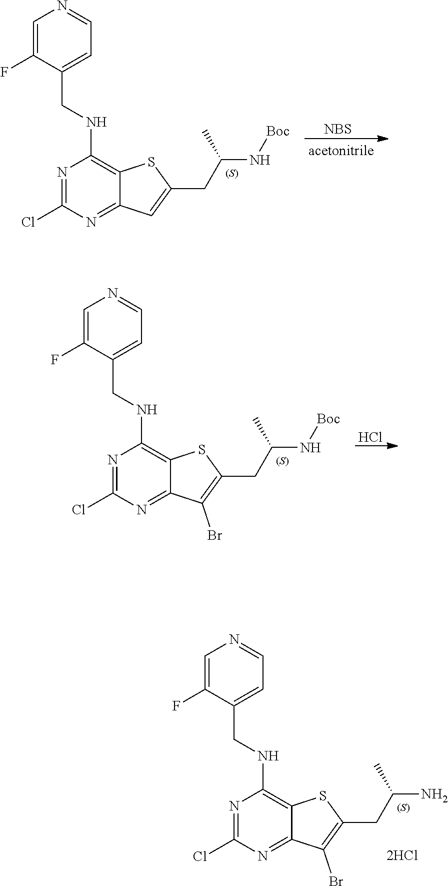

### Step 1: tert-Butyl N-[(1S)-2-[7-bromo-2-chloro-4-[(3-fluoro-4-pyridyl)methylamino]thieno[3,2-d]pyrimidin-6-yl]-1-methyl-ethyl]carbamate

A mixture of tert-butyl N-[(1S)-2-[2-chloro-4-[(3-fluoro-4-pyridyl)methylamino]thieno[3,2-d]pyrimidin-6-yl]-1-methyl-ethyl]carbamate (29 mg, 0.064 mmol, 1.0 eq., prepared according to the procedure in example 6), and NBS (14 mg, 0.077 mmol, 1.2 eq.) in acetonitrile (0.1 mL) was stirred at 80° C. for 8 h, then cooled, diluted with ethyl acetate and then washed with brine, dried and evaporated. The residue was purified over silica gel with ethyl acetate in dichloromethane (0 to 75% gradient) to give tert-butyl N-[(1S)-2-[7-bromo-2-chloro-4-[(3-fluoro-4-pyridyl)methylamino]thieno[3,2-d]pyrimidin-6-yl]-1-methyl-ethyl]carbamate (7 mg, 20% yield). MS m/z 530.0, 531.9, 533.9 [M+H]⁺.

### Step 2: 6-[(2S)-2-Aminopropyl]-7-bromo-2-chloro-N-[(3-fluoro-4-pyridyl)methyl]thieno[3,2-d]pyrimidin-4-amine dihydrochloride

A mixture of tert-butyl N-[(1S)-2-[7-bromo-2-chloro-4-[(3-fluoro-4-pyridyl)methylamino]thieno[3,2-d]pyrimidin-6-yl]-1-methyl-ethyl]carbamate (7.0 mg, 0.01 mmol, 1.0 eq.) and HCl (4 M in dioxane) (0.5 mL, 2 mmol, 200 eq.) was stirred at room temperature for 2 h, then diluted with ether and filtered. The solid was collected and dried to give 6-[(2S)-2-aminopropyl]-7-bromo-2-chloro-N-[(3-fluoro-4-pyridyl)methyl]thieno[3,2-d]pyrimidin-4-amine dihydrochloride (6.0 mg, 90% yield). ¹H NMR (methanol-d₄) δ: 9.00-9.14 (m, 1H), 8.66-8.78 (m, 1H), 8.08-8.22 (m, 1H), 5.11 (s, 2H), 3.76-3.84 (m, 1H), 3.43-3.49 (m, 1H), 3.35-3.41 (m, 1H), 1.41 (d, J=6.4 Hz, 3H); 3 NHs not observed.

### Example 13 (Compound 77)

### 6-[(2S)-2-Aminopropyl]-2-chloro-7-ethyl-N-[(furan-2-yl)methyl]thieno[3,2-d]pyrimidin-4-amine

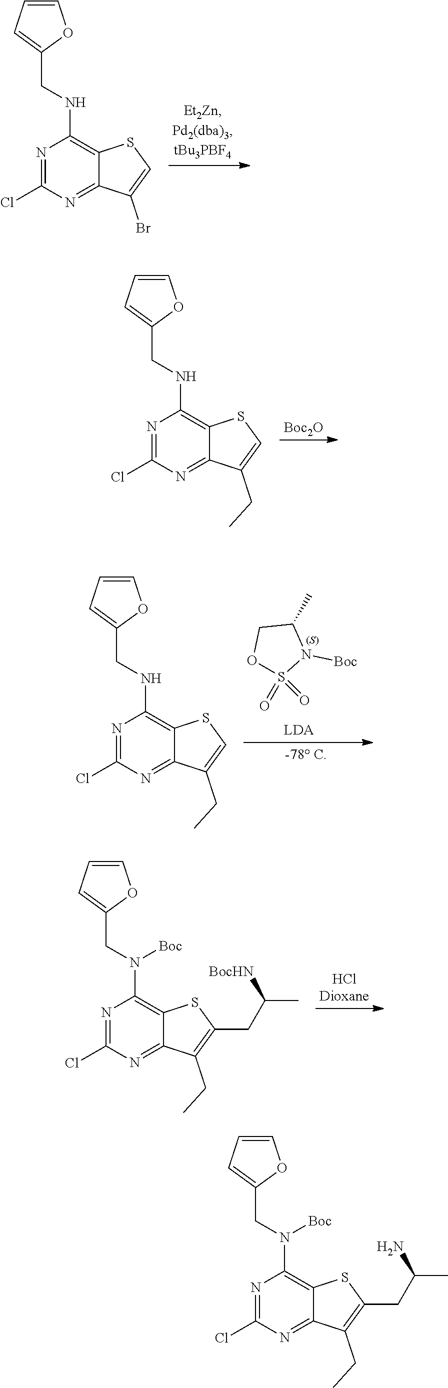

### Step 1: 2-Chloro-7-ethyl-N-(furan-2-ylmethyl)thieno[3,2-d]pyrimidin-4-amine

7-Bromo-2-chloro-N-(2-furylmethyl)thieno[3,2-d]pyrimidin-4-amine (400 mg, 1.2 mmol, 1.0 eq., prepared according to the procedure in example 1), tri-tert-butylphosphonium tetrafluoroborate (15 mg, 0.05 mmol, 0.03 eq.), and tris(dibenzylideneacetone)dipalladium (24 mg, 0.03 mmol, 0.015 eq.) were weighed into a 20 mL scintillation vial. THF (5 mL) was added followed by diethylzinc (1.0 mol/L) in hexanes (1.3 mL, 1.3 mmol, 1.1 eq.). After stirring at room temperature for 1 h, the reaction was quenched with saturated NaHCO₃ and poured into H₂O. The aqueous phase was extracted with EtOAc. The combined organic layers were washed with brine, dried over MgSO₄, filtered, and concentrated. The crude residue was purified on silica gel eluting with 0-30% EtOAc in hexanes to afford 2-chloro-7-ethyl-N-(2-furylmethyl)thieno[3,2-d]pyrimidin-4-amine (210 mg, 62% yield) as a white solid. MS m/z 293.9, 295.9 [M+H]⁺; ¹H NMR (CDCl₃) δ: 7.40-7.42 (m, 1H), 7.38-7.39 (m, 1H), 6.38 (s, 2H), 4.83-4.89 (m, 2H), 2.90 (dd, J=7.5, 1.1 Hz, 2H), 1.34 (t, J=7.5 Hz, 3H); 1 NH not observed.

### Step 2: tert-Butyl (2-chloro-7-ethylthieno[3,2-d]pyrimidin-4-yl)(furan-2-ylmethyl)carbamate

A solution of 2-chloro-7-ethyl-N-(2-furylmethyl)thieno[3,2-d]pyrimidin-4-amine (210 mg, 0.7 mmol, 1.0 eq.), di-tert-butyl dicarbonate (200 mg, 0.9 mmol, 1.1 eq.), 4-dimethylaminopyridine (25 mg, 0.2 mmol, 0.2 eq.), and dichloromethane (2 mL) was stirred at room temperature for 30 min. After concentration under reduced pressure, the crude residue was purified on silica gel eluting with 0-10% EtOAc in hexanes to afford tert-butyl N-(2-chloro-7-ethyl-thieno[3,2-d]pyrimidin-4-yl)-N-(2-furylmethyl)carbamate (260 mg, 92% yield) as a white solid. MS m/z 393.9, 395.9 [M+H]⁺; ¹H NMR (CDCl₃) δ: 7.59 (t, J=1.1 Hz, 1H), 7.27-7.31 (m, 1H), 6.19-6.36 (m, 2H), 5.21 (s, 2H), 2.93 (qd, J=7.5, 0.9 Hz, 2H), 1.52 (s, 9H), 1.35 (t, J=7.5 Hz, 3H).

### Step 3: tert-Butyl (S)-(6-(2-((tert-butoxycarbonyl)amino)propyl)-2-chloro-7-ethylthieno[3,2-d]pyrimidin-4-yl)(furan-2-ylmethyl)carbamate

To a solution of tert-butyl N-(2-chloro-7-ethyl-thieno[3,2-d]pyrimidin-4-yl)-N-(2-furylmethyl)carbamate (260 mg, 0.7 mmol, 1.0 eq.) in THF (4 mL) at −78° C., was added lithium diisopropylamide (2.0 mol/L) in THF/heptane/ethylbenzene (0.36 mL, 0.72 mmol, 1.1 eq.). After stirring at −78° C. for 1 h, a solution of tert-butyl (4S)-4-methyl-2,2-dioxo-oxathiazolidine-3-carboxylate (216 mg, 9.1 mmol, 1.3 eq.) in THF (4 mL) was added dropwise. The bath was removed, and the reaction mixture was stirred at room temperature for 1 h at which point it was quenched with 1 M citric acid (4 mL) and allowed to stir at room temperature for 0.5 h. The reaction mixture was diluted with EtOAc and washed with H₂O. The organic layer was dried over MgSO₄, filtered, and concentrated under reduced pressure. The crude residue was purified on silica gel eluting with 0-10% EtOAc in dichloromethane to afford tert-butyl N-[6-[(2S)-2-(tert-butoxycarbonylamino)propyl]-2-chloro-7-ethyl-thieno[3,2-d]pyrimidin-4-yl]-N-(2-furylmethyl)carbamate (226 mg, 62% yield) as a light yellow oil. MS m/z 573.3, 575.2 [M+Na]⁺; ¹H NMR (CDCl₃) δ: 7.28 (s, 1H), 6.26-6.29 (m, 2H), 5.17 (s, 2H), 3.95-4.07 (m, 1H), 3.18 (dd, J=14.3, 5.5 Hz, 1H), 3.00-3.10 (m, 1H), 2.87 (q, J=7.5 Hz, 2H), 1.51 (s, 9H), 1.43 (s, 9H), 1.22 (t, J=7.5 Hz, 3H), 1.17 (d, J=6.7 Hz, 3H); 1 NH not observed.

### Step 4: (S)-6-(2-Aminopropyl)-2-chloro-7-ethyl-N-(furan-2-ylmethyl)thieno[3,2-d]pyrimidin-4-amine

A solution of tert-butyl N-[6-[(2S)-2-(tert-butoxycarbonylamino)propyl]-2-chloro-7-ethyl-thieno[3,2-d]pyrimidin-4-yl]-N-(2-furylmethyl)carbamate (200 mg, 0.4 mmol, 1.0 eq) in HCl (4 M in dioxane) (2 mL) was stirred at room temperature for 4 h. The precipitate was filtered and rinsed with diethyl ether to afford 6-[(2S)-2-aminopropyl]-2-chloro-7-ethyl-N-(2-furylmethyl)thieno[3,2-d]pyrimidin-4-amine hydrochloride (137 mg, 97% yield) as an off-white solid. MS m/z 351.1, 353.1 [M+H]⁺; ¹H NMR (DMSO-d₆) δ: 8.80 (br t, J=5.6 Hz, 1H), 8.31 (br s, 3H), 7.59 (d, J=0.9 Hz, 1H), 6.40 (dd, J=3.1, 1.8 Hz, 1H), 6.32 (d, J=3.1 Hz, 1H), 4.65 (d, J=5.5 Hz, 2H), 3.36-3.43 (m, 1H), 3.31-3.35 (m, 1H), 3.03-3.19 (m, 1H), 2.63-2.82 (m, 2H), 1.22 (d, J=6.4 Hz, 3H), 1.12 (t, J=7.5 Hz, 3H).

### Example 14 (Compound 87)

### 6-[(2S)-2-Aminopropyl]-2-chloro-7-phenyl-N-[(thiophen-2-yl)methyl]thieno[3,2-d]pyrimidin-4-amine

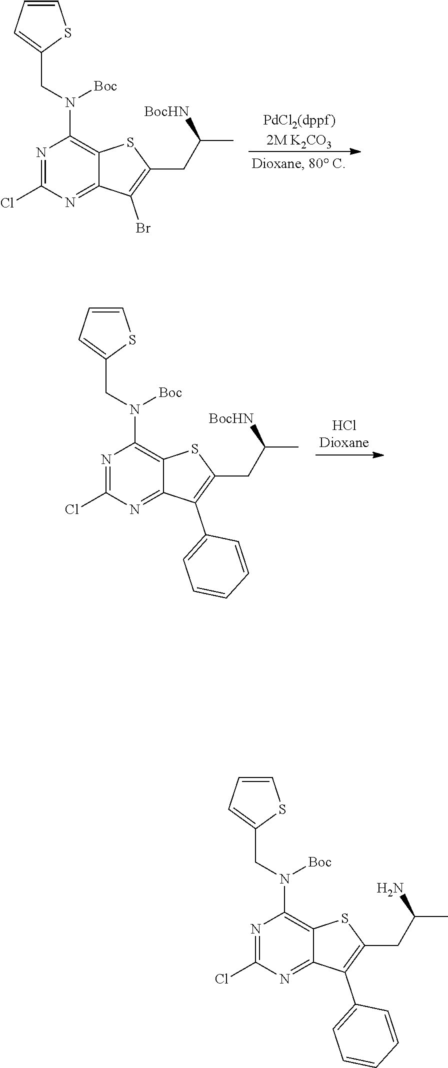 [SMILES: C[C@@H](Cc1sc2c(N(Cc3cccs3)C(=O)OC(C)(C)C)nc(Cl)nc2c1Br)NC(=O)OC(C)(C)C ; "PdCl2(dppf)2M K2CO3" ; C[C@@H](Cc1sc2c(N(Cc3cccs3)C(=O)OC(C)(C)C)nc(Cl)nc2c1-c1ccccc1)NC(=O)OC(C)(C)C ; C[C@H](N)Cc1sc2c(N(Cc3cccs3)C(=O)OC(C)(C)C)nc(Cl)nc2c1-c1ccccc1]

### Step 1: tert-Butyl (S)-(6-(2-((tert-butoxycarbonyl)amino)propyl)-2-chloro-7-phenylthieno[3,2-d]pyrimidin-4-yl)(thiophen-2-ylmethyl)carbamate

A mixture of tert-butyl N-[7-bromo-6-[(2S)-2-(tert-butoxycarbonylamino)propyl]-2-chloro-thieno[3,2-d]pyrimidin-4-yl]-N-(2-thienylmethyl)carbamate (100 mg, 0.2 mmol, 1.0 eq., prepared according to the procedure in example 3), 1,1′-bis(diphenylphosphino)ferrocene-palladium dichloride dichloromethane complex (7 mg, 0.008 mmol, 0.04 eq.), phenylboronic acid (23 mg, 0.2 mmol, 1.0 eq.), 1,4-dioxane (1 mL), and aqueous potassium carbonate (2 M) (0.25 mL, 0.50 mmol, 2.5 eq.) was heated at 80° C. for 2 h, cooled, and then diluted with ethyl acetate, washed with brine, dried over MgSO₄, filtered, and concentrated. The crude residue was purified on silica eluting with 0-20% EtOAc in hexanes to afford tert-butyl N-[6-[(2S)-2-(tert-butoxycarbonylamino)propyl]-2-chloro-7-phenyl-thieno[3,2-d]pyrimidin-4-yl]-N-(2-thienylmethyl)carbamate (75 mg, 75% yield) as an off white solid. MS m/z 615.3, 617.4 [M+H]⁺.

### Step 2: (S)-6-(2-Aminopropyl)-2-chloro-7-phenyl-N-(thiophen-2-ylmethyl)thieno[3,2-d]pyrimidin-4-amine

A solution of tert-butyl N-[6-[(2S)-2-(tert-butoxycarbonylamino)propyl]-2-chloro-7-phenyl-thieno[3,2-d]pyrimidin-4-yl]-N-(2-thienylmethyl)carbamate (75 mg, 0.12 mmol, 1.0 eq.) in HCl (4 M in dioxane) (1 mL) was stirred at room temperature for 2 h. The reaction was concentrated and the crude residue was purified on silica gel eluting with 0-10% MeOH in dichloromethane to afford 6-[(2S)-2-aminopropyl]-2-chloro-7-phenyl-N-(2-thienylmethyl)thieno[3,2-d]pyrimidin-4-amine hydrochloride (18 mg, 25% yield) as a light yellow solid. MS m/z 415.3, 417.4 [M+H]⁺;¹H NMR (methanol-d₄) δ: 7.54 (m, 2H), 7.45-7.50 (m, 1H), 7.39-7.45 (m, 2H), 7.28-7.33 (m, 1H), 7.09-7.15 (m, 1H), 6.95-7.00 (m, 1H), 4.96 (s, 2H), 3.44-3.54 (m, 1H), 3.34-3.37 (m, 1H), 3.21-3.28 (m, 1H), 1.19 (d, J=6.7 Hz, 3H); 3 NHs not observed.

The compounds below were prepared according to the procedure of Example 14 by substituting the appropriate starting materials, reagents and reaction conditions.

| Com- | |
|---|---|
| pound | Spectral Data |
| 86 | MS m/z 379.1, 381.2 [M + H]⁺; ¹H NMR (methanol-d₄) δ: 8.43 |
| | (s, 1H), 7.15 (dd, J = 5.2, 1.2 Hz, 1H), 6.92-7.01 (m, 1H), 6.83 |
| | (dd, J = 5.0, 3.5 Hz, 1H), 4.78-4.80 (s, 2H), 3.48-3.59 (m, 1H), |
| | 3.11-3.18 (m, 2H), 1.70-1.79 (m, 1H), 1.24 (d, J = 6.4 Hz, 3H), |
| | 1.03-1.14 (m, 2H), 0.83-0.95 (m, 2H); 3 NHs not observed. |
| 88 | MS m/z 449.2, 451.2 [M + H]⁺; ¹H NMR (methanol-d₄) δ: 7.48- |
| | 7.55 (m, 2H), 7.36-7.44 (m, 2H), 7.27 (dd, J = 5.2, 1.2 Hz, 1H), |
| | 7.06-7.12 (m, 1H), 6.94 (dd, J = 5.2, 3.4 Hz, 1H), 4.92-4.95 (m, |
| | 2H), 3.48 (dt, J = 8.7, 6.3 Hz, 1H), 3.33-3.36 (m, 1H), 3.17-3.24 |
| | (m, 1H), 1.18 (d, J = 6.4 Hz, 3H); 3 NHs not observed. |

### Example 15 (Compounds 51 and 52)

### 6-[(2S)-2-Amino-4-fluorobutyl]-2-chloro-N-[(furan-2-yl)methyl]-7-methylthieno[3,2-d]pyrimidin-4-amine dihydrochloride

### and

### (4S)-4-[(2-Chloro-4-{[(furan-2-yl)methyl]amino}-7-methylthieno[3,2-d]pyrimidin-6-yl)methyl]-1,3-oxazinan-2-one

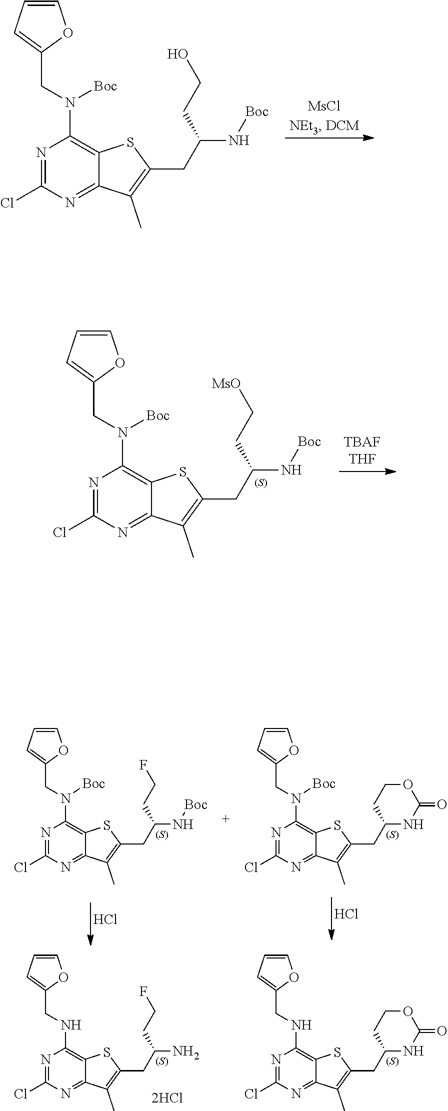

### Step 1: [(3S)-3-(tert-Butoxycarbonylamino)-4-[4-[tert-butoxycarbonyl(2-furylmethyl)amino]-2-chloro-7-methyl-thieno[3,2-d]pyrimidin-6-yl]butyl]methanesulfonate

To a solution of tert-butyl N-[6-[(2S)-2-(tert-butoxycarbonylamino)-4-hydroxy-butyl]-2-chloro-7-methyl-thieno[3,2-d]pyrimidin-4-yl]-N-(2-furylmethyl)carbamate (125 mg, 0.220 mmol, 1.0 eq., prepared according to the procedure in example 4), and DIPEA (58.1 mg, 0.0770 mL, 0.441 mmol, 2.0 eq.) in CH₂Cl₂ (2.0 mL) cooled to 0° C. was added methanesulfonyl chloride (1.0 M in CH₂Cl₂) (490 mg, 0.33 mL, 0.331 mmol, 1.50 eq.). The reaction was completed instantly as shown by LC/MS. The mixture was diluted with cold water and CH₂Cl₂, washed with 1.0 M KHSO₄,sodium bicarbonate and brine, dried over sodium sulfate and evaporated to give [(3S)-3-(tert-butoxycarbonylamino)-4-[4-[tert-butoxycarbonyl(2-furylmethyl)amino]-2-chloro-7-methyl-thieno[3,2-d]pyrimidin-6-yl]butyl]methanesulfonate (148 mg, 104% yield), which was used in the next step without further purification. MS m/z 646.0, 648.0 [M+H]⁺.

### Step 2: tert-Butyl (S)-(6-(2-((tert-butoxycarbonyl)amino)-4-fluorobutyl)-2-chloro-7-methylthieno[3,2-d]pyrimidin-4-yl)(furan-2-ylmethyl)carbamate and tert-butyl (S)-(2-chloro-7-methyl-6-((2-oxo-1,3-oxazinan-4-yl)methyl)thieno[3,2-d]pyrimidin-4-yl)(furan-2-ylmethyl)carbamate

A mixture of [(3S)-3-(tert-butoxycarbonylamino)-4-[4-[tert-butoxycarbonyl(2-furylmethyl)amino]-2-chloro-7-methyl-thieno[3,2-d]pyrimidin-6-yl]butyl]methanesulfonate (88.0 mg, 0.14 mmol) and TBAF (1.0 M, 1.0 mL) was stirred at 65° C. for 1 h, then cooled and concentrated. The residue was purified over silica gel eluting with ethyl acetate in dichloromethane (0 to 10% gradient) to give tert-butyl (S)-(6-(2-((tert-butoxycarbonyl)amino)-4-fluorobutyl)-2-chloro-7-methylthieno[3,2-d]pyrimidin-4-yl)(furan-2-ylmethyl)carbamate (20 mg, 26% yield). MS m/z 569.1, 571.1 [M+H]⁺; ¹H NMR (CDCl₃) δ: 7.29-7.32 (m, 1H), 6.24-6.34 (m, 2H), 5.20 (s, 2H), 4.45-4.68 (m, 3H), 3.99-4.09 (m, 1H), 3.22-3.33 (m, 1H), 3.10-3.21 (m, 1H), 2.41 (s, 3H), 1.95-2.05 (m, 1H), 1.79-1.94 (m, 1H), 1.53 (s, 9H), 1.43 (s, 9H); and tert-butyl (S)-(2-chloro-7-methyl-6-((2-oxo-1,3-oxazinan-4-yl)methyl)thieno[3,2-d]pyrimidin-4-yl)(furan-2-ylmethyl)carbamate (20 mg, 30% yield). [MS m/z 515.1, 517.1 [M+Na]⁺; ¹H NMR (CDCl₃) δ: 7.30-7.34 (m, 1H), 6.25-6.35 (m, 2H), 5.55-5.69 (m, 1H), 5.22 (s, 2H), 4.35-4.44 (m, 1H), 4.22-4.31 (m, 1H), 3.83-3.93 (m, 1H), 3.08-3.21 (m, 2H), 2.36-2.44 (m, 3H), 2.07-2.14 (m, 1H), 1.79-1.91 (m, 1H), 1.55 (s, 9H).

### Step 3: (S)-6-(2-Amino-4-fluorobutyl)-2-chloro-N-(furan-2-ylmethyl)-7-methylthieno[3,2-d]pyrimidin-4-amine dihydrochloride

The general de-Boc procedure using HCl in dioxane was applied to tert-butyl (S)-(6-(2-((tert-butoxycarbonyl)amino)-4-fluorobutyl)-2-chloro-7-methylthieno[3,2-d]pyrimidin-4-yl)(furan-2-ylmethyl)carbamate to provide (S)-6-(2-amino-4-fluorobutyl)-2-chloro-N-(furan-2-ylmethyl)-7-methylthieno[3,2-d]pyrimidin-4-amine dihydrochloride (16 mg, 90% yield). MS m/z 369.1, 371.1 [M+H]⁺; ¹H NMR (methanol-d₄) δ: 7.45 (s, 1H), 6.36 (s, 2H), 4.78 (s, 2H), 4.57-4.76 (m, 2H), 3.71-3.82 (m, 1H), 3.34-3.42 (m, 2H), 2.37 (s, 3H), 2.07-2.19 (m, 2H); 3 NHs not observed.

### Step 4: (S)-4-((2-Chloro-4-((furan-2-ylmethyl)amino)-7-methylthieno[3,2-d]pyrimidin-6-yl)methyl)-1,3-oxazinan-2-one

The general de-Boc procedure using HCl in dioxane was applied to tert-butyl (S)-(2-chloro-7-methyl-6-((2-oxo-1,3-oxazinan-4-yl)methyl)thieno[3,2-d]pyrimidin-4-yl)(furan-2-ylmethyl)carbamate to provide (S)-4-((2-chloro-4-((furan-2-ylmethyl)amino)-7-methylthieno[3,2-d]pyrimidin-6-yl)methyl)-1,3-oxazinan-2-one (10 mg, 63% yield). MS m/z 393.1, 395.1 [M+H]⁺; ¹H NMR (CDCl₃) d: 7.41 (s, 1H), 6.38 (s, 2H), 5.64 (br s, 1H), 5.39 (br s, 1H), 4.79-4.93 (m, 2H), 4.32-4.41 (m, 1H), 4.21-4.30 (m, 1H), 3.79-3.91 (m, 1H), 3.13 (d, J=6.7 Hz, 2H), 2.38 (s, 3H), 2.03-2.13 (m, 1H), 1.78-1.89 (m, 1H).

### Example 16 (Compound 76)

### 6-[(2R)-2-amino-3-methoxypropyl]-2-chloro-N-[(furan-2-yl)methyl]-7-methylthieno[3,2-d]pyrimidin-4-amine

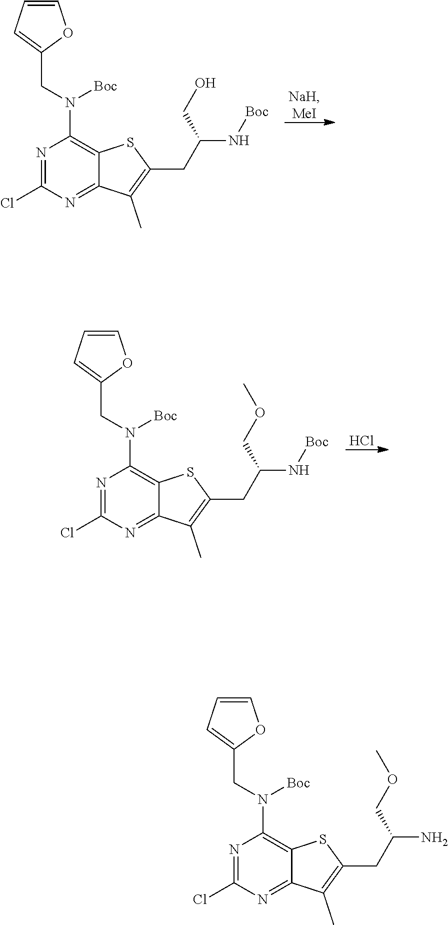

### Step 1: tert-Butyl (R)-(6-(2-((tert-butoxycarbonyl)amino)-3-methoxypropyl)-2-chloro-7-methylthieno[3,2-d]pyrimidin-4-yl)(furan-2-ylmethyl)carbamate

To a stirred solution of tert-butyl N-[6-[(2R)-2-(tert-butoxycarbonylamino)-3-hydroxy-propyl]-2-chloro-7-methyl-thieno[3,2-d]pyrimidin-4-yl]-N-(2-furylmethyl)carbamate (100.0 mg, 0.2 mmol, 1.0 eq., prepared according to the procedure in example 4), in a mixture of DMF (0.5 mL) and THF (1.5 mL) was added sodium hydride (60 mass %) in mineral oil (10 mg, 0.25 mmol, 1.3 eq.) at 0° C. After stirring at 0° C. for 30 min, a solution of iodomethane (2.0 M) in tert-butyl methyl ether (100 μL, 0.20 mmol, 1.1 eq.) was added. The reaction was warmed to room temperature and stirred for an additional 12 h. The reaction was quenched with H₂O (˜5 mL) and then extracted with EtOAc. The combined organic layers were washed with brine, dried over MgSO₄, filtered, and concentrated under reduced pressure. The crude residue was purified on silica gel eluting with 0-30% EtOAc in hexanes doped with 10% dichloromethane to afford tert-butyl N-[6-[(2R)-2-(tert-butoxycarbonylamino)-3-methoxy-propyl]-2-chloro-7-methyl-thieno[3,2-d]pyrimidin-4-yl]-N-(2-furylmethyl)carbamate (49.0 mg, 43% yield) as an off-white foam. MS m/z 567.2, 569.2 [M+H]⁺; ¹H NMR (acetone-d₆) δ: 7.36-7.45 (m, 1H), 6.27-6.36 (m, 2H), 5.17-5.24 (m, 2H), 3.99-4.05 (m, 1H), 3.41-3.47 (m, 2H), 3.33-3.37 (m, 3H), 3.27-3.33 (m, 1H), 3.11-3.19 (m, 1H), 2.76 (s, 3H), 1.53 (s, 9H), 1.32-1.37 (m, 9H); 1 NH not observed.

### Step 2: (R)-6-(2-Amino-3-methoxypropyl)-2-chloro-N-(furan-2-ylmethyl)-7-methylthieno[3,2-d]pyrimidin-4-amine

A mixture of tert-butyl N-[(1R)-1-[[2-chloro-4-(2-furylmethylamino)-7-methyl-thieno[3,2-d]pyrimidin-6-yl]methyl]-2-methoxy-ethyl]carbamate (30.0 mg, 0.06 mmol, 1.0 eq.) in anisole (0.40 mL, 3.6 mmol, 57 eq.) and hydrochloric acid (4 M) in 1,4-dioxane (3.0 mL) was stirred at room temperature for 2 h. The reaction mixture was diluted with ether (˜10 mL) and the resulting heterogeneous mixture was stirred at room temperature for 15 min. The precipitate was filtered and washed with diethyl ether to give an off-white solid which was dried under high vacuum to afford 6-[(2R)-2-amino-3-methoxy-propyl]-2-chloro-N-(2-furylmethyl)-7-methyl-thieno[3,2-d]pyrimidin-4-amine dihydrochloride (16.0 mg, 57% yield) as an off-white solid. MS m/z 367.2, 369.2 [M+H]⁺; ¹H NMR (DMSO-d₆) δ: 8.72-8.84 (m, 1H), 8.19-8.24 (m, 2H), 7.59-7.62 (m, 1H), 6.40-6.44 (m, 1H), 6.32-6.35 (m, 1H), 4.67 (d, J=5.5 Hz, 2H), 3.51-3.55 (m, 1H), 3.47-3.51 (m, 1H), 3.36-3.41 (m, 1H), 3.33 (s, 3H), 3.18-3.25 (m, 2H), 2.24 (s, 3H).

### Example 17 (Compound 78)

### 2-Chloro-6-[(2S)-2-(cyclobutylamino)propyl]-N-[(furan-2-yl)methyl]-7-methylthieno[3,2-d]pyrimidin-4-amine

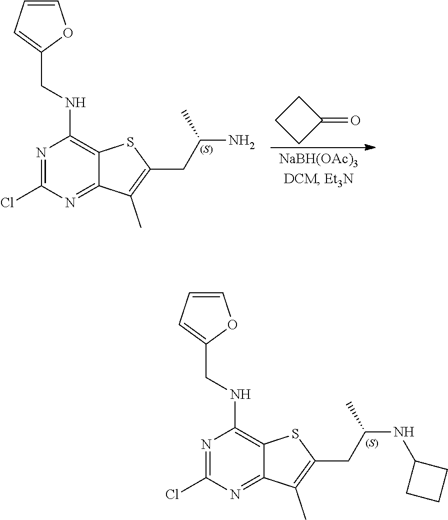

A mixture of (S)-6-(2-aminopropyl)-2-chloro-N-(furan-2-ylmethyl)-7-methylthieno[3,2-d]pyrimidin-4-amine (HClsalt, 63 mg, 0.17 mmol, 1.0 eq. prepared according to the procedure in example 3, cyclobutanone (24 mg, 0.34 mmol, 2.0 eq.), triethylamine (34 mg, 2.0 eq.) and acetic acid (31 mg, 3.0 eq.) in dichloroethane (0.3 mL) was stirred at room temperature for 30 min. Sodium triacetoxyborohydride (111 mg, 0.51 mmol, 3.0 eq.) was added and the mixture was stirred at 50° C. for 16 h. After cooling, the reaction was quenched by addition of water (a few drops). The crude product was filtered, washed with methanol, and the filtrate was concentrated and purified by prep-HPLC eluting with 5-60% CH₃CN in water containing 0.1% formic acid to afford (S)-2-chloro-6-(2-(cyclobutylamino)propyl)-N-(furan-2-ylmethyl)-7-methylthieno[3,2-d]pyrimidin-4-amine (36 mg, 55% yield) as the formic acid salt. MS m/z 391.2, 393.2 [M+H]⁺; ¹H NMR (methanol-d₄) δ: 8.43 (s, 1H), 7.32 (d, J=1.2 Hz, 1H), 6.18-6.30 (m, 2H), 4.63 (s, 2H), 3.50-3.60 (m, 1H), 3.06-3.15 (m, 2H), 2.78-2.85 (m, 1H), 2.12-2.25 (m, 5H), 1.78-1.93 (m, 2H), 1.63-1.73 (m, 2H), 1.04 (d, J=6.1 Hz, 3H), 2 NHs not observed.

### Example 18 (Compound 65)

### 6-[(2S)-2-Aminopropyl]-2-chloro-N-[(furan-2-yl)methyl]-7-(4-methoxyphenyl)thieno[3,2-d]pyrimidin-4-amine

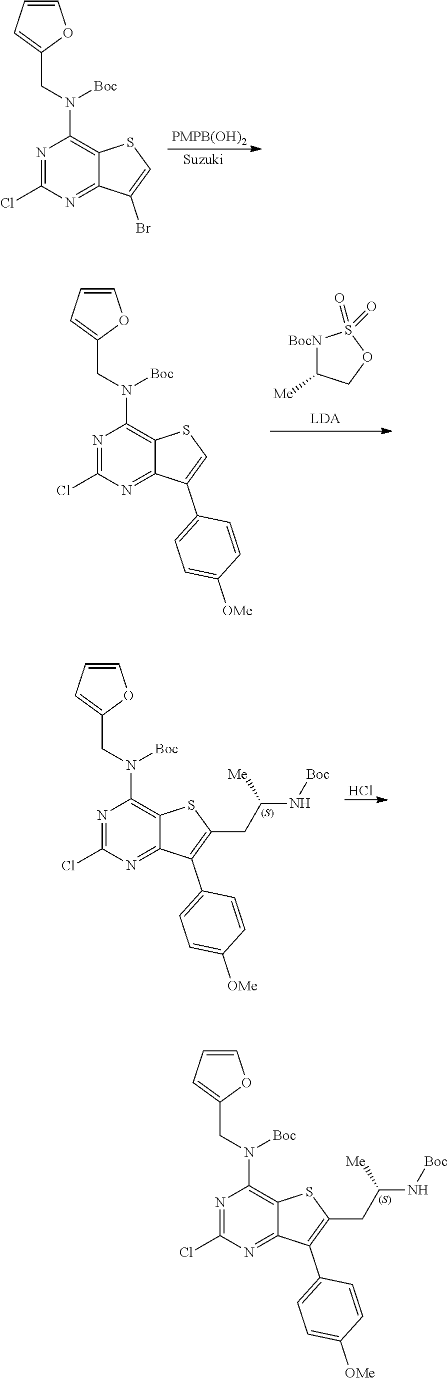

### Step 1: tert-Butyl N-[2-chloro-7-(4-methoxyphenyl)thieno[3,2-d]pyrimidin-4-yl]-N-(2-furylmethyl)carbamate

To a mixture of tert-butyl N-(7-bromo-2-chloro-thieno[3,2-d]pyrimidin-4-yl)-N-(2-furylmethyl)carbamate (500 mg, 1.1 mmol, 1.0 eq.) prepared according to the procedure in example 3,4-methoxyphenylboronic acid (193 mg, 1.2 mmol, 1.1 eq.), potassium carbonate (3.0 eq.), and 1,1′-bis(diphenylphosphino) ferrocene-palladium(II)dichloride dichloromethane complex (94 mg, 0.11 mmol, 0.1 eq.), was added dioxane (5 mL), and the reaction was stirred at 100° C. for 24 h. The mixture was cooled to room temperature and diluted with ethyl acetate, washed with water, sodium bicarbonate and brine. The organic layer was dried over sodium sulfate, filtered and concentrated under reduced pressure. The crude residue was purified over silica gel with ethyl acetate in hexanes (0-20%) to then give tert-butyl N-[2-chloro-7-(4-methoxyphenyl)thieno[3,2-d]pyrimidin-4-yl]-N-(2-furylmethyl)carbamate (306 mg, 57% yield). MS m/z 472.4, 474.4 [M+H]⁺.

### Step 2: tert-Butyl N-[6-[(2S)-2-(tert-butoxycarbonylamino)propyl]-2-chloro-7-(4-methoxyphenyl)thieno[3,2-d]pyrimidin-4-yl]-N-(2-furylmethyl)carbamate

To a solution of tert-butyl N-[2-chloro-7-(4-methoxyphenyl)thieno[3,2-d]pyrimidin-4-yl]-N-(2-furylmethyl)carbamate (93 mg, 0.19 mmol, 1.0 eq.) in THF (0.4 mL) at −78° C. was added n-butyllithium (2.5 mol/L) in hexanes (0.08 mL, 0.21 mmol, 1.1 eq.) dropwise. After 15 min a solution of tert-butyl (4S)-4-methyl-2,2-dioxo-oxathiazolidine-3-carboxylate (51 mg, 0.21 mmol, 1.1 eq.) in THF (0.4 mL) was added dropwise. The mixture was stirred at −78° C. for 10 min and was then quenched with 1.0 M citric acid, followed by stirring at room temperature for 15 min. The mixture was diluted with ethyl acetate, washed with water, sodium bicarbonate and brine. The organic layer was dried over sodium sulfate, filtered and concentrated under reduced pressure. The crude residue was purified over silica gel with ethyl acetate in hexanes (2-30%) to afford tert-butyl N-[6-[(2S)-2-(tert-butoxycarbonylamino)propyl]-2-chloro-7-(4-methoxyphenyl)thieno[3,2-d]pyrimidin-4-yl]-N-(2-furylmethyl)carbamate (55 mg, 44% yield). MS m/z 629.6, 631.6 [M+H]⁺; ¹H NMR (CDCl₃) δ: 7.32 (d, J=8.7 Hz, 2H), 7.30-7.31 (m, J=0.9 Hz, 1H), 7.02 (d, J=8.7 Hz, 2H), 6.28-6.32 (m, 2H), 5.20 (s, 2H), 4.39 (br s, 1H), 3.97 (br s, 1H), 3.88 (s, 3H), 3.07-3.20 (m, 2H), 1.54 (s, 9H), 1.41 (s, 9H), 1.03 (d, J=6.7 Hz, 3H).

### Step 3: 6-[(2S)-2-Aminopropyl]-2-chloro-N-(2-furylmethyl)-7-(4-methoxyphenyl)thieno[3,2-d]pyrimidin-4-amine

To a reaction tube with tert-butyl N-[6-[(2S)-2-(tert-butoxycarbonylamino)propyl]-2-chloro-7-(4-methoxyphenyl)thieno[3,2-d]pyrimidin-4-yl]-N-(2-furylmethyl)carbamate (55 mg, 0.31 mmol, 1.0 eq.) was added hydrochloric acid (4 M) in dioxane (3 mL) and then stirred at room temperature for 1 h. The reaction mixture was diluted with diethyl ether and was filtered and rinsed with diethyl ether. The solid was placed under vacuum for 24 h to give 6-[(2S)-2-aminopropyl]-2-chloro-N-(2-furylmethyl)-7-(4-methoxyphenyl)thieno[3,2-d]pyrimidin-4-amine (18 mg, 48% yield) as an off-white solid. MS m/z 429.4, 431.4 [M+H]⁺; ¹H NMR (methanol-d₄) δ: 7.44 (d, J=0.9 Hz, 1H), 7.33 (d, J=8.7 Hz, 2H), 7.07 (d, J=8.7 Hz, 2H), 6.33-6.37 (m, 2H), 4.77 (s, 2H), 3.86 (s, 3H), 3.43-3.49 (m, 1H), 3.28 (br d, J=6.3 Hz, 1H), 3.23 (br d, J=8.5 Hz, 1H), 1.17 (d, J=6.4 Hz, 3H); 3 NHs not observed.

### Example 19 (Compound 99)

### (2R)-2-amino-3-(2-chloro-7-methoxy-4-{[(thiophen-2-yl)methyl]amino}thieno[3,2-d]pyrimidin-6-yl)propan-1-ol

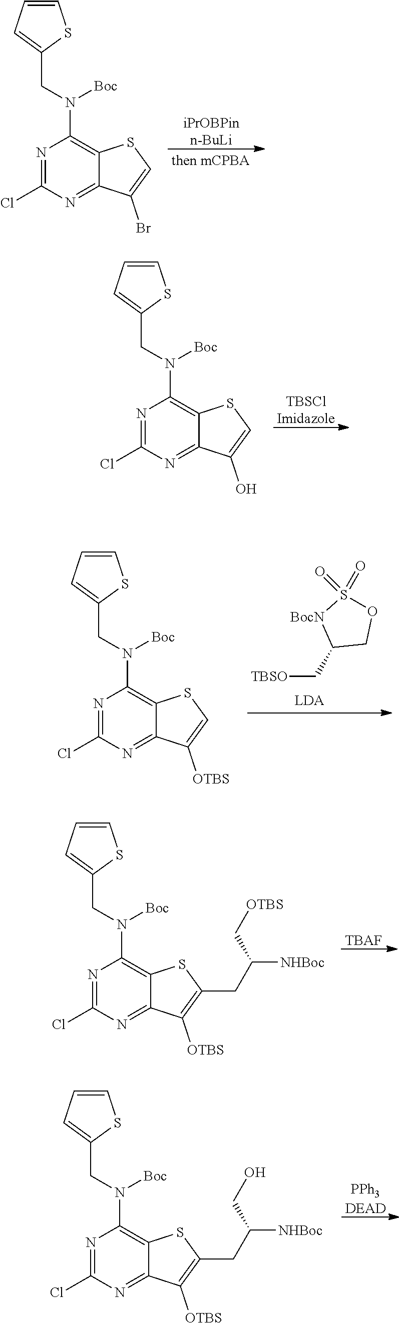

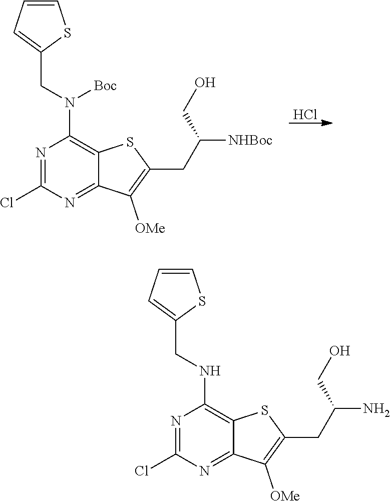

### Step 1: tert-Butyl N-(2-chloro-7-hydroxy-thieno[3,2-d]pyrimidin-4-yl)-N-(2-thienylmethyl)carbamate

To a mixture of tert-butyl N-(7-bromo-2-chloro-thieno[3,2-d]pyrimidin-4-yl)-N-(2-thienylmethyl)carbamate (1.85 g, 1.12 mmol, 1.0 eq.), prepared according to the procedure in example 3, and 2-isopropoxy-4,4,5,5-tetramethyl-1,3,2-dioxoboralane (1 mL, 4.82 mmol, 1.2 eq.) in THF (4 mL) at −78° C., was added n-BuLi (2.5 mol/L) in hexanes (1.9 mL) dropwise. The reaction was stirred at −78° C. for 1 h before removing the bath and warming the reaction to room temperature. The reaction was quenched with sat. NH₄Cl (3 ml), diluted with water and then extracted with ethyl acetate (3×20 mL). The combined organic layers were washed with brine, dried over MgSO₄, filtered, and then concentrated. The crude residue was dissolved in diethyl ether (40 mL) and hydrogen peroxide (35 mass %) in water (1.2 mL, 12.0 mmol, 3.0 eq.) was added. The reaction mixture was stirred for 12 h, cooled to 0° C. and quenched with Na₂SO₃. The crude mixture was then poured into water and extracted with ethyl acetate. The combined organic layers were washed with brine and dried over MgSO₄, filtered and then concentrated under reduced pressure. The crude residue was purified over silica gel with ethyl acetate in hexanes (0-50%) to give tert-butyl N-(2-chloro-7-hydroxy-thieno[3,2-d]pyrimidin-4-yl)-N-(2-thienylmethyl)carbamate (540 mg, 34% yield). MS m/z 398.1, 400.1[M+H]⁺; ¹H NMR (CDCl₃) δ: 7.22 (dd, J=5.1, 1.1 Hz, 1H), 7.12 (d, J=2.9 Hz, 1H), 7.02 (s, 1H), 6.93 (dd, J=5.0, 3.5 Hz, 1H), 5.38 (s, 2H), 1.59 (s, 9H); 1 OH not observed.

### Step 2: tert-ButylN-[7-[tert-butyhdimethyl)silyl]oxy-2-chloro-thieno[3,2-d]pyrimidin-4-yl]-N-(2-thienylmethyl)carbamate

To a mixture of tert-butyl N-(2-chloro-7-hydroxy-thieno[3,2-d]pyrimidin-4-yl)-N-(2-thienylmethyl)carbamate (540 mg, 1.3 mmol, 1.0 eq), tert-butyldimethylsilyl chloride (253 mg, 1.6 mmol, 1.2 eq.), imidazole (0.1 mL, 1.7 mmol, 1.3 eq.) was added CH₂Cl₂ (5.5 mL). The mixture was stirred at room temperature for 1 h, then diluted with CH₂C₁₂, washed with water, sodium bicarbonate and brine. The organic layer was dried over sodium sulfate, filtered and concentrated under reduced pressure. The crude residue was purified over silica gel with ethyl acetate in hexanes (0-20%) to give tert-butyl N-[7-[tert-butyl(dimethyl)silyl]oxy-2-chloro-thieno[3,2-d]pyrimidin-4-yl]-N-(2-thienylmethyl)carbamate (590 mg, 84% yield). MS m/z 512.3, 514.3 [M+H]⁺; ¹H NMR (CDCl₃) δ: 7.20 (dd, J=5.0, 0.9 Hz, 1H), 7.08 (d, J=3.1 Hz, 1H), 6.99 (s, 1H), 6.90 (dd, J=5.0, 3.7 Hz, 1H), 5.35 (s, 2H), 1.56 (s, 9H), 1.03 (s, 9H), 0.27 (s, 6H).

### Step 3: tert-Butyl N-[6-[(2R)-2-(tert-butoxycarbonylamino)-3-[tert-butyl(dimethyl)silyl]oxy-propyl]-7-[tert-butyhdimethyl)silyl]oxy-2-chloro-thieno[3,2-d]pyrimidin-4-yl]-N-(2-thienylmethyl)carbamate

To a solution of tert-butyl N-[7-[tert-butyl(dimethyl)silyl]oxy-2-chloro-thieno[3,2-d]pyrimidin-4-yl]-N-(2-thienylmethyl)carbamate (590 mg, 1.1 mmol, 1.0 eq.) in THF (2.0 mL) at−78° C. was added LDA (2.0 M in THF/heptane/ethylbenzene) (0.7 mL, 1.3 mmol, 1.2 eq.). After 15 min a solution of tert-butyl (4S)-4-[[tert-butyl(dimethyl)silyl]oxymethyl]-2,2-dioxo-oxathiazolidine-3-carboxylate (550 mg, 1.5 mmol, 1.3 eq.) in THF (2.0 mL) was added dropwise. The mixture was stirred at −78° C. for 10 min and was then quenched with 1.0 M citric acid, followed by stirring at room temperature for 15 min. The mixture was diluted with ethyl acetate, washed with water, sodium bicarbonate and brine. The organic layer was dried over sodium sulfate, filtered and then concentrated under reduced pressure. The crude residue was purified over silica gel with ethyl acetate in hexanes (2-35%) to give tert-butyl N-[6-[(2R)-2-(tert-butoxycarbonylamino)-3-[tert-butyl(dimethyl)silyl]oxy-propyl]-7-[tert-butyl(dimethyl)silyl]oxy-2-chloro-thieno[3,2-d]pyrimidin-4-yl]-N-(2-thienylmethyl)carbamate (267 mg, 29% yield). MS m/z 799.6, 801.6 [M+H]⁺; ¹H NMR (CDCl₃) δ: 7.19 (d, J=5.0 Hz, 1H), 7.08 (s, 1H), 6.89-6.92 (m, J=3.1 Hz, 1H), 5.32 (s, 2H), 4.92 (br s, 1H), 3.91 (br s, 1H), 3.56-3.68 (m, 2H), 3.05-3.17 (m, 2H), 1.55 (s, 9H), 1.37 (s, 9H), 1.06 (s, 9H), 0.91 (s, 9H), 0.31 (s, 6H), 0.06 (s, 6H).

### Step 4: tert-Butyl N-[6-[(2R)-2-(tert-butoxycarbonylamino)-3-hydroxy-propyl]-2-chloro-7-hydroxy-thieno[3,2-d]pyrimidin-4-yl]-N-(2-thienylmethyl)carbamate

To a mixture of tert-butyl N-[6-[(2R)-2-(tert-butoxycarbonylamino)-3-[tert-butyl(dimethyl)silyl]oxy-propyl]-7-[tert-butyl(dimethyl)silyl]oxy-2-chloro-thieno[3,2-d]pyrimidin-4-yl]-N-(2-thienylmethyl)carbamate (267 mg, 0.3 mmol, 1.0 eq.) in THF (2.7 mL) at 0° C. was added tetrabutylammonium fluoride (1 M) in THF (0.6 mL, 0.6 mmol, 2 eq.) dropwise. The reaction mixture was stirred for 24 h and then concentrated under reduced pressure. The crude residue was purified over silica gel with ethyl acetate in hexanes (0-50%) to give tert-butyl N-[6-[(2R)-2-(tert-butoxycarbonylamino)-3-hydroxy-propyl]-2-chloro-7-hydroxy-thieno[3,2-d]pyrimidin-4-yl]-N-(2-thienylmethyl)carbamate (152 mg, 90% yield). MS m/z 571.3, 573.3 [M+H]⁺; ¹H NMR (CDCl₃) δ: 7.21 (d, J=5.0 Hz, 1H), 7.11 (s, 1H), 6.93 (d, J=3.2 Hz, 1H), 5.36 (s, 2H), 5.09-5.15 (m, 1H), 3.85-3.91 (m, J=3.7, 1.7, 1.7 Hz, 1H), 3.67 (s, 2H), 3.18-3.26 (m, 1H), 3.10 (dd, J=14.0, 5.3 Hz, 1H), 1.77-2.18 (m, 2H), 1.59 (s, 9H), 1.45 (s, 9H).

### Step 5: tert-Butyl N-[6-[(2R)-2-(tert-butoxycarbonylamino)-3-hydroxy-propyl]-2-chloro-7-methoxy-thieno[3,2-d]pyrimidin-4-yl]-N-(2-thienylmethyl)carbamate

To a mixture of tert-butyl N-[6-[(2R)-2-(tert-butoxycarbonylamino)-3-hydroxy-propyl]-2-chloro-7-hydroxy-thieno[3,2-d]pyrimidin-4-yl]-N-(2-thienylmethyl)carbamate (152 mg, 0.26 mmol, 1.0 eq.) and triphenylphosphine (77 mg, 0.29 mmol, 1.1 eq) was added THF (2.6 mL). The mixture was then cooled 0° C., to which was added diethyl azodicarboxylate (40 mass %) in toluene (0.1 mL, 0.29 mmol, 1.1 eq.) dropwise. After 1 h, the reaction mixture was concentrated under reduced pressure. The crude residue was purified over silica gel with ethyl acetate and hexanes (0-50%) to give tert-butyl N-[6-[(2R)-2-(tert-butoxycarbonylamino)-3-hydroxy-propyl]-2-chloro-7-methoxy-thieno[3,2-d]pyrimidin-4-yl]-N-(2-thienylmethyl)carbamate (30 mg, 19% yield). MS m/z 585.1, 587.1 [M+H]⁺; ¹H NMR (methanol-d₄) δ: 7.22 (br d, J=5.0 Hz, 1H), 7.02 (br s, 1H), 6.86 (t, J=3.8 Hz, 1H), 5.27 (br s, 2H), 4.05 (s, 3H), 3.80 (br d, J=3.5 Hz, 1H), 3.46-3.57 (m, 2H), 3.27 (br d, J=1.2 Hz, 1H), 2.86 (br dd, J=14.6, 9.3 Hz, 1H), 1.51 (s, 9H), 1.31 (s, 9H); 1 NH and 1 OH not observed.

### Step 6: (2R)-2-Amino-3-[2-chloro-7-methoxy-4-(2-thienylmethylamino)thieno[3,2-d]pyrimidin-6-yl]propan-1-ol

To a reaction tube with tert-butyl N-[6-[(2R)-2-(tert-butoxycarbonylamino)-3-hydroxy-propyl]-2-chloro-7-methoxy-thieno[3,2-d]pyrimidin-4-yl]-N-(2-thienylmethyl)carbamate (30 mg, 0.05 mmol, 1.0 eq.) was added hydrochloric acid (4 M) in dioxane (1 mL). The reaction mixture was stirred at room temperature for 1 h, diluted with diethyl ether, and then filtered and rinsed with diethyl ether. The solid was dried under vacuum for 24 h to give (2R)-2-amino-3-[2-chloro-7-methoxy-4-(2-thienylmethylamino)thieno[3,2-d]pyrimidin-6-yl]propan-1-ol (18 mg, 91% yield) as a yellow solid. MS m/z 385.1, 387.1 [M+H]⁺; ¹H NMR (methanol-d₄) δ: 7.30 (d, J=5.0 Hz, 1H), 7.11 (br s, 1H), 6.96 (br t, J=3.4 Hz, 1H), 4.96 (s, 2H), 4.07 (s, 3H), 3.78 (br dd, J=11.4, 2.7 Hz, 1H), 3.59-3.65 (m, 1H), 3.57 (br d, J=3.1 Hz, 1H), 3.25-3.27 (m, 1H), 1 H obscured by MeOD peak; 3 NHs and 1 OH not observed.

### Example 20 (Compounds 66 and 70)

### (2S)-3-(2-Chloro-4-{[(furan-2-yl)methyl]amino}-7-methylthieno[3,2-d]pyrimidin-6-yl)-2-methylpropan-1-ol

### and

### 6-[(2S)-3-Amino-2-methylpropyl]-2-chloro-N-[(furan-2-yl)methyl]-7-methylthieno[3,2-d]pyrimidin-4-amine

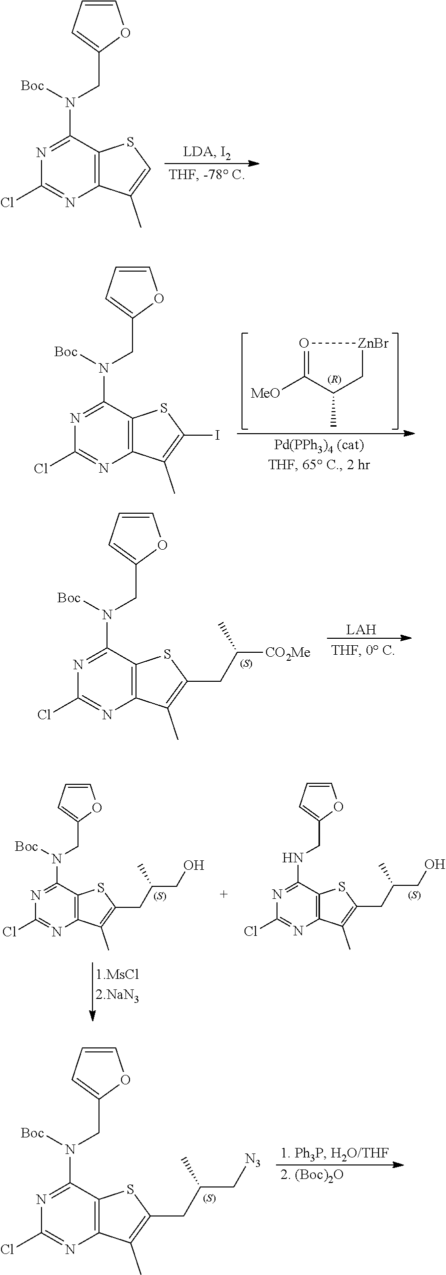

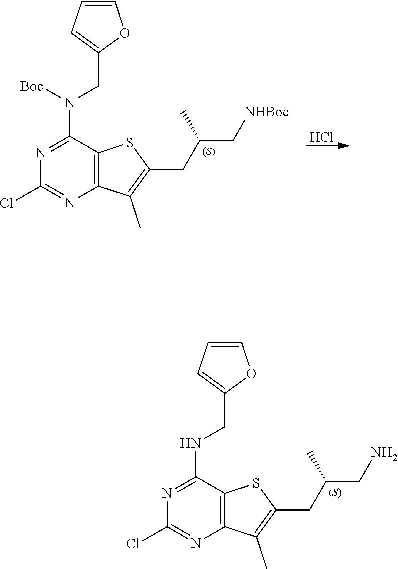

### Step 1: tert-Butyl (2-chloro-6-iodo-7-methylthieno[3,2-d]pyrimidin-4-yl)(furan-2-ylmethyl)carbamate

To a solution of tert-butyl (2-chloro-7-methylthieno[3,2-d]pyrimidin-4-yl)(furan-2-ylmethyl)carbamate (890 mg, 2.34 mmol, 1.0 eq.), prepared according to the procedure in Example 3, in THF (8 mL) was added LDA (2.0 M in THF, 1.3 mL, 1.1 eq.) at −78° C. After stirring for 45 min, a solution of iodine (624 mg, 2.46 mmol, 1.05 eq.) in THF (5 mL) was added dropwise and stirring was continued for 1 h at −78° C. The reaction was quenched by addition of EtOAc and NH₄Cl (sat. aq.) and warmed to room temperature. The organic layers were washed with sodium thiosulfate solution, water and brine, dried over sodium sulfate and evaporated. The residue was purified by flash column chromatography on silica gel eluting with 0-15% EtOAc in hexanes to provide tert-butyl (2-chloro-6-iodo-7-methylthieno[3,2-d]pyrimidin-4-yl)(furan-2-ylmethyl)carbamate (1100 mg, 93% yield). ¹H NMR (acetone-d6) δ: 7.40-7.50 (m, 1H), 6.37 (s, 2H), 5.26 (s, 2H), 2.38 (s, 3H), 1.58 (s, 9H).

### Step 2: Methyl (S)-3-(4-((tert-butoxycarbonyl)(furan-2-ylmethyl)amino)-2-chloro-7-methylthieno[3,2-d]pyrimidin-6-yl)-2-methylpropanoate

To a mixture of tert-butyl (2-chloro-6-iodo-7-methylthieno[3,2-d]pyrimidin-4-yl)(furan-2-ylmethyl)carbamate (134 mg, 0.26 mmol, 1.0 eq.) and Pd(PPh3)4 (18 mg, 0.016 mmol, 0.06 eq.) in THF (0.3 mL) was added (R)-(3-methoxy-2-methyl-3-oxopropyl)zinc(II) bromide (from Rieke Metals, 0.5 M in THF, 0.7 mL, 1.3 eq) under Ar. The mixture was heated at 65° C. for 2 h. After cooling, the mixture was quenched by addition of NH₄Cl (sat. aq.) and diluted with EtOAc. The organic layers were washed with water and brine, dried over sodium sulfate and evaporated. The crude material was purified by flash column chromatography on silica gel eluting with 0-30% EtOAc in hexanes to provide methyl (S)-3-(4-((tert-butoxycarbonyl)(furan-2-ylmethyl)amino)-2-chloro-7-methylthieno[3,2-d]pyrimidin-6-yl)-2-methylpropanoate (72 mg, 57% yield) as a colorless oil. MS m/z 502.3, 504.3 [M+Na]⁺.

### Step 3: tert-Butyl (S)-(2-chloro-6-(3-hydroxy-2-methylpropyl)-7-methylthieno[3,2-d]pyrimidin-4-yl)(furan-2-ylmethyl)carbamate and (S)-3-(2-chloro-4-((furan-2-ylmethyl)amino)-7-methylthieno[3,2-d]pyrimidin-6-yl)-2-methylpropan-1-ol

To a solution of methyl (S)-3-(4-((tert-butoxycarbonyl)(furan-2-ylmethyl)amino)-2-chloro-7-methylthieno[3,2-d]pyrimidin-6-yl)-2-methylpropanoate (72 mg, 0.15 mmol, 1.0 eq.) in THF (1.2 mL) was added LAH (1.0 M in THF, 0.18 mL, 1.2 eq.) dropwise at 0° C. The reaction was continued at 0° C. for 1 h, then quenched with citric acid (1.0 M, aq., 1 mL), and extracted with EtOAc. The organic layers were washed with water and brine, dried over sodium sulfate and evaporated. The crude material was purified by flash column chromatography on silica gel eluting with 0-60% EtOAc in hexanes to provide tert-butyl (S)-(2-chloro-6-(3-hydroxy-2-methylpropyl)-7-methylthieno[3,2-d]pyrimidin-4-yl)(furan-2-ylmethyl)carbamate (41 mg, 60% yield) MS m/z 452.3, 454.3 [M+H]⁺, and (S)-3-(2-chloro-4-((furan-2-ylmethyl)amino)-7-methylthieno[3,2-d]pyrimidin-6-yl)-2-methylpropan-1-ol (19 mg, 36% yield). MS m/z 352.3, 354.3 [M+H]⁺. ¹H NMR (methanol-d) 4 6: 7.44 (s, 1H), 6.30-6.50 (m, 2H), 4.75 (s, 2H), 3.47-3.52 (m, 2H), 3.08-3.14 (m, 1H), 2.73 (dd, J=14.6, 8.9 Hz, 1H), 2.29 (s, 3H), 1.96-2.08 (m, 1H), 0.97 (d, J=6.7 Hz, 3H); 1 NH and 1 OH not observed.

### Step 4: tert-Butyl (S)-(6-(3-azido-2-methylpropyl)-2-chloro-7-methylthieno[3,2-d]pyrimidin-4-yl)(furan-2-ylmethyl)carbamate

To a solution of tert-butyl (S)-(2-chloro-6-(3-hydroxy-2-methylpropyl)-7-methylthieno[3,2-d]pyrimidin-4-yl)(furan-2-ylmethyl)carbamate (80 mg, 0.17 mmol, 1.0 eq.) and DIPEA (47 mg, 2.0 eq.) in CH₂Cl₂ (1 mL) was added slowly a solution of MsCl (30 mg, 0.26 mmol, 1.5 eq.) in CH₂Cl₂ (1 mL) at 0° C. The mixture was stirred at 0° C. for 1 h, then quenched with NaHCO₃ (sat. aq.), and extracted with CH₂Cl₂. The organic layers were washed with water and brine, dried over sodium sulfate and evaporated. The crude product was used directly in the next step without further purification. A mixture of the above crude product (93 mg, 0.17 mmol, 1.0 eq.) and sodium azide (35 mg, 3.0 eq.) in DMSO (0.3 mL) was stirred at room temperature overnight, then quenched with NaHCO₃ (sat. aq). The mixture was diluted with EtOAc. The organic layers were washed with water and brine, dried over sodium sulfate and evaporated. The crude material was purified by flash column chromatography on silica gel eluting with 0-30% EtOAc in hexanes to provide tert-butyl (S)-(6-(3-azido-2-methylpropyl)-2-chloro-7-methylthieno[3,2-d]pyrimidin-4-yl)(furan-2-ylmethyl)carbamate (66 mg, 79% yield). ¹H NMR (acetone-d₆) δ: 7.42 (dd, J=1.8, 0.9 Hz, 1H), 6.28-6.37 (m, 2H), 5.21 (s, 2H), 3.44 (qd, J=12.4, 6.0 Hz, 2H), 3.14 (dd, J=14.6, 6.4 Hz, 1H), 2.93 (dd, J=14.6, 8.2 Hz, 1H), 2.36 (s, 3H), 2.21 (dt, J=7.9, 6.6 Hz, 1H), 1.53 (s, 9H), 1.06 (d, J=6.7 Hz, 3H).

### Step 5: tert-Butyl (S)-(6-(3-((tert-butoxycarbonyl)amino)-2-methylpropyl)-2-chloro-7-methylthieno[3,2-d]pyrimidin-4-yl)(furan-2-ylmethyl)carbamate

To a solution of tert-butyl (S)-(6-(3-azido-2-methylpropyl)-2-chloro-7-methylthieno[3,2-d]pyrimidin-4-yl)(furan-2-ylmethyl)carbamate (66 mg, 0.14 mmol, 1.0 eq.) in THF (1.3 mL) was added triphenylphosphine (110 mg, 0.42 mmol, 3.0 eq.) and water (25 mg, 1.4 mmol, 10 eq.). The mixture was stirred at room temperature overnight. The reaction was quenched with NH₄Cl (sat. aq.) and extracted with EtOAc. The organic layers were washed with water and brine and dried over sodium sulfate. After concentration, the crude material was used in the next step without further purification. MS m/z 451.3, 453.3 [M+H]⁺. To a solution of the above crude intermediate (62 mg, 0.14 mmol, 1.0 eq.) in CH₂Cl₂ (1 mL) was added 4-DMAP (12 mg, 0.097 mmol, 0.50 eq.), followed by di-tert-butyl dicarbonate (47 mg, 0.21 mmol, 1.5 eq.). After stirring at room temperature for 1 h, the reaction was quenched with NaHCO₃ (sat. aq.), then extracted with CH₂Cl₂. The organic layers were washed with water and brine, dried over sodium sulfate and evaporated. The crude material was purified by flash column chromatography on silica gel eluting with 0-30% EtOAc in hexanes to provide tert-butyl (S)-(6-(3-((tert-butoxycarbonyl)amino)-2-methylpropyl)-2-chloro-7-methylthieno[3,2-d]pyrimidin-4-yl)(furan-2-ylmethyl)carbamate (30 mg, 39% yield). MS m/z 573.2, 574.2 [M+Na]⁺.

### Step 6: (S)-6-(3-Amino-2-methylpropyl)-2-chloro-N-(furan-2-ylmethyl)-7-methylthieno[3,2-d]pyrimidin-4-amine

A solution of tert-butyl (S)-(6-(3-((tert-butoxycarbonyl)amino)-2-methylpropyl)-2-chloro-7-methylthieno[3,2-d]pyrimidin-4-yl)(furan-2-ylmethyl)carbamate (30 mg, 0.054 mmol, 1.0 eq) in HCl (4 M in dioxane) (1 mL) was stirred at room temperature for 1 h. The precipitate was filtered and rinsed with diethyl ether to afford (S)-6-(3-amino-2-methylpropyl)-2-chloro-N-(furan-2-ylmethyl)-7-methylthieno[3,2-d]pyrimidin-4-amine (6 mg, 32% yield) as a hydrochloride salt. MS m/z 351.2, 353.2 [M+H]⁺; ¹H NMR (methanol-d₄) δ: 7.46 (dd, J=1.5, 0.9 Hz, 1H), 6.24-6.45 (m, 2H), 4.81 (s, 2H), 3.00-3.14 (m, 2H), 2.84-2.97 (m, 2H), 2.34 (s, 3H), 2.21-2.29 (m, 1H), 1.09 (d, J=6.7 Hz, 3H); 3 NHs not observed.

The compounds below were prepared according to the procedure of example 20 by substituting the appropriate starting materials, reagents and reaction conditions.

| Com- | |
|---|---|
| pound | Spectral Data |
| 63 | MS m/z 338.3, 340.3 [M + H]⁺; ¹H NMR (methanol-d4) δ: 7.44 |
| | (s, 1H), 6.24-6.41 (m, 2H), 4.75 (s, 2H), 3.64 (t, J = 6.3 Hz, 2H), |
| | 3.02 (t, J = 7.6 Hz, 2H), 2.29 (s, 3H), 1.79-1.99 (m, 2H); 1 NH |
| | and 1 OH not observed. |
| 67 | MS m/z 337.2, 339.2 [M + H]⁺; ¹H NMR (methanol-d4) δ: 7.46 |
| | (d, J = 0.9 Hz, 1H), 6.28-6.43 (m, 2H), 4.80 (s, 2H), 3.07 (dt, |
| | J = 18.8, 7.8 Hz, 4H), 2.34 (s, 3H), 2.07 (br t, J = 7.8 Hz, 2H); |
| | 3 NHs not observed. |
| 71 | MS m/z 352.1, 354.2 [M + H]⁺; ¹H NMR (methanol-d4) δ: 7.44 |
| | (s, 1H), 6.25-6.45 (m, 2H), 4.75 (s, 2H), 3.48-3.51 (m, 2H), |
| | 3.07-3.14 (m, 1H), 2.73 (dd, J = 14.6, 8.9 Hz, 1H), 2.29 (s, 3H), |
| | 1.88-2.07 (m, 1H), 0.97 (d, J = 6.7 Hz, 3H); 1 NH and 1 OH not |
| | observed. |
| 72 | MS m/z 351.1, 353.1 [M + H]⁺; ¹H NMR (methanol-d4) δ: 7.45 |
| | (d, J = 0.9 Hz, 1H), 6.28-6.42 (m, 2H), 4.81 (s, 2H), 2.99-3.14 |
| | (m, 2H), 2.76-2.98 (m, 2H), 2.34 (s, 3H), 2.19-2.29 (m, 1H), |
| | 1.09 (d, J = 6.7 Hz, 3H); 3 NHs not observed. |

### Example 21 (Compound 41)

### 6-(Azetidin-3-yl)-2-chloro-N-[furan-2-yl)methyl]-7-methylthieno[3,2-d]pyrimidin-4-amine

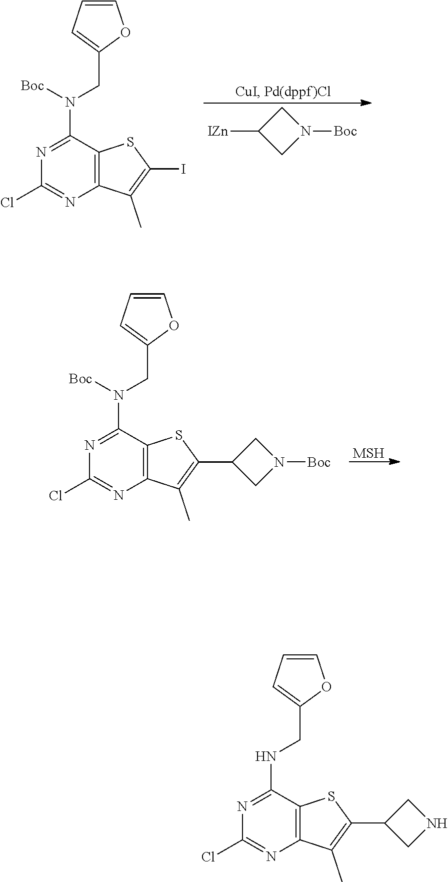

### Step 1: tert-butyl 3-(4-((tert-butoxycarbonyl)(furan-2-ylmethyl)amino)-2-chloro-7-methylthieno[3,2-d]pyrimidin-6-yl)azetidine-1-carboxylate

Preparation of (1-(tert-butoxycarbonyl)azetidin-3-yl)zinc(II) iodide: An oven-dried, nitrogen-filled flask was charged with zinc powder (243 mg, 3.7 mmol, 2.0 eq.) and DMA (0.5 mL) under argon. This grey suspension was heated to 40° C. and a solution of 1,2-dibromoethane (113 mg, 0.32 eq.) in DMA (0.5 mL) was added dropwise, followed by a solution of TMSC1 (26 mg, 0.13 eq.) in DMAc (0.5 mL). After stirring at 40° C. for 10 min, a solution of tert-butyl 3-iodoazetidine-1-carboxylate (520 mg, 1.84 mmol, 1.0 eq.) in DMA (2 mL) was added and stirring was continued at 40° C. for 30 minutes. After cooling, this organozinc reagent (˜0.5 M in DMA) was used immediately in the next step.

To a mixture of tert-butyl (2-chloro-6-iodo-7-methylthieno[3,2-d]pyrimidin-4-yl)(furan-2-ylmethyl)carbamate (105 mg, 0.21 mmol, 1.0 eq.) prepared according to the procedure in example 20, step 1, Pd(dppf)Cl₂ (8.6 mg, 0.05 eq.) and Cul (4.0 mg, 0.10 eq.) in DMA (0.5 mL) under argon was added the above organozinc reagent (˜0.5 M in DMA, 0.8 mL, 1.5 eq.). The mixture was then stirred at 90° C. for 1 h. After cooling, the reaction was quenched with NH₄Cl (sat. aq.), extracted with EtOAc, dried over sodium sulfate and evaporated. The crude material was purified by flash column chromatography on silica gel eluting with 0-100% EtOAc in hexanes to provide tert-butyl 3-(4-((tert-butoxycarbonyl)(furan-2-ylmethyl)amino)-2-chloro-7-methylthieno[3,2-d]pyrimidin-6-yl)azetidine-1-carboxylate (88 mg, 79% yield). MS m/z 535.2, 537.3 [M+H]⁺.

### Step 2: 6-(Azetidin-3-yl)-2-chloro-N-(furan-2-ylmethyl)-7-methylthieno[3,2-d]pyrimidin-4-amine

tert-Butyl 3-(4-((tert-butoxycarbonyl)(furan-2-ylmethyl)amino)-2-chloro-7-methylthieno[3,2-d]pyrimidin-6-yl)azetidine-1-carboxylate (88 mg, 0.2 mmol) was stirred in a solution of methanesulfonic acid (422 mg, 20 eq.) in dioxane (2 mL) at room temperature for 1 h, then triturated with diethyl ether and filtered. The crude solid was purified on prep-HPLC eluting with 5-50% CH₃CN in water with 0.1% TFA to afford 6-(azetidin-3-yl)-2-chloro-N-(furan-2-ylmethyl)-7-methylthieno[3,2-d]pyrimidin-4-amine (15 mg, 28% yield) as the trifluoroacetic acid salt. MS m/z 335.3, 337.3, [M+H]⁺;¹H NMR (DMSO-d₆) δ: 9.05 (br s, 1H), 8.82 (t, J=5.6 Hz, 1H), 8.73 (br s, 1H), 7.54 (dd, J=1.7, 0.8 Hz, 1H), 6.35 (dd, J=3.1, 1.8 Hz, 1H), 6.26 (d, J=2.7 Hz, 1H), 4.54-4.64 (m, 3H), 4.32 (br s, 2H), 4.02 (br s, 2H), 2.13 (s, 3H).

The compound below was prepared according to the procedure of example 21 by substituting the appropriate starting materials, reagents and reaction conditions.

| Com- | |
|---|---|
| pound | Spectral Data |
| 57 | MS m/z 349.3, 351.3 [M + H]⁺; ¹H NMR (methanol-d4) δ: 8.44 |
| | (br s, 1H), 7.32 (s, 1H), 6.18-6.36 (m, 2H), 4.63 (s, 2H), 3.88- |
| | 3.96 (m, 1H), 3.56-3.63 (m, 1H), 3.33-3.41 (m, 1H), 3.23-3.27 |
| | (m, 1H), 2.94-3.01 (m, 1H), 2.39-2.47 (m, 1H), 2.23 (s, 3H), |
| | 1.96-2.05 (m, 1H), 2 NH not observed. |

### Example 22 (Compounds 85 and 83)

### 6-[(2S)-2-Aminopropyl]-4-{[(thiophen-2-yl)methyl]amino}thieno[3,2-d]pyrimidine-2,7-dicarbonitrile

### and

### 6-[(2S)-2-Aminopropyl]-7-bromo-4-{[(thiophen-2-yl)methyl]amino}thieno[3,2-d]pyrimidine-2-carbonitrile

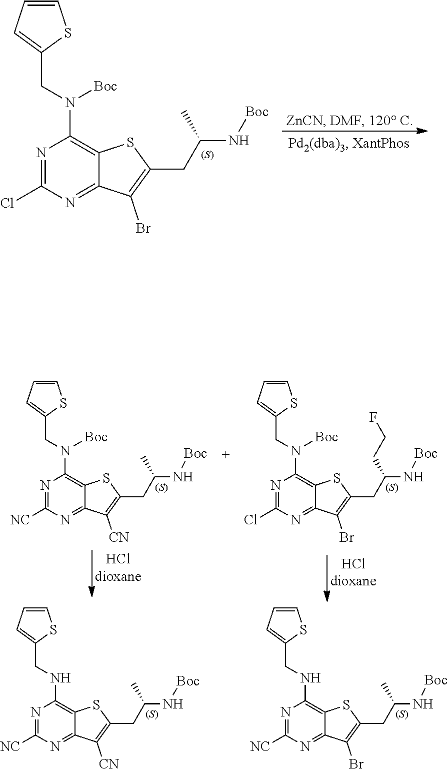

### Step 1: tert-Butyl (S)-(6-(2-((tert-butoxycarbonyl)amino)propyl)-2,7-dicyanothieno[3,2-d]pyrimidin-4-yl)(thiophen-2-ylmethyl)carbamate and tert-butyl (S)-(7-bromo-6-(2-((tert-butoxycarbonyl)amino)propyl)-2-cyanothieno[3,2-d]pyrimidin-4-yl)(thiophen-2-ylmethyl)carbamate

To a degassed solution of tert-butyl (S)-(7-bromo-6-(2-((tert-butoxycarbonyl)amino)propyl)-2-chlorothieno[3,2-d]pyrimidin-4-yl)(thiophen-2-ylmethyl)carbamate (136 mg, 0.22 mmol, 1.0 eq.), prepared according to the procedure in example 3, in DMF (1 mL) was added zinc cyanide (15.8 mg, 0.60 eq.), Pd₂(dba)₃ (10.4 mg, 0.05 eq.) and Xantphos (13.1 mg, 0.10 eq.) under argon. The sealed tube was stirred at 120° C. for 1 h and then cooled. The reaction was quenched with NH₄Cl (sat. aq.) and extracted with EtOAc. The combined organic phases were dried and concentrated. The crude material was purified by flash column chromatography on silica gel eluting with 0-20% EtOAc in CH₂Cl₂ to provide tert-butyl (S)-(6-(2-((tert-butoxycarbonyl)amino)propyl)-2,7-dicyanothieno[3,2-d]pyrimidin-4-yl)(thiophen-2-ylmethyl)carbamate, MS m/z 553.3 [M−H]⁻; and tert-butyl (S)-(7-bromo-6-(2-((tert-butoxycarbonyl)amino)propyl)-2-cyanothieno[3,2-d]pyrimidin-4-yl)(thiophen-2-ylmethyl)carbamate, MS m/z 506.1, 508.1 [M−H−Boc]⁻, respectively.

### Step 2: (S)-6-(2-Aminopropyl)-4-((thiophen-2-ylmethyl)amino)thieno[3,2-d]pyrimidine-2,7-dicarbonitrile

tert-Butyl (S)-(6-(2-((tert-butoxycarbonyl)amino)propyl)-2,7-dicyanothieno[3,2-d]pyrimidin-4-yl)(thiophen-2-ylmethyl)carbamate, obtained from step 1, was stirred in a solution of HCl (4 M in dioxane, 1 mL) at room temperature for 1 h and then the organic volatiles were removed. The crude solid was triturated with diethyl ether and filtered to afford (S)-6-(2-aminopropyl)-4-((thiophen-2-ylmethyl)amino)thieno[3,2-d]pyrimidine-2,7-dicarbonitrile (5 mg, 5% overall yield for 2 steps) as the hydrochloride salt. MS m/z 355.1 [M+H]⁺; ¹H NMR (methanol-d₄) δ: 7.32 (dd, J=5.2, 1.2 Hz, 1H), 7.06-7.21 (m, 1H), 6.98 (dd, J=5.2, 3.4 Hz, 1H), 4.99 (s, 2H), 3.73-3.89 (m, 1H), 3.56-3.63 (m, 1H), 3.45-3.55 (m, 1H), 1.42 (d, J=6.4 Hz, 3H); 3 NHs not observed.

### Step 3: (S)-6-(2-Aminopropyl)-7-bromo-4-((thiophen-2-ylmethyl)amino)thieno[3,2-d]pyrimidine-2-carbonitrile

tert-Butyl (S)-(7-bromo-6-(2-((tert-butoxycarbonyl)amino)propyl)-2-cyanothieno[3,2-d]pyrimidin-4-yl)(thiophen-2-ylmethyl)carbamate, obtained from step 1, was stirred in a solution of HCl (4 M in dioxane, 1 mL) at room temperature for 1 h and then the organic volatiles were removed. The crude solid was purified by preparative HPLC with 5-40% CH₃CN in water containing 0.1% formic acid to provide (S)-6-(2-aminopropyl)-7-bromo-4-((thiophen-2-ylmethyl)amino)thieno[3,2-d]pyrimidine-2-carbonitrile (10 mg, 11% overall yield for 2 steps) as the formic acid salt. MS m/z 408.1, 410.1 [M+H]⁺;¹H NMR (methanol-d₄) δ: 7.31 (dd, J=5.2, 1.2 Hz, 1H), 7.06-7.20 (m, 1H), 6.98 (dd, J=5.0, 3.5 Hz, 1H), 4.99 (s, 2H), 3.71-3.89 (m, 1H), 3.40-3.47 (m, 1H), 3.35-3.39 (m, 1H), 1.40 (d, J=6.7 Hz, 3H); 3 NHs not observed.

### Example 23 (Compound 84)

### 2-Chloro-4-{[(thiophen-2-yl)methyl]amino}thieno[3,2-d]pyrimidine-7-carbonitrile

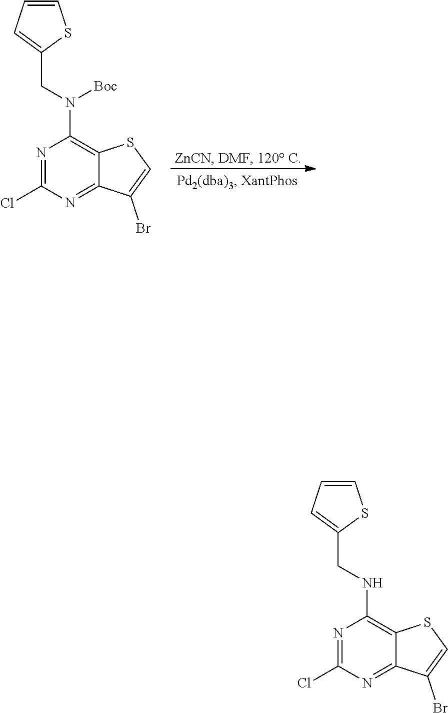 [SMILES: CC(C)(C)OC(=O)N(Cc1cccs1)c1nc(Cl)nc2c(Br)csc12 ; "ZnCN, DMF, 120° C." ; Clc1nc(NCc2cccs2)c2scc(Br)c2n1]

To a degassed solution of tert-butyl (7-bromo-2-chlorothieno[3,2-d]pyrimidin-4-yl)(thiophen-2-ylmethyl)carbamate (101 mg, 0.22 mmol, 1.0 eq.) in DMF (1 mL) was added zinc cyanide (15.8 mg, 0.60 eq.), Pd₂(dba)₃ (10.4 mg, 0.05 eq.) and Xantphos (13.1 mg, 0.10 eq.) under argon, then the sealed tube was stirred at 120° C. for 1 h. After cooling, the mixture was quenched with NH₄Cl (sat. aq.), then extracted with EtOAc. The combined organic phases were dried and concentrated. The crude material was purified by flash column chromatography on silica gel eluting with 0-10% MeOH in CH₂C₁₂,followed by further purification on HPLC with 10-100% CH₃CN in water containing 0.1% formic acid to provide 2-chloro-4-((thiophen-2-ylmethyl)amino)thieno[3,2-d]pyrimidine-7-carbonitrile (5 mg, 7% yield). MS m/z 306.9, 308.9 [M+H]⁺; ¹H NMR (DMSO-d₆) δ: 9.42 (br t, J=5.2 Hz, 1H), 9.07 (s, 1H), 7.35 (dd, J=5.0, 1.1 Hz, 1H), 7.03 (d, J=2.7 Hz, 1H), 6.91 (dd, J=5.0, 3.5 Hz, 1H), 4.77 (d, J=5.2 Hz, 2H).

### Example 24 (Compound 46)

### 6-[(1S)-1-Aminoethyl]-7-bromo-2-chloro-N-[furan-2-yl)methyl]thieno[3,2-d]pyrimidin-4-amine

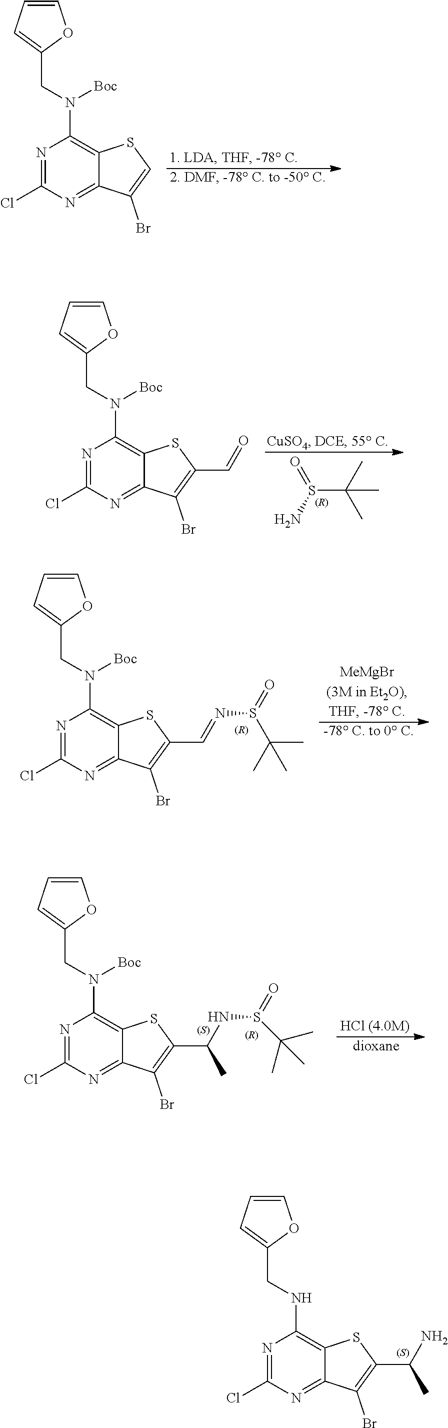

### Step 1: tert-Butyl (7-bromo-2-chloro-6-formylthieno[3,2-d]pyrimidin-4-yl)(furan-2-ylmethyl)carbamate

To a solution of tert-butyl (7-bromo-2-chlorothieno[3,2-d]pyrimidin-4-yl)(furan-2-ylmethyl)carbamate (500 mg, 1.12 mmol, 1.0 eq.), prepared according to the procedure in example 3, in THF (4 mL) at −78° C. was added LDA (2.0 M in THF, 0.67 mL, 1.2 eq.). After 30 min, DMF (823 mg, 11.2 mmol, 10 eq.) was added dropwise. The temperature was allowed to rise to −50° C., and the reaction was quenched with saturated aqueous NH₄Cl and then diluted with EtOAc. The mixture was washed with water followed by brine, and the organic layer was dried over sodium sulfate and evaporated. The residue was purified by flash column chromatography on silica gel eluting with 0-25% EtOAc in hexanes to provide tert-butyl (7-bromo-2-chloro-6-formylthieno[3,2-d]pyrimidin-4-yl)(furan-2-ylmethyl)carbamate (399 mg, 75% yield) as a yellow solid. ¹H NMR (acetone-d₆) δ ppm 10.41 (s, 1H), 7.47-7.49 (m, 1H), 6.43-6.44 (m, 1H), 6.39-6.41 (m, 1H), 5.30-5.31 (m, 2H), 1.58-1.62 (m, 9H).

### Step 2: tert-Butyl (R,E)-(7-bromo-6-(((tert-butylsulfinyl)imino)methyl)-2-chlorothieno[3,2-d]pyrimidin-4-yl)(furan-2-ylmethyl)carbamate

A mixture of tert-butyl (7-bromo-2-chloro-6-formylthieno[3,2-d]pyrimidin-4-yl)(furan-2-ylmethyl)carbamate (162 mg, 0.34 mmol, 1.0 eq.), R-(+)-2-methylpropane-2-sulfinamide (50 mg, 0.41 mmol, 1.2 eq.) and CuSO₄ (85 mg, 0.51 mmol, 1.5 eq.) in DCE (0.4 mL) was stirred at 55° C. for 18 h. After cooling, the mixture was purified by flash column chromatography on silica gel eluting with 0-50% EtOAc in hexanes to provide tert-butyl (R,E)-(7-bromo-6-(((tert-butylsulfinyl)imino)methyl)-2-chlorothieno[3,2-d]pyrimidin-4-yl)(furan-2-ylmethyl)carbamate as a yellow solid (121 mg, 61% yield). MS m/z 577.4, 579.4 [M-FH]⁺.

### Step 3: tert-Butyl (7-bromo-6-((S)-1-(((R)-tert-butylsulfinyl)amino)ethyl)-2-chlorothieno[3,2-d]pyrimidin-4-yl)(furan-2-ylmethyl)carbamate

To a solution of tert-butyl (R,E)-(7-bromo-6-(((tert-butylsulfinyl)imino)methyl)-2-chlorothieno[3,2-d]pyrimidin-4-yl)(furan-2-ylmethyl)carbamate (110 mg, 0.19 mmol, 1.0 eq.) in THF (1.0 mL) was added MeMgBr (3.0 M in Et₂O, 0.096 mL, 1.5 eq.) at −78° C. The mixture was gradually warmed to −20° C. over 1 h, then quenched with a saturated solution of NH₄Cl and then diluted with EtOAc. The combined organic layers were dried and concentrated. The residue was purified by flash column chromatography on silica gel eluting with 0-100% EtOAc in hexanes to provide tert-butyl (7-bromo-6((S)-1-(((R)-tert-butylsulfinyl)amino)ethyl)-2-chlorothieno[3,2-d]pyrimidin-4-yl)(furan-2-ylmethyl)carbamate (86 mg, 76% yield). ¹H NMR (acetone-d₆) δ 7.45 (s, 1H), 6.31-6.40 (m, 2H), 5.45-5.49 (m, 1H), 5.26 (s, 2H), 5.13-5.19 (m, 1H), 1.68 (d, J=6.6 Hz, 3H), 1.56 (s, 9H), 1.23 (s, 9H).

### Step 4: (S)-6-(1-Aminoethyl)-7-bromo-2-chloro-N-(furan-2-ylmethyl)thieno[3,2-d]pyrimidin-4-amine hydrochloride

A solution of tert-butyl (7-bromo-6((S)-1-(((R)-tert-butylsulfinyl)amino)ethyl)-2-chlorothieno[3,2-d]pyrimidin-4-yl)(furan-2-ylmethyl)carbamate (86 mg, 0.14 mmol) in HCl (4 M in dioxane, 1 mL) was stirred at room temperature for 1 h. The organic volatiles were removed, and the residue was triturated with diethyl ether and filtered to afford (S)-6-(1-aminoethyl)-7-bromo-2-chloro-N-(furan-2-ylmethyl)thieno[3,2-d]pyrimidin-4-amine (12 mg, 74% yield) as the hydrochloride salt. MS m/z 387.2, 389.2 [M+H]⁺;¹H NMR (DMSO-d₆) δ: 9.35 (br t, J=5.5 Hz, 1H), 8.87 (br s, 1H), 8.82 (br s, 2H), 7.62 (s, 1H), 6.43 (dd, J=3.1, 1.8 Hz, 1H), 6.37 (d, J=3.1 Hz, 1H), 4.84-5.06 (m, 1H), 4.70 (br t, J=5.2 Hz, 2H), 1.65 (d, J=6.7 Hz, 3H).

The compounds below were prepared according to the procedure of Example 24 by substituting the appropriate starting materials, reagents and reaction conditions.

| Com- | |
|---|---|
| pound | Spectral Data |
| 47 | MS m/z 323.3, 325.3 [M + H]⁺; ¹H NMR (DMSO-d₆) δ: 8.99 |
| | (br t, J = 5.5 Hz, 1H), 8.53 (br s, 3H), 7.61 (s, 1H), 6.42 (br dd, |
| | J = 3.4, 1.8 Hz, 1H), 6.33 (d, J = 3.1 Hz, 1H), 4.93-5.10 (m, 1H), |
| | 4.69 (t, J = 6.1 Hz, 2H), 2.34 (s, 3H), 1.62 (d, J = 7.0 Hz, 3H). |
| 48 | MS m/z 337.3, 339.3 [M + H]⁺; ¹H NMR (methanol-d₄) δ: 7.46 |
| | (s, 1H), 6.38 (br s, 2H), 4.74-4.85 (m, 3H), 2.44 (s, 3H), 2.16- |
| | 2.22 (m, 1H), 1.92-2.12 (m, 1H), 1.02 (t, J = 7.3 Hz, 3H); 3 NHs |
| | not observed. |
| 49 | MS m/z 337.3, 339.3 [M + H]⁺; ¹H NMR (methanol-d₄) δ: 7.45 |
| | (s, 1H), 6.37 (br d, J = 3.7 Hz, 2H), 4.74-4.85 (m, 3H), 2.43 |
| | (s, 3H), 2.16-2.22 (m, 1H), 1.92-2.12 (m, 1H), 1.02 (t, J = 7.3 |
| | Hz, 3H); 3 NHs not observed. |
| 94 | MS m/z 323.3, 325.3 [M + H]⁺; ¹H NMR (DMSO-d₆) δ: 8.99 |
| | (t, J = 5.6 Hz, 1H), 8.53 (br s, 3H), 7.61 (s, 1H), 6.42 (br dd, J = |
| | 3.4, 1.8 Hz, 1H), 6.33 (d, J = 3.1 Hz, 1H), 4.85-5.08 (m, 1H), |
| | 4.69 (t, J = 5.6 Hz, 2H), 2.34 (s, 3H), 1.62 (d, J = 6.7 Hz, 3H). |

### Example 25 (Compounds 58 and 61)

### 6-[(1 S)-1-Aaminoethyl]-2-chloro-N-[(furan-2-yl)methyl]-7-phenylthieno[3,2-d]pyrimidin-4-amine

### and

### 6-[(1S)-1-Aminoethyl]-N-[(furan-2-yl)methyl]-2,7-diphenylthieno[3,2-d]pyrimidin-4-amine

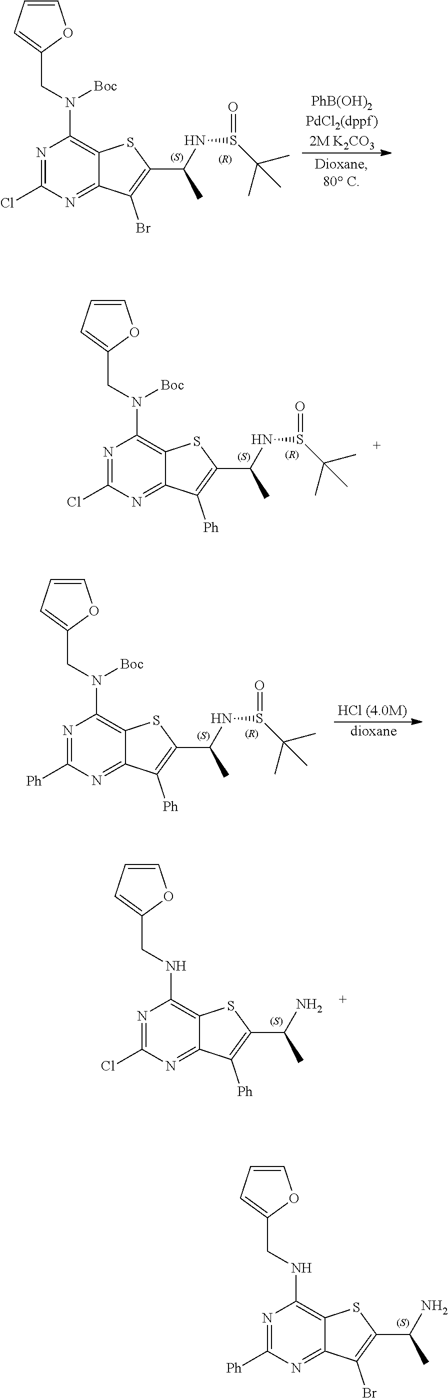

### Step 1: tert-Butyl (6-((S)-1-(((R)-tert-butylsulfinyl)amino)ethyl)-2-chloro-7-phenylthieno[3,2-d]pyrimidin-4-yl)(furan-2-ylmethyl)carbamate and tert-butyl (6-((S)-1-(((R)-tert-butylsulfinyl)amino)ethyl)-2,7-diphenylthieno[3,2-d]pyrimidin-4-yl)(furan-2-ylmethyl)carbamate

A mixture of tert-butyl (7-bromo-6((S)-1-(((R)-tert-butylsulfinyl)amino)ethyl)-2-chlorothieno[3,2-d]pyrimidin-4-yl)(furan-2-ylmethyl)carbamate (55 mg, 0.093 mmol, 1.0 eq.) prepared according to the procedure in example 24, 1,1′-bis(diphenylphosphino)ferrocene-palladium dichloride dichloromethane complex (3.8 mg, 0.005 mmol, 0.05 eq.), phenylboronic acid (13 mg, 0.1 mmol, 1.1 eq.), 1,4-dioxane (0.8 mL), and aqueous potassium carbonate (2.0 M in water, 0.14 mL, 3.0 eq.) was heated at 75° C. for 3 h. After cooling, the mixture was quenched with a saturated solution of NH₄Cl, then diluted with EtOAc. The combined organic phases were dried and concentrated. The residue was purified by flash column chromatography on silica gel eluting with 0-100% EtOAc in hexanes to provide a mixture of two intermediates, tert-butyl (6-((S)-1-(((R)-tert-butylsulfinyl)amino)ethyl)-2-chloro-7-phenylthieno[3,2-d]pyrimidin-4-yl)(furan-2-ylmethyl)carbamate (MS m/z 611.5, 613.5 [M+Na]⁺) and tert-butyl (6-((S)-1-(((R)-tert-butylsulfinyl)amino)ethyl)-2,7-diphenylthieno[3,2-d]pyrimidin-4-yl)(furan-2-ylmethyl)carbamate (MS m/z 631.6 [M+H]⁺), which was used in the next step without further purification.

### Step 2: (S)-6-(1-Aminoethyl)-2-chloro-N-(furan-2-ylmethyl)-7-phenylthieno[3,2-d]pyrimidin-4-amine and (S)-6-(1-aminoethyl)-2-chloro-N-(furan-2-ylmethyl)-7-phenylthieno[3,2-d]pyrimidin-4-amine

The mixture of products from step 1 was stirred in a solution of HCl (4 M in dioxane, 1 mL) at room temperature for 1 h and then the organic volatiles were removed. The crude solid was purified on prep-HPLC eluting with 5-50% ACN in water containing 0.1% formic acid to afford (S)-6-(1-aminoethyl)-2-chloro-N-(furan-2-ylmethyl)-7-phenylthieno[3,2-d]pyrimidin-4-amine (10 mg, 28% yield over two steps) and (S)-6-(1-aminoethyl)-2-chloro-N-(furan-2-ylmethyl)-7-phenylthieno[3,2-d]pyrimidin-4-amine (7 mg, 18% yield over two steps), respectively. (S)-6-(1-aminoethyl)-2-chloro-N-(furan-2-ylmethyl)-7-phenylthieno[3,2-d]pyrimidin-4-amine (formic acid salt): MS m/z 385.4, 387.4 [M+H]⁺;¹H NMR (methanol-d₄) δ: 8.38 (s, 1H), 7.39-7.43 (m, 2H), 7.29-7.37 (m, 4H), 6.19-6.30 (m, 2H), 4.64-4.69 (m, 2H), 4.58-4.63 (m, 1H), 1.46 (d, J=6.4 Hz, 3H); 3 NHs not observed. (S)-6-(1-aminoethyl)-2-chloro-N-(furan-2-ylmethyl)-7-phenylthieno[3,2-d]pyrimidin-4-amine (formic acid salt): MS m/z 427.5 [M+H]⁺;¹H NMR (methanol-d₄) δ: 8.44 (s, 1H), 8.26 (dd, J=6.6, 3.2 Hz, 2H), 7.40-7.52 (m, 4H), 7.32-7.39 (m, 2H), 7.25-7.32 (m, 3H), 6.26 (bs, 2H), 4.81-4.85 (m, 2H), 4.62 (q, J=6.5 Hz, 1H), 1.41 (d, J=6.7 Hz, 3H); 3 NHs not observed.

### Example 26 (Compound 121)

### 6-[(S)-(1-(1-Aminoethyl)cyclopropyl)]-2-chloro-N-[(furan-2-yl)methyl]-7-methylthieno[3,2-d]pyrimidin-4-amine

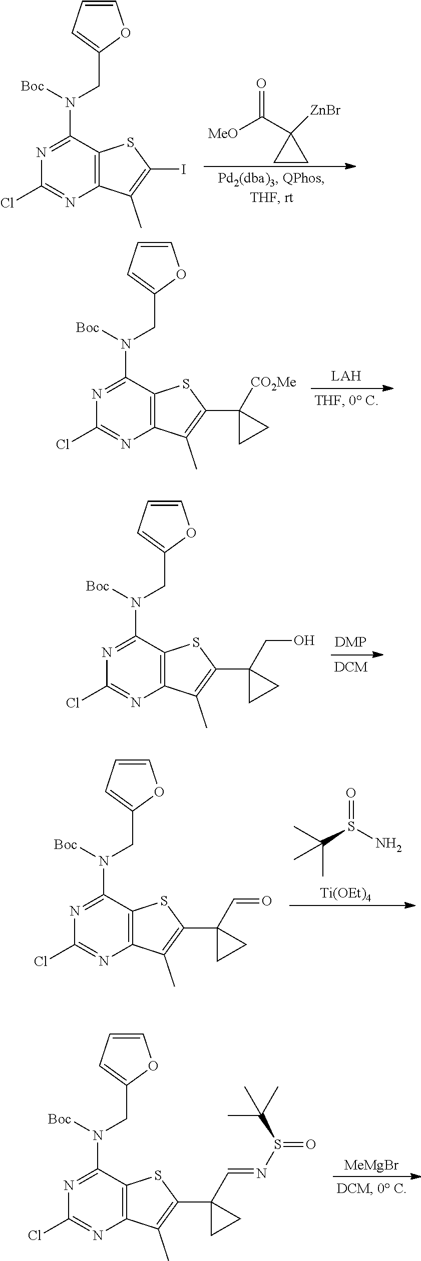

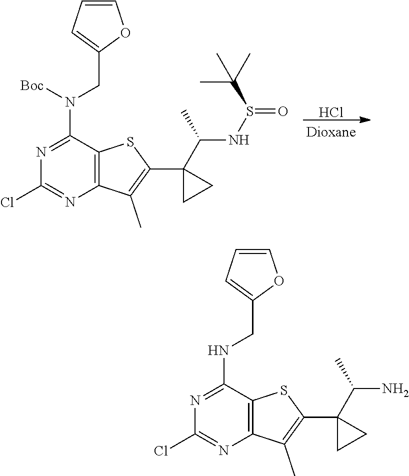 [SMILES: Cc1c(C2([C@H](C)N[S@](=O)C(C)(C)C)CC2)sc2c(N(Cc3ccco3)C(=O)OC(C)(C)C)nc(Cl)nc12 ; Cl ; Cc1c(C2([C@H](C)N)CC2)sc2c(NCc3ccco3)nc(Cl)nc12]

### Step 1: Methyl 1-(4-((tert-butoxycarbonyl)(furan-2-ylmethyl)amino)-2-chloro-7-methylthieno[3,2-d]pyrimidin-6-yl)cyclopropane-1-carboxylate

To a solution of tert-butyl N-(2-chloro-6-iodo-7-methyl-thieno[3,2-d]pyrimidin-4-yl)-N-(2-furylmethyl)carbamate (70 mg, 0.1384 mmol, 1.0 eq, prepared according to the procedure in example 20, step 1), tris(dibenzylideneacetone)dipalladium (8 mg, 0.009 mmol), and 1,2,3,4,5-pentaphenyl-1′-(di-tert-butylphosphino)ferrocene (8 mg, 0.01 mmol) in THF (1 mL) was added bromo-(1-methoxycarbonylcyclopropyl)zinc (1 mL, 0.4 mmol, 0.4 mol/L) at rt. Stirring was continued for 1 h then quenched with NH₄Cl (sat. aq.). The reaction mixture was extracted with EtOAc. The combined organic phases were dried over magnesium sulfate, filtered, and concentrated. The crude residue was purified by flash column chromatography on silica gel eluting with 0-30% EtOAc in hexanes to afford methyl 1-[4-[tert-butoxycarbonyl(2-furylmethyl)amino]-2-chloro-7-methyl-thieno[3,2-d]pyrimidin-6-yl]cyclopropanecarboxylate (40 mg, 60% yield) as a clear oil. MS m/z 478.3, 480.3 [M+H]⁺; ¹H NMR (chloroform-d) δ: 7.29 (s, 1H), 6.28-6.33 (m, 2H), 5.19 (s, 2H), 3.68 (s, 3H), 2.39 (s, 3H), 1.82-1.87 (m, 2H), 1.54 (s, 9H), 1.39-1.44 (m, 2H).

### Step 2: tert-Butyl (2-chloro-6-(1-(hydroxymethyl)cyclopropyl)-7-methylthieno[3,2-d]pyrimidin-4-yl)(furan-2-ylmethyl)carbamate

To a solution of methyl 1-[4-[tert-butoxycarbonyl(2-furylmethyl)amino]-2-chloro-7-methyl-thieno[3,2-d]pyrimidin-6-yl]cyclopropanecarboxylate (600 mg, 1.255 mmol, 1.0 eq) in THF (10 mL), cooled to 0° C., was added LiA₁H₄ (2.0 M in THF, 1 mL, 2 mmol, 1.5 eq) dropwise. After stirring at UPLC shows complete conversion to product after 5 min. Quenched with NH₄Cl (sat. aq.) and diluted with EtOAc. Organics were washed with water and brine and dried over MgSO₄, filtered, and concentrated. The crude residue was purified by flash column chromatography on silica gel eluting with 0-60% EtOAc in hexanes to afford tert-butyl N-[2-chloro-6-[1-(hydroxymethyl)cyclopropyl]-7-methyl-thieno[3,2-d]pyrimidin-4-yl]-N-(2-furylmethyl)carbamate (400 mg, 71% yield) as clear oil. MS m/z 450.3 [M+H]⁺; ¹H NMR (chloroform-d) δ: 7.29 (s, 1H), 6.28-6.32 (m, 2H), 5.19 (s, 2H), 3.72 (s, 2H), 2.50 (s, 3H), 1.54 (s, 9H), 1.08-1.14 (m, 4H); 1 OH not observed.

### Step 3: tert-Butyl (2-chloro-6-(1-formylcyclopropyl)-7-methylthieno[3,2-d]pyrimidin-4-yl)(furan-2-ylmethyl)carbamate

To a solution of tert-butyl N-[2-chloro-6-[1-(hydroxymethyl)cyclopropyl]-7-methyl-thieno[3,2-d]pyrimidin-4-yl]-N-(2-furylmethyl)carbamate (400 mg, 0.8 mmol, 1.0 eq) in dichloromethane (6 mL) at rt was added Dess-Martin periodinane (560 mg, 1.3 mmol, 1.3 eq). After stirring at rt for 20 min, the reaction mixture was diluted with dichloromethane (20 mL) and washed with NaHCO₃ (sat. aq.). Combined organics were dried over NaSO₄, filtered, and concentrated. The crude residue was purified by flash column chromatography on silica gel eluting with 0-40% EtOAc in hexanes to afford tert-butyl N-[2-chloro-6-(1-formylcyclopropyl)-7-methyl-thieno[3,2-d]pyrimidin-4-yl]-N-(2-furylmethyl)carbamate (340 mg, 85% yield) as a clear oil. MS m/z 448.3 [M+H]⁺; ¹H NMR (chloroform-d) δ: 9.17 (s, 1H), 7.29 (s, 1H), 6.28-6.35 (m, 2H), 5.22 (s, 2H), 2.40 (s, 3H), 1.80-1.87 (m, 2H), 1.61-1.67 (m, 2H), 1.55 (s, 9H).

### Step 4: tert-Butyl (R,E)-(6-(1-(((tert-butylsulfinyl)imino)methyl)cyclopropyl)-2-chloro-7-methylthieno[3,2-d]pyrimidin-4-yl)(furan-2-ylmethyl)carbamate

To a solution of tert-butyl N-[2-chloro-6-(1-formylcyclopropyl)-7-methyl-thieno[3,2-d]pyrimidin-4-yl]-N-(2-furylmethyl)carbamate (100 mg, 0.2 mmol, 1.0 eq.) and (R)-(+)-2-methyl-2-propanesulfinamide (42 mg, 0.3 mmol, 1.5 eq.) in THF (2 mL) at rt was added titanium(IV) ethoxide (0.1 mL, 0.5 mmol, 2.5 eq). After stirring at rt for 8 h, the reaction was quenched with water (1 mL), filtered through celite, and concentrated. The crude residue was purified by flash column chromatography on silica gel eluting with 0-50% EtOAc in hexanes to afford tert-butyl (R,E)-(6-(1-(((tert-butylsulfinyl)imino)methyl)cyclopropyl)-2-chloro-7-methylthieno[3,2-d]pyrimidin-4-yl)(furan-2-ylmethyl)carbamate (110 mg, 82% yield) as a clear oil. MS m/z 573.2, 575.2 [M+Na]⁺; ¹H NMR (chloroform-d) δ: 9.10 (s, 1H), 7.53-7.62 (m, 1H), 6.17-6.27 (m, 2H), 5.12 (s, 2H), 2.30 (s, 3H), 1.54-1.70 (m, 4H), 1.44 (s, 9H), 1.04 (s, 9H).

### Step 5: tert-Butyl (6-(1-((S)-1-(((R)-tert-butylsulfinyl)amino)ethyl)cyclopropyl)-2-chloro-7-methylthieno[3,2-d]pyrimidin-4-yl)(furan-2-ylmethyl)carbamate

To a solution of tert-butyl (R,E)-(6-(1-(((tert-butylsulfinyl)imino)methyl)cyclopropyl)-2-chloro-7-methylthieno[3,2-d]pyrimidin-4-yl)(furan-2-ylmethyl)carbamate (122 mg, 0.2 mmol, 1.0 eq.) in dichloromethane (3 mL), cooled to 0° C., was added methylmagnesium bromide (0.1 mL, 3.0 M in diethylether, 1.5 eq.). After stirring at 0° C. for 1 h, the reaction was quenched with NH₄Cl (sat. aq.) and diluted with EtOAc. Organics were dried over MgSO₄, filtered, and concentrated to afford tert-butyl (6-(1-((S)-1-(((R)-tert-butylsulfinyl)amino)ethyl)cyclopropyl)-2-chloro-7-methylthieno[3,2-d]pyrimidin-4-yl)(furan-2-ylmethyl)carbamate (82 mg, 65% yield) as a clear oil. Used in the next step without further purification. MS m/z 589.2, 591.2 [M+Na]⁺.

### Step 6: 6-[(S)-(1-(1-Aminoethyl)cyclopropyl)]-2-chloro-N-[(furan-2-yl)methyl]-7-methylthieno[3,2-d]pyrimidin-4-amine

A mixture of tert-butyl (6-(1-((S)-1-(((R)-tert-butylsulfinyl)amino)ethyl)cyclopropyl)-2-chloro-7-methylthieno[3,2-d]pyrimidin-4-yl)(furan-2-ylmethyl)carbamate (82 mg, 0.15 mmol, 1.0 eq.) in HCl (4 M in dioxane) (1 mL) was stirred at rt for 3 h. The mixture was concentrated and the crude residue was purified by preparative HPLC with 5-40% CH₃CN in water containing 0.1% formic acid to provide (S)-6-(1-(1-aminoethyl)cyclopropyl)-2-chloro-N-(furan-2-ylmethyl)-7-methylthieno[3,2-d]pyrimidin-4-amine (46 mg, 80%) as the formic acid salt. MS m/z 363.2, 365.2 [M+H]⁺;¹H NMR (methanol-d₄) δ: 8.41-8.49 (m, 1H), 7.26 (s, 1H), 6.18-6.32 (m, 2H), 4.64 (s, 2H), 2.66-2.77 (m, 1H), 2.30 (s, 3H) 1.16 (br d, J=6.4 Hz, 3H), 1.14-0.99 (br m, 4H); 3 NHs not observed.

The compounds below were prepared according to the procedure of Example 26 by substituting the appropriate starting materials, reagents, and reaction conditions.

| Com- | |
|---|---|
| pound | Spectral Data |
| 122 | MS m/z 389.3, 391.3 [M + H]⁺; ¹H NMR (methanol-d₄) δ: 8.38- |
| | 8.54 (m, 1H), 7.32 (s, 1H), 6.17-6.30 (m, 2H), 4.63 (s, 2H), 2.33 |
| | (s, 2H), 1.73-1.80 (m, 1H), 1.07-1.14 (m, 1H), 1.02-1.07 (m, |
| | 1H), 0.96-1.02 (m, 1H), 0.88-0.95 (m, 1H), 0.65-0.74 (m, 1H), |
| | 0.52-0.60 (m, 1H), 0.39-0.48 (m, 1H), 0.31-0.39 (m, 1H), 0.13- |
| | 0.20 (m, 1H); 3 NHs not observed |

### BIOLOGICAL EXAMPLES

The following in vitro biological examples demonstrate the usefulness of the compounds of the present description for treating familial dysautonomia.

To describe in more detail and assist in understanding the present description, the following non-limiting biological examples are offered to more fully illustrate the scope of the description and are not to be construed as specifically limiting the scope thereof. Such variations of the present description that may be now known or later developed, which would be within the purview of one skilled in the art to ascertain, are considered to fall within the scope of the present description and as hereinafter claimed.

### Example 1

### IKBKAP-HTRF Assay

The assay is used for the quantitative determination of Elongator complex protein 1 (ELP1, also referred to as IKBKAP) concentration in cell lysates using the HTRF® (Homogeneous Time-Resolved Fluorescence) technology. IKBKAP is detected in a sandwich HTRF assay by use of an anti-IKAP antibody labeled with a donor and an anti-IKAP antibody labeled with an acceptor.

| Materials | Source |
|---|---|
| FD Patient-derived fibroblasts | GM04589 (Coriell Institute) |
| DMEM | Invitrogen Catalogue No. 11960-044 |
| LB4 (4×) Lysis Buffer | Cisbio |
| Protease Inhibitor Cocktail | Roche Catalogue No. 11836145001 |
| Anti IKAP-K(9 + 8) 50× | Cisbio |
| Anti IKAP-d2 50× | Cisbio |
| IKAP Detection Buffer | Cisbio |
| EnVision Plate Reader | Perkin Elmer Model No. 2103 |

### PROTOCOL

Cells were thawed and incubated in DMEM-10% FBS for 72 hours. Cells were trypsinized, counted, and re-suspended to a concentration of 50,000 cells/mL in DMEM-10% FBS. A 199 μL, aliquot of the cell suspensions were plated at 10,000 cells per well in a 96 well microtiter plate and incubated for 3 to 5 hours. To provide a control signal, three wells did not receive cells and served as Blank control wells. Test compounds were serially diluted 3.16-fold in 100% DMSO to generate a 7-point concentration curve. A 1 μL aliquot of 200× compound solution was transferred to cell-containing wells, and cells were incubated for 48 hours in a cell culture incubator (37° C., 5% CO₂, 100% relative humidity). Triplicate samples were set up for each compound concentration. After 48 hours, the supernatant was removed from the cells and 50 μL of the 1× LB4 lysis buffer, containing protease inhibitors, was added to the cells and incubated with shaking at room temperature for 1 hour. A 36 μL aliquot of this lysate was subsequently transferred to the 384-well plate containing 4 μL of the antibody solution (1:50 dilution of anti IKAP d2 and anti-IKAP K(9+8) in detection buffer). The 384-well plate was then centrifuged for 1 minute to bring the solutions to the bottom of the plate and incubated overnight at 4° C. Fluorescence for each well of the plate at 665 nm and 620 nm was measured was on the EnVision plate reader (Perkin Elmer). The AF for each sample is calculated by:

$$Δ F = \frac{( Signal - Blank ) ⨯ 100}{Blank}$$

wherein Signal is the normalized fluorescence for each sample well and Blank is the average normalized average fluorescence for the Blank control wells.

The maximum fold increase (MFI) in IKBKAP protein abundance for compounds of Formula (I) or a form thereof relative to the vehicle control are provided in Table 1. MFI was calculated by dividing the AF value for each sample well by the sample AF for the vehicle control wells.

An MFI<1.9 is indicated by one star (*), between >1.9 and <2.9 is indicated by two stars (**), between >2.9 and <3.9 is indicated by three stars (***), between >3.9 and <4.9 is indicated by four stars (****), and >4.9 is indicated by five stars (*****).

The EC_{2×} for IKBKAP protein expression obtained from the 7-point concentration curve generated for each test compound according to the protocol in Biological Example 1 are also provided in Table 1. The term “EC_{2×} for IKBKAP protein expression” is defined as the concentration of test compound that is effective in producing two times the amount of IKBKAP protein in a FD patient cell compared to the amount produced from the DMSO vehicle control.

An EC_{2×}>1 μM is indicated by one star (*), between >0.5 μL and ≤1 μM is indicated by two stars (**), between >0.02 μM and ≤0.5 μM is indicated by three stars (***), between >0.005 μM and ≤0.02 μM is indicated by four stars (****), and <0.005 μM is indicated by five stars (*****).

TABLE 1

| Cpd | MFI | EC₂ₓ |
|---|---|---|
| 1 | **** | * |
| 2 | *** | * |
| 3 | **** | **** |
| 4 | **** | **** |
| 5 | *** | *** |
| 6 | *** | *** |
| 7 | * | ** |
| 8 | *** | *** |
| 9 | ***** | **** |
| 10 | **** | *** |
| 11 | **** | *** |
| 12 | ***** | ***** |
| 13 | ***** | ***** |
| 14 | ***** | **** |
| 15 | ***** | **** |
| 16 | *** | *** |
| 17 | * | * |
| 18 | ***** | *** |
| 19 | ***** | ***** |
| 20 | ** | * |
| 21 | ***** | **** |
| 22 | ***** | ***** |
| 23 | ***** | *** |
| 24 | *** | *** |
| 25 | * | * |
| 26 | **** | *** |
| 27 | *** | ** |
| 28 | ***** | ***** |
| 29 | ***** | ***** |
| 30 | **** | *** |
| 31 | *** | *** |
| 32 | **** | *** |
| 33 | **** | **** |
| 34 | ***** | ***** |
| 35 | ***** | ***** |
| 36 | ***** | **** |
| 37 | ***** | ***** |
| 38 | ***** | ***** |
| 39 | ***** | *** |
| 40 | ***** | ***** |
| 41 | ** | *** |
| 42 | ***** | *** |
| 43 | **** | *** |
| 44 | ***** | ***** |
| 45 | ***** | ***** |
| 46 | * | * |
| 47 | *** | *** |
| 48 | ** | *** |
| 49 | * | * |
| 50 | ****** | ****** |
| 51 | ***** | ***** |
| 52 | ** | * |
| 53 | ***** | ***** |
| 54 | ***** | ***** |
| 55 | *** | *** |
| 56 | ***** | ***** |
| 57 | *** | *** |
| 58 | * | * |
| 59 | **** | *** |
| 60 | *** | *** |
| 61 | * | * |
| 62 | ***** | **** |
| 63 | * | * |
| 64 | ***** | **** |
| 65 | * | * |
| 66 | * | * |
| 67 | * | * |
| 68 | ***** | ***** |
| 69 | ***** | **** |
| 70 | **** | *** |
| 71 | * | * |
| 72 | *** | *** |
| 73 | ** | *** |
| 74 | **** | *** |
| 75 | ***** | *** |
| 76 | ***** | ***** |
| 77 | ***** | **** |
| 78 | *** | *** |
| 79 | **** | *** |
| 80 | ***** | *** |
| 81 | * | * |
| 82 | ***** | ***** |
| 83 | ***** | **** |
| 84 | **** | *** |
| 85 | *** | *** |
| 86 | **** | **** |
| 87 | * | * |
| 88 | * | * |
| 89 | ***** | ***** |
| 90 | ***** | ***** |
| 91 | ***** | **** |
| 92 | ***** | ***** |
| 93 | ***** | **** |
| 94 | * | * |
| 95 | *** | * |
| 96 | ** | * |
| 97 | ***** | ***** |
| 98 | ***** | **** |
| 99 | *** | **** |

Without regard to whether a document cited herein was specifically and individually indicated as being incorporated by reference, all documents referred to herein are incorporated by reference into the present application for any and all purposes to the same extent as if each individual reference was fully set forth herein.

Having now fully described the subject matter of the claims, it will be understood by those having ordinary skill in the art that the same can be performed within a wide range of equivalents without affecting the scope of the subject matter or particular aspects described herein. It is intended that the appended claims be interpreted to include all such equivalents.

1. A compound of Formula (I): 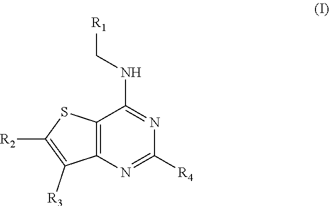
or a form thereof, wherein
R₁ is selected from the group consisting of aryl and heteroaryl, optionally substituted with one, two, three, or four independently selected R₁ₐ substituents;
R₁ₐ selected from the group consisting of cyano, halo, hydroxy, C₁₋₆alkyl, halo-C₁₋₆alkyl, deutero-C₁₋₆alkyl, and C₁₋₆alkoxy;
R₂ selected from the group consisting of hydrogen, C₁₋₆alkyl, C₂₋₆alkenyl, C₂₋₆alkynyl, C₃₋₁₀cycloalkyl, aryl, heterocyclyl, and heteroaryl,
wherein each instance of C₁₋₆alkyl, C₂₋₆alkenyl, C₂₋₆alkynyl, C₃₋₁₀cycloalkyl, aryl, heterocyclyl, and heteroaryl is optionally substituted with one, two, three, or four independently selected R₂ₐ substituents, and
wherein each instance of C₁₋₆alkyl, C₂₋₆alkenyl, C₂₋₆alkynyl and heterocyclyl optionally contains a chiral carbon having an (R) or (S) configuration;
R₂ₐ selected from the group consisting of cyano, halo, hydroxy, oxo, C₁₋₆alkyl, halo-C₁₋₆alkyl, deutero-C₁₋₆alkyl, C₂₋₆alkenyl, C₂₋₆alkynyl, C₁₋₆alkoxy, halo-C₁₋₆alkoxy, carboxyl, amino, C₁₋₆alkyl-amino, halo-C₁₋₆alkyl-amino, deutero-C₁₋₆alkyl-amino, (C₁₋₆alkyl)₂-amino, C₃₋₁₀cycloalkyl-amino, aryl-amino, heterocyclyl-amino, heteroaryl-amino, C₁₋₆alkyl-thio, C₁₋₆alkyl-sulfonyl, C₃₋₁₀cycloalkyl, aryl, heterocyclyl, and heteroaryl,
wherein each instance of C₃₋₁₀cycloalkyl, aryl, heterocyclyl and heteroaryl is optionally substituted with one, two, three or four independently selected R_{2a′} substituents;
R_{2a′} selected from the group consisting of cyano, halo, hydroxy, oxo, C₁₋₆alkyl, halo-C₁₋₆alkyl, deutero-C₁₋₆alkyl, and C₁₋₆alkoxy;
R₃ selected from the group consisting of hydrogen, cyano, halo, hydroxy, C₁₋₆alkyl, halo-C₁₋₆alkyl, C₁₋₆alkoxy, amino, C₁₋₆alkyl-amino, (C₁₋₆alkyl)₂-amino, C₃₋₁₀ocycloalkyl, aryl, heterocyclyl, and heteroaryl,
wherein each instance of C₁₋₆alkyl, C₃₋₁₀cycloalkyl, aryl, heterocyclyl, or heteroaryl are optionally substituted with one, two, three, or four independently selected R₃ₐ substituents;
R₃ₐ selected from the group consisting of cyano, halo, hydroxy, C₁₋₆alkyl, halo-C₁₋₆alkyl, and C₁₋₆alkoxy; and
R₄ selected from the group consisting of hydrogen, cyano, halo, hydroxy, C₁₋₆alkyl, halo-C₁₋₆alkyl, C₁₋₆alkoxy, carbamoyl, C₃₋₁₀cycloalkyl, aryl, and heterocyclyl;
wherein the form of the compound is selected from the group consisting of a salt, hydrate, solvate, racemate, enantiomer, diastereomer, stereoisomer, and tautomer form thereof.

2. The compound of claim 1, wherein R₁ is phenyl optionally substituted with one, two, three, or four independently selected R₁ₐ substituents.

3. The compound of claim 1, wherein R₁ is heteroaryl selected from the group consisting of furanyl, thiophenyl, 1H-pyrazolyl, 1H-imidazolyl, isoxazolyl, 1,2-thiazolyl, 1,3-thiazolyl, 1,2-oxazolyl, 1,3-oxazolyl, tetrazolyl, 1,2,3-triazolyl, 1,2,4-oxadiazolyl, 1,3,4-oxadiazolyl, 1,2,3-thiadiazolyl, pyridinyl, pyrimidinyl, pyrazinyl, pyridazinyl, benzofuranyl, and quinolinyl, wherein heteoraryl is optionally substituted with one, two, three, or four independently selected R₁ₐ substituents.

4. The compound of claim 1, wherein R₁ is heteroaryl selected from the group consisting of furanyl, thiophenyl, 1H-pyrazolyl, 1H-imidazolyl, 1,2-thiazolyl, 1,3-thiazolyl, 1,2-oxazolyl, 1,3-oxazolyl, pyridinyl, pyrimidinyl, and pyrazinyl, wherein heteroaryl is optionally substituted with one, two, three, or four independently selected R₁ₐ substituents.

5. The compound of claim 1, wherein R₁ₐ is selected from the group consisting of halo and C₁₋₆alkyl.

6. The compound of claim 1, wherein R₂ is selected from the group consisting of hydrogen, C₁₋₆alkyl, and heterocyclyl, wherein each instance of C₁₋₆alkyl and heterocyclyl is optionally substituted with one, two, three, or four independently selected Rea substituents, and wherein each instance of C₁₋₆alkyl and heterocyclyl optionally contains a chiral carbon having an (R) or (S) configuration.

7. The compound of claim 1, wherein R₂ is C₁₋₆alkyl selected from the group consisting of methyl, ethyl, propyl, butyl, and pentyl.

8. The compound of claim 1, wherein R₂ is C₁₋₆alkyl, wherein C₁₋₆alkyl contains a chiral carbon having the (R) configuration.

9. The compound of claim 1, wherein R₂ is C₁₋₆alkyl, wherein C₁₋₆alkyl contains a chiral carbon having the (S) configuration.

10. A compound, or a form thereof, selected from the group consisting of:
2-chloro-N-[(pyridin-4-yl)methyl]thieno[3,2-d]pyrimidin-4-amine;
2-chloro-N-[(furan-2-yl)methyl]thieno[3,2-d]pyrimidin-4-amine;
6-[(2S)-2-aminopropyl]-2-chloro-N-[(furan-2-yl)methyl]thieno[3,2-d]pyrimidin-4-amine;
6-[(2S)-2-aminobutyl]-2-chloro-N-[(furan-2-yl)methyl]thieno[3,2-d]pyrimidin-4-amine;
6-[(2R)-2-amino-3-methylbutyl]-2-chloro-N-[(furan-2-yl)methyl]thieno[3,2-d]pyrimidin-4-amine;
6-[(2R,3S)-2-amino-3-methylpentyl]-2-chloro-N-[(furan-2-yl)methyl]thieno[3,2-d]pyrimidin-4-amine;
6-[(2R)-2-amino-3,3-dimethylbutyl]-2-chloro-N-[(furan-2-yl)methyl]thieno[3,2-d]pyrimidin-4-amine;
6-[(2S)-2-aminopropyl]-N-[furan-2-yl)methyl]thieno[3,2-d]pyrimidin-4-amine;
6-[(2S)-2-aminopropyl]-2-chloro-N-[(pyridin-4-yl)methyl]thieno[3,2-d]pyrimidin-4-amine;
6-[(2S)-2-aminobutyl]-2-chloro-N-[(thiophen-2-yl)methyl]thieno[3,2-d]pyrimidin-4-amine;
6-[(2S)-2-aminopropyl]-N-[pyridin-4-yl)methyl]thieno[3,2-d]pyrimidin-4-amine;
6-[(2S)-2-aminopropyl]-2-chloro-N-[(furan-2-yl)methyl]-7-methylthieno[3,2-d]pyrimidin-4-amine;
6-[(2S)-2-aminobutyl]-2-chloro-N-[(furan-2-yl)methyl]-7-methylthieno[3,2-d]pyrimidin-4-amine;
6-[(2S)-2-aminopropyl]-2-chloro-N-[(thiophen-2-yl)methyl]thieno[3,2-d]pyrimidin-4-amine;
6-[(2S)-2-aminopropyl]-N-[(furan-2-yl)methyl]-2,7-dimethylthieno[3,2-d]pyrimidin-4-amine;
6-[(2S)-2-aminopropyl]-2-ethyl-N-[(furan-2-yl)methyl]-7-methylthieno[3,2-d]pyrimidin-4-amine;
6-[(2S)-2-aminopropyl]-2-cyclopropyl-N-[(furan-2-yl)methyl]-7-methylthieno[3,2-d]pyrimidin-4-amine;
2-chloro-N-[(furan-2-yl)methyl]-7-methylthieno[3,2-d]pyrimidin-4-amine;
(2R)-2-amino-3-(2-chloro-4-{[(furan-2-yl)methyl]amino}-7-methylthieno[3,2-d]pyrimidin-6-yl)propan-1-ol;
6-[(2S)-2-aminopropyl]-4-{[(furan-2-yl)methyl]amino}-7-methylthieno[3,2-d]pyrimidine-2-carboxamide;
6-[(2S)-2-aminopropyl]-4-{[(furan-2-yl)methyl]amino}-7-methylthieno[3,2-d]pyrimidine-2-carbonitrile;
(2R)-2-amino-3-(2-chloro-4-{[(furan-2-yl)methyl]amino}thieno[3,2-d]pyrimidin-6-yl)propan-1-ol;
2-chloro-7-methyl-N-[(pyridin-4-yl)methyl]thieno[3,2-d]pyrimidin-4-amine;
6-[(2S)-2-aminopropyl]-2-chloro-7-methyl-N-[(5-methylfuran-2-yl)methyl]thieno[3,2-d]pyrimidin-4-amine;
N-[(furan-2-yl)methyl]-7-methyl-2-(trifluoromethyl)thieno[3,2-d]pyrimidin-4-amine;
6-[(2S)-2-aminopropyl]-N-[(furan-2-yl)methyl]-7-methyl-2-(trifluoromethyl)thieno[3,2-d]pyrimidin-4-amine;
6-[(2S)-2-aminopropyl]-2-chloro-7-methyl-N-[(4-methyl-1,3-thiazol-2-yl)methyl]thieno[3,2-d]pyrimidin-4-amine;
6-[(2S)-2-aminopropyl]-2-chloro-7-methyl-N-[(thiophen-2-yl)methyl]thieno[3,2-d]pyrimidin-4-amine;
6-[(2S)-2-aminopropyl]-2-chloro-7-methyl-N-[(1,3-thiazol-2-yl)methyl]thieno[3,2-d]pyrimidin-4-amine;
6-[(2S)-2-aminopropyl]-2-chloro-7-methyl-N-[(3-methylfuran-2-yl)methyl]thieno[3,2-d]pyrimidin-4-amine;
6-[(2S)-2-aminopropyl]-2-chloro-7-methyl-N-[(5-methyl-1,3-thiazol-2-yl)methyl]thieno[3,2-d]pyrimidin-4-amine;
6-[(2S)-2-aminopropyl]-2-chloro-7-methyl-N-[(pyrazin-2-yl)methyl]thieno[3,2-d]pyrimidin-4-amine;
6-[(2S)-2-aminopropyl]-2-chloro-N-[(5-fluorothiophen-2-yl)methyl]-7-methylthieno[3,2-d]pyrimidin-4-amine
6-[(2S)-2-aminopropyl]-N-benzyl-2-chloro-7-methylthieno[3,2-d]pyrimidin-4-amine;
6-[(2S)-2-aminopropyl]-2-chloro-N-[(3-fluoropyridin-4-yl)methyl]-7-methylthieno[3,2-d]pyrimidin-4-amine;
6-[(2S)-2-aminopropyl]-2-chloro-7-cyclopropyl-N-[(furan-2-yl)methyl]thieno[3,2-d]pyrimidin-4-amine;
6-[(2S)-2-aminobutyl]-2-chloro-7-methyl-N-[(thiophen-2-yl)methyl]thieno[3,2-d]pyrimidin-4-amine;
6-[(2S)-2-aminopropyl]-2-bromo-N-[(furan-2-yl)methyl]-7-methylthieno[3,2-d]pyrimidin-4-amine;
6-[(2S)-2-aminopropyl]-2-chloro-7-methyl-N-[(1,2-oxazol-5-yl)methyl]thieno[3,2-d]pyrimidin-4-amine;
6-[(2S)-2-aminopropyl]-7-bromo-2-chloro-N-[(furan-2-yl)methyl]thieno[3,2-d]pyrimidin-4-amine;
6-(azetidin-3-yl)-2-chloro-N-[(furan-2-yl)methyl]-7-methylthieno[3,2-d]pyrimidin-4-amine;
7-bromo-2-chloro-N-[(furan-2-yl)methyl]thieno[3,2-d]pyrimidin-4-amine;
7-bromo-2-chloro-N-[(pyridin-4-yl)methyl]thieno[3,2-d]pyrimidin-4-amine;
6-[(2S)-2-aminopropyl]-2-chloro-N-[(2-fluorophenyl)methyl]-7-methylthieno[3,2-d]pyrimidin-4-amine;
6-[(2S)-2-aminopropyl]-2-chloro-7-methyl-N-[(pyridin-4-yl)methyl]thieno[3,2-d]pyrimidin-4-amine;
6-[(1 S)-1-aminoethyl]-7-bromo-2-chloro-N-[(furan-2-yl)methyl]thieno[3,2-d]pyrimidin-4-amine;
6-[(1S)-1-aminoethyl]-2-chloro-N-[(furan-2-yl)methyl]-7-methylthieno[3,2-d]pyrimidin-4-amine;
6-[(1S)-1-aminopropyl]-2-chloro-N-[(furan-2-yl)methyl]-7-methylthieno[3,2-d]pyrimidin-4-amine;
6-[(1R)-1-aminopropyl]-2-chloro-N-[(furan-2-yl)methyl]-7-methylthieno[3,2-d]pyrimidin-4-amine;
6-[(2S)-2-aminopropyl]-2-chloro-7-methyl-N-[(pyrimidin-4-yl)methyl]thieno[3,2-d]pyrimidin-4-amine;
6-[(2S)-2-amino-4-fluorobutyl]-2-chloro-N-[(furan-2-yl)methyl]-7-methylthieno[3,2-d]pyrimidin-4-amine;
(4S)-4-[(2-chloro-4-{[(furan-2-yl)methyl]amino}-7-methylthieno[3,2-d]pyrimidin-6-yl)methyl]-1,3-oxazinan-2-one;
6-[(2S)-2-aminobutyl]-2-chloro-N-[(3-fluoropyridin-4-yl)methyl]-7-methylthieno[3,2-d]pyrimidin-4-amine;
6-[(2S)-2-aminobutyl]-2-chloro-7-methyl-N-[(1,3-thiazol-2-yl)methyl]thieno[3,2-d]pyrimidin-4-amine;
6-[(2S)-2-aminopropyl]-2-chloro-7-methyl-N-[(pyrimidin-2-yl)methyl]thieno[3,2-d]pyrimidin-4-amine;
(2R)-2-amino-3-(2-chloro-7-methyl-4-{[(thiophen-2-yl)methyl]amino}thieno[3,2-d]pyrimidin-6-yl)propan-1-ol;
2-chloro-N-[(furan-2-yl)methyl]-7-methyl-6-(pyrrolidin-3-yl)thieno[3,2-d]pyrimidin-4-amine;
6-[(1S)-1-aminoethyl]-2-chloro-N-[(furan-2-yl)methyl]-7-phenylthieno[3,2-d]pyrimidin-4-amine;
6-[(2S)-2-aminopropyl]-2-chloro-N-[(3-fluoropyridin-2-yl)methyl]-7-methylthieno[3,2-d]pyrimidin-4-amine;
6-[(2S)-2-aminopropyl]-2-chloro-N-[(2-fluoropyridin-3-yl)methyl]-7-methylthieno[3,2-d]pyrimidin-4-amine;
6-[(1S)-1-aminoethyl]-N-[(furan-2-yl)methyl]-2,7-diphenylthieno[3,2-d]pyrimidin-4-amine;
6-[(2S)-2-aminobutyl]-2-chloro-7-methyl-N-[(1,2-thiazol-5-yl)methyl]thieno[3,2-d]pyrimidin-4-amine;
3-(2-chloro-4-{[(furan-2-yl)methyl]amino}-7-methylthieno[3,2-d]pyrimidin-6-yl)propan-1-ol;
6-[(2S)-2-aminopropyl]-2-chloro-N-[(3,5-difluoropyridin-4-yl)methyl]-7-methylthieno[3,2-d]pyrimidin-4-amine;
6-[(2S)-2-aminopropyl]-2-chloro-N-[(furan-2-yl)methyl]-7-(4-methoxyphenyl)thieno[3,2-d]pyrimidin-4-amine;
(2S)-3-(2-chloro-4-{[(furan-2-yl)methyl]amino}-7-methylthieno[3,2-d]pyrimidin-6-yl)-2-methylpropan-1-ol;
6-(3-aminopropyl)-2-chloro-N-[(furan-2-yl)methyl]-7-methylthieno[3,2-d]pyrimidin-4-amine;
6-[(2S)-2-aminopropyl]-7-bromo-2-chloro-N-[(3-fluoropyridin-4-yl)methyl]thieno[3,2-d]pyrimidin-4-amine;
6-[(2S)-2-aminobutyl]-2-chloro-7-methyl-N-[(1,3-oxazol-2-yl)methyl]thieno[3,2-d]pyrimidin-4-amine;
6-[(2S)-3-amino-2-methylpropyl]-2-chloro-N-[(furan-2-yl)methyl]-7-methylthieno[3,2-d]pyrimidin-4-amine;
(2R)-3-(2-chloro-4-{[(furan-2-yl)methyl]amino}-7-methylthieno[3,2-d]pyrimidin-6-yl)-2-methylpropan-1-ol;
6-[(2R)-3-amino-2-methylpropyl]-2-chloro-N-[(furan-2-yl)methyl]-7-methylthieno[3,2-d]pyrimidin-4-amine;
6-[(2S)-2-aminopropyl]-2-chloro-N-[(1H-imidazol-2-yl)methyl]-7-methylthieno[3,2-d]pyrimidin-4-amine;
6-[(2S)-2-aminopropyl]-2-chloro-7-methyl-N-[(1,3-thiazol-5-yl)methyl]thieno[3,2-d]pyrimidin-4-amine;
6-[(2S)-2-aminopropyl]-2-chloro-7-methyl-N-[(1,3-oxazol-5-yl)methyl]thieno[3,2-d]pyrimidin-4-amine;
6-[(2R)-2-amino-3-methoxypropyl]-2-chloro-N-[(furan-2-yl)methyl]-7-methylthieno[3,2-d]pyrimidin-4-amine;
6-[(2S)-2-aminopropyl]-2-chloro-7-ethyl-N-[(furan-2-yl)methyl]thieno[3,2-d]pyrimidin-4-amine;
2-chloro-6-[(2S)-2-(cyclobutylamino)propyl]-N-[(furan-2-yl)methyl]-7-methylthieno[3,2-d]pyrimidin-4-amine;
2-chloro-N-[(furan-2-yl)methyl]-7-methyl-6-[(2S)-2-(methylamino)propyl]thieno[3,2-d]pyrimidin-4-amine;
7-bromo-2-chloro-N-[(thiophen-2-yl)methyl]thieno[3 ,2-d]pyrimidin-4-amine;
6-[(2S)-2-aminopropyl]-2-chloro-7-methyl-N-[(1-methyl-1H-pyrazol-5-yl)methyl]thieno[3 ,2-d]pyrimidin-4-amine;
6-[(2S)-2-aminopropyl]-7-bromo-2-chloro-N-[(thiophen-2-yl)methyl]thieno[3,2-d]pyrimidin-4-amine;
6-[(2S)-2-aminopropyl]-7-bromo-4-{[(thiophen-2-yl)methyl]amino}thieno[3,2-d]p yrimidine-2-c arbonitrile;
2-chloro-4-{[(thiophen-2-yl)methyl]amino}thieno[3 ,2-d]pyrimidine-7-carbonitrile;
6-[(2S)-2-aminopropyl]-4-{[(thiophen-2-yl)methyl]amino}thieno[3 ,2-d]pyrimidine-2,7-dicarbonitrile;
6-[(2S)-2-aminopropyl]-2-chloro-7-cyclopropyl-N-[(thiophen-2-yl)methyl]thieno[3,2-d]pyrimidin-4-amine;
6-[(2S)-2-aminopropyl]-2-chloro-7-phenyl-N-[(thiophen-2-yl)methyl]thieno[3,2-d]pyrimidin-4-amine;
6-[(2S)-2-aminopropyl]-2-chloro-7-(4-chlorophenyl)-N-[(thiophen-2-yl)methyl]thieno[3 ,2-d]pyrimidin-4-amine;
6-[(2S)-2-aminobutyl]-2-chloro-7-methyl-N-[(pyrimidin-4-yl)methyl]thieno[3,2-d]pyrimidin-4-amine;
6-[(2S)-2-aminopropyl]-2-chloro-N-[(3-fluorothiophen-2-yl)methyl]-7-methylthieno[3 ,2-d]pyrimidin-4-amine;
6-[(2S)-2-aminobutyl]-2-chloro-N-[(3-fluorothiophen-2-yl)methyl]-7-methylthieno[3,2-d]pyrimidin-4-amine;
6-[(2S)-2-aminopropyl]-2-chloro-N-[(4-fluoro-1,3-thiazol-2-yl)methyl]-7-methylthieno[3,2-d]pyrimidin-4-amine;
6-[(2S)-2-aminopropyl]-2-chloro-N-[(5-fluoro-1,3-thiazol-2-yl)methyl]-7-methylthieno[3,2-d]pyrimidin-4-amine;
6-[(1R)-1-aminoethyl]-2-chloro-N-[(furan-2-yl)methyl]-7-methylthieno[3,2-d]pyrimidin-4-amine;
2-chloro-N-[(pyrimidin-4-yl)methyl]thieno[3,2-d]pyrimidin-4-amine;
2-chloro-N-[(1,3-thiazol-2-yl)methyl]thieno[3,2-d]pyrimidin-4-amine;
(3S)-3-amino-4-(2-chloro-4-{[(furan-2-yl)methyl]amino}-7-methylthieno[3,2-d]pyrimidin-6-yl)butan-1-ol;
6-[(2S)-2-aminopropyl]-2-chloro-N-[(5-fluoropyrimidin-4-yl)methyl]-7-methylthieno[3,2-d]pyrimidin-4-amine, and
(2R)-2-amino-3-(2-chloro-7-methoxy-4-{[(thiophen-2-yl)methyl]amino}thieno[3,2-d]pyrimidin-6-yl)propan-1-ol;
wherein the form of the compound is selected from the group consisting of a salt, hydrate, solvate, racemate, enantiomer, diastereomer, stereoisomer, and tautomer form thereof.

11. The compound of claim 1, or a form thereof, wherein the compound is selected from the group consisting of:
6-[(2S)-2-aminopropyl]-2-chloro-7-methyl-N-[(thiophen-3-yl)methyl]thieno[3,2-d]pyrimidin-4-amine
6-[(2S)-2-aminopropyl]-2-chloro-7-methyl-N-[(1H-pyrazol-5-yl)methyl]thieno[3,2-d]pyrimidin-4-amine
6-[(2S)-2-aminopropyl]-2-chloro-7-methyl-N-[(1-methyl-1H-imidazol-2-yl)methyl]thieno[3,2-d]pyrimidin-4-amine
6-[(2S)-2-aminopropyl]-2-chloro-N-[(furan-3-yl)methyl]-7-methylthieno[3,2-d]pyrimidin-4-amine
6-[(2S)-2-aminopropyl]-2-chloro-7-methyl-N-[(1H-pyrrol-2-yl)methyl]thieno[3,2-d]pyrimidin-4-amine
6-[(2S)-2-aminopropyl]-2-chloro-7-methyl-N-[(1,2-oxazol-3-yl)methyl]thieno[3,2-d]pyrimidin-4-amine
6-[(2S)-2-aminopropyl]-2-chloro-7-methyl-N-[(1-methyl-1H-pyrazol-3-yl)methyl]thieno[3,2-d]pyrimidin-4-amine
6-[(2S)-2-aminopropyl]-2-chloro-7-methyl-N-[(1,2-oxazol-4-yl)methyl]thieno[3,2-d]pyrimidin-4-amine
6-[(2S)-2-aminopropyl]-2-chloro-7-methyl-N-[(1,2-thiazol-4-yl)methyl]thieno[3,2-d]pyrimidin-4-amine
6-[(2S)-2-aminopropyl]-2-chloro-7-methyl-N-[(1H-pyrazol-4-yl)methyl]thieno[3,2-d]pyrimidin-4-amine
6-[(2S)-2-aminopropyl]-2-chloro-7-methyl-N-[(1-methyl-1H-pyrazol-4-yl)methyl]thieno[3,2-d]pyrimidin-4-amine
6-[(2S)-2-aminopropyl]-2-chloro-7-methyl-N-[(1,3-oxazol-2-yl)methyl]thieno[3,2-d]pyrimidin-4-amine
6-[(2S)-2-aminopropyl]-2-chloro-7-methyl-N-[(1,3-oxazol-4-yl)methyl]thieno[3,2-d]pyrimidin-4-amine
6-[(2S)-2-aminopropyl]-2-chloro-7-methyl-N-[(1,3-thiazol-4-yl)methyl]thieno[3,2-d]pyrimidin-4-amine
6-[(2S)-2-aminopropyl]-2-chloro-N-[(1H-imidazol-5-yl)methyl]-7-methylthieno[3,2-d]pyrimidin-4-amine
6-[(2S)-2-aminopropyl]-2-chloro-7-methyl-N-[(1-methyl-1H-imidazol-5-yl)methyl]thieno[3,2-d]pyrimidin-4-amine
6-[(2S)-2-aminopropyl]-2-chloro-7-methyl-N-[(1-methyl-1H-imidazol-4-yl)methyl]thieno[3,2-d]pyrimidin-4-amine
6-[(2S)-2-aminopropyl]-2-chloro-7-methyl-N-[(2H-1,2,3-triazol-4-yl)methyl]thieno[3,2-d]pyrimidin-4-amine
6-[(2S)-2-aminopropyl]-2-chloro-7-methyl-N-[(1H-tetrazol-5-yl)methyl]thieno[3,2-d]pyrimidin-4-amine
6-[(2S)-2-aminopropyl]-2-chloro-7-methyl-N-[(1-methyl-1H-pyrrol-2-yl)methyl]thieno[3,2-d]pyrimidin-4-amine
6-[(2S)-2-aminopropyl]-2-chloro-7-methyl-N-[(1H-pyrrol-3-yl)methyl]thieno[3,2-d]pyrimidin-4-amine
6-[(S)-(1-(1-aminoethyl)cyclopropyl)]-2-chloro-N-[(furan-2-yl)methyl]-7-methylthieno[3,2-d]pyrimidin-4-amine
6-[(S)-(1-(amino(cyclopropyl)methyl)cyclopropyl)]-2-chloro-N-[(furan-2-yl)methyl]-7-methylthieno[3,2-d]pyrimidin-4-amine
6-[(2R)-2-amino-2-cyclopropylethyl]-7-bromo-2-chloro-N-[(thiophen-2-yl)methyl]thieno[3,2-d]pyrimidin-4-amine, and
6-[(2R)-2-amino-2-cyclopropylethyl]-2-chloro-7-methyl-N-[(thiophen-2-yl)methyl]thieno[3,2-d]pyrimidin-4-amine;
wherein the form of the compound is selected from the group consisting of a salt, hydrate, solvate, racemate, enantiomer, diastereomer, stereoisomer, and tautomer form thereof.

12. A compound, or a form thereof, selected from the group consisting of:
6-[(2S)-2-aminopropyl]-2-chloro-N-[(furan-2-yl)methyl]thieno[3,2-d]pyrimidin-4-amine dihydrochloride;
6-[(2S)-2-aminobutyl]-2-chloro-N-[(furan-2-yl)methyl]thieno[3,2-d]pyrimidin-4-amine dihydrochloride;
6-[(2R)-2-amino-3-methylbutyl]-2-chloro-N-[(furan-2-yl)methyl]thieno[3,2-d]pyrimidin-4-amine dihydrochloride;
6-[(2R,3S)-2-amino-3-methylpentyl]-2-chloro-N-[(furan-2-yl)methyl]thieno[3,2-d]pyrimidin-4-amine dihydrochloride;
6-[(2R)-2-amino-3,3-dimethylbutyl]-2-chloro-N-[(furan-2-yl)methyl]thieno[3,2-d]pyrimidin-4-amine dihydrochloride;
6-[(2S)-2-aminopropyl]-N-[furan-2-yl)methyl]thieno[3,2-d]pyrimidin-4-amine dihydrochloride;
6-[(2S)-2-aminopropyl]-2-chloro-N-[(pyridin-4-yl)methyl]thieno[3,2-d]pyrimidin-4-amine dihydrochloride;
6-[(2S)-2-aminobutyl]-2-chloro-N-[(thiophen-2-yl)methyl]thieno[3,2-d]pyrimidin-4-amine dihydrochloride;
6-[(2S)-2-aminopropyl]-N-[pyridin-4-yl)methyl]thieno[3,2-d]pyrimidin-4-amine dihydrochloride;
6-[(2S)-2-aminopropyl]-2-chloro-N-[(furan-2-yl)methyl]-7-methylthieno[3,2-d]pyrimidin-4-amine dihydrochloride;
6-[(2S)-2-aminobutyl]-2-chloro-N-[(furan-2-yl)methyl]-7-methylthieno[3,2-d]pyrimidin-4-amine dihydrochloride;
6-[(2S)-2-aminopropyl]-2-chloro-N-[(thiophen-2-yl)methyl]thieno[3,2-d]pyrimidin-4-amine dihydrochloride;
6-[(2S)-2-aminopropyl]-N-[furan-2-yl)methyl]-2,7-dimethylthieno[3,2-d]pyrimidin-4-amine dihydrochloride;
6-[(2S)-2-aminopropyl]-2-ethyl-N-[(furan-2-yl)methyl]-7-methylthieno[3,2-d]pyrimidin-4-amine dihydrochloride;
6-[(2S)-2-aminopropyl]-2-cyclopropyl-N-[(furan-2-yl)methyl]-7-methylthieno[3,2-d]pyrimidin-4-amine dihydrochloride;
2-chloro-N-[(furan-2-yl)methyl]-7-methylthieno[3,2-d]pyrimidin-4-amine hydrochloride;
(2R)-2-amino-3-(2-chloro-4-{[(furan-2-yl)methyl]amino}-7-methylthieno[3,2-d]pyrimidin-6-yl)propan-1-ol dihydrochloride;
6-[(2S)-2-aminopropyl]-4-{[(furan-2-yl)methyl]amino}-7-methylthieno[3,2-d]pyrimidine-2-carboxamide trifluoroacetate;
6-[(2S)-2-aminopropyl]-4-{[(furan-2-yl)methyl]amino}-7-methylthieno[3,2-d]pyrimidine-2-carbonitrile trifluoroacetate;
6-[(2S)-2-aminopropyl]-2-chloro-7-methyl-N-[(5-methylfuran-2-yl)methyl]thieno[3,2-d]pyrimidin-4-amine dihydrochloride;
6-[(2S)-2-aminopropyl]-N-[furan-2-yl)methyl]-7-methyl-2-(trifluoromethyl)thieno[3,2-d]pyrimidin-4-amine dihydrochloride;
6-[(2S)-2-aminopropyl]-2-chloro-7-methyl-N-[(4-methyl-1,3-thiazol-2-yl)methyl]thieno[3,2-d]pyrimidin-4-amine dihydrochloride;
6-[(2S)-2-aminopropyl]-2-chloro-7-methyl-N-[(thiophen-2-yl)methyl]thieno[3,2-d]pyrimidin-4-amine dihydrochloride;
6-[(2S)-2-aminopropyl]-2-chloro-7-methyl-N-[(1,3-thiazol-2-yl)methyl]thieno[3,2-d]pyrimidin-4-amine trifluoroacetate;
6-[(2S)-2-aminopropyl]-2-chloro-7-methyl-N-[(3-methylfuran-2-yl)methyl]thieno[3,2-d]pyrimidin-4-amine dihydrochloride;
6-[(2S)-2-aminopropyl]-2-chloro-7-methyl-N-[(5-methyl-1,3-thiazol-2-yl)methyl]thieno[3,2-d]pyrimidin-4-amine dihydrochloride;
6-[(2S)-2-aminopropyl]-2-chloro-7-methyl-N-[(pyrazin-2-yl)methyl]thieno[3,2-d]pyrimidin-4-amine dihydrochloride;
6-[(2S)-2-aminopropyl]-2-chloro-N-[(5-fluorothiophen-2-yl)methyl]-7-methylthieno[3,2-d]pyrimidin-4-amine dihydrochloride;
6-[(2S)-2-aminopropyl]-N-benzyl-2-chloro-7-methylthieno[3,2-d]pyrimidin-4-amine dihydrochloride;
6-[(2S)-2-aminopropyl]-2-chloro-N-[(3-fluoropyridin-4-yl)methyl]-7-methylthieno[3,2-d]pyrimidin-4-amine dihydrochloride;
6-[(2S)-2-aminopropyl]-2-chloro-7-cyclopropyl-N-[(furan-2-yl)methyl]thieno[3,2-d]pyrimidin-4-amine trifluoroacetate;
6-[(2S)-2-aminobutyl]-2-chloro-7-methyl-N-[(thiophen-2-yl)methyl]thieno[3,2-d]pyrimidin-4-amine dihydrochloride;
6-[(2S)-2-aminopropyl]-2-bromo-N-[(furan-2-yl)methyl]-7-methylthieno[3,2-d]pyrimidin-4-amine trifluoroacetate;
6-[(2S)-2-aminopropyl]-2-chloro-7-methyl-N-[(1,2-oxazol-5-yl)methyl]thieno[3,2-d]pyrimidin-4-amine hydrochloride;
6-[(2S)-2-aminopropyl]-7-bromo-2-chloro-N-[(furan-2-yl)methyl]thieno[3,2-d]pyrimidin-4-amine dihydrochloride;
6-(azetidin-3-yl)-2-chloro-N-[(furan-2-yl)methyl]-7-methylthieno[3,2-d]pyrimidin-4-amine trifluoroacetate;
6-[(2S)-2-aminopropyl]-2-chloro-N-[(2-fluorophenyl)methyl]-7-methylthieno[3,2-d]pyrimidin-4-amine dihydrochloride;
6-[(2S)-2-aminopropyl]-2-chloro-7-methyl-N-[(pyridin-4-yl)methyl]thieno[3,2-d]pyrimidin-4-amine dihydrochloride;
6-[(1S)-1-aminoethyl]-7-bromo-2-chloro-N-[(furan-2-yl)methyl]thieno[3,2-d]pyrimidin-4-amine hydrochloride;
6-[(1S)-1-aminoethyl]-2-chloro-N-[(furan-2-yl)methyl]-7-methylthieno[3,2-d]pyrimidin-4-amine hydrochloride;
6-[(1S)-1-aminopropyl]-2-chloro-N-[(furan-2-yl)methyl]-7-methylthieno[3,2-d]pyrimidin-4-amine hydrochloride;
6-[(1R)-1-aminopropyl]-2-chloro-N-[(furan-2-yl)methyl]-7-methylthieno[3,2-d]pyrimidin-4-amine hydrochloride;
6-[(2S)-2-aminopropyl]-2-chloro-7-methyl-N-[(pyrimidin-4-yl)methyl]thieno[3,2-d]pyrimidin-4-amine dihydrochloride;
6-[(2S)-2-amino-4-fluorobutyl]-2-chloro-N-[(furan-2-yl)methyl]-7-methylthieno[3,2-d]pyrimidin-4-amine dihydrochloride;
6-[(2S)-2-aminobutyl]-2-chloro-N-[(3-fluoropyridin-4-yl)methyl]-7-methylthieno[3,2-d]pyrimidin-4-amine dihydrochloride;
6-[(2S)-2-aminobutyl]-2-chloro-7-methyl-N-[(1,3-thiazol-2-yl)methyl]thieno[3,2-d]pyrimidin-4-amine dihydrochloride;
6-[(2S)-2-aminopropyl]-2-chloro-7-methyl-N-[(pyrimidin-2-yl)methyl]thieno[3,2-d]pyrimidin-4-amine dihydrochloride;
(2R)-2-amino-3-(2-chloro-7-methyl-4-{[(thiophen-2-yl)methyl]amino}thieno[3,2-d]pyrimidin-6-yl)propan-1-ol dihydrochloride;
2-chloro-N-[(furan-2-yl)methyl]-7-methyl-6-(pyrrolidin-3-yl)thieno[3,2-d]pyrimidin-4-amine formate;
6-[(1S)-1-aminoethyl]-2-chloro-N-[(furan-2-yl)methyl]-7-phenylthieno[3,2-d]pyrimidin-4-amine formate;
6-[(2S)-2-aminopropyl]-2-chloro-N-[(3-fluoropyridin-2-yl)methyl]-7-methylthieno[3,2-d]pyrimidin-4-amine dihydrochloride;
6-[(2S)-2-aminopropyl]-2-chloro-N-[(2-fluoropyridin-3-yl)methyl]-7-methylthieno[3,2-d]pyrimidin-4-amine dihydrochloride;
6-[(1S)-1-aminoethyl]-N-[(furan-2-yl)methyl]-2,7-diphenylthieno[3,2-d]pyrimidin-4-amine formate;
6-[(2S)-2-aminobutyl]-2-chloro-7-methyl-N-[(1,2-thiazol-5-yl)methyl]thieno[3,2-d]pyrimidin-4-amine formate;
6-[(2S)-2-aminopropyl]-2-chloro-N-[(3,5-difluoropyridin-4-yl)methyl]-7-methylthieno[3,2-d]pyrimidin-4-amine dihydrochloride;
6-[(2S)-2-aminopropyl]-2-chloro-N-[(furan-2-yl)methyl]-7-(4-methoxyphenyl)thieno[3,2-d]pyrimidin-4-amine formate;
6-(3-aminopropyl)-2-chloro-N-[(furan-2-yl)methyl]-7-methylthieno[3,2-d]pyrimidin-4-amine hydrochloride;
6-[(2S)-2-aminopropyl]-7-bromo-2-chloro-N-[(3-fluoropyridin-4-yl)methyl]thieno[3,2-d]pyrimidin-4-amine dihydrochloride;
6-[(2S)-2-aminobutyl]-2-chloro-7-methyl-N-[(1,3-oxazol-2-yl)methyl]thieno[3,2-d]pyrimidin-4-amine dihydrochloride;
6-[(2S)-3-amino-2-methylpropyl]-2-chloro-N-[(furan-2-yl)methyl]-7-methylthieno[3,2-d]pyrimidin-4-amine hydrochloride;
6-[(2R)-3-amino-2-methylpropyl]-2-chloro-N-[(furan-2-yl)methyl]-7-methylthieno[3,2-d]pyrimidin-4-amine hydrochloride;
6-[(2S)-2-aminopropyl]-2-chloro-N-[(1H-imidazol-2-yl)methyl]-7-methylthieno[3,2-d]pyrimidin-4-amine dihydrochloride;
6-[(2S)-2-aminopropyl]-2-chloro-7-methyl-N-[(1,3-thiazol-5-yl)methyl]thieno[3,2-d]pyrimidin-4-amine dihydrochloride;
6-[(2S)-2-aminopropyl]-2-chloro-7-methyl-N-[(1,3-oxazol-5-yl)methyl]thieno[3,2-d]pyrimidin-4-amine dihydrochloride;
6-[(2R)-2-amino-3-methoxypropyl]-2-chloro-N-[(furan-2-yl)methyl]-7-methylthieno[3,2-d]pyrimidin-4-amine dihydrochloride;
6-[(2S)-2-aminopropyl]-2-chloro-7-ethyl-N-[(furan-2-yl)methyl]thieno[3,2-d]pyrimidin-4-amine hydrochloride;
2-chloro-6-[(2S)-2-(cyclobutylamino)propyl]-N-[(furan-2-yl)methyl]-7-methylthieno[3,2-d]pyrimidin-4-amine formate;
2-chloro-N-[(furan-2-yl)methyl]-7-methyl-6-[(2S)-2-(methylamino)propyl]thieno[3,2-d]pyrimidin-4-amine hydrochloride;
6-[(2S)-2-aminopropyl]-2-chloro-7-methyl-N-[(1-methyl-1H-pyrazol-5-yl)methyl]thieno[3,2-d]pyrimidin-4-amine hydrochloride;
6-[(2S)-2-aminopropyl]-7-bromo-2-chloro-N-[(thiophen-2-yl)methyl]thieno[3,2-d]pyrimidin-4-amine hydrochloride;
6-[(2S)-2-aminopropyl]-7-bromo-4-{[(thiophen-2-yl)methyl]amino}thieno[3,2-d]pyrimidine-2-carbonitrile formate;
6-[(2S)-2-aminopropyl]-4-{[(thiophen-2-yl)methyl]amino}thieno[3,2-d]pyrimidine-2,7-dicarbonitrile hydrochloride;
6-[(2S)-2-aminopropyl]-2-chloro-7-cyclopropyl-N-[(thiophen-2-yl)methyl]thieno[3,2-d]pyrimidin-4-amine formate;
6-[(2S)-2-aminopropyl]-2-chloro-7-phenyl-N-[(thiophen-2-yl)methyl]thieno[3,2-d]pyrimidin-4-amine hydrochloride;
6-[(2S)-2-aminopropyl]-2-chloro-7-(4-chlorophenyl)-N-[(thiophen-2-yl)methyl]thieno[3,2-d]pyrimidin-4-amine hydrochloride;
6-[(2S)-2-aminobutyl]-2-chloro-7-methyl-N-[(pyrimidin-4-yl)methyl]thieno[3,2-d]pyrimidin-4-amine dihydrochloride;
6-[(2S)-2-aminopropyl]-2-chloro-N-[(3-fluorothiophen-2-yl)methyl]-7-methylthieno[3,2-d]pyrimidin-4-amine dihydrochloride;
6-[(2S)-2-aminobutyl]-2-chloro-N-[(3-fluorothiophen-2-yl)methyl]-7-methylthieno[3,2-d]pyrimidin-4-amine dihydrochloride;
6-[(2S)-2-aminopropyl]-2-chloro-N-[(4-fluoro-1,3-thiazol-2-yl)methyl]-7-methylthieno[3,2-d]pyrimidin-4-amine dihydrochloride;
6-[(2S)-2-aminopropyl]-2-chloro-N-[(5-fluoro-1,3-thiazol-2-yl)methyl]-7-methylthieno[3,2-d]pyrimidin-4-amine dihydrochloride;
6-[(1R)-1-aminoethyl]-2-chloro-N-[(furan-2-yl)methyl]-7-methylthieno[3,2-d]pyrimidin-4-amine hydrochloride;
(3S)-3-amino-4-(2-chloro-4-{[(furan-2-yl)methyl]amino}-7-methylthieno[3,2-d]pyrimidin-6-yl)butan-1-ol dihydrochloride;
6-[(2S)-2-aminopropyl]-2-chloro-N-[(5-fluoropyrimidin-4-yl)methyl]-7-methylthieno[3,2-d]pyrimidin-4-amine formate, and
(2R)-2-amino-3-(2-chloro-7-methoxy-4-{[(thiophen-2-yl)methyl]amino}thieno[3,2-d]pyrimidin-6-yl)propan-1-ol dihydrochloride;
or form thereof, wherein the form of the compound salt is selected from the group consisting of a hydrate, solvate, racemate, enantiomer, diastereomer, stereoisomer, and tautomer form thereof.

13. The compound of claim 1, or a form thereof, wherein the compound salt is selected from the group consisting of:
6-[(S)-(1-(1-aminoethyl)cyclopropyl)]-2-chloro-N-[(furan-2-yl)methyl]-7-methylthieno[3,2-d]pyrimidin-4-amine formate
6-[(S)-(1-(amino(cyclopropyl)methyl)cyclopropyl)]-2-chloro-N-[(furan-2-yl)methyl]-7-methylthieno[3,2-d]pyrimidin-4-amine formate
6-[(2R)-2-amino-2-cyclopropylethyl]-7-bromo-2-chloro-N-[(thiophen-2-yl)methyl]thieno[3,2-d]pyrimidin-4-amine dihydrochloride, and
6-[(2R)-2-amino-2-cyclopropylethyl]-2-chloro-7-methyl-N-[(thiophen-2-yl)methyl]thieno[3,2-d]pyrimidin-4-amine dihydrochloride;
wherein the form of the compound salt is selected from the group consisting of a hydrate, solvate, racemate, enantiomer, diastereomer, stereoisomer, and tautomer form thereof.

14. A method of treating familial dysautonomia comprising administering to a subject in need thereof an effective amount of the compound of claim 1.

15. A method of treating familial dysautonomia comprising administering to a subject in need thereof an effective amount of the compound of claim 10.

16. A method of treating familial dysautonomia comprising administering to a subject in need thereof an effective amount of the compound of claim 12.

17. A pharmaceutical composition comprising the compound of claim 1 in an admixture with a pharmaceutically acceptable excipient.

18. A pharmaceutical composition comprising the compound of claim 10 in an admixture with a pharmaceutically acceptable excipient.

19. A pharmaceutical composition comprising the compound of claim 12 in an admixture with a pharmaceutically acceptable excipient.

20.-25. (canceled)
